SEQUENCE LISTING (49 sequences)

SEQ ID NO: 1 - DNA, 10560 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgagcgcgcaaagccactactgccacttttggagactgtgtacgtcgagggcctctgccagtgtcgaacagacattcgcctacggccctcgtctgttcgggctcagggcgcgtcagcgggtgttggcgggtgtcggggctggcttaactatgcggcatcagagcagattgtactgagagtgcaccatatgcggtgtgaaataagtcccgcgcagtcgcccacaaccgcccacagccccgaccgaattgatacgccgtagtctcgtctaacatgactctcacgtggtatacgccacactttatccgcacagatgcgtaaggagaaaataccgcatcaggcgccattcgccattcaggctgcgcaactgttgggaagggcgatcggtgcgggcctcttcgctatggcgtgtctacgcattcctcttttatggcgtagtccgcggtaagcggtaagtccgacgcgttgacaacccttcccgctagccacgcccggagaagcgatatacgccagctggcgaaagggggatgtgctgcaaggcgattaagttgggtaacgccagggttttcccagtcacgacgttgtaaaacgacggccagtgccaaatgcggtcgaccgctttccccctacacgacgttccgctaattcaacccattgcggtcccaaaagggtcagtgctgcaacattttgctgccggtcacggttgcttgcatgcctgcaggtcgacgggcccgggatccgatgctcttccgctaagatctgccgcggccgcgtcctcagaagaactcgtcaagaaggcgatagacgaacgtacggacgtccagctgcccgggccctaggctacgagaaggcgattctagacggcgccggcgcaggagtcttcttgagcagttcttccgctatctaggcgatgcgctgcgaatcgggagcggcgataccgtaaagcacgaggaagcggtcagcccattcgccgccaagctcttcagcaatatcacgggtagccaatccgctacgcgacgcttagccctcgccgctatggcatttcgtgctccttcgccagtcgggtaagcggcggttcgagaagtcgttatagtgcccatcggttcgctatgtcctgatagcggtccgccacacccagccggccacagtcgatgaatccagaaaagcggccattttccaccatgatattcggcaagcaggcatcggcgatacaggactatcgccaggcggtgtgggtcggccggtgtcagctacttaggtcttttcgccggtaaaaggtggtactataagccgttcgtccgtagcccatgggtcacgacgagatcctcgccgtcgggcatgcgcgccttgagcctggcgaacagttcggctggcgcgagcccctgatgctcttcgtccagatcatggtacccagtgctgctctaggagcggcagcccgtacgcgcggaactcggaccgcttgtcaagccgaccgcgctcggggactacgagaagcaggtctagtacctgatcgacaagaccggcttccatccgagtacgtgctcgctcgatgcgatgtttcgcttggtggtcgaatgggcaggtagccggatcaagcgtatgcagggactagctgttctggccgaaggtaggctcatgcacgagcgagctacgctacaaagcgaaccaccagcttacccgtccatcggcctagttcgcatacgtcccgccgcattgcatcagccatgatggatactttctcggcaggagcaaggtgggatgacaggagatcctgccccggcacttcgcccaatagcagccagtccggcggcgtaacgtagtcggtactacctatgaaagagccgtcctcgttccaccctactgtcctctaggacggggccgtgaagcgggttatcgtcggtcaggcttcccgcttcagtgacaacgtcgagcacagctgcgcaaggaacgcccgtcgtggccagccacgatagccgcgctgcctcgtcctgcagttcattcaggggaagggcgaagtcactgttgcagctcgtgtcgacgcgttccttgcgggcagcaccggtcggtgctatcggcgcgacggagcaggacgtcaagtaagtccccaccggacaggtcggtcttgacaaaaagaaccgggcgcccctgcgctgacagccggaacacggcggcatcagagcagccgattgtctgttgtgcccagtcgtggcctgtccagccagaactgtttttcttggcccgcggggacgcgactgtcggccttgtgccgccgtagtctcgtcggctaacagacaacacgggtcagatagccgaatagcctctccacccaagcggccggagaacctgcgtgcaatccatcttgttcaatcatgatatcccttaattaaccgttaacactagttcagtatcggcttatcggagaggtgggttcgccggcctcttggacgcacgttaggtagaacaagttagtactatagggaattaattggcaattgtgatcaagtctccatctcgccgtgtatgcgggcctgacggatcaacgttcccaccgagccagtcgagatgttcatctggtcggcgatctgccggtacttcaaaccttgttaggtagagcggcacatacgcccggactgcctagttgcaagggtggctcggtcagctctacaagtagaccagccgctagacggccatgaagtttggaacaatgcgcagttccacagccttcttgcggcgttcctgcgcacgagcgatgtagtcgcctcggtcttcggcgacgagccgtttgatggtgcttttcgagacgccacgcgtcaaggtgtcggaagaacgccgcaaggacgcgtgctcgctacatcagcggagccagaagccgctgctcggcaaactaccacgaaaagctctgcgggaacttgtcagccaactcctgcgcggtctgcgtgcgacgcatcacgcgttctgcagcacccatcagtccgtcccctctgctgctgcgaacagtgccgatccttgaacagtcggttgaggacgcgccagacgcacgctgcgtagtgcgcaagacgtcgtgggtagtcaggcaggggagacgacgacgcttgtcacggctaggatcgaccttcttgagcttcggccgcggcgcggtggcgttcttccgtaccgcttccgtttttgcgctgctgctcactttgccgcggcgtgcctggattttctagctggaagaactcgaagccggcgccgcgccaccgcaagaaggcatggcgaaggcaaaaacgcgacgacgagtgaaacggcgccgcacggacctaaaacgagaactcggcggcggtgaaggtgcggtgggtccagtgggcgactgatttgccgatctgctcggcctcggcccgactcatggggccgatcccgtcgttggctcttgagccgccgccacttccacgccacccaggtcacccgctgactaaacggctagacgagccggagccgggctgagtaccccggctagggcagcaacgcgtcgagggtgaagttggtcagggcggtgaagtcggtgaccatctgccgccacacagtgatcgacgggtagttctgtttccggatctcgcggtaggccccgcagctcccacttcaaccagtcccgccacttcagccactggtagacggcggtgtgtcactagctgcccatcaagacaaaggcctagagcgccatccgggattcccgggtgcggtcgaacagttcgacgttccggcccgtttcggtcctgacctgtgtcttgcggccgtagtccggtggggcggggaaacggtcaccgagtaagggcccacgccagcttgtcaagctgcaaggccgggcaaagccaggactggacacagaacgccggcatcaggccaccccgcccctttgccagtggctccgcttttgcgaggcctttgagcgagtacggatccgagggaccccagaccgtcgtccagtgcgggtggatcgggttctgggtgagctgctgcgcgtagcccgcgaaaacgctccggaaactcgctcatgcctaggctccctggggtctggcagcaggtcacgcccacctagcccaagacccactcgacgacgcgcatcgggtgatcggcgccgaccaccgaggcgatcagcccctggttcacccggtcgtagagccgcagcgggccctgtcgggctgcctggagggtgtagaccgggctttactagccgcggctggtggctccgctagtcggggaccaagtgggccagcatctcggcgtcgcccgggacagcccgacggacctcccacatctggcccgaaacgagcagccaccacaggtgcgcgtgctcggtcgcgggattgatcgtcatcacggtcggatcgggcagatccgcgttacgtgcggcccactgcgcctggtcgctcgtcggtggtgtccacgcgcacgagccagcgccctaactagcagtagtgccagcctagcccgtctaggcgcaatgcacgccgggtgacgcggaccaggtcgtccacgtcgagcaccaagcccaacctgatcgacggggtgcgggccgcaatgtagcggcgggtgagcgcctccgcgcgcggctgcggccactgcccgcagcaggtgcagctcgtggttcgggttggactagctgccccacgcccggcgttacatcgccgcccactcgcggaggcgcgcgccgacgccggtgacgggctcccggacgtagtcatccgtcgcgtgcgggtatttgaaccgccagcggtccaaccaggcgtcaacagcagcggtcatgaccgccaagctagggccggatcagggcctgcatcagtaggcagcgcacgcccataaacttggcggtcgccaggttggtccgcagttgtcgtcgccagtactggcggttcgatcccggcctagtgtaccgatcgggggaggcgcgccgcaaattatttaagagtctcgctagcaaaccatgtcaggtgttgcggtgggttccgggtaaacctccacccgaattacatggctagccccctccgcgcggcgtttaataaattctcagagcgatcgtttggtacagtccacaacgccacccaaggcccatttggaggtgggcttaaatttaagagtctcgctagctaagccctatctgatgctgcgcggggggtccttcgcactgaatctcaaaggtggccggctgaatttcgtcgcgcgaaaacctaaattctcagagcgatcgattcgggatagactacgacgcgccccccaggaagcgtgacttagagtttccaccggccgacttaaagcagcgcgcttttggtccctggacagttctggaattcagcaagaggtgtgtctgaacttcggtgtttttttggggggtgactccagcggggtgggcacaacgcgaacagagacctagggacctgtcaagaccttaagtcgttctccacacagacttgaagccacaaaaaaaccccccactgaggtcgccccacccgtgttgcgcttgtctctggatgtgtgtacgacggcgggaggtaagtcgggtacggctcggactgcggtagagcaaccgtcgaatcgatttcgagcagagcgagcagagcaagatattccaacacacatgctgccgccctccattcagcccatgccgagcctgacgccatctcgttggcagcttagctaaagctcgtctcgctcgtctcgttctataaggtaaactccggggttcctcggcggcctcccccgtctgtttgctcaaccgagggagacctggcggtcccgcgtttccggacgcgcgggaccgcctaccgctcgtttgaggccccaaggagccgccggagggggcagacaaacgagttggctccctctggaccgccagggcgcaaaggcctgcgcgccctggcggatggcgagcagagcggaagagcatctagatgcattcgcgaggtaccgagctcgaattcgtaatcatggtcatagctgtttcctgtgtgaaattgttatccgctcacaattctcgccttctcgtagatctacgtaagcgctccatggctcgagcttaagcattagtaccagtatcgacaaaggacacactttaacaataggcgagtgttatccacacaacatacgagccggaagcataaagtgtaaagcctggggtgcctaatgagtgagctaactcacattaattgcgttgcgctcactgcccgctttcaggtgtgttgtatgctcggccttcgtatttcacatttcggaccccacggattactcactcgattgagtgtaattaacgcaacgcgagtgacgggcgaaagcagtcgggaaacctgtcgtgccagctgcattaatgaatcggccaacgcgcggggagaggcggtttgcgtattgggcgctcttccgcttcctcgctcactggtcagccctttggacagcacggtcgacgtaattacttagccggttgcgcgcccctctccgccaaacgcataacccgcgagaaggcgaaggagcgagtgacactcgctgcgctcggtcgttcggctgcggcgagcggtatcagctcactcaaaggcggtaatacggttatccacagaatcaggggataacgcaggaaagaatgagcgacgcgagccagcaagccgacgccgctcgccatagtcgagtgagtttccgccattatgccaataggtgtcttagtcccctattgcgtcctttcttcatgtgagcaaaaggccagcaaaaggccaggaaccgtaaaaaggccgcgttgctggcgtttttccataggctccgcccccctgacgagcatcacaaaaatgtacactcgttttccggtcgttttccggtccttggcatttttccggcgcaacgaccgcaaaaaggtatccgaggcggggggactgctcgtagtgtttttacgacgctcaagtcagaggtggcgaaacccgacaggactataaagataccaggcgtttccccctggaagctccctcgtgcgctctcctgttccgaccctgcgctgcgagttcagtctccaccgctttgggctgtcctgatatttctatggtccgcaaagggggaccttcgagggagcacgcgagaggacaaggctgggacgcgcttaccggatacctgtccgcctttctcccttcgggaagcgtggcgctttctcatagctcacgctgtaggtatctcagttcggtgtaggtcgttcgctcgcgaatggcctatggacaggcggaaagagggaagcccttcgcaccgcgaaagagtatcgagtgcgacatccatagagtcaagccacatccagcaagcgagcaagctgggctgtgtgcacgaaccccccgttcagcccgaccgctgcgccttatccggtaactatcgtcttgagtccaacccggtaagacacgacttatcggttcgacccgacacacgtgcttggggggcaagtcgggctggcgacgcggaataggccattgatagcagaactcaggttgggccattctgtgctgaatagcccactggcagcagccactggtaacaggattagcagagcgaggtatgtaggcggtgctacagagttcttgaagtggtggcctaactacggctacactagaaggtgaccgtcgtcggtgaccattgtcctaatcgtctcgctccatacatccgccacgatgtctcaagaacttcaccaccggattgatgccgatgtgatcttggacagtatttggtatctgcgctctgctgaagccagttaccttcggaaaaagagttggtagctcttgatccggcaaacaaaccaccgctggtagcggtggcctgtcataaaccatagacgcgagacgacttcggtcaatggaagcctttttctcaaccatcgagaactaggccgtttgtttggtggcgaccatcgccacctttttttgtttgcaagcagcagattacgcgcagaaaaaaaggatctcaagaagatcctttgatcttttctacggggtctgacgctcagtggaacgaaaacaaaaaaacaaacgttcgtcgtctaatgcgcgtctttttttcctagagttcttctaggaaactagaaaagatgccccagactgcgagtcaccttgcttttgtcacgttaagggattttggtcatgagattatcaaaaaggatcttcacctagatccttttaaattaaaaatgaagttttaaatcaatctaaagtatatatgagtgcaattccctaaaaccagtactctaatagtttttcctagaagtggatctaggaaaatttaatttttacttcaaaatttagttagatttcatatatacagtaaacttggtctgacagttaccaatgcttaatcagtgaggcacctatctcagcgatctgtctatttcgttcatccatagttgcctgactccccgtcgttcatttgaaccagactgtcaatggttacgaattagtcactccgtggatagagtcgctagacagataaagcaagtaggtatcaacggactgaggggcagcagtagataactacgatacgggagggcttaccatctggccccagtgctgcaatgataccgcgagacccacgctcaccggctccagatttatcagcaataaaccatctattgatgctatgccctcccgaatggtagaccggggtcacgacgttactatggcgctctgggtgcgagtggccgaggtctaaatagtcgttatttgcagccagccggaagggccgagcgcagaagtggtcctgcaactttatccgcctccatccagtctattaattgttgccgggaagctagagtaagtagttcgcgtcggtcggccttcccggctcgcgtcttcaccaggacgttgaaataggcggaggtaggtcagataattaacaacggcccttcgatctcattcatcaagcgcagttaatagtttgcgcaacgttgttgccattgctacaggcatcgtggtgtcacgctcgtcgtttggtatggcttcattcagctccggttcccaacgatcgtcaattatcaaacgcgttgcaacaacggtaacgatgtccgtagcaccacagtgcgagcagcaaaccataccgaagtaagtcgaggccaagggttgctagaaggcgagttacatgatcccccatgttgtgcaaaaaagcggttagctccttcggtcctccgatcgttgtcagaagtaagttggccgcagtgttatcactcttccgctcaatgtactagggggtacaacacgttttttcgccaatcgaggaagccaggaggctagcaacagtcttcattcaaccggcgtcacaatagtgagatggttatggcagcactgcataattctcttactgtcatgccatccgtaagatgcttttctgtgactggtgagtactcaaccaagtcattctgagaatagttaccaataccgtcgtgacgtattaagagaatgacagtacggtaggcattctacgaaaagacactgaccactcatgagttggttcagtaagactcttatcagtatgcggcgaccgagttgctcttgcccggcgtcaatacgggataataccgcgccacatagcagaactttaaaagtgctcatcattggaaaacgttcttccatacgccgctggctcaacgagaacgggccgcagttatgccctattatggcgcggtgtatcgtcttgaaattttcacgagtagtaaccttttgcaagaagggggcgaaaactctcaaggatcttaccgctgttgagatccagttcgatgtaacccactcgtgcacccaactgatcttcagcatcttttactttcaccagcccccgcttttgagagttcctagaatggcgacaactctaggtcaagctacattgggtgagcacgtgggttgactagaagtcgtagaaaatgaaagtggtcggtttctgggtgagcaaaaacaggaaggcaaaatgccgcaaaaaagggaataagggcgacacggaaatgttgaatactcatactcttcctttttcaatattcaaagacccactcgtttttgtccttccgttttacggcgttttttcccttattcccgctgtgcctttacaacttatgagtatgagaaggaaaaagttataaattgaagcatttatcagggttattgtctcatgagcggatacatatttgaatgtatttagaaaaataaacaaataggggttccgcgcacatttccccgaaataacttcgtaaatagtcccaataacagagtactcgcctatgtataaacttacataaatctttttatttgtttatccccaaggcgcgtgtaaaggggctttagtgccacctgacgtctaagaaaccattattatcatgacattaacctataaaaataggcgtatcacgaggccctttcgtctcacggtggactgcagattctttggtaataatagtactgtaattggatatttttatccgcatagtgctccgggaaagcag

SEQ ID NO: 2 - DNA, 10288 bp, Artificial Sequence
  Synthetic Polynucleotide
ggggagccgcgccgaaggcgtgggggaaccccgcaggggtgcccttctttgggcaccaaagaactagatatagggcgaaatgcgaaagacttaaaaatcacccctcggcgcggcttccgcacccccttggggcgtccccacgggaagaaacccgtggtttcttgatctatatcccgctttacgctttctgaatttttagtacaacttaaaaaaggggggtacgcaacagctcattgcggcaccccccgcaatagctcattgcgtaggttaaagaaaatctgtaattgactgccacttttatgttgaattttttccccccatgcgttgtcgagtaacgccgtggggggcgttatcgagtaacgcatccaatttcttttagacattaactgacggtgaaaatcgcaacgcataattgttgtcgcgctgccgaaaagttgcagctgattgcgcatggtgccgcaaccgtgcggcaccctaccgcatggagataagcatggccagcgttgcgtattaacaacagcgcgacggcttttcaacgtcgactaacgcgtaccacggcgttggcacgccgtgggatggcgtacctctattcgtaccggtcgcagtccagagaaatcggcattcaagccaagaacaagcccggtcactgggtgcaaacggaacgcaaagcgcatgaggcgtgggccgggcttattgcgaggcgtcaggtctctttagccgtaagttcggttcttgttcgggccagtgacccacgtttgccttgcgtttcgcgtactccgcacccggcccgaataacgctcgaaacccacggcggcaatgctgctgcatcacctcgtggcgcagatgggccaccagaacgccgtggtggtcagccagaagacactttccaagctcatcggactttgggtgccgccgttacgacgacgtagtggagcaccgcgtctacccggtggtcttgcggcaccaccagtcggtcttctgtgaaaggttcgagtagcctcgttctttgcggacggtccaatacgcagtcaaggacttggtggccgagcgctggatctccgtcgtgaagctcaacggccccggcaccgtgtcggcctacggcaagaaacgcctgccaggttatgcgtcagttcctgaaccaccggctcgcgacctagaggcagcacttcgagttgccggggccgtggcacagccggatgctggtcaatgaccgcgtggcgtggggccagccccgcgaccagttgcgcctgtcggtgttcagtgccgccgtggtggttgatcacgacgaccaggacgaatcaccagttactggcgcaccgcaccccggtcggggcgctggtcaacgcggacagccacaagtcacggcggcaccaccaactagtgctgctggtcctgcttaggctgttggggcatggcgacctgcgccgcatcccgaccctgtatccgggcgagcagcaactaccgaccggccccggcgaggagccgcccagccagcccggccgacaaccccgtaccgctggacgcggcgtagggctgggacataggcccgctcgtcgttgatggctggccggggccgctcctcggcgggtcggtcgggccgattccgggcatggaaccagacctgccagccttgaccgaaacggaggaatgggaacggcgcgggcagcagcgcctgccgatgcccgatgagccgtgttttctaaggcccgtaccttggtctggacggtcggaactggctttgcctccttacccttgccgcgcccgtcgtcgcggacggctacgggctactcggcacaaaagtggacgatggcgagccgttggagccgccgacacgggtcacgctgccgcgccggtagcacttgggttgcgcagcaacccgtaagtgcgctgttccagactaacctgctaccgctcggcaacctcggcggctgtgcccagtgcgacggcgcggccatcgtgaacccaacgcgtcgttgggcattcacgcgacaaggtctgattcggctgtagccgcctcgccgccctataccttgtctgcctccccgcgttgcgtcgcggtgcatggagccgggccacctcgacctgaatggaagccggcggagccgacatcggcggagcggcgggatatggaacagacggaggggcgcaacgcagcgccacgtacctcggcccggtggagctggacttaccttcggccgcccacctcgctaacggattcaccgtttttatcaggctctgggaggcagaataaatgatcatatcgtcaattattacctccacggggagagcctgagcaaactgtggagcgattgcctaagtggcaaaaatagtccgagaccctccgtcttatttactagtatagcagttaataatggaggtgcccctctcggactcgtttgaggcctcaggcatttgagaagcacacggtcacactgcttccggtagtcaataaaccggtaaaccagcaatagacataagcggctatttaacgaccctgcccccggagtccgtaaactcttcgtgtgccagtgtgacgaaggccatcagttatttggccatttggtcgttatctgtattcgccgataaattgctgggacgggtgaaccgacgaccgggtcgaatttgctttcgaatttctgccattcatccgcttattatcacttattcaggcgtagcaccaggcgtttaagggcaccaataacttggctgctggcccagcttaaacgaaagcttaaagacggtaagtaggcgaataatagtgaataagtccgcatcgtggtccgcaaattcccgtggttatactgccttaaaaaaattacgccccgccctgccactcatcgcagtcggcctattggttaaaaaatgagctgatttaacaaaaatttaacgcgaattttaactgacggaatttttttaatgcggggcgggacggtgagtagcgtcagccggataaccaattttttactcgactaaattgtttttaaattgcgcttaaaattgaaaatattaacgcttacaatttccattcgccattcaggctgcgcaactgttgggaagggcgatcggtgcgggcctcttcgctattacgccagctggcgaattttataattgcgaatgttaaaggtaagcggtaagtccgacgcgttgacaacccttcccgctagccacgcccggagaagcgataatgcggtcgaccgcttagggggatgtgctgcaaggcgattaagttgggtaacgccagggttttcccagtcacgacgttgtaaaacgacggccagtgagcgcgcgtaatacgactcatccccctacacgacgttccgctaattcaacccattgcggtcccaaaagggtcagtgctgcaacattttgctgccggtcactcgcgcgcattatgctgagtctatagggcgaattggagctccaccgcggtggcggccgctctagaactagtggatcccccgggctgcaggaattcgatatcaagcttatcgataccgtcggatatcccgcttaacctcgaggtggcgccaccgccggcgagatcttgatcacctagggggcccgacgtccttaagctatagttcgaatagctatggcagcacctcgagggggggcccggtacccagcttttgttccctttagtgagggttaattgcgcgcttggcgtaatcatggtcatagctgtttcctgtgtgaaatttggagctcccccccgggccatgggtcgaaaacaagggaaatcactcccaattaacgcgcgaaccgcattagtaccagtatcgacaaaggacacactttaagttatccgctcacaattccacacaacatacgagccggaagcataaagtgtaaagcctggggtgcctaatgagtgagctaactcacattaattgcgttgcgcaataggcgagtgttaaggtgtgttgtatgctcggccttcgtatttcacatttcggaccccacggattactcactcgattgagtgtaattaacgcaacgcctcactgcccgctttccagtcgggaaacctgtcgtgccagctgcattaatgaatcggccaacgcgcggggagaggcggtttgcgtattgggcgcatgcatgagtgacgggcgaaaggtcagccctttggacagcacggtcgacgtaattacttagccggttgcgcgcccctctccgccaaacgcataacccgcgtacgtaaaaaactgttgtaattcattaagcattctgccgacatggaagccatcacaaacggcatgatgaacctgaatcgccagcggcatcagcaccttgtcgcctttttttgacaacattaagtaattcgtaagacggctgtaccttcggtagtgtttgccgtactacttggacttagcggtcgccgtagtcgtggaacagcggaagcgtataatatttgcccatgggggtgggcgaagaactccagcatgagatccccgcgctggaggatcatccagccggcgtcccggaaaacgattccgaagccgcatattataaacgggtacccccacccgcttcttgaggtcgtactctaggggcgcgacctcctagtaggtcggccgcagggccttttgctaaggcttcgccaacctttcatagaaggcggcggtggaatcgaaatctcgtgatggcaggttgggcgtcgcttggtcggtcatttcgaaccccagagtcccgctcagaagggttggaaagtatcttccgccgccaccttagctttagagcactaccgtccaacccgcagcgaaccagccagtaaagcttggggtctcagggcgagtcttcaactcgtcaagaaggcgatagaaggcgatgcgctgcgaatcgggagcggcgataccgtaaagcacgaggaagcggtcagcccattcgccgccaagctcttttgagcagttcttccgctatcttccgctacgcgacgcttagccctcgccgctatggcatttcgtgctccttcgccagtcgggtaagcggcggttcgagaacagcaatatcacgggtagccaacgctatgtcctgatagcggtccgccacacccagccggccacagtcgatgaatccagaaaagcggccattttccaccatgtcgttatagtgcccatcggttgcgatacaggactatcgccaggcggtgtgggtcggccggtgtcagctacttaggtcttttcgccggtaaaaggtggtagatattcggcaagcaggcatcgccatgggtcacgacgagatcctcgccgtcgggcatgcgcgccttgagcctggcgaacagttcggctggcgcgagccccctataagccgttcgtccgtagcggtacccagtgctgctctaggagcggcagcccgtacgcgcggaactcggaccgcttgtcaagccgaccgcgctcggggtgatgctcttcgtccagatcatcctgatcgacaagaccggcttccatccgagtacgtgctcgctcgatgcgatgtttcgcttggtggtcgaatgggcaggactacgagaagcaggtctagtaggactagctgttctggccgaaggtaggctcatgcacgagcgagctacgctacaaagcgaaccaccagcttacccgtcctagccggatcaagcgtatgcagccgccgcattgcatcagccatgatggatactttctcggcaggagcaaggtgagatgacaggagatcctgccccggcacatcggcctagttcgcatacgtcggcggcgtaacgtagtcggtactacctatgaaagagccgtcctcgttccactctactgtcctctaggacggggccgtgttcgcccaatagcagccagtcccttcccgcttcagtgacaacgtcgagcacagctgcgcaaggaacgcccgtcgtggccagccacgatagccgcgctgccaagcgggttatcgtcggtcagggaagggcgaagtcactgttgcagctcgtgtcgacgcgttccttgcgggcagcaccggtcggtgctatcggcgcgacggtcgtcctgcagttcattcagggcaccggacaggtcggtcttgacaaaaagaaccgggcgcccctgcgctgacagccggaacacggcggcatcagagcagcagcaggacgtcaagtaagtcccgtggcctgtccagccagaactgtttttcttggcccgcggggacgcgactgtcggccttgtgccgccgtagtctcgtcgcgattgtctgttgtgcccagtcatagccgaatagcctctccacccaagcggccggagaacctgcgtgcaatccatcttgttcaatcatgcgaaacgatccgctaacagacaacacgggtcagtatcggcttatcggagaggtgggttcgccggcctcttggacgcacgttaggtagaacaagttagtacgctttgctaggtcatcctgtctcttgatcagatcttgatcccctgcgccatcagatccttggcggcaagaaagccatccagtttactttgcagggcttcccaaccttaccaagtaggacagagaactagtctagaactaggggacgcggtagtctaggaaccgccgttctttcggtaggtcaaatgaaacgtcccgaagggttggaatggtgagggcgccccagctggcaattccggttcgcttgctgtccataaaaccgcccagtctagctatcgccatgtaagcccactgcaagctacctgctttctctctcccgcggggtcgaccgttaaggccaagcgaacgacaggtattttggcgggtcagatcgatagcggtacattcgggtgacgttcgatggacgaaagagattgcgcttgcgttttcccttgtccagatagcccagtagctgacattcatcccaggtggcacttttcggggaaatgtgcgcgcccgcgttcctgctggcgcaacgcgaacgcaaaagggaacaggtctatcgggtcatcgactgtaagtagggtccaccgtgaaaagcccctttacacgcgcgggcgcaaggacgaccgcgtgggcctgtttctggcgctggacttcccgctgttccgtcagcagcttttcgcccacggccttgatgatcgcggcggccttggcctgcatatcccgattcaacccggacaaagaccgcgacctgaagggcgacaaggcagtcgtcgaaaagcgggtgccggaactactagcgccgccggaaccggacgtatagggctaagtacggccccagggcgtccagaacgggcttcaggcgctcccgaaggtctcgggccgtctcttgggcttgatcggccttcttgcgcatctcacgcgctcctgctgccggggtcccgcaggtcttgcccgaagtccgcgagggcttccagagcccggcagagaacccgaactagccggaagaacgcgtagagtgcgcgaggacgggcggcctgtagggcaggctcatacccctgccgaaccgcttttgtcagccggtcggccacggcttccggcgtctcaacgcgctttgagattcccagctttccgccggacatcccgtccgagtatggggacggcttggcgaaaacagtcggccagccggtgccgaaggccgcagagttgcgcgaaactctaagggtcgaaatcggccaatccctgcggtgcataggcgcgtggctcgaccgcttgcgggctgatggtgacgtggcccactggtggccgctccagggcctcgtagaacgcctagccggttagggacgccacgtatccgcgcaccgagctggcgaacgcccgactaccactgcaccgggtgaccaccggcgaggtcccggagcatcttgcggagaatgcgcgtgtgacgtgccttgctgccctcgatgccccgttgcagccctagatcggccacagcggccgcaaacgtggtctggtcgcgggtcatctgcgccttacgcgcacactgcacggaacgacgggagctacggggcaacgtcgggatctagccggtgtcgccggcgtttgcaccagaccagcgcccagtagacgcgtttgttgccgatgaactccttggccgacagcctgccgtcctgcgtcagcggcaccacgaacgcggtcatgtgcgggctggtttcgtcacggtggatgctgaaacaacggctacttgaggaaccggctgtcggacggcaggacgcagtcgccgtggtgcttgcgccagtacacgcccgaccaaagcagtgccacctacgacgccgtcacgatgcgatccgccccgtacttgtccgccagccacttgtgcgccttctcgaagaacgccgcctgctgttcttggctggccgacttccaccattcggcagtgctacgctaggcggggcatgaacaggcggtcggtgaacacgcggaagagcttcttgcggcggacgacaagaaccgaccggctgaaggtggtaaccgggctggccgtcatgacgtactcgaccgccaacacagcgtccttgcgccgcttctctggcagcaactcgcgcagtcggcccatcgcttcatcggtgctggcccgaccggcagtactgcatgagctggcggttgtgtcgcaggaacgcggcgaagagaccgtcgttgagcgcgtcagccgggtagcgaagtagccacgagctggccgcccagtgctcgttctctggcgtcctgctggcgtcagcgttgggcgtctcgcgctcgcggtaggcgtgcttgagactggccgccacgttgccccgaccggcgggtcacgagcaagagaccgcaggacgaccgcagtcgcaacccgcagagcgcgagcgccatccgcacgaactctgaccggcggtgcaacgggattttcgccagcttcttgcatcgcatgatcgcgtatgccgccatgcctgcccctcccttttggtgtccaaccggctcgacgggggcagcgcaaggcggtgtaaaagcggtcgaagaacgtagcgtactagcgcatacggcggtacggacggggagggaaaaccacaggttggccgagctgcccccgtcgcgttccgccaccctccggcgggccactcaatgcttgagtatactcactagactttgcttcgcaaagtcgtgaccgcctacggcggctgcggcgccctacgggcttgctctcggaggccgcccggtgagttacgaactcatatgagtgatctgaaacgaagcgtttcagcactggcggatgccgccgacgccgcgggatgcccgaacgagagcgggcttcgccctgcgcggtcgctgcgctcccttgccagcccgtggatatgtggacgatggccgcgagcggccaccggctggctcgcttcgctcggcccggcccgaagcgggacgcgccagcgacgcgagggaacggtcgggcacctatacacctgctaccggcgctcgccggtggccgaccgagcgaagcgagccgggctggacaaccctgctggacaagctgatggacaggctgcgcctgcccacgagcttgaccacagggattgcccaccggctacccagccttcgaccacatacccacctgttgggacgacctgttcgactacctgtccgacgcggacgggtgctcgaactggtgtccctaacgggtggccgatgggtcggaagctggtgtatgggaccggctccaactgcgcggcctgcggccttgccccatcaatttttttaattttctctggggaaaagcctccggcctgcggcctgcgcgcttcgcttgccgtggccgaggttgacgcgccggacgccggaacggggtagttaaaaaaattaaaagagaccccttttcggaggccggacgccggacgcgcgaagcgaacggcgttggacaccaagtggaaggcgggtcaaggctcgcgcagcgaccgcgcagcggcttggccttgacgcgcctggaacgacccaagcctatgcgagtgggggcaacctgtggttcaccttccgcccagttccgagcgcgtcgctggcgcgtcgccgaaccggaactgcgcggaccttgctgggttcggatacgctcaccccccagtcgaaggcgaagcccgcccgcctgccccccgagcctcacggcggcgagtgcgggggttccaagggggcagcgccaccttgggcaaggccgaaggccggtcagcttccgcttcgggcgggcggacggggggctcggagtgccgccgctcacgcccccaaggttcccccgtcgcggtggaacccgttccggcttccggccgcagtcgatcaacaagccccggaggggccactttttgccggaggcgtcagctagttgttcggggcctccccggtgaaaaacggcctc

SEQ ID NO: 3 - DNA, 12758 bp, Artificial Sequence
  Synthetic Polynucleotide
ggggagccgcgccgaaggcgtgggggaaccccgcaggggtgcccttctttgggcaccaaagaactagatatagggcgaaatgcgaaagacttaaaaatcacccctcggcgcggcttccgcacccccttggggcgtccccacgggaagaaacccgtggtttcttgatctatatcccgctttacgctttctgaatttttagtacaacttaaaaaaggggggtacgcaacagctcattgcggcaccccccgcaatagctcattgcgtaggttaaagaaaatctgtaattgactgccacttttatgttgaattttttccccccatgcgttgtcgagtaacgccgtggggggcgttatcgagtaacgcatccaatttcttttagacattaactgacggtgaaaatcgcaacgcataattgttgtcgcgctgccgaaaagttgcagctgattgcgcatggtgccgcaaccgtgcggcaccctaccgcatggagataagcatggccagcgttgcgtattaacaacagcgcgacggcttttcaacgtcgactaacgcgtaccacggcgttggcacgccgtgggatggcgtacctctattcgtaccggtcgcagtccagagaaatcggcattcaagccaagaacaagcccggtcactgggtgcaaacggaacgcaaagcgcatgaggcgtgggccgggcttattgcgaggcgtcaggtctctttagccgtaagttcggttcttgttcgggccagtgacccacgtttgccttgcgtttcgcgtactccgcacccggcccgaataacgctcgaaacccacggcggcaatgctgctgcatcacctcgtggcgcagatgggccaccagaacgccgtggtggtcagccagaagacactttccaagctcatcggactttgggtgccgccgttacgacgacgtagtggagcaccgcgtctacccggtggtcttgcggcaccaccagtcggtcttctgtgaaaggttcgagtagcctcgttctttgcggacggtccaatacgcagtcaaggacttggtggccgagcgctggatctccgtcgtgaagctcaacggccccggcaccgtgtcggcctacggcaagaaacgcctgccaggttatgcgtcagttcctgaaccaccggctcgcgacctagaggcagcacttcgagttgccggggccgtggcacagccggatgctggtcaatgaccgcgtggcgtggggccagccccgcgaccagttgcgcctgtcggtgttcagtgccgccgtggtggttgatcacgacgaccaggacgaatcaccagttactggcgcaccgcaccccggtcggggcgctggtcaacgcggacagccacaagtcacggcggcaccaccaactagtgctgctggtcctgcttaggctgttggggcatggcgacctgcgccgcatcccgaccctgtatccgggcgagcagcaactaccgaccggccccggcgaggagccgcccagccagcccggccgacaaccccgtaccgctggacgcggcgtagggctgggacataggcccgctcgtcgttgatggctggccggggccgctcctcggcgggtcggtcgggccgattccgggcatggaaccagacctgccagccttgaccgaaacggaggaatgggaacggcgcgggcagcagcgcctgccgatgcccgatgagccgtgttttctaaggcccgtaccttggtctggacggtcggaactggctttgcctccttacccttgccgcgcccgtcgtcgcggacggctacgggctactcggcacaaaagtggacgatggcgagccgttggagccgccgacacgggtcacgctgccgcgccggtagcacttgggttgcgcagcaacccgtaagtgcgctgttccagactaacctgctaccgctcggcaacctcggcggctgtgcccagtgcgacggcgcggccatcgtgaacccaacgcgtcgttgggcattcacgcgacaaggtctgattcggctgtagccgcctcgccgccctataccttgtctgcctccccgcgttgcgtcgcggtgcatggagccgggccacctcgacctgaatggaagccggcggagccgacatcggcggagcggcgggatatggaacagacggaggggcgcaacgcagcgccacgtacctcggcccggtggagctggacttaccttcggccgcccacctcgctaacggattcaccgtttttatcaggctctgggaggcagaataaatgatcatatcgtcaattattacctccacggggagagcctgagcaaactgtggagcgattgcctaagtggcaaaaatagtccgagaccctccgtcttatttactagtatagcagttaataatggaggtgcccctctcggactcgtttgaggcctcaggcatttgagaagcacacggtcacactgcttccggtagtcaataaaccggtaaaccagcaatagacataagcggctatttaacgaccctgcccccggagtccgtaaactcttcgtgtgccagtgtgacgaaggccatcagttatttggccatttggtcgttatctgtattcgccgataaattgctgggacgggtgaaccgacgaccgggtcgaatttgctttcgaatttctgccattcatccgcttattatcacttattcaggcgtagcaccaggcgtttaagggcaccaataacttggctgctggcccagcttaaacgaaagcttaaagacggtaagtaggcgaataatagtgaataagtccgcatcgtggtccgcaaattcccgtggttatactgccttaaaaaaattacgccccgccctgccactcatcgcagtcggcctattggttaaaaaatgagctgatttaacaaaaatttaacgcgaattttaactgacggaatttttttaatgcggggcgggacggtgagtagcgtcagccggataaccaattttttactcgactaaattgtttttaaattgcgcttaaaattgaaaatattaacgcttacaatttccattcgccattcaggctgcgcaactgttgggaagggcgatcggtgcgggcctcttcgctattacgccagctggcgaattttataattgcgaatgttaaaggtaagcggtaagtccgacgcgttgacaacccttcccgctagccacgcccggagaagcgataatgcggtcgaccgcttagggggatgtgctgcaaggcgattaagttgggtaacgccagggttttcccagtcacgacgttgtaaaacgacggccagtgagcgcgcgtaatacgactcatccccctacacgacgttccgctaattcaacccattgcggtcccaaaagggtcagtgctgcaacattttgctgccggtcactcgcgcgcattatgctgagtctatagggcgaattggagctccaccgcggtggcggccgctctagaactagtggatcccccgggctgcaggaattcgatatcaagcttatcgataccgtcggatatcccgcttaacctcgaggtggcgccaccgccggcgagatcttgatcacctagggggcccgacgtccttaagctatagttcgaatagctatggcagcacgggcccgggatccgatgctcttccgctaagatcttttactagttcagtccatctcgccgtgtatgcgggcctgacggatcaacgttcccaccgagccatgcccgggccctaggctacgagaaggcgattctagaaaatgatcaagtcaggtagagcggcacatacgcccggactgcctagttgcaagggtggctcggtgtcgagatgttcatctggtcggcgatctgccggtacttcaaaccttgtttgcgcagttccacagccttcttgcggcgttcctgcgcacgagcgatgtagtcagctctacaagtagaccagccgctagacggccatgaagtttggaacaaacgcgtcaaggtgtcggaagaacgccgcaaggacgcgtgctcgctacatcacgcctcggtcttcggcgacgagccgtttgatggtgcttttcgagacgccgaacttgtcagccaactcctgcgcggtctgcgtgcgacgcatcacgcgttcgcggagccagaagccgctgctcggcaaactaccacgaaaagctctgcggcttgaacagtcggttgaggacgcgccagacgcacgctgcgtagtgcgcaagtgcagcacccatcagtccgtcccctctgctgctgcgaacagtgccgatcgatcgaccttcttgagcttcggccgcggcgcggtggcgttcttccgtaccgacgtcgtgggtagtcaggcaggggagacgacgacgcttgtcacggctagctagctggaagaactcgaagccggcgccgcgccaccgcaagaaggcatggccttccgtttttgcgctgctgctcactttgccgcggcgtgcctggattttcgagaactcggcggcggtgaaggtgcggtgggtccagtgggcgactgatttgaaggcaaaaacgcgacgacgagtgaaacggcgccgcacggacctaaaagctcttgagccgccgccacttccacgccacccaggtcacccgctgactaaagccgatctgctcggcctcggcccgactcatggggccgatcccgtcgttggcgtcgagggtgaagttggtcagggcggtgaagtcggtgaccatctgccgccggctagacgagccggagccgggctgagtaccccggctagggcagcaaccgcagctcccacttcaaccagtcccgccacttcagccactggtagacggcgcacacagtgatcgacgggtagttctgtttccggatctcgcggtaggcccattcccgggtgcggtcgaacagttcgacgttccggcccgtttcggtcctgagtgtgtcactagctgcccatcaagacaaaggcctagagcgccatccgggtaagggcccacgccagcttgtcaagctgcaaggccgggcaaagccaggactcctgtgtcttgcggccgtagtccggtggggcggggaaacggtcaccgagcgcttttgcgaggcctttgagcgagtacggatccgagggaccccagaccgtggacacagaacgccggcatcaggccaccccgcccctttgccagtggctcgcgaaaacgctccggaaactcgctcatgcctaggctccctggggtctggcacgtccagtgcgggtggatcgggttctgggtgagctgctgcgcgtagccctgatcggcgccgaccaccgaggcgatcagcccctggttcacccggtcgtaggcaggtcacgcccacctagcccaagacccactcgacgacgcgcatcgggactagccgcggctggtggctccgctagtcggggaccaagtgggccagcatcagccgcagcgggccctgtcgggctgcctggagggtgtagaccgggctttcgagcagccaccacaggtgcgcgtgctcggtcgcgggattgatcgtcatcatcggcgtcgcccgggacagcccgacggacctcccacatctggcccgaaagctcgtcggtggtgtccacgcgcacgagccagcgccctaactagcagtagtcggtcggatcgggcagatccgcgttacgtgcggcccactgcgcctggtcgtcgtccacgtcgagcaccaagcccaacctgatcgacggggtgcgggccgcgccagcctagcccgtctaggcgcaatgcacgccgggtgacgcggaccagcagcaggtgcagctcgtggttcgggttggactagctgccccacgcccggcgaatgtagcggcgggtgagcgcctccgcgcgcggctgcggccactgcccgtcccggacgtagtcatccgtcgcgtgcgggtatttgaaccgccagcggtccttacatcgccgcccactcgcggaggcgcgcgccgacgccggtgacgggcagggcctgcatcagtaggcagcgcacgcccataaacttggcggtcgccaggaaccaggcgtcaacagcagcggtcatgaccgccaagctagggccggatctgtaccgatcgggggaggcgcgccgcaaattatttaagagtctcgctagcattggtccgcagttgtcgtcgccagtactggcggttcgatcccggcctagacatggctagccccctccgcgcggcgtttaataaattctcagagcgatcgtaaccatgtcaggtgttgcggtgggttccgggtaaacctccacccgaattatttaagagtctcgctagctaagccctatctgatgctgcgcggggggtcctttggtacagtccacaacgccacccaaggcccatttggaggtgggcttaataaattctcagagcgatcgattcgggatagactacgacgcgccccccaggatcgcactgaatctcaaaggtggccggctgaatttcgtcgcgcgaaaacctccctggacagttctggaattcagcaagaggtgtgtctgaacttcggtgttagcgtgacttagagtttccaccggccgacttaaagcagcgcgcttttggagggacctgtcaagaccttaagtcgttctccacacagacttgaagccacaatttttggggggtgactccagcggggtgggcacaacgcgaacagagaccttgtgtgtacgacggcgggaggtaagtcgggtacggctcggactgcggtagaaaaaaccccccactgaggtcgccccacccgtgttgcgcttgtctctggaacacacatgctgccgccctccattcagcccatgccgagcctgacgccatctgcaaccgtcgaatcgatttcgagcagagcgagcagagcaagatattccaaaactccggggttcctcggcggcctcccccgtctgtttgctcaaccgagggcgttggcagcttagctaaagctcgtctcgctcgtctcgttctataaggttttgaggccccaaggagccgccggagggggcagacaaacgagttggctcccagacctggcggtcccgcgtttccggacgcgcgggaccgcctaccgctcgagagcggaagagcatctagatgcattcgcgaggtacccagcttttgttccctctggaccgccagggcgcaaaggcctgcgcgccctggcggatggcgagctctcgccttctcgtagatctacgtaagcgctccatgggtcgaaaacaagggtttagtgagggttaattgcgcgcttggcgtaatcatggtcatagctgtttcctgtgtgaaattgttatccgctcacaattccacacaacatacgagccggaaatcactcccaattaacgcgcgaaccgcattagtaccagtatcgacaaaggacacactttaacaataggcgagtgttaaggtgtgttgtatgctcggccaagcataaagtgtaaagcctggggtgcctaatgagtgagctaactcacattaattgcgttgcgctcactgcccgctttccagtcgggaaacctgtcgtgcttcgtatttcacatttcggaccccacggattactcactcgattgagtgtaattaacgcaacgcgagtgacgggcgaaaggtcagccctttggacagcacgcagctgcattaatgaatcggccaacgcgcggggagaggcggtttgcgtattgggcgcatgcataaaaactgttgtaattcattaagcattctgccgacatgtcgacgtaattacttagccggttgcgcgcccctctccgccaaacgcataacccgcgtacggaagccatcacaaacggcatgatgaacctgaatcgccagcggcatcagcaccttgtcgccttgcgtataatatttgcccatgggggtgggcgaagaactccttcggtagtgtttgccgtactacttggacttagcggtcgccgtagtcgtggaacagcggaacgcatattataaacgggtacccccacccgcttcttgaccagcatgagatccccgcgctggaggatcatccagccggcgtcccggaaaacgattccgaagcccaacctttcatagaaggcggcggtggaatcgaaatcggtcgtactctaggggcgcgacctcctagtaggtcggccgcagggccttttgctaaggcttcgggttggaaagtatcttccgccgccaccttagctttagtcgtgatggcaggttgggcgtcgcttggtcggtcatttcgaaccccagagtcccgctcagaagaactcgtcaagaaggcgatagaaggcgatgcgctgcgagcactaccgtccaacccgcagcgaaccagccagtaaagcttggggtctcagggcgagtcttcttgagcagttcttccgctatcttccgctacgcgacgcaatcgggagcggcgataccgtaaagcacgaggaagcggtcagcccattcgccgccaagctcttcagcaatatcacgggtagccaacgctatgtcctgatattagccctcgccgctatggcatttcgtgctccttcgccagtcgggtaagcggcggttcgagaagtcgttatagtgcccatcggttgcgatacaggactatgcggtccgccacacccagccggccacagtcgatgaatccagaaaagcggccattttccaccatgatattcggcaagcaggcatcgccatgggtcacgacgcgccaggcggtgtgggtcggccggtgtcagctacttaggtcttttcgccggtaaaaggtggtactataagccgttcgtccgtagcggtacccagtgctgcagatcctcgccgtcgggcatgcgcgccttgagcctggcgaacagttcggctggcgcgagcccctgatgctcttcgtccagatcatcctgatcgacaagactctaggagcggcagcccgtacgcgcggaactcggaccgcttgtcaagccgaccgcgctcggggactacgagaagcaggtctagtaggactagctgttctgcggcttccatccgagtacgtgctcgctcgatgcgatgtttcgcttggtggtcgaatgggcaggtagccggatcaagcgtatgcagccgccgcattgcatcgccgaaggtaggctcatgcacgagcgagctacgctacaaagcgaaccaccagcttacccgtccatcggcctagttcgcatacgtcggcggcgtaacgtagagccatgatggatactttctcggcaggagcaaggtgagatgacaggagatcctgccccggcacttcgcccaatagcagccagtcccttcccgcttcagttcggtactacctatgaaagagccgtcctcgttccactctactgtcctctaggacggggccgtgaagcgggttatcgtcggtcagggaagggcgaagtcacacaacgtcgagcacagctgcgcaaggaacgcccgtcgtggccagccacgatagccgcgctgcctcgtcctgcagttcattcagggcaccggacaggtcggtgttgcagctcgtgtcgacgcgttccttgcgggcagcaccggtcggtgctatcggcgcgacggagcaggacgtcaagtaagtcccgtggcctgtccagcctcttgacaaaaagaaccgggcgcccctgcgctgacagccggaacacggcggcatcagagcagccgattgtctgttgtgcccagtcatagccgaatagcctagaactgtttttcttggcccgcggggacgcgactgtcggccttgtgccgccgtagtctcgtcggctaacagacaacacgggtcagtatcggcttatcggactccacccaagcggccggagaacctgcgtgcaatccatcttgttcaatcatgcgaaacgatcctcatcctgtctcttgatcagatcttgatcccctgcgcgaggtgggttcgccggcctcttggacgcacgttaggtagaacaagttagtacgctttgctaggagtaggacagagaactagtctagaactaggggacgcgcatcagatccttggcggcaagaaagccatccagtttactttgcagggcttcccaaccttaccagagggcgccccagctggcaattccggttcgcttgctggtagtctaggaaccgccgttctttcggtaggtcaaatgaaacgtcccgaagggttggaatggtctcccgcggggtcgaccgttaaggccaagcgaacgactccataaaaccgcccagtctagctatcgccatgtaagcccactgcaagctacctgctttctctttgcgcttgcgttttcccttgtccagatagcccagtaaggtattttggcgggtcagatcgatagcggtacattcgggtgacgttcgatggacgaaagagaaacgcgaacgcaaaagggaacaggtctatcgggtcatgctgacattcatcccaggtggcacttttcggggaaatgtgcgcgcccgcgttcctgctggcgctgggcctgtttctggcgctggacttcccgctgttccgcgactgtaagtagggtccaccgtgaaaagcccctttacacgcgcgggcgcaaggacgaccgcgacccggacaaagaccgcgacctgaagggcgacaaggctcagcagcttttcgcccacggccttgatgatcgcggcggccttggcctgcatatcccgattcaacggccccagggcgtccagaacgggcttcaggcgctcagtcgtcgaaaagcgggtgccggaactactagcgccgccggaaccggacgtatagggctaagttgccggggtcccgcaggtcttgcccgaagtccgcgaccgaaggtctcgggccgtctcttgggcttgatcggccttcttgcgcatctcacgcgctcctgcggcggcctgtagggcaggctcatacccctgccgaaccggcttccagagcccggcagagaacccgaactagccggaagaacgcgtagagtgcgcgaggacgccgccggacatcccgtccgagtatggggacggcttgggcttttgtcagccggtcggccacggcttccggcgtctcaacgcgctttgagattcccagcttttcggccaatccctgcggtgcataggcgcgtggctcgacgaaaacagtcggccagccggtgccgaaggccgcagagttgcgcgaaactctaagggtcgaaaagccggttagggacgccacgtatccgcgcaccgagctccgcttgcgggctgatggtgacgtggcccactggtggccgctccagggcctcgtagaacgcctgaatgcgcgtgtgacgtgccttgctgccctcgatgccggcgaacgcccgactaccactgcaccgggtgaccaccggcgaggtcccggagcatcttgcggacttacgcgcacactgcacggaacgacgggagctacggccgttgcagccctagatcggccacagcggccgcaaacgtggtctggtcgcgggtcatctgcgctttgttgccgatgaactccttggccgacagcctgccgggcaacgtcgggatctagccggtgtcgccggcgtttgcaccagaccagcgcccagtagacgcgaaacaacggctacttgaggaaccggctgtcggacggctcctgcgtcagcggcaccacgaacgcggtcatgtgcgggctggtttcgtcacggtggatgctggccgtcacgatgcgatccgccccgtacttgtccgccaaggacgcagtcgccgtggtgcttgcgccagtacacgcccgaccaaagcagtgccacctacgaccggcagtgctacgctaggcggggcatgaacaggcggtgccacttgtgcgccttctcgaagaacgccgcctgctgttcttggctggccgacttccaccattccgggctggccgtcatgacgtactcgaccgccaacaccggtgaacacgcggaagagcttcttgcggcggacgacaagaaccgaccggctgaaggtggtaaggcccgaccggcagtactgcatgagctggcggttgtgagcgtccttgcgccgcttctctggcagcaactcgcgcagtcggcccatcgcttcatcggtgctgctggccgcccagtgctcgttctctggcgtcctgctgtcgcaggaacgcggcgaagagaccgtcgttgagcgcgtcagccgggtagcgaagtagccacgacgaccggcgggtcacgagcaagagaccgcaggacgacgcgtcagcgttgggcgtctcgcgctcgcggtaggcgtgcttgagactggccgccacgttgcccattttcgccagcttcttgcatcgcatgatcgcgtatgcgcagtcgcaacccgcagagcgcgagcgccatccgcacgaactctgaccggcggtgcaacgggtaaaagcggtcgaagaacgtagcgtactagcgcatacccgccatgcctgcccctcccttttggtgtccaaccggctcgacgggggcagcgcaaggcggtgcctccggcgggccactcaatgcttgagtatactcactggcggtacggacggggagggaaaaccacaggttggccgagctgcccccgtcgcgttccgccacggaggccgcccggtgagttacgaactcatatgagtgaagactttgcttcgcaaagtcgtgaccgcctacggcggctgcggcgccctacgggcttgctctccgggcttcgccctgcgcggtcgctgcgctcccttgcctctgaaacgaagcgtttcagcactggcggatgccgccgacgccgcgggatgcccgaacgagaggcccgaagcgggacgcgccagcgacgcgagggaacgg

SEQ ID NO: 4 - DNA, 15158 bp, Artificial Sequence
  Synthetic Polynucleotide
ggggagccgcgccgaaggcgtgggggaaccccgcaggggtgcccttctttgggcaccaaagaactagatatagggcgaaatgcgaaagacttaaaaatcacccctcggcgcggcttccgcacccccttggggcgtccccacgggaagaaacccgtggtttcttgatctatatcccgctttacgctttctgaatttttagtacaacttaaaaaaggggggtacgcaacagctcattgcggcaccccccgcaatagctcattgcgtaggttaaagaaaatctgtaattgactgccacttttatgttgaattttttccccccatgcgttgtcgagtaacgccgtggggggcgttatcgagtaacgcatccaatttcttttagacattaactgacggtgaaaatcgcaacgcataattgttgtcgcgctgccgaaaagttgcagctgattgcgcatggtgccgcaaccgtgcggcaccctaccgcatggagataagcatggccagcgttgcgtattaacaacagcgcgacggcttttcaacgtcgactaacgcgtaccacggcgttggcacgccgtgggatggcgtacctctattcgtaccggtcgcagtccagagaaatcggcattcaagccaagaacaagcccggtcactgggtgcaaacggaacgcaaagcgcatgaggcgtgggccgggcttattgcgaggcgtcaggtctctttagccgtaagttcggttcttgttcgggccagtgacccacgtttgccttgcgtttcgcgtactccgcacccggcccgaataacgctcgaaacccacggcggcaatgctgctgcatcacctcgtggcgcagatgggccaccagaacgccgtggtggtcagccagaagacactttccaagctcatcggactttgggtgccgccgttacgacgacgtagtggagcaccgcgtctacccggtggtcttgcggcaccaccagtcggtcttctgtgaaaggttcgagtagcctcgttctttgcggacggtccaatacgcagtcaaggacttggtggccgagcgctggatctccgtcgtgaagctcaacggccccggcaccgtgtcggcctacggcaagaaacgcctgccaggttatgcgtcagttcctgaaccaccggctcgcgacctagaggcagcacttcgagttgccggggccgtggcacagccggatgctggtcaatgaccgcgtggcgtggggccagccccgcgaccagttgcgcctgtcggtgttcagtgccgccgtggtggttgatcacgacgaccaggacgaatcaccagttactggcgcaccgcaccccggtcggggcgctggtcaacgcggacagccacaagtcacggcggcaccaccaactagtgctgctggtcctgcttaggctgttggggcatggcgacctgcgccgcatcccgaccctgtatccgggcgagcagcaactaccgaccggccccggcgaggagccgcccagccagcccggccgacaaccccgtaccgctggacgcggcgtagggctgggacataggcccgctcgtcgttgatggctggccggggccgctcctcggcgggtcggtcgggccgattccgggcatggaaccagacctgccagccttgaccgaaacggaggaatgggaacggcgcgggcagcagcgcctgccgatgcccgatgagccgtgttttctaaggcccgtaccttggtctggacggtcggaactggctttgcctccttacccttgccgcgcccgtcgtcgcggacggctacgggctactcggcacaaaagtggacgatggcgagccgttggagccgccgacacgggtcacgctgccgcgccggtagcacttgggttgcgcagcaacccgtaagtgcgctgttccagactaacctgctaccgctcggcaacctcggcggctgtgcccagtgcgacggcgcggccatcgtgaacccaacgcgtcgttgggcattcacgcgacaaggtctgattcggctgtagccgcctcgccgccctataccttgtctgcctccccgcgttgcgtcgcggtgcatggagccgggccacctcgacctgaatggaagccggcggagccgacatcggcggagcggcgggatatggaacagacggaggggcgcaacgcagcgccacgtacctcggcccggtggagctggacttaccttcggccgcccacctcgctaacggattcaccgtttttatcaggctctgggaggcagaataaatgatcatatcgtcaattattacctccacggggagagcctgagcaaactgtggagcgattgcctaagtggcaaaaatagtccgagaccctccgtcttatttactagtatagcagttaataatggaggtgcccctctcggactcgtttgaggcctcaggcatttgagaagcacacggtcacactgcttccggtagtcaataaaccggtaaaccagcaatagacataagcggctatttaacgaccctgcccccggagtccgtaaactcttcgtgtgccagtgtgacgaaggccatcagttatttggccatttggtcgttatctgtattcgccgataaattgctgggacgggtgaaccgacgaccgggtcgaatttgctttcgaatttctgccattcatccgcttattatcacttattcaggcgtagcaccaggcgtttaagggcaccaataacttggctgctggcccagcttaaacgaaagcttaaagacggtaagtaggcgaataatagtgaataagtccgcatcgtggtccgcaaattcccgtggttatactgccttaaaaaaattacgccccgccctgccactcatcgcagtcggcctattggttaaaaaatgagctgatttaacaaaaatttaacgcgaattttaactgacggaatttttttaatgcggggcgggacggtgagtagcgtcagccggataaccaattttttactcgactaaattgtttttaaattgcgcttaaaattgaaaatattaacgcttacaatttccattcgccattcaggctgcgcaactgttgggaagggcgatcggtgcgggcctcttcgctattacgccagctggcgaattttataattgcgaatgttaaaggtaagcggtaagtccgacgcgttgacaacccttcccgctagccacgcccggagaagcgataatgcggtcgaccgcttagggggatgtgctgcaaggcgattaagttgggtaacgccagggttttcccagtcacgacgttgtaaaacgacggccagtgagcgcgcgtaatacgactcatccccctacacgacgttccgctaattcaacccattgcggtcccaaaagggtcagtgctgcaacattttgctgccggtcactcgcgcgcattatgctgagtctatagggcgaattggagctccaccgcggtggcggccgctctagaactagtggatcccccgggctgcaggaattcgatatcaagcttttacgccccgcccgatatcccgcttaacctcgaggtggcgccaccgccggcgagatcttgatcacctagggggcccgacgtccttaagctatagttcgaaaatgcggggcgggtgccactcatcgcagtactgttgtaattcattaagcattctgccgacatggaagccatcacaaacggcatgatgaacctgaatcgccagcggcatcagcaacggtgagtagcgtcatgacaacattaagtaattcgtaagacggctgtaccttcggtagtgtttgccgtactacttggacttagcggtcgccgtagtcgtccttgtcgccttgcgtataatatttgcccatggtgaaaacgggggcgaagaagttgtccatattggccacgtttaaatcaaaactggtgaaactcacccaggaacagcggaacgcatattataaacgggtaccacttttgcccccgcttcttcaacaggtataaccggtgcaaatttagttttgaccactttgagtgggtgggattggctgagacgaaaaacatattctcaataaaccctttagggaaataggccaggttttcaccgtaacacgccacatcttgcgaatatatgtgtagaccctaaccgactctgctttttgtataagagttatttgggaaatccctttatccggtccaaaagtggcattgtgcggtgtagaacgcttatatacacatctaactgccggaaatcgtcgtggtattcactccagagcgatgaaaacgtttcagtttgctcatggaaaacggtgtaacaagggtgaacactatcccatatcattgacggcctttagcagcaccataagtgaggtctcgctacttttgcaaagtcaaacgagtaccttttgccacattgttcccacttgtgatagggtatagtccagctcaccgtctttcattgccatacgaaattccggatgagcattcatcaggcgggcaagaatgtgaataaaggccggataaaacttgtgcttatttttggtcgagtggcagaaagtaacggtatgctttaaggcctactcgtaagtagtccgcccgttcttacacttatttccggcctattttgaacacgaataaaaactttacggtctttaaaaaggccgtaatatccagctgaacggtctggttataggtacattgagcaactgactgaaatgcctcaaaatgttctttacgatgcgaaatgccagaaatttttccggcattataggtcgacttgccagaccaatatccatgtaactcgttgactgactttacggagttttacaagaaatgctacgcattgggatatatcaacggtggtatatccagtgatttttttctccatatggttaaccttaattaaggggtcgacgggcccgggatccgatgctcttccgcgtaaccctatatagttgccaccatataggtcactaaaaaaagaggtataccaattggaattaattccccagctgcccgggccctaggctacgagaaggcgtaagatcttttactagttcagtccatctcgccgtgtatgcgggcctgacggatcaacgttcccaccgagccagtcgagatgttcatctggtcggcgatctattctagaaaatgatcaagtcaggtagagcggcacatacgcccggactgcctagttgcaagggtggctcggtcagctctacaagtagaccagccgctagagccggtacttcaaaccttgtttgcgcagttccacagccttcttgcggcgttcctgcgcacgagcgatgtagtcgcctcggtcttcggcgacgagccgtttcggccatgaagtttggaacaaacgcgtcaaggtgtcggaagaacgccgcaaggacgcgtgctcgctacatcagcggagccagaagccgctgctcggcaaagatggtgcttttcgagacgccgaacttgtcagccaactcctgcgcggtctgcgtgcgacgcatcacgcgttctgcagcacccatcagtccgtcccctctgctaccacgaaaagctctgcggcttgaacagtcggttgaggacgcgccagacgcacgctgcgtagtgcgcaagacgtcgtgggtagtcaggcaggggagacctgctgcgaacagtgccgatcgatcgaccttcttgagcttcggccgcggcgcggtggcgttcttccgtaccgcttccgtttttgcgctgctgctcactttgacgacgcttgtcacggctagctagctggaagaactcgaagccggcgccgcgccaccgcaagaaggcatggcgaaggcaaaaacgcgacgacgagtgaaagccgcggcgtgcctggattttcgagaactcggcggcggtgaaggtgcggtgggtccagtgggcgactgatttgccgatctgctcggcctcggcccgactccggcgccgcacggacctaaaagctcttgagccgccgccacttccacgccacccaggtcacccgctgactaaacggctagacgagccggagccgggctgagatggggccgatcccgtcgttggcgtcgagggtgaagttggtcagggcggtgaagtcggtgaccatctgccgccacacagtgatcgacgggtagttctgtttaccccggctagggcagcaaccgcagctcccacttcaaccagtcccgccacttcagccactggtagacggcggtgtgtcactagctgcccatcaagacaatccggatctcgcggtaggcccattcccgggtgcggtcgaacagttcgacgttccggcccgtttcggtcctgacctgtgtcttgcggccgtagtccggtggaggcctagagcgccatccgggtaagggcccacgccagcttgtcaagctgcaaggccgggcaaagccaggactggacacagaacgccggcatcaggccaccggcggggaaacggtcaccgagcgcttttgcgaggcctttgagcgagtacggatccgagggaccccagaccgtcgtccagtgcgggtggatcgggttctggccgcccctttgccagtggctcgcgaaaacgctccggaaactcgctcatgcctaggctccctggggtctggcagcaggtcacgcccacctagcccaagaccgtgagctgctgcgcgtagccctgatcggcgccgaccaccgaggcgatcagcccctggttcacccggtcgtagagccgcagcgggccctgtcgggctgcctcactcgacgacgcgcatcgggactagccgcggctggtggctccgctagtcggggaccaagtgggccagcatctcggcgtcgcccgggacagcccgacggaggagggtgtagaccgggctttcgagcagccaccacaggtgcgcgtgctcggtcgcgggattgatcgtcatcacggtcggatcgggcagatccgcgttacgcctcccacatctggcccgaaagctcgtcggtggtgtccacgcgcacgagccagcgccctaactagcagtagtgccagcctagcccgtctaggcgcaatgctgcggcccactgcgcctggtcgtcgtccacgtcgagcaccaagcccaacctgatcgacggggtgcgggccgcaatgtagcggcgggtgagcgcctccgcgacgccgggtgacgcggaccagcagcaggtgcagctcgtggttcgggttggactagctgccccacgcccggcgttacatcgccgcccactcgcggaggcgccgcggctgcggccactgcccgtcccggacgtagtcatccgtcgcgtgcgggtatttgaaccgccagcggtccaaccaggcgtcaacagcagcggtcatgagcgccgacgccggtgacgggcagggcctgcatcagtaggcagcgcacgcccataaacttggcggtcgccaggttggtccgcagttgtcgtcgccagtactccgccaagctagggccggatctgtaccgatcgggggaggcgcgccgcaaattatttaagagtctcgctagcaaaccatgtcaggtgttgcggtgggttccggcggttcgatcccggcctagacatggctagccccctccgcgcggcgtttaataaattctcagagcgatcgtttggtacagtccacaacgccacccaagggggtaaacctccacccgaattatttaagagtctcgctagctaagccctatctgatgctgcgcggggggtccttcgcactgaatctcaaaggtggccggctcccatttggaggtgggcttaataaattctcagagcgatcgattcgggatagactacgacgcgccccccaggaagcgtgacttagagtttccaccggccgagaatttcgtcgcgcgaaaacctccctggacagttctggaattcagcaagaggtgtgtctgaacttcggtgtttttttggggggtgactccagcggggtggcttaaagcagcgcgcttttggagggacctgtcaagaccttaagtcgttctccacacagacttgaagccacaaaaaaaccccccactgaggtcgccccaccgcacaacgcgaacagagaccttgtgtgtacgacggcgggaggtaagtcgggtacggctcggactgcggtagagcaaccgtcgaatcgatttcgagcagagcgtgttgcgcttgtctctggaacacacatgctgccgccctccattcagcccatgccgagcctgacgccatctcgttggcagcttagctaaagctcgtctccgagcagagcaagatattccaaaactccggggttcctcggcggcctcccccgtctgtttgctcaaccgagggagacctggcggtcccgcgtttccggacggctcgtctcgttctataaggttttgaggccccaaggagccgccggagggggcagacaaacgagttggctccctctggaccgccagggcgcaaaggcctgccgcgggaccgcctaccgctcgagagcggaagagcatctagatgcattcgcgaggtacccagcttttgttccctttagtgagggttaattgcgcgcttggcgcgccctggcggatggcgagctctcgccttctcgtagatctacgtaagcgctccatgggtcgaaaacaagggaaatcactcccaattaacgcgcgaaccggtaatcatggtcatagctgtttcctgtgtgaaattgttatccgctcacaattccacacaacatacgagccggaagcataaagtgtaaagcctggggtgcccattagtaccagtatcgacaaaggacacactttaacaataggcgagtgttaaggtgtgttgtatgctcggccttcgtatttcacatttcggaccccacggtaatgagtgagctaactcacattaattgcgttgcgctcactgcccgctttccagtcgggaaacctgtcgtgccagctgcattaatgaatcggccaacgcgattactcactcgattgagtgtaattaacgcaacgcgagtgacgggcgaaaggtcagccctttggacagcacggtcgacgtaattacttagccggttgcgccggggagaggcggtttgcgtattgggcgcatgcataaaaactgttgtaattcattaagcattctgccgacatggaagccatcacaaacggcatgatgaacgcccctctccgccaaacgcataacccgcgtacgtatttttgacaacattaagtaattcgtaagacggctgtaccttcggtagtgtttgccgtactacttgctgaatcgccagcggcatcagcaccttgtcgccttgcgtataatatttgcccatgggggtgggcgaagaactccagcatgagatccccgcgctggaggatgacttagcggtcgccgtagtcgtggaacagcggaacgcatattataaacgggtacccccacccgcttcttgaggtcgtactctaggggcgcgacctcctacatccagccggcgtcccggaaaacgattccgaagcccaacctttcatagaaggcggcggtggaatcgaaatctcgtgatggcaggttgggcgtcgcttgggtaggtcggccgcagggccttttgctaaggcttcgggttggaaagtatcttccgccgccaccttagctttagagcactaccgtccaacccgcagcgaacctcggtcatttcgaaccccagagtcccgctcagaagaactcgtcaagaaggcgatagaaggcgatgcgctgcgaatcgggagcggcgataccgtaaagcacagccagtaaagcttggggtctcagggcgagtcttcttgagcagttcttccgctatcttccgctacgcgacgcttagccctcgccgctatggcatttcgtggaggaagcggtcagcccattcgccgccaagctcttcagcaatatcacgggtagccaacgctatgtcctgatagcggtccgccacacccagccggccacagctccttcgccagtcgggtaagcggcggttcgagaagtcgttatagtgcccatcggttgcgatacaggactatcgccaggcggtgtgggtcggccggtgtctcgatgaatccagaaaagcggccattttccaccatgatattcggcaagcaggcatcgccatgggtcacgacgagatcctcgccgtcgggcatgcgcgcctagctacttaggtcttttcgccggtaaaaggtggtactataagccgttcgtccgtagcggtacccagtgctgctctaggagcggcagcccgtacgcgcggatgagcctggcgaacagttcggctggcgcgagcccctgatgctcttcgtccagatcatcctgatcgacaagaccggcttccatccgagtacgtgctcgctcactcggaccgcttgtcaagccgaccgcgctcggggactacgagaagcaggtctagtaggactagctgttctggccgaaggtaggctcatgcacgagcgaggatgcgatgtttcgcttggtggtcgaatgggcaggtagccggatcaagcgtatgcagccgccgcattgcatcagccatgatggatactttctcggcaggactacgctacaaagcgaaccaccagcttacccgtccatcggcctagttcgcatacgtcggcggcgtaacgtagtcggtactacctatgaaagagccgtcctgcaaggtgagatgacaggagatcctgccccggcacttcgcccaatagcagccagtcccttcccgcttcagtgacaacgtcgagcacagctgcgcaaggaacgttccactctactgtcctctaggacggggccgtgaagcgggttatcgtcggtcagggaagggcgaagtcactgttgcagctcgtgtcgacgcgttccttcgcccgtcgtggccagccacgatagccgcgctgcctcgtcctgcagttcattcagggcaccggacaggtcggtcttgacaaaaagaaccgggcgcccctggcgggcagcaccggtcggtgctatcggcgcgacggagcaggacgtcaagtaagtcccgtggcctgtccagccagaactgtttttcttggcccgcggggaccgctgacagccggaacacggcggcatcagagcagccgattgtctgttgtgcccagtcatagccgaatagcctctccacccaagcggccggagaacctgcggcgactgtcggccttgtgccgccgtagtctcgtcggctaacagacaacacgggtcagtatcggcttatcggagaggtgggttcgccggcctcttggacgctgcaatccatcttgttcaatcatgcgaaacgatcctcatcctgtctcttgatcagatcttgatcccctgcgccatcagatccttggcggcaagaaagccaacgttaggtagaacaagttagtacgctttgctaggagtaggacagagaactagtctagaactaggggacgcggtagtctaggaaccgccgttctttcggttccagtttactttgcagggcttcccaaccttaccagagggcgccccagctggcaattccggttcgcttgctgtccataaaaccgcccagtctagctatcgaggtcaaatgaaacgtcccgaagggttggaatggtctcccgcggggtcgaccgttaaggccaagcgaacgacaggtattttggcgggtcagatcgatagcccatgtaagcccactgcaagctacctgctttctctttgcgcttgcgttttcccttgtccagatagcccagtagctgacattcatcccaggtggcacttttggtacattcgggtgacgttcgatggacgaaagagaaacgcgaacgcaaaagggaacaggtctatcgggtcatcgactgtaagtagggtccaccgtgaaaacggggaaatgtgcgcgcccgcgttcctgctggcgctgggcctgtttctggcgctggacttcccgctgttccgtcagcagcttttcgcccacggccttgatgcccctttacacgcgcgggcgcaaggacgaccgcgacccggacaaagaccgcgacctgaagggcgacaaggcagtcgtcgaaaagcgggtgccggaactagatcgcggcggccttggcctgcatatcccgattcaacggccccagggcgtccagaacgggcttcaggcgctcccgaaggtctcgggccgtctcttgggctctagcgccgccggaaccggacgtatagggctaagttgccggggtcccgcaggtcttgcccgaagtccgcgagggcttccagagcccggcagagaacccgatgatcggccttcttgcgcatctcacgcgctcctgcggcggcctgtagggcaggctcatacccctgccgaaccgcttttgtcagccggtcggccacggcttactagccggaagaacgcgtagagtgcgcgaggacgccgccggacatcccgtccgagtatggggacggcttggcgaaaacagtcggccagccggtgccgaaccggcgtctcaacgcgctttgagattcccagcttttcggccaatccctgcggtgcataggcgcgtggctcgaccgcttgcgggctgatggtgacgtggccggccgcagagttgcgcgaaactctaagggtcgaaaagccggttagggacgccacgtatccgcgcaccgagctggcgaacgcccgactaccactgcaccggcactggtggccgctccagggcctcgtagaacgcctgaatgcgcgtgtgacgtgccttgctgccctcgatgccccgttgcagccctagatcggccacagcggtgaccaccggcgaggtcccggagcatcttgcggacttacgcgcacactgcacggaacgacgggagctacggggcaacgtcgggatctagccggtgtcgcgccgcaaacgtggtctggtcgcgggtcatctgcgctttgttgccgatgaactccttggccgacagcctgccgtcctgcgtcagcggcaccacgaacgcggcggcgtttgcaccagaccagcgcccagtagacgcgaaacaacggctacttgaggaaccggctgtcggacggcaggacgcagtcgccgtggtgcttgcgcctcatgtgcgggctggtttcgtcacggtggatgctggccgtcacgatgcgatccgccccgtacttgtccgccagccacttgtgcgccttctcgaagaacgcagtacacgcccgaccaaagcagtgccacctacgaccggcagtgctacgctaggcggggcatgaacaggcggtcggtgaacacgcggaagagcttcttgcgcgcctgctgttcttggctggccgacttccaccattccgggctggccgtcatgacgtactcgaccgccaacacagcgtccttgcgccgcttctctggcagcgcggacgacaagaaccgaccggctgaaggtggtaaggcccgaccggcagtactgcatgagctggcggttgtgtcgcaggaacgcggcgaagagaccgtcgaactcgcgcagtcggcccatcgcttcatcggtgctgctggccgcccagtgctcgttctctggcgtcctgctggcgtcagcgttgggcgtctcgcgctcgcttgagcgcgtcagccgggtagcgaagtagccacgacgaccggcgggtcacgagcaagagaccgcaggacgaccgcagtcgcaacccgcagagcgcgagcgggtaggcgtgcttgagactggccgccacgttgcccattttcgccagcttcttgcatcgcatgatcgcgtatgccgccatgcctgcccctcccttttggtgccatccgcacgaactctgaccggcggtgcaacgggtaaaagcggtcgaagaacgtagcgtactagcgcatacggcggtacggacggggagggaaaaccactccaaccggctcgacgggggcagcgcaaggcggtgcctccggcgggccactcaatgcttgagtatactcactagactttgcttcgcaaagtcgtgaccgcaggttggccgagctgcccccgtcgcgttccgccacggaggccgcccggtgagttacgaactcatatgagtgatctgaaacgaagcgtttcagcactggcgctacggcggctgcggcgccctacgggcttgctctccgggcttcgccctgcgcggtcgctgcgctcccttgccagcccgtggatatgtggacgatggccgcgatgccgccgacgccgcgggatgcccgaacgagaggcccgaagcgggacgcgccagcgacgcgagggaacggtcgggcacctatacacctgctaccggcggagcggccaccggctggctcgcttcgctcggcccgtggacaaccctgctggacaagctgatggacaggctgcgcctgcccacgagcttgaccacagggatctcgccggtggccgaccgagcgaagcgagccgggcacctgttgggacgacctgttcgactacctgtccgacgcggacgggtgctcgaactggtgtccctatgcccaccggctacccagccttcgaccacatacccaccggctccaactgcgcggcctgcggccttgccccatcaatttttttaattttctctggggaaaaacgggtggccgatgggtcggaagctggtgtatgggtggccgaggttgacgcgccggacgccggaacggggtagttaaaaaaattaaaagagaccccttttgcctccggcctgcggcctgcgcgcttcgcttgccggttggacaccaagtggaaggcgggtcaaggctcgcgcagcgaccgcgcagcggcttggccttgaccggaggccggacgccggacgcgcgaagcgaacggccaacctgtggttcaccttccgcccagttccgagcgcgtcgctggcgcgtcgccgaaccggaactggcgcctggaacgacccaagcctatgcgagtgggggcagtcgaaggcgaagcccgcccgcctgccccccgagcctcacggcggcgagtgcgggggttccaacgcggaccttgctgggttcggatacgctcacccccgtcagcttccgcttcgggcgggcggacggggggctcggagtgccgccgctcacgcccccaaggttgggggcagcgccaccttgggcaaggccgaaggccgcgcagtcgatcaacaagccccggaggggccactttttgccggagcccccgtcgcggtggaacccgttccggcttccggcgcgtcagctagttgttcggggcctccccggtgaaaaacggcctc

SEQ ID NO: 5 - protein, 616 aa, Artificial Sequence
  Synthetic Polypeptide
MEALFLSSSSSSIVASNKLTRLHNHCVWSTVIRDKKRFGPTWCRVGGGGDGGRNSNAERPIRVSSLLKDRGQVLIREQSSPAMDAETLVLSPNGNGRTIEINGVKTLMPFSGASMVGMKEGLGIISFLQGKKFLITGSTGFLAKVLIEKVLRMAPDVSKIYLLIKAKSKEAAIERLKNEVLDAELFNTLKETHGASYMSFMLTKLIPVTGNICDSNIGLQADSAEEIAKEVDVIINSAANTTFNERYDVALDINTRGPGNLMGFAKKCKKLKLFLQVSTAYVNGQRQGRIMEKPFSMGDCIATENFLEGNRKALDVDREMKLALEAARKGTQNQDEAQKMKDLGLERARSYGWQDTYVFTKAMGEMMINSTRGDVPVVIIRPSVIESTYKDPFPGWMEGNRMMDPIVLCYGKGQLTGFLVDPKGVLDVVPADMVVNATLAAIAKHGMAMSDPEPEINVYQIASSAINPLVFEDLAELLYNHYKTSPCMDSKGDPIMVRLMKLFNSVDDFSDHLWRDAQERSGLMSGMSSVDSKMMQKLKFICKKSVEQAKHLATIYEPYTFYGGRFDNSNTQRLMENMSEDEKREFGFDVGSINWTDYITNVHIPGLRRHVLKGRA

SEQ ID NO: 6 - protein, 548 aa, Artificial Sequence
  Synthetic Polypeptide
MATTNVLATSHAFKLNGVSYFSSFPRKPNHYMPRRRLSHTTRRVQTSCFYGETSFEAVTSLVTPKTETSRNSDGIGIVRFLEGKSYLVTGATGFLAKVLIEKLLRESLEIGKIFLLMRSKDQESANKRLYDEIISSDLFKLLKQMHGSSYEAFMKRKLIPVIGDIEEDNLGIKSEIANMISEEIDVIISCGGRTTFDDRYDSALSVNALGPGRLLSFGKGCRKLKLFLHFSTAYVTGKREGTVLETPLCIGENITSDLNIKSELKLASEAVRKFRGREEIKKLKELGFERAQHYGWENSYTFTKAIGEAVIHSKRGNLPVVIIRPSIIESSYNEPFPGWIQGTRMADPIILAYAKGQISDFWADPQSLMDIIPVDMVANAAIAAMAKHGCGVPEFKVYNLTSSSHVNPMRAGKLIDLSHQHLCDFPLEETVIDLEHMKIHSSLEGFTSALSNTIIKQERVIDNEGGGLSTKGKRKLNYFVSLAKTYEPYTFFQARFDNTNTTSLIQEMSMEEKKTFGFDIKGIDWEHYIVNVHLPGLKKEFLSKKKTE

SEQ ID NO: 7 - protein, 491 aa, Artificial Sequence
  Synthetic Polypeptide
MESNCVQFLGNKTILITGAPGFLAKVLVEKILRLQPNVKKIYLLLRAPDEKSAMQRLRSEVMEIDLFKVLRNNLGEDNLNALMREKIVPVPGDISIDNLGLKDTDLIQRMWSEIDIIINIAATTNFDERYDIGLGINTFGALNVLNFAKKCVKGQLLLHVSTAYISGEQPGLLLEKPFKMGETLSGDRELDINIEHDLMKQKLKELQDCSDEEISQTMKDFGMARAKLHGWPNTYVFTKAMGEMLMGKYRENLPLVIIRPTMITSTIAEPFPGWIEGLKTLDSVIVAYGKGRLKCFLADSNSVFDLIPADMVVNAMVAAATAHSGDTGIQAIYHVGSSCKNPVTFGQLHDFTARYFAKRPLIGRNGSPIIVVKGTILSTMAQFSLYMTLRYKLPLQILRLINIVYPWSHGDNYSDLSRKIKLAMRLVELYQPYLLFKGIFDDLNTERLRMKRKENIKELDGSFEFDPKSIDWDNYITNTHIPGLITHVLKQ

SEQ ID NO: 8 - protein, 231 aa, Artificial Sequence
  Synthetic Polypeptide
MPELAVRTEFDYSSEIYKDAYSRINAIVIEGEQEAYSNYLQMAELLPEDKEELTRLAKMENRHKKGFQACGNNLQVNPDMPYAQEFFAGLHGNFQHAFSEGKVVTCLLIQALIIEAFAIAAYNIYIPVADDFARKITEGVVKDEYTHLNYGEEWLKANFATAKEELEQANKENLPLVWKMLNQVQGDAKVLGMEKEALVEDFMISYGEALSNIGFSTREIMRMSSYGLAGV

SEQ ID NO: 9 - protein, 340 aa, Artificial Sequence
  Synthetic Polypeptide
MFGLIGHLTSLEHAQAVAEDLGYPEYANQGLDFWCSAPPQVVDNFQVKSVTGQVIEGKYVESCFLPEMLTQRRIKAAIRKILNAMALAQKVGLDITALGGFSSIVFEEFNLKQNNQVRNVELDFQRFTTGNTHTAYVICRQVESGAKQLGIDLSQATVAVCGATGDIGSAVCRWLDSKHQVKELLLIARNRQRLENLQEELGRGKIMDLETALPQADIIVWVASMPKGVEIAGEMLKKPCLIVDGGYPKNLDTRVKADGVHILKGGIVEHSLDITWEIMKIVEMDIPSRQMFACFAEAILLEFEGWRTNFSWGRNQISVNKMEAIGEASVKHGFCPLVAL

SEQ ID NO: 10 - DNA, 410 bp, Artificial Sequence
  Synthetic Polynucleotide
cagtcaatggagagcattgccataagtaaaggcatcccctgcgtgataagattaccttcagaaaacagatagttgctgggttatcgcagatttttctcgcgtcagttacctctcgtaacggtattcatttccgtaggggacgcactattctaatggaagtcttttgtctatcaacgacccaatagcgtctaaaaagagcgaaccaaataactgtaaataataactgtctctggggcgacggtaggctttatattgccaaatttcgcccgtgggagaaagctaggctattcaatgtttatgttggtttattgacatttattattgacagagaccccgctgccatccgaaatataacggtttaaagcgggcaccctctttcgatccgataagttacaaatacgaggactcct

SEQ ID NO: 11 - DNA, 2962 bp, Rhodococcus opacus
  misc_feature (1070)..(1070): n is a, c, g, or t
cctggctcaggacgaacgctggcggcgtgcttaacacatgcaagtcgagcggtaaggcccttcggggtacacgagcggcgaacgggtgagtaacacgtggggaccgagtcctgcttgcgaccgccgcacgaattgtgtacgttcagctcgccattccgggaagccccatgtgctcgccgcttgcccactcattgtgcaccgtgatctgccctgcacttcgggataagcctgggaaactgggtctaataccggatatgaccttcggctgcatggctgagggtggaaaggtttactggtgcacactagacgggacgtgaagccctattcggaccctttgacccagattatggcctatactggaagccgacgtaccgactcccacctttccaaatgaccacgtggatgggcccgcggcctatcagcttgttggtggggtaatggcctaccaaggcgacgacgggtagccgacctgagagggtgaccggccacactgggactgacctacccgggcgccggatagtcgaacaaccaccccattaccggatggttccgctgctgcccatcggctggactctcccactggccggtgtgaccctgactgacacggcccagactcctacgggaggcagcagtggggaatattgcacaatgggcgaaagcctgatgcagcgacgccgcgtgagggatgacggccttcgggctgtgccgggtctgaggatgccctccgtcgtcaccccttataacgtgttacccgctttcggactacgtcgctgcggcgcactccctactgccggaagcccttgtaaacctctttcagcagggacgaagcgaaagtgacggtacctgcagaagaagcaccggccaactacgtgccagcagccgcggtaatacgtagggtgcaacatttggagaaagtcgtccctgcttcgctttcactgccatggacgtcttcttcgtggccggttgatgcacggtcgtcggcgccattatgcatcccacgaagcgttgtccggaattactgggcgtaaagagctcgtaggcggtttgtcgcgtcgtctgtgaaaactcanagctcaacctcgagcttgcaggcgatacggttcgcaacaggccttaatgacccgcatttctcgagcatccgccaaacagcgcagcagacacttttgagtntcgagttggagctcgaacgtccgctatgccgcagacttgagtactgcaggggagactggaattcctggtgtagcggtgaaatgcgcagatatcaggaggaacaccggtggcgaaggcgggtctctgggcacgtctgaactcatgacgtcccctctgaccttaaggaccacatcgccactttacgcgtctatagtcctccttgtggccaccgcttccgcccagagacccgtgtaactgacgctgaggagcgaaagcgtgggtagcaaacaggattagataccctggtagtccacgccgtaaacggtgggcgctaggtgtgggtttccttcccattgactgcgactcctcgctttcgcacccatcgtttgtcctaatctatgggaccatcaggtgcggcatttgccacccgcgatccacacccaaaggaaggacgggatccgtgccgtagttaacgcattaagcgccccgcctggggagtacggccgcaaggttaaaactcaaaggaattgacgggggcccgcacaagcggctgccctaggcacggcatcaattgcgtaattcgcggggcggacccctcatgccggcgttccaattttgagtttccttaactgcccccgggcgtgttcgccgggagcatgtggattaattcgatgcaacgcgaagaaccttacctgggtttgacatataccggaaagccgtagagataccgccccccttgtggtcggtataccctcgtacacctaattaagctacgttgcgcttcttggaatggacccaaactgtatatggcctttcggcatctctatggcggggggaacaccagccatatgaggtggtgcatggctgtcgtcagctcgtgtcgtgagatgttgggttaagtcccgcaacgagcgcaacccttgtcttatgttgccagcacgtaatggtgggtccaccacgtaccgacagcagtcgagcacagcactctacaacccaattcagggcgttgctcgcgttgggaacagaatacaacggtcgtgcattaccacccgactcgtaagagactgccggggtcaactcggaggaaggtggggacgacgtcaagtcatcatgccccttatgtccagggcttcacacatgctacaatggccctgagcattctctgacggccccagttgagcctccttccacccctgctgcagttcagtagtacggggaatacaggtcccgaagtgtgtacgatgttaccggggtacagagggctgcgataccgtgaggtggagcgaatcccttaaagccggtctcagttcggatcggggtctgcaactcgaccccgtgaagtcggagtcgcccatgtctcccgacgctatggcactccacctcgcttagggaatttcggccagagtcaagcctagccccagacgttgagctggggcacttcagcctcagcgtagtaatcgcagatcagcaacgctgcggtgaatacgttcccgggccttgtacacaccgcccgtcacgtcatgaaagtcggtaacacccgaagccggtggcatcattagcgtctagtcgttgcgacgccacttatgcaagggcccggaacatgtgtggcgggcagtgcagtactttcagccattgtgggcttcggccaccgctaaccccttgtgggagggagccgtcgaaggtgggatcggcgattgggacgaagtcgtaacaaggtagccgtaccggaagggattggggaacaccctccctcggcagcttccaccctagccgctaaccctgcttcagcattgttccatcggcatggccttcc

SEQ ID NO: 12 - DNA, 3042 bp, Rhodococcus opacus
tcaacggagagtttgatcctggctcaggacgaacgctggcggcgtgcttaacacatgcaagtcgagcggtaaggcccttcggggtacacgagcggcgaacagttgcctctcaaactaggaccgagtcctgcttgcgaccgccgcacgaattgtgtacgttcagctcgccattccgggaagccccatgtgctcgccgcttggggtgagtaacacgtgggtgatctgccctgcacttcgggataagcctgggaaactgggtctaataccggatatgaccttcggctgcatggccgttggtggcccactcattgtgcacccactagacgggacgtgaagccctattcggaccctttgacccagattatggcctatactggaagccgacgtaccggcaaccaccaaaggtttactggtgcaggatgggcccgcggcctatcagcttgttggtggggtaatggcctaccaaggcgacgacgggtagccgacctgagagggtgacctttccaaatgaccacgtcctacccgggcgccggatagtcgaacaaccaccccattaccggatggttccgctgctgcccatcggctggactctcccactggggccacactgggactgagacacggcccagactcctacgggaggcagcagtggggaatattgcacaatgggcgaaagcctgatgcagcgacgccgcgtgagccggtgtgaccctgactctgtgccgggtctgaggatgccctccgtcgtcaccccttataacgtgttacccgctttcggactacgtcgctgcggcgcactcggatgacggccttcgggttgtaaacctctttcagcagggacgaagcgaaagtgacggtacctgcagaagaagcaccggccaactacgtgccagcagccgccctactgccggaagcccaacatttggagaaagtcgtccctgcttcgctttcactgccatggacgtcttcttcgtggccggttgatgcacggtcgtcggcgggtaatacgtagggtgcaagcgttgtccggaattactgggcgtaaagagctcgtaggcggtttgtcgcgtcgtctgtgaaaactcgaggctcaacctcgaccattatgcatcccacgttcgcaacaggccttaatgacccgcatttctcgagcatccgccaaacagcgcagcagacacttttgagctccgagttggagctgcttgcaggcgatacgggcagacttgagtactgcaggggagactggaattcctggtgtagcggtgaaatgcgcagatatcaggaggaacaccggtggcgacgaacgtccgctatgcccgtctgaactcatgacgtcccctctgaccttaaggaccacatcgccactttacgcgtctatagtcctccttgtggccaccgctaggcgggtctctgggcagtaactgacgctgaggagcgaaagcgtgggtagcgaacaggattagataccctggtagtccacgccgtaaacggtgggcgctatccgcccagagacccgtcattgactgcgactcctcgctttcgcacccatcgcttgtcctaatctatgggaccatcaggtgcggcatttgccacccgcgatggtgtgggtttccttccacgggatccgtgccgtagctaacgcattaagcgccccgcctggggagtacggccgcaaggctaaaactcaaaggaattgacggccacacccaaaggaaggtgccctaggcacggcatcgattgcgtaattcgcggggcggacccctcatgccggcgttccgattttgagtttccttaactgccgggcccgcacaagcggcggagcatgtggattaattcgatgcaacgcgaagaaccttacctgggtttgacatataccggaaagctgcagagatgtggcccccccgggcgtgttcgccgcctcgtacacctaattaagctacgttgcgcttcttggaatggacccaaactgtatatggcctttcgacgtctctacaccggggccttgtggtcggtatacaggtggtgcatggctgtcgtcagctcgtgtcgtgagatgttgggttaagtcccgcaacgagcgcaacccttgtcttatgttgcggaacaccagccatatgtccaccacgtaccgacagcagtcgagcacagcactctacaacccaattcagggcgttgctcgcgttgggaacagaatacaacgcagcacgtaatggtggggactcgtaagagactgccggggtcaactcggaggaaggtggggacgacgtcaagtcatcatgccccttatgtccagggcttcagtcgtgcattaccacccctgagcattctctgacggccccagttgagcctccttccacccctgctgcagttcagtagtacggggaatacaggtcccgaagtcacatgctacaatggccggtacagagggctgcgataccgtgaggtggagcgaatcccttaaagccggtctcagttcggatcggggtctgcaactcgacccgtgtacgatgttaccggccatgtctcccgacgctatggcactccacctcgcttagggaatttcggccagagtcaagcctagccccagacgttgagctgggcgtgaagtcggagtcgctagtaatcgcagatcagcaacgctgcggtgaatacgttcccgggccttgtacacaccgcccgtcacgtcatgaaagtcggtaagcacttcagcctcagcgatcattagcgtctagtcgttgcgacgccacttatgcaagggcccggaacatgtgtggcgggcagtgcagtactttcagccattcacccgaagccggtggcctaacccctcgtgggagggagccgtcgaaggtgggatcggcgattgggacgaagtcgtaacaaggtagccgtaccggaaggtggtgggcttcggccaccggattggggagcaccctccctcggcagcttccaccctagccgctaaccctgcttcagcattgttccatcggcatggccttccaccggctggatcacctcctttctgccgacctagtggaggaaaga

SEQ ID NO: 13 - DNA, 2924 bp, Rhodococcus Ralstonia
acgtggcggcatgccttacacatgcaagtcgaacggcagcgcggacttcggtctggcggcgagtggcgaacgggtgagtaatacatcggaacgtaccctgtgcaccgccgtacggaatgtgtacgttcagcttgccgtcgcgcctgaagccagaccgccgctcaccgcttgcccactcattatgtagccttgcatgggacttgtgggggataactagtcgaaagattagctaataccgcatacgacctgagggtgaaagtgggggaccgcaaggcctcacgcagcaggagcggccgatgtaacaccccctattgatcagctttctaatcgattatggcgtatgctggactcccactttcaccccctggcgttccggagtgcgtcgtcctcgccggctacactgattagctagttggtggggtaaaggcccaccaaggcgacgatcagtagctggtctgagaggacgatcagccacactgggactgagacacggcccagacgactaatcgatcaaccaccccatttccgggtggttccgctgctagtcatcgaccagactctcctgctagtcggtgtgaccctgactctgtgccgggtctgtcctacgggaggcagcagtggggaattttggacaatgggggcaaccctgatccagcaatgccgcgtgtgtgaagaaggccttcgggttgtaaagcactttaggatgccctccgtcgtcaccccttaaaacctgttacccccgttgggactaggtcgttacggcgcacacacttcttccggaagcccaacatttcgtgaaatgtccggaaagaaatcgcgctggttaatacctgcgtgatgacggtaccggaagaataagcaccggctaactacgtgccagcagccgcggtaatacgtaggacaggcctttctttagcgcgaccaattatggacgcactactgccatggccttcttattcgtggccgattgatgcacggtcgtcggcgccattatgcatccgtgcgagcgttaatcggaattactgggcgtaaagcgtgcgcaggcggttttgtaagacaggcgtgaaatccccgggcttaacctgggaattgcgcttgtgcacgctcgcaattagccttaatgacccgcatttcgcacgcgtccgccaaaacattctgtccgcactttaggggcccgaattggacccttaacgcgaacacactgcaaggctagagtgcgtcagaggggggtagaattccacgtgtagcagtgaaatgcgtagagatgtggaggaataccgatggcgaaggcgagcccccttgacgttccgatctcacgcagtctccccccatcttaaggtgcacatcgtcactttacgcatctctacacctccttatggctaccgcttccgctcgggggaggaccttgactgacgctcatgcacgaaagcgtggggagcaaacaggattagataccctggtagtccacgccctaaacgatgtcaactagttgttgggattcctggaactgactgcgagtacgtgctttcgcacccctcgtttgtcctaatctatgggaccatcaggtgcgggatttgctacagttgatcaacaaccctaacattttctcagtaacgtagctaacgcgtgaagttgaccgcctggggagtacggctgcaagattaaaactcaaaggaattgacggggacccgcacaagcgggtaaaagagtcattgcatcgattgcgcacttcaactggcggacccctcatgccgacgttctaattttgagtttccttaactgcccctgggcgtgttcgcctggatgatgtggattaattcgatgcaacgcgaaaaaccttacctacccttgacatgccctaacgaagcagagatgcattagtgcccgcaaagggaaagtgacctactacacctaattaagctacgttgcgctttttggaatggatgggaactgtacgggattgcttcgtctctacgtaatcacgggcgtttccctttcacggacacaggtgctgcatggctgtcgtcagctcgtgtcgtgagatgttgggttaagtcccgcaacgagcgcaacccttgtctctagttgcctacgcaagagcctgtgtccacgacgtaccgacagcagtcgagcacagcactctacaacccaattcagggcgttgctcgcgttgggaacagagatcaacggatgcgttctccactctagagagactgccggtgacaaaccggaggaaggtggggatgacgtcaagtcctcatggcccttatgggtagggcttcacacgtcatacaatggtggtgagatctctctgacggccactgtttggcctccttccacccctactgcagttcaggagtaccgggaatacccatcccgaagtgtgcagtatgttaccaccgtacagagggttgccaacccgcgagggggagctaatcccagaaaacgcatcgtagtccggatcgtagtctgcaactcgactacgtgaagctggaatcgcgcatgtctcccaacggttgggcgctccccctcgattagggtcttttgcgtagcatcaggcctagcatcagacgttgagctgatgcacttcgaccttagcgtagtaatcgcggatcagcatgccgcggtgaatacgttcccgggtcttgtacacaccgcccgtcacaccatgggagtgggttttgccagaagtagttagccatcattagcgcctagtcgtacggcgccacttatgcaagggcccagaacatgtgtggcgggcagtgtggtaccctcacccaaaacggtcttcatcaatcggtaaccgcaaggagggcgattaccacggcagggttcatgactggggtgaagtcgtaacaaggtattggcgttcctcccgctaatggtgccgtcccaagtactgaccccacttcagcattgttcca

SEQ ID NO: 14 - protein, 284 aa, Rhodococcus opacus
MASIEDILELEALEKDIFRGAVHPSVLKRTFGGQVAGQSLVSAVRTVDERFEVHSLHGYFLRPGNPTEPTVYLVDRIRDGRSFCTRRVTGIQDGKAIFTMSASFHSQDEGIEHQDTMPSVPEPEELVDAQTVEEMAATDLYREWKEWDVRIVPAGCTGKTPGIAAKQRVWMRYRNKLPDDQVFHICTLAYLSDMTLLGASKVPHPGVVTQTASLDHAMWFLRPFRADEWLLYDQTSPSAGFGRALTQGRMFDRKGTMVAAVVQEGLTRIQRDQDQRDIETGNMA

SEQ ID NO: 15 - protein, 87 aa, Artificial Sequence
  Synthetic Polypeptide
MSQAEFDKAAEEVKHLKTKPADEEMLFIYSHYKQATVGDINTERPGMLDFKGKAKWDAWNELKGTSKEDAMKAYIDKVEELKKKYGI

SEQ ID NO: 16 - DNA, 291 bp, Artificial Sequence
  Synthetic Polynucleotide
ggtaccgggccccccctcgagatgtcccaggccgagttcgacaaggccgccgaggaagttaagcacctcaagaccaagccggcagacgaggagatgctgttcatctactcccactacaagcaggcaaccgtgggtgacatcaacacagaacggcccggcatgctcgacttcaagggcaaggccaagtgggatgcctggaatgagctgaaagggacctccaaagaagatgccatgaaggcgtacattgacaaggtagaagaactcaagaaaaaatacggcatctaggtcgac

SEQ ID NO: 17 - protein, 533 aa, Artificial Sequence
  Synthetic Polypeptide
MFQFHAGSWESWCCCCCLIPGDRPWDRGRRWRLEMRHTRSVHETRFEAAVKVIQSLPKNGSFQPTNEMMLKFYSFYKQATEGPCKLSKPGFWDPVGRYKWDAWSSLGDMTKEEAMIAYVEEMKKILETMPMTEKVEELLHVIGPFYEIVEDKKSGRSSDLTSVRLEKISKCLEDLGNVLASTPNAKTVNGKAESSDSGAESEEEAAQEDPKRPEPRDSDKKMMKKSADHKNLEIIVTNGYDKDSFVQGVQNSIHTSPSLNGRCTEEVKSVDENLEQTGKTVVFVHQDVNSDHVEDISGIQHLTSDSDSEVYCDSMEQFGQEESLDGFISNNGPFSYYLGGNPSQPLESSGFPEAVQGLPGNGSPEDMQGAVVEGKGEVKRGGEDGGSNSGAPHREKRAGESEEFSNIRRGRGHRMQHLSEGSKGRQVGSGGDGERWGSDRGSRGSLNEQIALVLMRLQEDMQNVLQRLHKLEMLAASQAKSSALQTSNQPTSPRPSWWPFEMSPGALTFAIIWPFIAQWLVHLYYQRRRRKLN

SEQ ID NO: 18 - DNA, 1750 bp, Artificial Sequence
  Synthetic Polynucleotide
gaggagctgaccagctgcgctttggagtcctcctcccttcgggaatgttgatccgcggctgcgctccatgtttcagtttcatgcaggctcctgggaaagctggtgctgctgctgctgcctgattccaggcgacagaccttgggaccgcggccggcgctggcggctggagatgcggcacacgagatccgttcacgaaacccggtttgaggcggctgtgaaggtgatacagagcttgccgaaaaatggttcattccagccaacaaatgaaatgatgctcaagttctatagcttctataagcaggcaactgaaggaccttgtaaactgtcaaagcctggcttctgggatcctgttggaagatacaaatgggatgcgtggagttctttgggtgatatgaccaaagaggaagccatgattgcttatgttgaagaaatgaaaaagattcttgaaactatgccgatgactgaaaaagttgaagaattgctacatgtcattggtccattttatgaaattgtagaagacaaaaaaagtggcagaagttctgatttaacctcagtccgactggagaaaatctctaaatgcttagaagatcttggtaatgttctagcttctactccaaatgccaaaactgttaatggtaaagctgaaagcagtgatagtggagctgaatctgaggaagaagcagcccaagaagacccgaaaagaccagaaccacgtgatagcgataagaaaatgatgaagaaatctgcagaccataagaatttggaaatcattgtcactaatggctatgataaagacagctttgtgcagggcgtacagaatagcattcataccagtccttccctgaatggccgatgcactgaggaagtaaaatctgtagatgaaaacttggagcaaactggaaaaactgttgtcttcgttcaccaagatgtaaacagtgatcatgttgaagatatttcaggaattcagcatttgacaagtgattcagacagtgaagtttactgtgattccatggagcaatttgggcaagaagagtctttagacggctttatatcaaacaatggaccattttcctattacttgggtggtaatcccagtcaaccgttggaaagttctggttttcctgaagctgttcaaggacttcctgggaacggcagccctgaggacatgcagggcgcagtggttgaaggcaaaggtgaagtaaagcgtgggggagaggacggcgggagtaacagtggagccccgcaccgcgagaaacgggctggagaaagtgaggagttctctaacattaggagagggagagggcacaggatgcagcatttgagtgaaggaagcaagggtcggcaagtgggaagtggaggtgatggggaacgctggggttcggacagaggctcaaggggcagcctgaacgagcagatcgcgcttgtgctcatgcgcctgcaggaggacatgcagaacgtcctccagagactccacaaactggagatgctggcggcatcacaggcaaaatcatcagcattacagaccagtaatcagcccacttcaccgagaccatcttggtggcccttcgagatgtctcctggtgcattaaccttcgctatcatatggccttttattgctcagtggttggtgcatttatattaccaaagaaggagaagaaaattgaactaaagaaaatgacattttgttgaagaaatctactggccctggataacctcgggatgataccaattgtggagcttacacgaggga

SEQ ID NO: 19 - DNA, 608 bp, Artificial Sequence
  Synthetic Polynucleotide
gagcaccggtggagaggcctaaggttgcgcttctaaaatcgctgccagttgagtctcttgtgctgctgctaccttctcttcgccgcctccgcgggcttcctggaatctttgcaacaccgccggcatgtctcaggctgagtttgacaaagctgctgaggaagttaagcatcttaagaccaagccagcagatgaggagatgctgttcatctacagccactacaaacaagcaactgtgggtgacataaatacagaacgtcctggaatgttggacttcaaaggcaaggccaagtgggatgcctggaatgagctgaaagggacttctaaagaagatgccatgaaagcttacattgacaaagtagaagaactaaagaaaaaatatggaatataagagactgagtttggctgccagccattcatttcacctaaactgatttaatgccttgtttttctaatactggggatgaagttcataaataactagctaagccagaagctcaagacagcccaggatatgactaacagattaggagctgaaacggttactaatccttgctgagtaatttttatcagtagatgaattaaaagtatctttgttactttacttcgat

SEQ ID NO: 20 - DNA, 1481 bp, Rhodococcus opacus
  misc_feature (570)..(570): n is a, c, g, or t
cctggctcaggacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggccaactacgtgccagcagccgcggtaatacgtagggtgcaagcgttgtccggaattactgggcgtaaagagctcgtaggcggtttgtcgcgtcgtctgtgaaaactcanagctcaacctcgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtgggtagcaaacaggattagataccctggtagtccacgccgtaaacggtgggcgctaggtgtgggtttccttccacgggatccgtgccgtagttaacgcattaagcgccccgcctggggagtacggccgcaaggttaaaactcaaaggaattgacgggggcccgcacaagcggcggagcatgtggattaattcgatgcaacgcgaagaaccttacctgggtttgacatataccggaaagccgtagagataccgccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccggaagg

SEQ ID NO: 21 - DNA, 1460 bp, Rhodococcus opacus
  misc_feature (799)..(799): n is a, c, g, or t
ggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggccaactacgtgccagcagccgcggtaatacgtagggtgcaagcgttgtccggaattactgggcgtaaagagctcgtaggcggtttgtcgcgtcgtctgtgaaaactcacagctcaacctcgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtgggtagcaaacaggattagataccctggtagtccacgccgtaaacggtgggcgctaggtgtgggtttccttccacgggatccgtgccgtagntaacgcattaagcgccccgcctggggagtacggccgcaaggttaaaactcaaaggaattgacgggggcccgcacaagcggcggagcatgtggattaattcgatgcaacgcgaagaaccttacctgggtttgacatataccggaaagccgtagagataccgccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccggaag

SEQ ID NO: 22 - DNA, 1473 bp, Rhodococcus opacus
  misc_feature (562)..(562): n is a, c, g, or t
aggacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgggcccgcggcctatcagcttgttggtggggtaatggcctaccaagccgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggccaactacgtgccagcagccgcggtaatacgtagggtgcaagcgttgtccggaattactgggcgtaaagagctcgtaggcggtttgtcgcgtcgtctgtgaaaactcanagctcaacctcgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtgggtagcaaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccggaagg

SEQ ID NO: 23 - DNA, 1462 bp, Cupriavidus necator
acgtggcggcatgccttacacatgcaagtcgaacggcagcgcggacttcggtctggcggcgagtggcgaacgggtgagtaatacatcggaacgtaccctgttgtgggggataactagtcgaaagattagctaataccgcatacgacctgagggtgaaagtgggggaccgcaaggcctcacgcagcaggagcggccgatgtctgattagctagttggtggggtaaaggcccaccaaggcgacgatcagtagctggtctgagaggacgatcagccacactgggactgagacacggcccagactcctacgggaggcagcagtggggaattttggacaatgggggcaaccctgatccagcaatgccgcgtgtgtgaagaaggccttcgggttgtaaagcacttttgtccggaaagaaatcgcgctggttaatacctgcgtgatgacggtaccggaagaataagcaccggctaactacgtgccagcagccgcggtaatacgtagggtgcgagcgttaatcggaattactgggcgtaaagcgtgcgcaggcggttttgtaagacaggcgtgaaatccccgggcttaacctgggaattgcgcttgtgactgcaaggctagagtgcgtcagaggggggtagaattccacgtgtagcagtgaaatgcgtagagatgtggaggaataccgatggcgaaggcgagccccctggaccttgactgacgctcatgcacgaaagcgtggggagcaaacaggattagataccctggtagtccacgccctaaacgatgtcaactagttgttgggattcattttctcagtaacgtagctaacgcgtgaagttgaccgcctggggagtacggctgcaagattaaaactcaaaggaattgacggggacccgcacaagcggtggatgatgtggattaattcgatgcaacgcgaaaaaccttacctacccttgacatgccctaacgaagcagagatgcattagtgcccgcaaagggaaagtgggacacaggtgctgcatggctgtcgtcagctcgtgtcgtgagatgttgggttaagtcccgcaacgagcgcaacccttgtctctagttgcctacgcaagagcactctagagagactgccggtgacaaaccggaggaaggtggggatgacgtcaagtcctcatggcccttatgggtagggcttcacacgtcatacaatggtgcgtacagagggttgccaacccgcgagggggagctaatcccagaaaacgcatcgtagtccggatcgtagtctgcaactcgactacgtgaagctggaatcgctagtaatcgcggatcagcatgccgcggtgaatacgttcccgggtcttgtacacaccgcccgtcacaccatgggagtgggttttgccagaagtagttagcctaaccgcaaggagggcgattaccacggcagggttcatgactggggtgaagtcgtaacaaggt

SEQ ID NO: 24 - DNA, 1479 bp, Ralstonia sp.
agtttgatcctggctcagattgaacgctggcggcatgccttacacatgcaagtcgaacggcagcgcggacttcggtctggcggcgagtggcgaacgggtgagtaatacatcggaacgtaccctgttgtgggggataactagtcgaaagattagctaataccgcatacgacctgagggtgaaagcgggggaccgtaaggcctcgcgcagcaggagcggccgatgtctgattagctagttggtggggtaaaggcccaccaaggcgacgatcagtagctggtctgagaggacgatcagccacactgggactgagacacggcccagactcctacgggaggcagcagtggggaattttggacaatgggggcaaccctgatccagcaatgccgcgtgtgtgaagaaggccttcgggttgtaaagcacttttgtccggaaagaaaacgctctggttaatacctggagtggatgacggtaccggaagaataagcaccggctaactacgtgccagcagccgcggtaatacgtagggtgcgagcgttaatcggaattactgggcgtaaagcgtgcgcaggcggttttgtaagacaggcgtgaaatccccgagctcaacttgggaattgcgcttgtgactgcaaggctagagtatgtcagaggggggtagaattccacgtgtagcagtgaaatgcgtagagatgtggaggaataccgatggcgaaggcagccccctgggacgtcactgacgctcatgcacgaaagcgtggggagcaaacaggattagataccctggtagtccacgccctaaacgatgtcaactagttgttggggattcatttcttcagtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgccactaacgaagcagagatgcatcaggtgcccgaaagggaaagtggacacaggtgctgcatggctgtcgtcagctcgtgtcgtgagatgttgggttaagtcccgcaacgagcgcaacccttatctttagttgctacgcaagggcactctagagagactgccggtgacaaaccggaggaaggtggggatgacgtcaagtcctcatggcccttatgggtagggcttcacacgtcatacaatggtgcgtacagagggttgccaacccgcgagggggagctaatcccagaaaacgcatcgtagtccggatcgcagtctgcaactcgactgcgtgaagctggaatcgctagtaatcgcggatcagcatgccgcggtgaatacgttcccgggtcttgtacacaccgcccgtcacaccatgggagtgggttttgccagaagtagttagcctaaccgcaaggagggcgattaccacggcagggttcatgactggggtgaagtcgt

SEQ ID NO: 25 - DNA, 1486 bp, Gordonia alkanivorans
gctcaggacgaacgctggcggcgtgcttaacacatgcaagtcgaacggaaaggcccagcttgctgggtactcgagtggcgaacgggtgagtaacacgtgggtgatctgccctgaactttgggataagcctgggaaactgggtctaataccggatatgaccttggagtgcatgctctggggtggaaagcttttgcggttcaggatgggcccgcggcctatcagcttgttggtggggtaatggcctaccaaggcgacgacgggtagccgacctgagagggtgatcggccacactgggactgagacacggcccagactcctacgggaggcagcagtggggaatattgcacaatgggcgcaagcctgatgcagcgacgccgcgtgagggatgacggccttcgggttgtaaacctctttcaccagggacgaagcgcaagtgacggtacctggagaagaagcaccggccaactacgtgccagcagccgcggtaatacgtagggtgcgagcgttgtccggaattactgggcgtaaagagctcgtaggcggtttgtcgcgtcgtctgtgaaattctgcaactcaattgtaggcgtgcaggcgatacgggcagacttgagtactacaggggagactggaattcctggtgtagcggtgaaatgcgcagatatcaggaggaacaccggtggcgaaggcgggtctctgggtagtaactgacgctgaggagcgaaagcgtgggtagcgaacaggattagataccctggtagtccacgccgtaaacggtgggtactaggtgtggggctcatttcacgagttccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatacaccagacgcatgtagagatacatgttcccttgtggttggtgtacaggtggtgcatggctgtcgtcagctcgtgtcgtgagatgttgggttaagtcccgcaacgagcgcaacccttgtcctgtattgccagcgggttatgccggggacttgcaggagactgccggggtcaactcggaggaaggtggggatgacgtcaagtcatcatgccccttatgtccagggcttcacacatgctacaatggctggtacagagggctgcgataccgtgaggtggagcgaatcccttaaagccagtctcagttcggattggggtctgcaactcgaccccatgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagctgtcgaaggtgggatcggcgattgggacgaagtcgtaacaaggtagccgtaccggaaggtgcgg

SEQ ID NO: 26 - DNA, 1491 bp, Gordonia sp
gatcatggctcaggacgaacgctggcggcgtgcttaacacatgcaagtcgaacggaaaggcccgcttgcgggtactcgagtggcgaacgggtgagtaacacgtgggtgatctgccctggactctgggataagcctgggaaactgggtctaataccggatatgaccttacatcgcatggtgtttggtggaaagcttttgcggttcaggatgggcccgcggcctatcagcttgttggtggggtaatggcctaccaaggcgacgacgggtagccgacctgagagggtgatcggccacactgggactgagacacggcccagactcctacgggaggcagcagtggggaatattgcacaatgggcgcaagcctgatgcagcgacgccgcgtgagggatgacggccttcgggttgtaaacctctttcaccagggacgaagcgcaagtgacggtacctggagaagaagcaccggccaactacgtgccagcagccgcggtaatacgtagggtgcgagcgttgtccggaattactgggcgtaaagagctcgtaggcggtttgtcgcgtcgtctgtgaaattctgcaactcaattgtaggcgtgcaggcgatacgggcagacttgagtactacaggggagactggaattcctggtgtagcggtgaaatgcgcagatatcaggaggaacaccggtggcgaaggcgggtctctgggtagtaactgacgctgaggagcgaaagcgtgggtagcgaacaggattagataccctggtagtccacgccgtaaacggtgggtactaggtgtggggctcatttcacgagttccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatacaccagaaagctatagagatatagccccccttgtggttggtgtacaggtggtgcatggctgtcgtcagctcgtgtcgtgagatgttgggttaagtcccgcaacgagcgcaacccttgtcctgtattgccagcgggttatgccggggacttgcaggagactgccggggtcaactcggaggaaggtggggatgacgtcaagtcatcatgccccttatgtccagggcttcacacatgctacaatggctggtacagagggctgcgataccgtgaggtggagcgaatcccttaaagccagtctcagttcggattggggtctgcaactcgaccccatgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagctgtcgaaggtgggatcggcgattgggacgaagtcgtaacaaggtagccgtaccggaaggtgcgg

SEQ ID NO: 27 - DNA, 1505 bp, Mycobacterium fortuitum
ttgatcctggctcaggacgaacgctggcggcgtgcttaacacatgcaagtcgaacggaaaggcccttcggggtactcgagtggcgaacgggtgagtaacacgtgggtgatctgccctgcactttgggataagcctgggaaactgggtctaataccgaatatgaccacgcgcttcatggtgtgtggtggaaagcttttgcggtgtgggatgggcccgcggcctatcagcttgttggtggggtaatggcctaccaaggcgacgacgggtagccggcctgagagggtgaccggccacactgggactgagatacggcccagactcctacgggaggcagcagtggggaatattgcacaatgggcgcaagcctgatgcagcgacgccgcgtgagggatgacggccttcgggttgtaaacctctttcaatagggacgaagcgcaagtgacggtacctatagaagaaggaccggccaactacgtgccagcagccgcggtaatacgtagggtccgagcgttgtccggaattactgggcgtaaagagctcgtaggtggtttgtcgcgttgttcgtgaaaactcacagcttaactgtgggcgtgcgggcgatacgggcagactagagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttgggatccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatgcacaggacgactgcagagatgtggtttcccttgtggcctgtgtgcaggtggtgcatggctgtcgtcagctcgtgtcgtgagatgttgggttaagtcccgcaacgagcgcaacccttgtctcatgttgccagcacgttatggtggggactcgtgagagactgccggggtcaactcggaggaaggtggggatgacgtcaagtcatcatgccccttatgtccagggcttcacacatgctacaatggccggtacaaagggctgcgatgccgtgaggtggagcgaatcctttcaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaacccttgtggagggagccgtcgaaggtgggatcggcgattgggacgaagtcgtaacaaggtagccgtaccggaaggtgcggctggatcacctcctt

SEQ ID NO: 28 - DNA, 1460 bp, Mycobacterium parafortuitum
cgaacgctggcggcgtgcttaacacatgcaagtcgaacggaaaggcccttcggggtactcgagtggcgaacgggtgagtaacacgtgggtgatctgccctgcactttgggataagcctgggaaactgggtctaataccgaatatgatcattggcttcctggctggtggtggaaagcttttgcggtgtgggatgggcccgcggcctatcagcttgttggtggggtaatggcctaccaaggcgacgacgggtagccggcctgagagggtgaccggccacactgggactgagatacggcccagactcctacgggaggcagcagtggggaatattgcacaatgggcgcaagcctgatgcagcgacgccgcgtgagggatgacggccttcgggttgtaaacctctttcgccagggacgaagcgcaagtgacggtacctggagaagaaggaccggccaactacgtgccagcagccgcggtaatacgtagggtccgagcgttgtccggaattactgggcgtaaagagctcgtaggtggtttgtcgcgttgttcgtgaaaactcacagcttaactgtgggcgtgcgggcgatacgggcagactagagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttgggatccgtgccgtagctaacgcattaagtaccccgcctggggagtacggccgcaaggctaaaactcaaagaaattgacgggggcccgcacaagcggcggagcatgtggattaattcgatgcaacgcgaagaaccttacctgggtttgacatgcacaggacgccggcagagatgtcggttcccttgtggcctgtgtgcaggtggtgcatggctgtcgtcagctcgtgtcgtgagatgttgggttaagtcccgcaacgagcgcaacccttgtctcatgttgccagcacgtaatggtggggactcgtgagagactgccggggtcaactcggaggaaggtggggatgacgtcaagtcatcatgccccttatgtccagggcttcacacatgctacaatggccggtacaaagggctgcgatgccgtgaggtggagcgaatcctttcaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccg

SEQ ID NO: 29 - DNA, 1480 bp, Mycobacterium sphagni
gagtttgatcctggctcaggacgaacgctggcggcgtgcttaacacatgcaagtcgaacggaaaggcccttcggggtactcgagtggcgaacgggtgagtaacacgtgggtgatctgccctgcactttgggataagcctgggaaactgggtctaataccgaataggaccgcatgcttcatggtgtgtggtggaaagcttttgcggtgtgggatgggcccgcggcctatcagcttgttggtggggtaatggcctaccaaggcgacgacgggtagccggcctgagagggtgtccggccacactgggactgagatacggcccagactcctacgggaggcagcagtggggaatattgcacaatgggcgcaagcctgatgcagcgacgccgcgtgagggatgacggccttcgggttgtaaacctctttcagcagggacgaagcgcaagtgacggtacctgtagaagaagcaccggccaactacgtgccagcagccgcggtaatacgtagggtgcgagcgttgtccggaattactgggcgtaaagagctcgtaggtggtttgtcgcgttgttcgtgaaaactcacagctcaactgtgggcgtgcgggcgatacgggcagacttgagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttgggatccgtgccgtagctaacgcattaagtaccccgcctggggagtacggccgcaaggctaaaactcaaagaaattgacgggggcccgcacaagcggcggagcatgtggattaattcgatgcaacgcgaagaaccttacctgggtttgacatgcacaggacgccggcagagatgtcggttcccttgtggcctgtgtgcaggtggtgcatggctgtcgtcagctcgtgtcgtgagatgttgggttaagtcccgcaacgagcgcaacccttgtctcatgttgccagcacgtaatggtggggactcgtgagagactgccggggtcaactcggaggaaggtggggatgacgtcaagtcatcatgccccttatgtccagggcttcacacatgctacaatggccggtacaaagggctgcgatgccgtgaggtggagcgaatcctttcaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagcc

SEQ ID NO: 30 - DNA, 1494 bp, Nocardia farcinica
gacgaacgctggcggcgtgcttaacacatgcaagtcgagcggtaaggcccttcggggtacacgagcggcgaacgggtgagtaacacgtgggtgatctgccctgtacttcgggataagcctgggaaactgggtctaataccggatatgaccttacatcgcatggtgtttggtggaaagatttatcggtacaggatgggcccgcggcctatcagcttgttggtggggtaatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcgacagggacgaagcgcaagtgacggtacctgtagaagaagcaccggccaactacgtgccagcagccgcggtaatacgtagggtgcgagcgttgtccggaattactgggcgtaaagagcttgtaggcggtttgtcgcgtcgtccgtgaaaacttggggctcaaccccaagcttgcgggcgatacgggcagacttgagtactgcaggggagactggaattcctggtgtagcggtgaaatgcgcagatatcaggaggaacaccggtggcgaaggcgggtctctgggcagtaactgacgctgagaagcgaaagcgtgggtagcgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatacaccggaaacctgcagagatgtaggcccccttgtggtcggtgtacaggtggtgcatggctgtcgtcagctcgtgtcgtgagatgttgggttaagtcccgcaacgagcgcaacccttgtcctgtgttgccagcgcgttatggcggggactcgcaggagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagttggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccggaaggtgcggctggatcacctcctttct

SEQ ID NO: 31 - DNA, 1513 bp, Nocardia sp.
gagtttgatcctggctcaggacgaacgctggcggcgtgcttaacacatgcaagtcgagcggtaaggcccttcggggtacacgagcggcgaacgggtgagtaacacgtgggtgatctgccctgtacttcgggataagcctgggaaactgggtctaataccggatatgaccttacatcgcatggtgtttggtggaaagatttatcggtacaggatgggcccgcggcctatcagcttgttggtggggtaatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcgacagggacgaagcgcaagtgacggtacctgtagaagaagcaccggccaactacgtgccagcagccgcggtaatacgtagggtgcgagcgttgtccggaattactgggcgtaaagagcttgtaggcggtttgtcgcgtcgtccgtgaaaacttggggctcaaccccaagcttgcgggcgatacgggcagacttgagtactgcaggggagactggaattcctggtgtagcggtgaaatgcgcagatatcaggaggaacaccggtggcgaaggcgggtctctgggcagtaaccgacgctgagaagcgaaagcgtgggtagcgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatacaccggaaacctgcagagatgtaggcccccttgtggtcggtgtacaggtggtgcatggccgtcgtcagctcgtgtcgtgagatgttgggttaagtcccgcaacgagcgcaacccttgtcctgtgttgccagcgcgttatggcggggactcgcaggagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagttggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccggaaggtgcggctggatcacctcctttct

SEQ ID NO: 32 - DNA, 1490 bp, Rhodococcus rhodochrous
gagtttgaatctggctcaggacgaacgctggcggcgtgcttaacacatgcaagtcgaacgatgaagcccagcttgctgggtggattagtggcgaacgggtgagtaacacgtgggtgatctgccctgcactctgggataagcctgggaaactgggtctaataccggatatgacctcttgctgcatggcgaggggtggaaagtttttcggtgcaggatgagcccgcggcctatcagcttgttggtggggtaatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggccaactacgtgccagcagccgcggtaatacgtagggtgcgagcgttgtccggaattactgggcgtaaagagctcgtaggcggtttgtcgcgtcgtctgtgaaatcccgcagctcaactgcgggcttgcaggcgatacgggcagactcgagtactgcaggggagactggaattcctggtgtagcggtgaaatgcgcagatatcaggaggaacaccggtggcgaaggcgggtctctgggcagtaactgacgctgaggagcgaaagcgtgggtagcgaacaggattagataccctggtagtccacgccgtaaacggtgggcgctaggtgtgggtttccttccacgggatccgtgccgtagccaacgcattaagcgccccgcctggggagtacggccgcaaggctaaaactcaaaggaattgacgggggcccgcacaagcggcggagcatgtggattaattcgatgcaacgcgaagaaccttacctgggtttgacatgtaccggacgactgcagagatgtggtttcccttgtggccggtagacaggtggtgcatggctgtcgtcagctcgtgtcgtgagatgttgggttaagtcccgcaacgagcgcaacccttgtcctgtgttgccagcacgtaatggtggggactcgcaggagactgccggggtcaactcggaggaaggtggggacgacgtcaagtcatcatgccccttatgtccagggcttcacacatgctacaatggtcggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccgga

SEQ ID NO: 33 - DNA, 1481 bp, Rhodococcus coprophilus
cctggctcaggacgaacgctggcggcgtgcttaacacatgcaagtcgaacgatgatgcccagcttgctgggcggattagtggcgaacgggtgagtaacacgtgggtgatctgccctgcacttcgggataagcctgggaaactgggtctaataccggatatgaccatgggatgcatgtcctgtggtggaaaggtttactggtgcaggatgagcccgcggcctatcagcttgttggtggggtaatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgcaagtgactgtacctgcagaagaagcaccggctaactacgtgccagcagccgcggtaatacgtagggtgcgagcgttgtccggaattactgggcgtaaagagttcgtaggcggtttgtcgcgtcgtgtgtgaaatcccgcagctcaactgcgggcttgcaggcgatacgggcagacttgagtactgcaggggagactggaattcctggtgtagcggtgaaatgcgcagatatcaggaggaacaccggtggcgaaggcgggtctctgggcagtaactgacgctgaggaacgaaagcgtgggtagcgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggacgactgcagagatgtggtttcccttgtggtcggtatacaggtggtgcatggctgtcgtcagctcgtgtcgtgagatgttgggttaagtcccgcaacgagcgcaacccttgtcttatgttgccagcacgtaatgggggggactcgtaagagactgccggggtcaactcggaggaaggtggggacgacgtcaagtcatcatgccccttatgtccagggcttcacacatgctacaatggtcggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccgg

SEQ ID NO: 34 - DNA, 1486 bp, Rhodococcus triatomae
ggcggcgtgcttaacacatgcaagtcgagcggtaaggcctttcggggtacacgagcggcgaacgggtgagtaacacgtgggtgatctgccctgcactctgggataagcctgggaaactgggtctaataccggatatgactaccggctgcatggtctggtggtggaaagatttatcggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcaacagggacgaagcgcaagtgacggtacctgtagaagaagcaccggccaactacgtgccagcagccgcggtaatacgtagggtgcgagcgttgtccggaattactgggcgtaaagagctcgtaggcggtttgtcgcgtcgtctgtgaaaaccagcagctcaactgctggcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtgggtagcgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatacaccggaaagccgtagagatacggccccccttgtggtcggtgtacaggtggtgcatggctgtcgtcagctcgtgtcgtgagatgttgggttaagtcccgcaacgagcgcaacccttgtcctgtgttgccagcacgtaatggtggggactcgcaggagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccggaaggtgcggctggatcacttcctttcta

SEQ ID NO: 35 - DNA, 1507 bp, Nocardia coeliaca
tttgatcctggctcaggacgaacgctggcggcgtgcttaacacatgcaagtcgagcggtaaggcctttcggggtacacgagcggcgaacgggtgagtaacacgtgggtgatctgccctgcacttcgggataagcctgggaaactgggtctaataccggatatgacctcaggttgcatgacttggggtggaaagatttatcggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgcaagtgacggtacctgcagaagaagcaccggctaactacgtgccagcagccgcggtaatacgtagggtgcaagcgttgtccggaattactgggcgtaaagagttcgtaggcggtttgtcgcgtcgtttgtgaaaaccagcagctcaactgctggcttgcaggcgatacgggcagacttgagtactgcaggggagactggaattcctggtgtagcggtgaaatgcgcagatatcaggaggaacaccggtggcgaaggcgggtctctgggcagtaactgacgctgaggaacgaaagcgtgggtagcgaacaggattagataccctggtagtccacgccgtaaacggtgggcgctaggtgtgggttccttccacggaatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagctgcagagatgtggccccccttgtggtcggtatacaggtggtgcatggctgtcgtcagctcgtgtcgtgagatgttgggttaagtcccgcaacgagcgcaacccctatcttatgttgccagcacgttatggtggggactcgtaagagactgccggggtcaactcggaggaaggtggggacgacgtcaagtcatcatgccccttatgtccagggcttcacacatgctacaatggccagtacagagggctgcgagaccgtgaggtggagcgaatcccttaaagctggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcttaaccccttgtgggagggagccgtcgaaggtgggatcggcgattgggacgaagtcgtaacaaggtagccgtaccggaaggtgcggctggatcacctccttt

SEQ ID NO: 36 - DNA, 1507 bp, Nocardia globerula
gtttgatcctggctcaggacgaacgctggcggcgtgcttaacacatgcaagtcgagcggtaaggcctttcggggtacacgagcggcgaacgggtgagtaacacgtgggtgatctgccctgcacttcgggataagcctgggaaactgggtctaataccggatatgacctcctatcgcatggtgggtggtggaaagatttatcggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggacgacggccttcgggttgtaaacctctttcagcagggacgaagcgcaagtgacggtacctgcagaagaagcaccggctaactacgtgccagcagccgcggtaatacgtagggtgcaagcgttgtccggaattactgggcgtaaagagttcgtaggcggtttgtcacgtcgtttgtgaaaactcacagctcaactgtgagcctgcaggcgatacgggcagacttgagtactgcaggggagactggaattcctggtgtagcggtgaaatgcgcagatatcaggaggaacaccggtggcgaaggcgggtctctgggcagtaactgacgctgaggaacgaaagcgtgggtagcgaacaggattagataccctggtagtccacgccgtaaacggtgggcgctaggtgtgggttccttccacggaatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccctatcttatgttgccagcacgttatggtggggactcgtaagagactgccggggtcaactcggaggaaggtggggacgacgtcaagtcatcatgccccttatgtccagggcttcacacatgctacaatggccagtacagagggctgcgagaccgtgaggtggagcgaatcccttaaagctggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcttaaccccttgtgggagggagccgtcgaaggtgggatcggcgattgggacgaagtcgtaacaaggtagccgtaccggaaggtgcggctggatcacctcctt

SEQ ID NO: 37 - DNA, 1508 bp, Rhodococcus equi
gagtttgatcctggctcaggacgaacgctggcggcgtgcttaacacatgcaagtcgagcggtagggcccttcggggtacacgagcggcgaacgggtgagtaacacgtgggtgatctgccctgcacttcgggataagcttgggaaactgggtctaataccggatatgagcctctactgcatggtggaggttggaaaggtttactggtgcaggatgggcccgcggcctatcagcttgttggtggggtaatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgagagtgacggtacctgcagaagaagcaccggccaactacgtgccagcagccgcggtaatacgtagggtgcgagcgttgtccggaattactgggcgtaaagagctcgtaggcggtttgtcgcgtcgtcggtgaaaaccagcagctcaactgctggcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtgggtagcgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcctgtgttgccagcacgtaatggtggggactcgcaggagaccgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaacccttgtggagggagccgtcgaaggtgggatcggcgattgggacgaagtcgtaacaaggtagccgtaccggaaggtgcggctggatcacctcctt

SEQ ID NO: 38 - DNA, 1442 bp, Rhodococcus sp.
ctggctcaggacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgagcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggctaactacgtgccagcagccgcggtaatacgtagggtgcaagcgttgtccggaattactgggcgtaaagagttcgtaggcggtttgtcgcgtcgtctgtgaaaactcacagctcaactgtgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggaacgaaagcgtgggtagcaaacaggattagataccctggtagtccacgccgtaaacggtgggcgctaggtgtgggttccttccacgggatctgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccagtacagagggctgcgaaccgtgaggtggagcgaatcccttaaagcyggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgatt

SEQ ID NO: 39 - DNA, 1474 bp, Rhodococcus sp.
agagtttgatcctggctcaggacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggctaactacgtgccagcagccgcggtaatacgtagggtgcaagcgttgtccggaattactgggcgtaaagagttcgtaggcggtttgtcgcgtcgtttgtgaaaactcamrgctcaactgtgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggaacgaaagcgtgggtagcaaacaggattagataccctggtagtccacgccgtaaacggtgggcgctaggtgtgggttccttccacgggatctgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccagtacagagggctgcgagaccgtgaggtggagcgaatcccttaaagctggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaagg

SEQ ID NO: 40 - DNA, 1437 bp, Rhodococcus jostii
aggacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggctaactacgtgccagcagccgcggtaatacgtagggtgcaagcgttgtccggaattactgggcgtaaagagttcgtaggcggtttgtcgcgtcgtttgtgaaaactcacagctcaactgtgagcctgcaggcgatacgggcagacttgagtactgcaggggagactggaattcctggtgtagcggtgaaatgcgcagatatcaggaggaacaccggtggcgaaggcgggtctctgggcagtaactgacgctgaggaacgaaagcgtgggtagcaaacaggattagataccctggtagtccacgccgtaaacggtgggcgctaggtgtgggttccttccacgggatctgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccagtacagagggctgcgagaccgtgaggtggagcgaatcccttaaagctggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattg

SEQ ID NO: 41 - DNA, 1485 bp, Rhodococcus opacus
  misc_feature (812)..(812): n is a, c, g, or t
gatcctggctcaggacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggccaactacgtgccagcagccgcggtaatacgtagggtgcaagcgttgtccggaattactgggcgtaaagagttcgtaggcggtttgtcgcgtcgtctgtgaaaactcaaagctcaacctcgagcctgcaggcgatacgggcagacttgagtactgcaggggagactggaattcctggtgtagcggtgaaatgcgcagatatcaggaggaacaccggtggcgaaggcgggtctctgggcagtaactgacgctgaggaacgaaagcgtgggtagcgaacaggattagataccctggtagtccacgccgtaaacggtgggcgctaggtgtgggtttccttccacgggatcngtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccggtacagagggctgcgataccgtgaggtggagcgaatcccttaaagctggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccggaaggt

SEQ ID NO: 42 - DNA, 1510 bp, Rhodococcus imtechensis
ttgatcctggctcaggacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggccaactacgtgccatcagccgcggtaatacgtagggtgcaagcgttgtccggaattactgggcgtaaagagctcgtaggcggtttgtcgtgtcgtctgtgaaaactcgaggctcaacctcgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtggaaaccgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtaagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccagtacagagggctgcgagaccgtgaggtggagcgaatcccttaaagctggtctcagttcggatcggggtctgcaactcgaccccgtgaagtcggagtcgctagtaatcgcagatcagcaacgctgcggtgaatacgttcccaggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccggaaggtgcggctggaaactgccgaggggg

SEQ ID NO: 43 - DNA, 1473 bp, Rhodococcus koreensis
gacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccaaggactgcatggtttttggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgagagtgacggtacctgcagaagaagcaccggccaactacgtgccagcagccgcggtaatacgtagggtgcaagcgttgtccggaattactgggcgtaaagagctcgtaggcggtttgtcgcgtcgtctgtgaaaactcgaggctcaacctcgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtgggtagcgaacaggattagataccctggtagtccacgccgtaaacggtgggcgctaggtgtgggttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccagtacagagggctgcgagaccgtgaggtggagcgaatcccttaaagctggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccggaaggtgc

SEQ ID NO: 44 - DNA, 1229 bp, Rhodococcus opacus
gggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggccaactacgtgccagcagccgcggtaatacgtagggtgcaagcgttgtccggaattactgggcgtaaagagctcgtaggcggtttgtcgcgtcgtctgtgaaaactcgaggctcaacctcgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtgggtagcgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagctgcagagatgtggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaacccctcgtgggagggagccgtcgaaggtgggatcggcgattgggacgaagtcgtaacaaggtagccgtaccggaaggtgcggctggatcacctcctttct

SEQ ID NO: 45 - DNA, 1450 bp, Rhodococcus sp.
tcctggctcaggacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgttggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgagagtgacggtacctgcagaagaagcaccggccaactacgtgccagcagccgcggtaatacgtagggtgcaagcgttgtccggaattactgggcgtaaagagctcgtaggcggtttgtcgcgtcgtctgtgaaaactcgaggctcaacctcgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtgggtagcgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaacccctcgtgggagggagccgtcgaaggtgggatcggcgattggga

SEQ ID NO: 46 - DNA, 1471 bp, Rhodococcus opacus
gacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgagagtgacggtacctgcagaagaagcaccggccaactacgtgccagcagccgcggtaatacgtagggtgcaagcgttgtccggaattactgggcgtaaagagctcgtaggcggtttgtcgcgtcgtctgtgaaaactcgaggctcaacctcgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtgggtagcgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaacccctcgtgggagggagccgtcgaaggtgggatcggcgattgggacgaagtcgtaacaaggtagccgtaccggaagg

SEQ ID NO: 47 - DNA, 1482 bp, Rhodococcus sp.
gagtttgatcctggctcaggacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacgaccttcgggttgtaaacctctttcagcagggacgaagcgaaagtgacggtacctgcagaagaagcaccggccaactacgtgccagcagccgcggtaatacgtagggtgcaagcgttgtccggaattactgggcgtaaagagctcgtaggcggtttgtcgcgtcgtctgtgaaaactcgaggctcaacctcgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtgggtagcgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgta

SEQ ID NO: 48 - DNA, 1446 bp, Rhodococcus sp.
gcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggccaactacgtgccagcagccgcggtaatacgtagggtgcaagcgttgtccggaattactgggcgtaaagagctcgtaggcggtttgtcgcgtcgtctgtgaaaactcgaggctcaacctcgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggggcgaaagcgtgggtagcgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccagtggcctaaccccttgtgggagggagccgtcgaaggtgggatcggcgattgggacgaagtcgtaacaaggta

SEQ ID NO: 49 - DNA, 1497 bp, Rhodococcus wratislaviensis
cctggctcaggacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggccaactacgtgccagcagccgcggtaatacgtagggtgcaagcgttgtccggaattactgggcgtaaagagctcgtaggcggtttgtcgcgtcgtctgtgaaaactcgaggctcaacctcgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtgggtagcgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccggaaggtgcggctggatcacct

(19) United States
(12) Patent Application Publication
(10) Pub. No.: US 2015/0017694 A1
(43) Pub. Date: Jan. 15, 2015

(54) Engineered CO2-Fixing Chemotrophic Microorganisms Producing Carbon-Based Products and Methods of Using the Same

(71) Applicant: Kiverdi, Inc., Berkley, CA (US)

(72) Inventors: Itzhak Kurek, San Francisco, CA (US); John S. Reed, Berkeley, CA (US); Lisa Dyson, Berkeley, CA (US); Michael Siani-Rose, San Francisco, CA (US); Henrik Fyrst, Oakland, CA (US); Christer Jansson, Berkeley, CA (US); David Galgoczy, San Francisco, CA (US)

(73) Assignee: Kiverdi, Inc., Berkeley, CA (US)

(21) Appl. No.: 14/388,756

(22) PCT Filed: Mar. 15, 2013
(86) PCT No.: PCT/US13/32362

§ 371 Date: Sep. 26, 2014

Related U.S. Application Data
(63) Continuation-in-part of application No. 13/623,089, filed on Sep. 19, 2012.
Application No. 13/623,089 is a continuation-in-part of application No. PCT/US2011/034218, filed on Apr. 27, 2011.
Application No. PCT/US2011/034218 is a continuation-in-part of application No. PCT/US2010/001402, filed on May 12, 2010.
Application No. PCT/US2010/001402 is a continuation-in-part of application No. 12/613,550, filed on Nov. 6, 2009, now abandoned.
(60) Provisional application No. 61/616,560, filed on Mar. 28, 2012.
(60) Provisional application No. 61/635,238, filed on Apr. 18, 2012.
(60) Provisional application No. 61/708,057, filed on Oct. 1, 2012.
(60) Provisional application No. 61/536,056, filed on Sep. 19, 2011.
(60) Provisional application No. 61/111,794, filed on Nov. 6, 2008.
(60) Provisional application No. 61/542,823, filed on Oct. 4, 2011.
(60) Provisional application No. 61/616,560, filed on Mar. 28, 2012.

(51) Int. Cl. C12P 7/64; C12N 15/74

(52) U.S. Cl. CPC C12P 7/6409; C12N 15/74
USPC 435/134; 435/252.3

(57) ABSTRACT

Disclosed herein are microorganisms containing exogenous or heterologous nucleic acid sequences, wherein the microorganisms are capable of growing on gaseous carbon dioxide, gaseous hydrogen, syngas, or combinations thereof. In some embodiments the microorganisms are chemotrophic bacteria that produce or secrete at least 10% of lipid by weight. Also disclosed are methods of fixing gaseous carbon into organic carbon molecules useful for industrial processes. Also disclosed are methods of manufacturing chemicals or producing precursors to chemicals useful in jet fuel, diesel fuel, and biodiesel fuel. Exemplary chemicals or precursors to chemicals useful in fuel production are alkanes, alkenes, alkynes, fatty acid alcohols, fatty acid aldehydes, desaturated hydrocarbons, unsaturated fatty acids, hydroxyl acids, or diacids with carbon chains between six and thirty carbon atoms long. Also disclosed are microorganisms and methods using disclosed microorganisms for the production of butanediol and its chemical precursors in low-oxygen or anaerobic fermentation. Also disclosed are microorganisms and methods using disclosed microorganisms for generating hydroxylated fatty acids in microbes through the transfer of enzymes that are known to hydroxylate fatty acids in plants or microbes. Also disclosed are microorganisms and methods using disclosed microorganisms for the production of shorter-chain fatty acids in microbes through the introduction of exogenous fatty acyl-CoA binding proteins.

### RELATED APPLICATIONS

This application claims the benefit under 35 U.S.C. §119(e) of U.S. Provisional Patent Application No. 61/616,560, filed Mar. 28, 2012 and entitled PROCESS FOR GENERATING HYDROXYLATED FATTY ACIDS; U.S. Provisional Patent Application No. 61/635,238, filed Apr. 18, 2012 and entitled PROCESS FOR GENERATING SHORTER FATTY ACIDS WITH AN EXOGENOUS FATTY ACYL-COA BINDING PROTEIN; U.S. Provisional Patent Application No. 61/708,057, filed Oct. 1, 2012 and entitled PROCESS FOR PRODUCING CARBON-BASED CHEMICALS, INCLUDING BUTANEDIOL, USING CHEMOTROPHIC MICROBES. This application is also a continuation-in-part of U.S. patent application Ser. No. 13/623,089, filed Sep. 19, 2012, and entitled “INDUSTRIAL FATTY ACID ENGINEERING GENERAL SYSTEM FOR MODIFYING FATTY ACIDS.” Each of these applications is incorporated herein by reference in its entirety for all purposes.

### FIELD OF THE INVENTION

This disclosure relates to compositions capable of producing and methods of the producing oils, fuels, and oleochemicals through cultivating bacteria that grow on carbon-containing gas such as syngas, producer gas, CO₂, carbon monoxide and mixtures of the same containing hydrogen gas. This disclosure further relates to methods of fixing carbon from gas into useful organic molecules such as diacids, hydroxy acids, fatty acid alcohols, fatty acid aldehydes, fatty acids, unsaturated fatty acids, esters, lipids, alkanes, alkenes, and alkynes. The bacteria of the invention can be genetically engineered for use in the methods or other aspects of the invention described herein. The present invention further describes mechanisms to confer and/or enhance production of carbon-based products to an organism such that it converts carbon dioxide, or other inorganic carbon sources, and inorganic energy, including chemical energy from an inorganic chemical or directly from an electrical source, into carbon-based products of commercial value.

### BACKGROUND OF THE INVENTION

Sustainable and renewable sources of liquid fuel to operate machinery, aircraft, and vehicles are necessary to reduce the amount of carbon dioxide emissions in the atmosphere, as well as to reduce global energy consumption based upon coal, oil, and natural gas economies.

Increased demand for energy by the global economy has placed increasing pressure on the cost of hydrocarbons. Aside from energy, many industries, including plastics and chemical manufacturers, rely heavily on the availability of fossil hydrocarbon sources as a feedstock for their manufacturing processes. Cost-effective alternatives to current sources of supply could help mitigate the upward pressure on fossil resource demand and raw material costs.

Biologic systems that fix carbon through natural biochemical metabolic processes are known. Algal systems have been developed to create hydrocarbons through photosynthetic reactions, as well as heterotrophic reactions fed by sugar that indirectly depend upon photosynthesis, but insufficient yields limit the effectiveness, economic feasibility, practicality and commercial adoption. Bacterial cells have been genetically engineered to process sugar feedstocks into useful hydrocarbons in heterotrophic fermentation systems, however, there are significant drawbacks for these systems.

Heterotrophic fermentations are vulnerable to contamination because heterotrophic microorganisms that can grow on fixed carbon nutrients are far more ubiquitous in the surface environment. Heterotrophic technologies also generally suffer limitations in terms of food versus fuel conflict and negative environmental impacts.

Gas-to-liquid (GTL) technologies have the benefit of allowing the utilization of waste carbon sources—including highly lignocellulosic waste through the conversion to synthesis gas (syngas) via gasification, as well as waste CO₂ through the provision of reduced hydrogen—in the production of liquid fuels and/or organic chemicals. Syngas is a mix of gases that generally contains H₂, CO, and CO₂ as major components, which can be generated through steam reforming of methane and/or liquid petroleum gas or through gasification of any organic material, including but not limited to biomass, waste organic matter, various polymers, and coal. Many gasification processes are available for the production of syngas. A number of gasification processes subject the carbonaceous feedstock to partial oxidation at high temperatures (500-1500° C.), with the oxygen supply restricted to prevent complete combustion, producing syngas with varying composition depending on feedstock and reaction conditions such that the ratio of H₂:CO can range from 0.5:1 to 3:1. The hydrogen component of syngas can be raised through the reaction of CO with steam in the water gas shift reaction with a concomitant increase in CO₂ in the syngas mix.

Some major technologies for syngas conversion to liquid fuels or chemicals include chemical catalytic processes such as the Fischer-Tropsch (F-T) as well as processes for the synthesis of methanol or other mixed alcohols, and biological gas fermentation processes. F-T has been worked on for almost one hundred years and relies on metal-based, inorganic catalysts for the conversion of syngas into longer chain hydrocarbons. Difficulties with F-T include: a wide chain length distribution of products resulting in the need to reprocess short chain length products such as methane and LPG and/or the need to perform additional costly post-processing steps on long chain waxes and tars such as hydrocracking; high catalyst sensitivity to syngas impurities such as sulfur containing compounds, tars, and particulates, generally necessitating multiple costly gas clean up steps; relatively low flexibility in terms of accommodating various ratios of syngas constituents i.e. H2:CO, and low tolerance of CO₂, often resulting in additional costly syngas conditioning steps such as water gas shift and CO₂ removal; the actual F-T step is relatively high temperature and pressure resulting in costly compression and heating requirements; the wide distribution of products generally necessitates the storage, handling, and transport of a wide array of products which is often uneconomic except for relatively large scale operations; F-T products (e.g. diesel, jet fuel, naphtha, waxes) are relatively low in value at current (2011) prices compared to many different higher value oils, lipids, and oleochemicals that can be produced biologically. The difficulties with F-T generally also apply to other chemical conversion processes such as methanol synthesis.

The gasification of biomass to generate syngas has a long history going back to World War II where biomass gasification was used for running modified automobiles, boats, buses, and trucks. Presently, a number of biomass gasification technologies are at, or near commercialization (able to gasify 10,000 or more tons of biomass per year), and are generally used for the production of heat and/or electricity. The synthesis of chemicals or fuels from syngas generated via biomass gasification is at an earlier stage of development, and is generally pre-commercial.

Using syngas and/or CO₂ and/or renewable H₂ in gas fermentation enables the utilization of cheaper and more flexible sources of energy and/or carbon for the biological synthesis of sustainable chemicals and fuels than is possible through heterotrophic or phototrophic synthesis. In gas fermentation, syngas acts as both a carbon and energy source for the microbial culture. Some of the advantages of syngas fermentation include: the production of a relatively narrow range of carbon chain length distribution compared to F-T; lower sensitivity to syngas impurities; greater tolerance of varying ratios of H2:CO and the presence of CO2; able to operate at much closer to ambient temperature and pressure; able to produce various higher value oleochemical products.

A fermentation process based upon a gaseous feedstock such as syngas can allow for far lower negative environmental and food production impacts in the biological synthesis of liquid fuels and/or chemicals than the highly land and water intensive heterotrophic or phototrophic-based technologies. However, current biological GTL technologies generally yield relatively short chain alcohols, or other short chain organic compounds, as products, which have relatively low energy density and infrastructure compatibility with current petrochemical and oleochemical processes.

The syngas-growing microorganisms used in current biological GTL technologies are generally inappropriate for the synthesis of high energy density, infrastructure compatible fuels, or other longer carbon chain lipid-based chemicals. Their short chain products are relatively low in value and they generally don't efficiently synthesize drop-in fuels such as diesel or jet fuel, or higher value lipid-based chemicals.

Furthermore the types of microorganisms used in current biological GTL technologies such as Clostridia have a relatively low tolerance for their short carbon chain gas fermentation products such as ethanol, butanol, or acetic acid, which limits titers and complicates product recovery, hurting the overall economics of the GTL process.

There is a need to identify a set of microorganisms that can grow in conventional and scalable contained reaction vessels and that produce commercially viable sets of organic carbon chains of at least eight carbon atoms long in a commercially feasible method. There is a need to identify microorganisms not limited metabolically by typically used carbon and energy inputs such as sugars, and a microorganism that can additionally utilize syngas, producer gas, as well as a wide array of abiotic sources of carbon and energy for the synthesis of drop-in fuels and chemicals, leading to a feedstock flexibility for the present technology that far exceeds comparable heterotrophic systems. There is a need to identify and use microorganisms that can utilize electron donors such as hydrogen, present in syngas, producer gas, as well as readily generated through a wide array of abiotic renewable energy technologies, for growth and carbon fixation.

The targeting of fatty acids produced through fatty acid biosynthesis to relatively shorter fatty acid chain lengths from C8-C14 has been achieved in heterotrophic microorganisms. This has been accomplished through the use of thioesterases to change populations of fatty acids C8-C14 and the over-expression of thioesterases to increase shorter chain length fatty acids. Examples in the prior art include C8-C14 thioesterase expression to produce shorter chain lengths in U.S. Pat. No. 7,883,882 Renewable chemical production from novel fatty acid feedstocks, Franklin et al. Solazyme, p. 58.

However there is a need to target the production of shorter chain length fatty acids in microorganisms that are capable of growing and producing lipids chemotrophically on syngas or H₂/CO₂ gas mixes to enable microbial GTL production of lipids with targeted, mid-length carbon chains.

Dicarboxylic acids (Diacids) such as dodecanedoic acid (n=10) are used in production of nylon (nylon-6,12), polyamides, coatings, adhesives, greases, polyesters, dyes, detergents, flame retardants and fragrances. Diacids can be produced by fermentation of long-chain alkanes by candida tropicalis (Kroha K, Infom 2004, 15, 568). Traumatic acid, monounsaturated dodecanedoic acid (10E-dodeca-1,12-dicarboxylic acid) has been produced from plant tissues English J et al., Science 1939, 90, 329. Pyrococcus furiosus produces an array of dicarboxylic acids (Carballeira, 1997). The total amount of dicarboxylic acids comprises only 3.4% of the total, however, this could be boosted by various literature methods.

There is a need for a biological, non-heterotrophic means of producing diacids from low-cost or sustainable syngas feedstocks.

Nutritionally important n-3 fatty acids include α-linolenic acid (ALA), eicosapentaenoic acid (EPA), and docosahexaenoic acid (DHA), all of which are polyunsaturated. N-3 fatty acids that are important in human physiology are α-linolenic acid (18:3, n-3; ALA), eicosapentaenoic acid (20:5, n-3; EPA), and docosahexaenoic acid (22:6, n-3; DHA). These three polyunsaturates have either 3, 5, or 6 double bonds in a carbon chain of 18, 20, or 22 carbon atoms, respectively. As with most naturally produced fatty acids, all double bonds are in the cis-configuration.

A fatty acid desaturase is an enzyme that removes two hydrogen atoms from a fatty acid, creating a carbon/carbon double bond. These desaturases are classified as delta—indicating that the double bond is created at a fixed position from the carboxyl group of a fatty acid (for example, Δ9 desaturase creates a double bond at the 9th position from the carboxyl end). omega (e.g. ω3desaturase)—indicating the double bond is created between the third and fourth carbon from the methyl end of the fatty acid. In the biosynthesis of essential fatty acids, an elongase alternates with different desaturases (for example, Δ6desaturase) repeatedly inserting an ethyl group, then forming a double bond.

Most polyunsaturated oils come from fish and there is a need for alternate, and particularly microbial sources of polyunsaturated fatty acids, given depleting fish stocks and increasing pollution in the oceans.

### SUMMARY OF THE INVENTION

The present invention allows microorganisms to be genetically engineered to convert CO₂ gas and/or syngas and/or producer gas to higher value and/or more infrastructure compatible products than current biologically based syngas and/or CO₂ conversion technologies. The present technology allows the development of new genetically enhanced strains of microorganisms that can be used for gas fermentation within biological gas-to-liquid (GTL) processes to produce and/or secrete drop-in liquid fuels directly from CO₂ or from syngas, as well as various other relatively long chain organic compounds that are drop-in, and are currently only produced in bulk from petroleum or higher plants.

The present invention relates to the engineering of microorganisms, including but not limited to hydrogen oxidizing, carbon monoxide oxidizing, and knallgas microorganisms, with a natural capability to grow and synthesize biomass on gaseous carbon sources such as syngas and/or CO₂, such that the engineered microorganisms synthesize targeted products, including chemicals and fuels, under gas fermentation. The microorganisms and methods of the present invention enable low cost synthesis of chemicals and fuels, which can compete on price with petrochemicals and higher-plant derived oleochemicals, and which will generally have a substantially lower price than oleochemicals produced through heterotrophic or phototrophic synthesis.

The invention relates to a composition comprising a microorganism that converts syngas and/or gaseous CO₂ and/or a mixture of CO₂ gas and H₂ gas into one or more lipids. In some embodiments, the composition comprises a microorganism, wherein the microorganism is a carbon monoxide-oxidizing microorganism. In some embodiments, the composition comprises a microorganism, wherein the microorganism is a knallgas microorganism. In some embodiments, the composition comprises a microorganism, wherein the microorganism is chosen from the genera Rhodococcus or Gordonia. In some embodiments, the composition comprises a microorganism, wherein the microorganism is Rhodococcus opacus. In some embodiments, the composition comprises a microorganism, wherein the microorganism is Rhodococcus opacus (DSM 43205) or Rhodococcus sp (DSM 3346). In some embodiments, the composition comprises a microorganism, wherein the microorganism is chosen from the genera Ralstonia or Cupriavidus. In some embodiments, the composition comprises a microorganism, wherein the microorganism is Cupriavidus necator.

In some embodiments, the composition comprises a microorganism wherein the microorganism can naturally grow on H₂/CO₂ and/or syngas, and wherein the microorganism can naturally accumulate lipid to 50% or more of the cell biomass by weight. In some embodiments the microorganisms have a native ability to send a high flux of carbon down the fatty acid biosynthesis pathway. In some embodiments the microorganism exhibiting these traits is Rhodococcus opacus (DSM 43205 or DSM 43206).

In some embodiments, the composition comprises a microorganism that can naturally grow on H₂/CO₂ and/or syngas, and wherein the microorganism can naturally accumulate polyhydroxybutyrate (PHB) or polyhydroxyalkanoate (PHA) to 50% or more of the cell biomass by weight. In some embodiments the microorganisms have a native ability to direct a high flux of carbon through the acetyl-CoA metabolic intermediate, which can lead into fatty acid biosynthesis, along with a number of other synthetic pathways including PHA and PHB synthesis. A microorganism is considered to direct a high flux of carbon through acetyl-CoA if a product of a synthesis pathway going through the acetyl-CoA metabolic intermediate, including but not limited to polyhydroxybutyrate (PHB) or polyhydroxyalkanoate (PHA), can represent 50% or more of the cell biomass by weight. In some embodiments the microorganism exhibiting these traits is Cupriavidus necator (DSM 531 or DSM 541).

In some embodiments, the invention relates to a non-naturally occurring microorganism capable of converting syngas or other gaseous carbon sources into targeted oleochemical and/or monomer products, where the wild-type microorganism is capable of growing on syngas or other gaseous carbon sources, but is either not capable of synthesizing said targeted oleochemical and/or monomer products, or is capable of synthesizing the targeted oleochemicals and/or monomers, but is not capable of synthesizing the targeted biochemical products at the concentration and/or efficiency of the non-natural microorganism. In such microorganisms, one or more proteins or enzymes are expressed in the microorganism, thereby modifying, extending, diverting, enhancing, promoting, or otherwise altering the lipid biosynthesis pathway or its regulation for the synthesis and/or enhanced synthesis of a targeted lipid-based product, oleochemical, monomer, or hydrocarbon.

In some embodiments, the invention relates to a non-naturally occurring microorganism capable of converting syngas or other gaseous carbon sources into targeted oleochemical and monomer products, where the wild-type microorganism is capable of growing on syngas or other gaseous carbon sources and is capable of synthesizing said targeted oleochemical and monomer products, but the non-naturally occurring microorganism is capable of synthesizing the targeted biochemical products at a higher concentration and/or efficiency than the wild-type microorganism due to the overexpression and/or underexpression of one or more proteins or enzymes.

In some embodiments, the invention relates to compositions comprising one or more bacterial cells that consist of one, two, or three exogenous nucleic acid sequences where said bacteria can grow using syngas and/or gaseous CO₂ and/or a mixture of CO₂ gas and H₂ gas as a source of carbon and/or energy.

In some embodiments, the invention relates to compositions comprising one or more bacterial cells of Rhodococcus opacus (DSM 43205) that consist of zero, one, two, or three exogenous nucleic acid sequences.

In some embodiments one, two, or three exogenous nucleic acid sequences encode one or more thioesterase proteins.

In some embodiments one, two, or three exogenous nucleic acid sequences encode one or more CYP52A proteins.

In some embodiments one, two, or three exogenous nucleic acid sequences encode a CYP709C1 and/or a CYP81B1 protein.

In some embodiments the source of thioesterase is inherent to the production organisms. In some embodiments the source of thioesterase is Rhodococcus opacus B4. In some embodiments the thioesterase is derived from bacteria or plants other than the host microorganism.

In some embodiments, the invention relates to compositions comprising one or more bacterial cells that consist of two exogenous nucleic acid sequences that encode the following proteins: fatty acid acyl-ACP reductase, a fatty acid aldehyde decarbonylase, where said bacteria can grow using syngas and/or gaseous CO₂ and/or a mixture of CO₂ gas and H₂ gas as a source of carbon and/or energy.

In some embodiments, the invention relates to compositions comprising one or more bacterial cells that consist of three exogenous nucleic acid sequences that encode the following proteins: fatty acid acyl-ACP reductase, a fatty acid aldehyde decarbonylase, and a thioesterase, where said bacteria can grow using syngas and/or gaseous CO₂ and/or a mixture of CO₂ gas and H₂ gas as a source of carbon and/or energy.

In some embodiments, the bacterial cell produces and/or secretes one or more lipids in an amount that is greater than the amount of lipids produced and/or secreted by the same cell not comprising the exogenous nucleic acid sequence.

In some embodiments, the bacterial cell produces and/or secretes one or more lipids having a given carbon chain length, where the amount of said lipid produced and/or secreted is greater than the amount produced and/or secreted by the same cell not comprising the exogenous nucleic acid sequence.

In some embodiments, the bacterial cell produces and/or secretes one or more lipid molecules in an amount that is less than the amount of lipids produced and/or secreted by the same cell not comprising the exogenous nucleic acid sequence.

In some embodiments, the bacterial cell produces and/or secretes one or more hydrocarbons in an amount that is greater than the amount of hydrocarbons produced and/or secreted by the same cell not comprising the exogenous nucleic acid sequence.

In some embodiments, the bacterial cell produces and/or secretes one or more lipids or hydrocarbons in a ratio greater than the ratio of lipids or hydrocarbons produced and/or secreted by the same cell not comprising the one or more exogenous nucleic acid sequences. In some embodiments, the bacterial cell produces and/or secretes one or more lipids or hydrocarbons, wherein at least 50% of the one or more lipids or hydrocarbons have 8 to 18 carbon atoms. In some embodiments, the bacterial cell produces and/or secretes one or more lipids or hydrocarbons, wherein at least 60% of the one or more lipids or hydrocarbons have 8 to 18 carbon atoms. In some embodiments, the bacterial cell produces and/or secretes one or more lipids or hydrocarbons, wherein at least 70% of the one or more lipids or hydrocarbons have 8 to 18 carbon atoms. In some embodiments, the bacterial cell produces and/or secretes one or more lipids or hydrocarbons, wherein at least 75% of the one or more lipids or hydrocarbons have 8 to 18 carbon atoms. In some embodiments, the bacterial cell produces and/or secretes one or more lipids or hydrocarbons, wherein at least 80% of the one or more lipids or hydrocarbons have 8 to 18 carbon atoms.

In some embodiments, the bacterial cell or compositions comprising the bacterial cell comprise at least one exogenous nucleic acid sequence that is integrated into the genome of the cell.

In some embodiments, the invention relates to a composition comprising a microorganism that converts syngas and/or gaseous CO₂ and/or a mixture of CO₂ gas and H₂ gas into one or more hydrocarbons including unsaturated hydrocarbons, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase. In some embodiments the microorganism is Rhodococcus opacus.

In some embodiments, the invention relates to a composition comprising a microorganism that converts syngas and/or gaseous CO₂ and/or a mixture of CO₂ gas and H₂ gas into one or more hydrocarbons, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase, wherein the one or more hydrocarbons have a carbon chain length of at least 8 carbon atoms. In some embodiments, The invention relates to a composition comprising a microorganism that converts syngas and/or gaseous CO₂ and/or a mixture of CO₂ gas and H₂ gas into one or more hydrocarbons, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein the one or more hydrocarbons comprise a mixture of hydrocarbon molecules having a carbon chain length from 8 carbon atoms to 18 carbon atoms. In some embodiments, the invention relates to a composition comprising a microorganism that converts syngas and/or gaseous CO₂ and/or a mixture of CO₂ gas and H₂ gas into one or more lipids, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein the one or more lipids comprise a quantity of at least one alkane, alkene, alkyne, fatty alcohol, and/or fatty aldehyde at a level higher than the quantity of the alkane, alkene, alkyne, fatty alcohol, and or fatty aldehyde in the same microorganism not comprising the heterologous nucleic acid sequences. In some embodiments, the invention relates to a composition comprising a microorganism that converts syngas and/or gaseous CO₂ and/or a mixture of CO₂ gas and H₂ gas into one or more lipids, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein the microorganism produces and/or secretes at least 10% of one or more lipids by weight.

In some embodiments, the invention relates to a composition comprising a microorganism that converts syngas and/or gaseous CO₂ and/or a mixture of CO₂ gas and H₂ gas into one or more lipids, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein the microorganism produces and/or secretes at least 20% of one or more lipids by weight.

In some embodiments, the invention relates to a composition comprising a microorganism that converts syngas and/or gaseous CO₂ and/or a mixture of CO₂ gas and H₂ gas into one or more lipids, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein the microorganism produces and/or secretes at least 30% of one or more lipids by weight.

In some embodiments, the invention relates to a composition comprising a microorganism that converts syngas and/or gaseous CO₂ and/or a mixture of CO₂ gas and H₂ gas into one or more lipids, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein the microorganism produces and/or secretes at least 40% of one or more lipids by weight.

In some embodiments, the invention relates to a composition comprising a microorganism that converts syngas and/or gaseous CO₂ and/or a mixture of CO₂ gas and H₂ gas into one or more lipids, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein the microorganism produces and/or secretes at least 50% of one or more lipids by weight.

In some embodiments, the invention relates to a composition comprising a microorganism that converts syngas and/or gaseous CO₂ and/or a mixture of CO₂ gas and H₂ gas into one or more lipids, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein the microorganism produces and/or secretes at least 60% of one or more lipids by weight. In some embodiments, the invention relates to a composition comprising a microorganism that converts syngas and/or gaseous CO₂ and/or a mixture of CO₂ gas and H₂ gas into one or more lipids, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein the microorganism produces and/or secretes at least 70% of one or more hydrocarbons by weight.

In some embodiments, the invention relates to a composition comprising a microorganism that converts syngas and/or gaseous CO₂ and/or a mixture of CO₂ gas and H₂ gas into one or more lipids, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein the microorganism produces and/or secretes at least 75% of one or more lipids by weight. In some embodiments, the invention relates to a composition comprising a microorganism that converts syngas and/or gaseous CO₂ and/or a mixture of CO₂ gas and H₂ gas into one or more lipids, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein the microorganism produces and/or secretes at least 80% of one or more lipids by weight.

In some embodiments, the invention relates to a composition comprising a microorganism that converts syngas and/or gaseous CO₂ and/or a mixture of CO₂ gas and H₂ gas into one or more lipids, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein the microorganism produces and/or secretes at least 85% of one or more lipids by weight. In some embodiments, the invention relates to a composition comprising a microorganism that converts syngas and/or gaseous CO₂ and/or a mixture of CO₂ gas and H₂ gas into one or more hydrocarbons, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein less than 10% by weight of the hydrocarbons produced is methane. In some embodiments, the invention relates to a composition comprising a microorganism that converts syngas and/or gaseous CO₂ and/or a mixture of CO₂ gas and H₂ gas into one or more organic compounds, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein less than 10% by weight of the organic compounds produced are organic acids with carbon chain length of four carbons or less.

In some embodiments, the invention relates to a composition comprising a microorganism that converts syngas and/or gaseous CO₂ and/or a mixture of CO₂ gas and H₂ gas into one or more lipids or hydrocarbons, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein at least one lipid produced is a component or a precursor of a component of jet fuel, diesel fuel, or biodiesel fuel.

In some embodiments, the invention relates to a composition comprising a microorganism that converts syngas and/or gaseous CO₂ and/or a mixture of CO₂ gas and H₂ gas into one or more hydrocarbons, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein the hydrocarbons produced comprise a mixture of at least two hydrocarbons having a carbon backbone from 8 to 18 carbon atoms.

The present invention also relates to a bacterial cell comprising at least two exogenous nucleic acid sequences, wherein the at least two exogenous nucleic acid sequences encode fatty acid acyl-ACP reductase and fatty acid aldehyde decarbonylase, and wherein the cell converts gaseous CO₂ and/or gaseous H₂ and/or syngas into lipids. In some embodiments, the invention relates to a bacterial cell comprising at least two exogenous nucleic acid sequences, wherein the at least two exogenous nucleic acid sequences encode fatty acid acyl-ACP reductase and fatty acid aldehyde decarbonylase, and wherein the cell converts gaseous CO₂ and/or gaseous H₂ and/or syngas into lipid; wherein the cell produces and/or secretes at least 75% of one or more hydrocarbons by weight. In some embodiments, the invention relates to a bacterial cell comprising at least two exogenous nucleic acid sequences, wherein the at least two exogenous nucleic acid sequences encode fatty acid acyl-ACP reductase and fatty acid aldehyde decarbonylase, and wherein the cell converts gaseous CO₂ and/or gaseous H₂ and/or syngas into lipid; wherein the cell produces and/or secretes at least 75% of one or more hydrocarbons by weight when cultured at least 42 degrees Celsius for at least 1 hour. In some embodiments, the bacterial cell is cultured without exposure to light.

In some embodiments, the invention relates to a bacterial cell comprising at least two exogenous nucleic acid sequences, wherein the at least two exogenous nucleic acid sequences encode fatty acid acyl-ACP reductase and fatty acid aldehyde decarbonylase, and wherein the cell converts gaseous CO₂ and/or gaseous H₂ and/or syngas into a hydrocarbon or mixture of hydrocarbons, and/or other lipids; wherein the cell is a strain of Rhodococcus opacus.

In some embodiments, the invention relates to a bacterial cell comprising at least two exogenous nucleic acid sequences, wherein the at least two exogenous nucleic acid sequences encode fatty acid aldehyde acyl-ACP and fatty acid aldehyde decarbonylase, and wherein the cell converts gaseous CO₂ and/or gaseous H₂ and/or syngas into a hydrocarbon or mixture of hydrocarbons, and/or other lipids; wherein the cell is a strain of Cupriavidus necator.

In some embodiments, the invention relates to a bacterial cell comprising a first, a second, and a third exogenous nucleic acid sequence, wherein the first exogenous nucleic acid sequence encodes fatty acid acyl-ACP reductase, the second exogenous nucleic acid sequence encodes a fatty acid aldehyde decarbonylase, and the third exogenous nucleic acid sequence encodes a thioesterase; and wherein the cell converts gaseous CO₂ and/or gaseous H₂ and/or syngas into a lipid or mixture of lipids. In some embodiments, the bacterial cell comprises no more than eight exogenous nucleic acids that encode a lipid pathway enzyme. In some embodiments, the bacterial cell comprises no more than seven exogenous nucleic acids that encode a lipid pathway enzyme. In some embodiments, the bacterial cell comprises no more than six exogenous nucleic acids that encode a lipid pathway enzyme. In some embodiments, the bacterial cell comprises no more than five exogenous nucleic acids that encode a lipid pathway enzyme. In some embodiments, the bacterial cell comprises no more than four exogenous nucleic acids that encode a lipid pathway enzyme. In some embodiments, the bacterial cell comprises no more than three exogenous nucleic acids that encode a lipid pathway enzyme. In some embodiments, the bacterial cell comprises no more than two exogenous nucleic acids that encode a lipid pathway enzyme. In some embodiments, the bacterial cell comprises no more than one exogenous nucleic acid that encodes a lipid pathway enzyme. In some embodiments, the bacterial cell comprises no more than eight exogenous nucleic acids that encode a protein. In some embodiments, the bacterial cell comprises no more than seven exogenous nucleic acids that encode a protein. In some embodiments, the bacterial cell comprises no more than six exogenous nucleic acids that encode a protein. In some embodiments, the bacterial cell comprises no more than five exogenous nucleic acids that encode a protein. In some embodiments, the bacterial cell comprises no more than four exogenous nucleic acids that encode a protein. In some embodiments, the bacterial cell comprises no more than three exogenous nucleic acids that encode a protein. In some embodiments, the bacterial cell comprises no more than two exogenous nucleic acids that encode a protein. In some embodiments, the bacterial cell comprises no more than one exogenous nucleic acid that encodes a protein.

In some embodiments the invention relates to a method of producing a lipid or mixture of lipids in a microorganism population comprising the cell or the composition described herein, wherein the method comprises: culturing a population of microorganisms comprising the cell or the composition described herein in a feedstock comprising syngas and/or gaseous CO₂ and/or a mixture of CO₂ gas and H₂ gas.

In some embodiments, the invention relates to a method of producing a lipid or mixture of lipids, wherein the method comprises: culturing a population of bacterial cells comprising the cell or the composition described herein in a feedstock comprising syngas and/or gaseous CO₂ and/or a mixture of CO₂ gas and H₂ gas. In some embodiments, the microorganism population comprises a bacterial strain of Rhodococcus opacus. In some embodiments, the microorganism population comprises a bacterial strain of Rhodococcus opacus (DSM 43205 or 43206).

In some embodiments, the invention relates to a method of producing a lipid or mixture of lipids, wherein the method comprises: culturing a population of bacterial cells comprising the cell or the composition described herein in a feedstock comprising methanol, a common impurity of syngas, with or without the addition of syngas and/or gaseous CO₂ and/or a mixture of CO₂ gas and H₂ gas. In some embodiments, the microorganism population comprises a bacterial strain of Rhodococcus opacus. In some embodiments, the microorganism population comprises a bacterial strain of Rhodococcus opacus (DSM 43205).

In some embodiments, the invention relates to a method of producing a lipid or mixture of lipids, wherein the method comprises: culturing a population of bacterial cells comprising the cell or the composition described herein in a feedstock comprising syngas and/or gaseous CO₂ and/or a mixture of CO₂ gas and H₂ gas. In some embodiments, the microorganism population comprises a bacterial strain of Cupriavidus necator.

In some embodiments, the molecule produced is one or more alkane, alkene, alkyne, fatty alcohol, and/or fatty aldehyde. In some embodiments, the method produces a lipid or mixture of lipids at a quantity higher than the quantity of lipid or mixture of lipids in the same bacterial cell population not comprising the exogenous nucleic acids described herein. In some embodiments the one or more lipids comprise a quantity of at least one alkane, alkene, alkyne, fatty alcohol, and/or fatty aldehyde at a level higher than the quantity of the alkane, alkene, alkyne, fatty alcohol, and or fatty aldehyde in the same microorganism not comprising the exogenous nucleic acid sequences. In some embodiments, the method comprises a population of microorganisms or bacterial cell described herein that produces and/or secretes lipids of a weight equal to or greater than 10% of the total percentage of cellular dry matter. In some embodiment, the method comprises a population of microorganisms or bacterial cell described herein that produces and/or secretes lipids of a weight equal to or greater than 20% of the total percentage of cellular dry matter. In some embodiment, the method comprises a population of microorganisms or bacterial cell described herein that produces and/or secretes lipids of a weight equal to or greater than 30% of the total percentage of cellular dry matter. In some embodiments, the method comprises a population of microorganisms or bacterial cell described herein that produces and/or secretes lipids of a weight equal to or greater than 40% of the total percentage of cellular dry matter. In some embodiment, the method comprises a population of microorganisms or bacterial cell described herein that produces and/or secretes lipids of a weight equal to or greater than 50% of the total percentage of cellular dry matter. In some embodiments, the method comprises a population of microorganisms or bacterial cells described herein that produces and/or secretes lipids of a weight equal to or greater than 60% of the total percentage of cellular dry matter. In some embodiments, the method comprises a population of microorganisms or bacterial cells described herein that produces and/or secretes lipids of a weight equal to or greater than 70% of the total percentage of cellular dry matter. In some embodiments, the method comprises a population of microorganisms or bacterial cell described herein that produces of secretes lipids of a weight equal to or greater than 75% of the total percentage of cellular dry matter. In some embodiment, the method comprises a population of microorganisms or bacterial cell described herein that produces of secretes lipids of a weight equal to or greater than 80% of the total percentage of cellular dry matter. In some embodiments, the method comprises a population of microorganisms or bacterial cell described herein that produces of secretes lipids of a weight equal to or greater than 85% of the total percentage of cellular dry matter. In some embodiments, the bacterial cell or composition comprising the bacterial cell produces and/or secretes at least 10% of the total percentage of the cellular dry matter or 10% by weight. In some embodiment, the method comprises a population of microorganisms comprising a bacterial cell described herein that produces or secretes lipids, wherein at least 5% of the lipids have carbon backbones from 8 to 18 carbon atoms in length. In some embodiment, the method comprises a population of microorganisms comprising a bacterial cell described herein that produces or secretes lipids, wherein at least 10% of the lipids have carbon backbones from 8 to 18 carbon atoms in length. In some embodiments, the method comprises a population of microorganisms comprising a bacterial cell described herein that produces or secretes lipids, wherein at least 15% of the lipids have carbon backbones from 8 to 18 carbon atoms in length. In some embodiments, the method comprises a population of microorganisms comprising a bacterial cell described herein that produces or secretes lipids, wherein at least 20% of the lipids have carbon backbones from 8 to 18 carbon atoms in length.

In some embodiments, the molecule is chosen from one or more alkene, alkyne, unsaturated fatty acid, hydroxyacid and/or dicarboxylic acid (diacid). In some embodiments the one or more lipids comprise a quantity of at least one alkene, alkyne, unsaturated fatty acid, hydroxyacid and/or diacid at a level higher than the quantity of the alkene, alkyne, unsaturated fatty acid, hydroxyacid and/or diacid in the same microorganism not comprising the exogenous nucleic acid sequences.

In some embodiments of the invention, the invention relates to a method of producing and/or secreting a lipid or mixture of lipids by culturing a population of microorganisms comprising a bacterial cell described herein, wherein the exogenous nucleic acid sequences are operably linked to a promoter that is inducible in response to a first stimulus, and wherein the method further comprises: culturing the population of bacterial cells for a first period of time in the presence of a first stimulus to produce one or more lipids chosen from an alkane, alkene, alkyne, fatty acid, unsaturated fatty acid, diacid, hydroxy acid, alcohol, and/or fatty acid aldehyde.

In some embodiments of the invention, the invention relates to a method of fixing carbon from a gaseous feedstock containing carbonaceous molecules, wherein the method comprises the step of exposing a composition comprising exposing a bacterial cell to syngas and/or gaseous CO₂ and/or gaseous H₂; wherein the bacterial cell comprises at least one exogenous nucleic acid sequence. In some embodiments the exogenous nucleic acid sequences are fatty acid acyl-ACP reductase or a fatty acid aldehyde decarbonylase. In some embodiments of the method, the bacterial cell comprises at least a first and a second exogenous nucleic acid sequence, wherein the first exogenous nucleic acid sequence encodes a fatty acid acyl-ACP reductase and the second exogenous nucleic acid sequence encodes a fatty acid aldehyde decarbonylase. In some embodiments, the bacterial cell is Rhodococcus opacus or the population of microorganisms comprises a Rhodococcus cell. In some embodiments, the bacterial cell is Cupriavidus necator or the population of microorganisms comprises a Cupriavidus cell. In some embodiments, the bacterial cell comprises at least a first, a second, and a third exogenous nucleic acid sequence, wherein the first exogenous nucleic acid sequence encodes a fatty acid acyl-ACP reductase, the second exogenous nucleic acid sequence encodes a fatty acid aldehyde decarbonylase, and the third exogenous nucleic acid sequence encodes a thioesterase. In some embodiments, the bacterial cell comprises at least a first exogenous nucleic acid sequence, wherein the first exogenous nucleic acid sequence encodes a thioesterase. In some embodiments, the bacterial cell comprises no more than five exogenous nucleic acid sequences that encode a lipid pathway enzyme. In some embodiments, the composition comprises a microorganism, wherein the microorganism is Rhodococcus opacus (DSM 43205 or 43206) or Rhodococcus sp (DSM 3346). In some embodiments, the composition comprises a microorganism, wherein the microorganism is chosen from the genera Ralstonia or Cupriavidus. In some embodiments, the composition comprises a microorganism, wherein the microorganism is Cupriavidus necator. In some embodiments the microorganism is from the suborder corynebacterineae or the family burkholderiaceae. In some embodiments the microorganism through its native machinery produces a complement of fatty acids described in the Fatty Acid Output section below. In some embodiments, the bacterial cell comprises at least a first and a second exogenous nucleic acid sequence but no more than five exogenous nucleic acid sequences, wherein the first exogenous nucleic acid sequence encodes fatty acid acyl-ACP reductase and the second exogenous nucleic acid sequence encodes a fatty acid aldehyde decarbonylase.

In some embodiments, the invention relates to a method of producing one or more hydroxyacid, diacid, or unsaturated fatty acid, alcohols, fatty acid aldehydes, alkanes, alkenes, alkynes, or any combination thereof comprising exposing a bacterial cell to syngas and/or gaseous CO₂ or a mixture of gaseous CO₂ and gaseous H₂; wherein the bacterial cell is capable of fixing gaseous CO₂ into one or more fatty acid alcohols, alkanes, alkenes, or alkynes and wherein the microorganism comprises at least a first exogenous nucleic acid and a second exogenous nucleic acid, wherein the first exogenous nucleic acid encodes fatty acid acyl-ACP reductase and the second exogenous nucleic acid encodes fatty acid aldehyde decarbonylase. In some embodiments, the first and second exogenous nucleic acids are heterologous nucleic acid sequences. In some embodiments, the bacterial cell comprises at least a first, a second, and a third exogenous nucleic acid sequence, wherein the first exogenous nucleic acid sequence encodes a fatty acid acyl-ACP reductase, the second exogenous nucleic acid sequence encodes a fatty acid aldehyde decarbonylase, and the third exogenous nucleic acid sequence encodes a thioesterase. In some embodiments, the bacterial cell comprises at least a first exogenous nucleic acid sequence, wherein the first exogenous nucleic acid sequence encodes a thioesterase. In some embodiments, the composition comprises a bacterial cell, wherein the bacteria is Rhodococcus opacus (DSM 43205 or 43206) or Rhodococcus sp (DSM 3346). In some embodiments, the bacterial cell is chosen from the genera Ralstonia or Cupriavidus. In some embodiments, the bacterial cell is Cupriavidus necator. In some embodiments the bacterial cell is from the suborder corynebacterineae or the family burkholderiaceae. In some embodiments the bacterial cell through its native machinery produces a complement of fatty acids described in the Fatty Acid Output section below.

In some embodiments, the invention relates to a method of producing one or more unsaturated fatty acids, comprising exposing a bacterial cell to syngas and/or gaseous CO2 or a mixture of gaseous CO₂ and gaseous H₂; wherein the bacterial cell is capable of fixing gaseous CO₂ into one or more unsaturated fatty acids and wherein the microorganism comprises at least a first exogenous nucleic acid, wherein the first exogenous nucleic acid encodes a desaturase that introduces double bonds to fatty acids. In some embodiments, the first exogenous nucleic acids is a heterologous nucleic acid sequence. In some embodiments, the bacterial cell comprises at least a first, and a second exogenous nucleic acid sequence, wherein the first exogenous nucleic acid sequence encodes a desaturase, the second exogenous nucleic acid sequence encodes a thioesterase. In some embodiments, the composition the bacterial cell comprises a microorganism, wherein the microorganism is Rhodococcus opacus (DSM 43205 or 43206) or Rhodococcus sp (DSM 3346). In some embodiments, the composition comprises a microorganism, wherein the microorganism is chosen from the genera Ralstonia or Cupriavidus. In some embodiments, the composition comprises a microorganism, wherein the microorganism is Cupriavidus necator. In some embodiments the microorganism is from the suborder corynebacterineae or the family burkholderiaceae. In some embodiments the microorganism through its native machinery produces a complement of fatty acids described in the Fatty Acid Output section below. In some embodiments, the invention relates to a method of producing one or more hydroxy fatty acids (hydroxy acids), comprising exposing a bacterial cell to syngas and/or gaseous CO₂ or a mixture of gaseous CO₂ and gaseous H₂; wherein the bacterial cell is capable of fixing gaseous CO₂ into one or more hydroxy acids and wherein the microorganism comprises at least a first exogenous nucleic acid, wherein the first exogenous nucleic acid encodes a P450-dependent fatty acid hydroxylase that introduces hydroxyl groups at positions along the fatty acid chain. In some embodiments, the first exogenous nucleic acids is a heterologous nucleic acid sequence. In some embodiments, the bacterial cell comprises at least a first, and a second exogenous nucleic acid sequence, wherein the first exogenous nucleic acid sequence encodes a P450-dependent fatty acid hydroxylase, the second exogenous nucleic acid sequence encodes a thioesterase. In some embodiments, the composition comprises a microorganism, wherein the microorganism is Rhodococcus opacus (DSM 43205 or 43206) or Rhodococcus sp (DSM 3346). In some embodiments, the composition comprises a microorganism, wherein the microorganism is chosen from the genera Ralstonia or Cupriavidus. In some embodiments, the composition comprises a microorganism, wherein the microorganism is Cupriavidus necator. In some embodiments the microorganism is from the suborder corynebacterineae or the family burkholderiaceae. In some embodiments the microorganism through its native machinery produces a complement of fatty acids described in the Fatty Acid Output section below.

In some embodiments, the invention relates to a method of producing one or more hydroxyacid, diacid, or unsaturated fatty acid, alcohols, fatty acid aldehydes, alkanes, alkenes, alkynes, or any combination thereof comprising exposing a bacterial cell to syngas and/or gaseous CO₂ or a mixture of gaseous CO₂ and gaseous H₂; wherein the bacterial cell is capable of fixing gaseous CO₂ into one or more lipids; wherein the lipids are recovered from the bioreactor and fed to a second bioreactor wherein the lipids are postprocessed to generate hydroxyacid, diacid, and/or unsaturated fatty acids via a second microorganism such as but not limited to Candida tropicalis.

In some embodiments, the invention relates to a method of manufacturing one or more lipids, comprising (a) culturing a cell described herein in a reaction vessel or bioreactor in the presence of syngas and/or gaseous CO₂ or a mixture of gaseous CO₂ and gaseous H₂, wherein the cell produces and/or secretes one or more lipids in an quantity equal to or greater than at least 10% of the cell's total dry cellular mass; and (b) separating the one or more lipids from reaction vessel. In some embodiments, the method further comprises purifying the one or more lipids after separation from the reaction vessel or bioreactor.

In some embodiments, the one or more lipids is a component of or a precursor to a component of jet fuel, diesel fuel, or biodiesel fuel.

In some embodiments, the invention relates to a method of producing a alkene, fatty alcohol, alkyne, or alkane in a bacterial cell comprising at least a first and a second exogenous nucleic acid sequence, wherein the first exogenous nucleic acid sequence encodes a fatty acid acyl-ACP reductase and the second exogenous nucleic acid encodes a fatty acid aldehyde decarbonylase.

In some embodiments, the bacterial cell producing a alkene, fatty alcohol, alkyne, or alkane comprises at least a first, a second, and a third exogenous nucleic acid sequences, wherein the first exogenous nucleic acid sequence encodes a fatty acid acyl-ACP reductase and the second exogenous nucleic acid encodes a fatty acid aldehyde decarbonylase, and the third exogenous nucleic acid encodes a thioesterase.

In some embodiments, the invention relates to a method of producing cycloalkanes in a bacterial cell comprising at least a first exogenous nucleic acid sequence, wherein the first exogenous nucleic acid sequence encodes a fatty acyl-CoA reductase. In some embodiments the cycloalkane is cyclotetradecane. In some embodiments, the bacterial cell is Cupriavidus necator or the population of microorganisms comprises a Cupriavidus cell. In some embodiments the nucleic acid sequence comprises or consists of SEQ ID NO:5 and/or SEQ ID NO: 6. In some embodiments the nucleic acid sequence has at least 50, 60, 70, 75, 80, 85, 90, 91, 92, 93, 94, 95, 96, 97, 98 or 99% nucleotide homology to one or more of SEQ ID NOs: 5 or 6.

In some embodiments, the invention relates to a bioreactor comprising the composition or bacterial cells described herein.

In some embodiments, the invention relates to a system for the production of one or more lipids or mixture of lipids, comprising a bioreactor, which comprises: (a) a microorganism population comprising a cell described herein; and (b) an inlet connected to a feedstock source allowing delivery of a feedstock comprising syngas and/or gaseous CO₂ or a mixture of gaseous CO₂ and gaseous H₂. In some embodiments, the lipid or mixture of lipids comprise at least one component of or one precursor to a component of jet fuel, diesel fuel, or biodiesel fuel.

In some embodiments, the invention relates to the population of fatty acids being modified to produce molecules of desired carbon chain length by incorporation of one or more thioesterases.

In some embodiments, the invention relates to the population of fatty acids being modified to add additional carboxylic acid (—COOH) groups using exogenous enzymes.

In some embodiments, the invention relates to the population of fatty acids being modified to add hydroxyl groups (—OH) using the exogenous enzymes (hydroxylases).

In some embodiments, the invention relates to the population of fatty acids being modified to add desaturation through the incorporation of one or more double bonds, using the exogenous enzymes (desaturases).

In some embodiments, the invention relates to a method for generating hydroxylated fatty acids in microbes through the transfer of enzymes that are known to hydroxylate fatty acids in plants or microbes into microorganisms where the enzyme is not native.

In some embodiments, the invention relates to a microorganism comprising at least a first exogenous nucleic acid sequence wherein the microorganism converts gaseous CO2 and/or gaseous H2 and/or syngas into one or more hydroxylated fatty acids. In some embodiments, the invention further provides a composition wherein the first exogenous nucleic acid sequence encodes a hydroxylating ezyme. In some embodiments, the invention further comprises a second exogenous nucleic acid sequence encoding a thioesterase enzyme. In some embodiments, the invention further provides a composition wherein the microorganism is the genera Rhodococcus or Gordonia. In some embodiments, the invention further provides a composition wherein the microorganism is Rhodococcus opacus. In some embodiments, the invention further provides a composigion wherein the microorganism is Rhodococcus opacus (DSM 43205) or Rhodococcus opacus (DSM 43206) or Rhodococcus opacus (DSM 44193). In some embodiments, the invention further provides a composition wherein the microorganism is of the family Burkholderiaceae. In some embodiments, the invention further provides a composition wherein the microorganism is Cupriavidus necator. In some embodiments, the invention further provides a composition wherein the microorganism is Cupriavidus metallidurans. In some embodiments, the invention further provides a composition wherein the microorganism is a knallgas microorganism, also known as an oxyhydrogen microorganism. In some embodiments, the invention further provides a composition wherein the microorganism is a chemoautotrophic microbe. In some embodiments, the invention further provides a composition wherein the wild-type or mutant of the microorganism naturally has a capability for accumulating and/or synthesizing high quantities of triacylglycerol where a high quantity is considered to be 10% or more of the dry cell mass; 20% or more of the dry cell mass; 30% or more of the dry cell mass; 40% or more of the dry cell mass; 50% or more of the dry cell mass; 60% or more of the dry cell mass; 70% or more of the dry cell mass. In some embodiments, the invention further provides a composition wherein the microorganism is a hydrogen-oxidizing chemoautotroph. In some embodiments, the invention further provides a composition wherein the microorganism is capable of growing on syngas as the sole energy and carbon source. In some embodiments, the invention further provides a composition wherein the microorganism is capable of growing on untreated crude glycerol as the sole energy and carbon source.

In some embodiments, the invention relates to a method for producing hydroxylated fatty acids wherein the method comprises culturing an engineered microorganism or a natural strain in a bioreactor or solution with a feedstock comprising syngas and/or gaseous CO2 and/or a mixture of CO2 gas and H2 gas. In some embodiments, the invention further provides a step of up-regulating an endogenous or exogenous thioesterase gene of the microorganism. In some embodiments, the invention further provides a step of down-regulating an endogenous or exogenous thioesterase gene of the microorganism. In some embodiments, the invention further provides a step of down-regulating an endogenous or exogenous acyl carrier protein gene of the microorganism.

In some embodiments, the invention relates to a microorganism comprising at least a first exogenous nucleic acid sequence wherein the microorganism converts gaseous CO₂ and/or gaseous H₂ and/or syngas into one or more shorter-chain fatty acids. In some embodiments, the invention further provides a composition wherein the first exogenous nucleic acid sequence encodes a fatty acyl-CoA binding protein. In some embodiments, the invention further comprises a second exogenous nucleic acid sequence encoding a thioesterase enzyme. In some embodiments, the invention further provides a composition wherein the microorganism is the genera Rhodococcus or Gordonia. In some embodiments, the invention further provides a composigion wherein the microorganism is Rhodococcus opacus. In some embodiments, the invention further provides a composition wherein the microorganism is Rhodococcus opacus (DSM 43205) or Rhodococcus opacus (DSM 43206) or Rhodococcus opacus (DSM 44193). In some embodiments, the invention further provides a composition wherein the microorganism is of the family Burkholderiaceae. In some embodiments, the invention further provides a composition wherein the microorganism is Cupriavidus necator. In some embodiments, the invention further provides a composition wherein the microorganism is Cupriavidus metallidurans. In some embodiments, the invention further provides a composition wherein the microorganism is a knallgas microorganism, also known as an oxyhydrogen microorganism. In some embodiments, the invention further provides a composition wherein the microorganism is a chemoautotrophic microbe. In some embodiments, the invention further provides a composition wherein the wild-type or mutant of the microorganism naturally has a capability for accumulating and/or synthesizing high quantities of triacylglycerol where a high quantity is considered to be 10% or more of the dry cell mass; 20% or more of the dry cell mass; 30% or more of the dry cell mass; 40% or more of the dry cell mass; 50% or more of the dry cell mass; 60% or more of the dry cell mass; 70% or more of the dry cell mass. In some embodiments, the invention further provides a composition wherein the microorganism is a hydrogen-oxidizing chemoautotroph. In some embodiments, the invention further provides a composition wherein the microorganism is capable of growing on syngas as the sole energy and carbon source. In some embodiments, the invention further provides a composition wherein the microorganism is capable of growing on untreated crude glycerol as the sole energy and carbon source.

In some embodiments, the invention relates to a method for producing shorter-chain fatty acids wherein the method comprises culturing an engineered microorganism or a natural strain in a bioreactor or solution with a feedstock comprising syngas and/or gaseous CO₂ and/or a mixture of CO₂ gas and H₂ gas. In some embodiments, the invention further provides a step of enhancing expression of enzymes through heat. In some embodiments, the invention further provides a step of up-regulating an endogenous or exogenous thioesterase gene of the microorganism. In some embodiments, the invention further provides a step of down-regulating an endogenous or exogenous thioesterase gene of the microorganism. In some embodiments, the invention further provides a step of down-regulating an endogenous or exogenous acyl carrier protein gene of the microorganism.

In one embodiment, the instant invention provides a method of producing butanediol, or other biochemical precursors to butanediol by microbial fermentation under microaerophilic or anaerobic conditions, including: supplying an inorganic substrate as a primary source of metabolic energy, fermentation in a bioreactor containing a culture of microorganisms utilizing an inorganic substrate as a primary source of metabolic energy and carbon dioxide or other inorganic carbon as the primary source of carbon. In some embodiments, the invention further provides a method wherein the inorganic substrate comprises hydrogen (H2). In some embodiments, the invention further provides a method wherein the butanediol product is 2,3 butanediol, 1,4 butanediol, and/or 1,3 butanediol. In some embodiments, the invention further provides a method wherein the level of hydrogen is supplied at such a level such that butanediol is produced. In some embodiments, the invention further provides a method wherein the level of CO₂ is supplied at a level such that butanediol is produced. In some embodiments, the invention further provides a method wherein the culture is propogated in the bioreactor in which oxygen is introduced at a certain flow rate, and the oxygen level is subsequently changed to a lower flow rate, and the oxygen level is subsequently changed to a lower flow rate such that butanediol is produced at enchanced levels. In some embodiments, the invention further provides a method wherein the electron donors include but are not limited to one or more of the following reducing agents: ammonia; ammonium; carbon monoxide; dithionite; elemental sulfur; hydrogen; metabisulfites; nitric oxide; nitrites; sulfates such as thiosulfates including but not limited to sodium thiosulfate (Na₂S₂O₃) or calcium thiosulfate (CaS₂O₃); sulfides such as hydrogen sulfide; sulfites; thionate; thionite. In some embodiments, the invention further provides a method wherein the primary fermentation microbe is of the genera Rhodococcus or Gordonia. In some embodiments, the invention further provides a method wherein the primary fermentation microbe is the species Rhodococcus sp. DSM 3346 or DSM364. In some embodiments, the invention further provides a method wherein the primary fermentation microbe is a Rhodococcus opacus. In some embodiments, the invention further provides a method wherein the primary fermentation microbe is a Rhodococcus opacus (DSM 43205) or a Rhodococcus opacus (DSM 43206) or a Rhodococcus opacus (DSM 44193). In some embodiments, the invention further provides a method wherein the primary fermentation microbe is of the family Burkholderiaceae. In some embodiments, the invention further provides a method wherein the primary fermentation microbe is Cupriavidus necator. In some embodiments, the invention further provides a method wherein the primary fermentation microbe is Cupriavidus metallidurans. In some embodiments, the invention further provides a method wherein the primary fermentation microbe is a knallgas microorganism, also known as an oxyhydrogen microorganism. In some embodiments, the invention further provides a method wherein the primary fermentation microbe is a chemoautotrophic microbe. In some embodiments, the invention further provides a method wherein the wild-type or mutant of the microorganism naturally has a capability for accumulating and/or synthesizing high quantities of triacylglycerol where a high quantity is considered to be 10% or more of the dry cell mass; 20% or more of the dry cell mass; 30% or more of the dry cell mass; 40% or more of the dry cell mass; 50% or more of the dry cell mass; 60% or more of the dry cell mass; 70% or more of the dry cell mass. In some embodiments, the invention further provides a method wherein the primary fermentation microbe is a hydrogen-oxidizing chemoautotroph. In some embodiments, the invention further provides a composition wherein the primary fermentation microbe is capable of growing on syngas as the sole energy and carbon source. In some embodiments, the invention further provides a composition wherein the primary fermentation microbe is capable of growing on untreated crude glycerol as the sole energy and carbon source. In some embodiments, the invention further provides a step of up-regulating an endogenous or exogenous gene regulating the pathway for the production of butanediol. In some embodiments, the invention further provides a step of down-regulating an endogenous or exogenous gene regulating the pathway for the production of butanediol.

In one aspect of the invention, a chemotroph capable of CO₂ fixation, is engineered to produce a carbon-based product having a desired chemical structure to a level sufficient for commercial production. The product generated may be native to the organism, but produced in non-optimal quantities in the absence of engineering, or completely lacking in the absence of engineering.

In some examples, a host cell is genetically modified with an exogenous nucleic acid sequence encoding a single protein involved in a biosynthetic pathway generating a carbon-based product or intermediate. In other examples, a host cell is genetically modified with an exogenous nucleic acid sequence encoding multiple proteins involved in a biosynthetic pathway generating a carbon-based product or intermediate. In still other examples, a host cell is genetically modified with multiple exogenous nucleic acid sequences encoding multiple proteins involved in a biosynthetic pathway generating a carbon-based product or intermediate, or multiple carbon-based products or intermediates.

In some examples, a host cell is genetically modified with an exogenous nucleic acid sequence encoding a single protein affecting the generation of a carbon-based product or intermediate, but in a manner that does not directly add to or modify the biosynthetic pathway protein sequences. In other examples, a host cell is genetically modified with an exogenous nucleic acid sequence encoding multiple proteins affecting the generation of a carbon-based product or intermediate, but in a manner that does not directly add to or modify the biosynthetic pathway protein sequences.

In one aspect of the invention, a chemotroph capable of CO₂ fixation is engineered to produce two or more carbon-based products having desired chemical structures to a level sufficient for commercial production. The products generated may be native to the organism, but produced in non-optimal quantities in the absence of engineering, or completely lacking in the absence of engineering.

In some embodiments, such organisms produce at least 1 mg of carbon-based product of interest per liter of fermentation suspension. In some examples, the product is secreted by the organism into culture medium. In other examples, the product is retained in the organism in the course of fermentation. In some cases, the product may be recovered by lysing the cells and separating the product. In other cases, the product may have commercial value in the intact organism without significant preparation or purification of the product from the organism.

In one embodiment, production of one of more other fermentation byproducts are attenuated or eliminated by downregulation of pathway genes that leads to its production by recombinant DNA methods, including gene knockouts, gene replacement, or partial or complete replacement of gene promoter sequences affecting genes in these pathways. In some examples, these include pathways leading to production of ethanol, acetate, lactate, succinate, butyrate, and butanol.

In one embodiment, production of alcohols (short or long chain, branched or straight-chain, saturated or unsaturated) is optimized by introduction of one or more exogenous nucleic acids encoding proteins in alcohol synthesis pathways. Alcohols can be used as products or used to create products comprised of fatty acid esters, alkyl esters, isoprenyl esters, or other esters.

In one embodiment, such organisms are modified such that they produce or upregulate production of polyhydroxybutyrate (PHB) or other products classified as polyhydroxyalkanoates (PHAs). Organisms that already produce a specific PHA may be modified to produce more of the same or of a different PHA under cultivation conditions appropriate for chemoautotrophic cultivation. Alternatively, organisms that do not produce PHAs may be modified to produce one or multiple types of PHAs. Examples of pathway genes that enable production of PHAs include the following, for production of PHB: a beta-ketothiolase (which converts acetyl-CoA to acetoacetyl-CoA and CoA), Acetoacetyl-CoA reductase (which converts acetoacetyl-CoA and NADPH to 3-hydroxybutyryl-CoA), and PHA synthase (which converts 3-hydroxybutyryl-CoA to PHB and CoA). An example of such a pathway, enabling production of PHB, is encoded by the Ralstonia eutropha phaCAB operon. In some embodiments, specific modifications are made by recombinant methods to knockout or attenuate genes that degrade or prevent the accumulation of PHAs. An example of such a gene is poly[(R)-3-hydroxybutanoate] hydrolase.

In one embodiment, such organisms are modified such that they produce detectable levels of hydrocarbons or fatty acids of desired structure from inorganic energy and CO₂. For production of specific products of commercial value, desired structures or characteristics includes carbon chain length, branching, and saturation levels. In preferred embodiments, such organisms are modified such that they produce high yields of desired hydrocarbons. In certain embodiments, hydrocarbons produced are secreted by passive transport proteins, active transport proteins or combinations thereof. In certain embodiments, secretion is optimized for maximum yield of secreted hydrocarbons by introducing one or more exogenous nucleic acid sequences encoding transport proteins or gene regulatory sequences (e.g., promoters) that directly modify expression of transport proteins. In certain embodiments, such organisms are optimized for maximum yield of secreted, desired hydrocarbons by introducing one or more exogenous nucleic acid sequences encoding proteins that regulate the expression of transport proteins or gene regulatory sequences (e.g., promoters) that directly modify expression of transport proteins. In certain embodiments, such organisms are optimized for maximum yield of secreted hydrocarbons by introduction of one or more nucleic acid sequences that knock out or attenuate expression of certain endogenous transport proteins or proteins that regulate endogenous transport proteins. In one embodiment, the microorganisms are introduced with one or more exogenous nucleic acid sequences encoding acetyl-CoA carboxylase activity (accBCAD), aldehyde dehydrogenase activity (adhA, adhB), alcohol dehydrogenase activity (ADH I), alkane 1-monooxygenase activity (alkB), 3-hydroxyacyl-ACP dehydratase activity (fabA), 3-ketoacyl-ACP synthase activity (fabB), malonyl-CoA:ACP transacylase activity (fabD), 3-ketoacyl-ACP reductase activity (fabG), acetyl-CoA:ACP transacylase activity (fabH), enoyl-ACP reductase activity (fabl), acyl-ACP hydrolase activity (FAS1), the E1p dehydrogense component of the pyruvate dehydrogenase complex, the E2p dihydrolipoamide acyltransferase component of the 2-oxoglutarate dehydrogenase complex, genes encoding fatty-acyl-coA reductases, fatty alcohol forming acyl-CoA reductases, pyridine nucleotide transhydrogenases, and genes encoding fatty-acyl-coA reductases, acyl-CoA synthetase, alcohol dehydrogenase, alcohol acetyltransferase (EC 2.3.1.84), thioesterase, (EC 3.1.2.14), aceE, aceF, acpP, fadD, cerl, fabA, fabB, fabD, fabG, fabH, fabl, fabZ, lipase, malonyl-CoA decarboxylase, panD, panK, pdh, udhA, and wax synthase (EC 2.3.1.75).

In one embodiment of the invention, such organisms are modified to secrete fatty acid chains by introduction of one or more exogenous nucleic acid sequences encoding an acyl-ACP-thioesterase, wherein the acyl-ACP-thioesterases liberate fatty acid chains from ACP-thioesters. In one example, production of fatty acids of specific lengths, or enriched for specific lengths and structure (including branching and degree of saturation), can be produced by the introduction of one or more nucleic acid sequences encoding specific acyl-ACP-thioesterases showing a bias for producing fatty acid chains of a specific length and structure. In some examples, an organism may be modified by introduction of one or multiple exogenous nucleic acid sequences encoding multiple acyl-ACP-thioesterase proteins into the same organism such that the organism produces fatty acids of multiple specific lengths and structures, or enriched for multiple specific lengths and structures. Several examples of such thioesterases are available in the art, published in the patent literature or in the open literature.

In one embodiment, such organisms are modified by the introduction of one or more nucleic acid sequences to enable or enhance the ability of the organism to utilize inorganic energy, CO2, and water to generate carbon-based products, including amino acids, acrylate, acrylic acid, adipic acid, alcohol, ascorbate, ascorbic acid, aspartate, aspartic acid, 1,3-butadiene, 1,3-butanediol, 2,3-butanediol, 1,4-butanediol, butanol, caprolactam, carotenoid, citrate, citric acid, DHA, diesel, docetaxel, e-caprolactone, erythromycin 7-ADCA/cephalosporin, ethanol, ethyl ester, ethylene, fatty acid ester, fatty alcohols, fuel oxygenates, gamma butyrolactone, gasoline, glucose, fructose, carbohydrate, glutamate, glutamic acid, HPA, hydrocarbons, hydroxybutyrate, 3-hydroxypropionate, isopentenol, isoprene, isoprenoid, isopropanol, itaconate, itaconic acid, JetA, JetA-1, JetB, JP4, JP8, lactate, lactic acid, lanosterol, levulinic acid, limonene, lycopene, lysine, malate, malonic acid, methyl ester, muconic acid, nucleic acids, n-alkanes, alkenes, octane, omega fatty acid, omega-3 DHA, paclitaxel, peptide, PHA, PHB, pharmaceutical products or pharmaceutical intermediates, polyketides, polymers, polyol, propane, 1,3-propanediol, propanol, propylene, pyrrolidones, rubber, serine, sorbitol, statin, steroid, succinate, sucrose, terephthalate, terpene, THF, γ-valerolactone, and wax ester.

In certain embodiments, such organisms provided by the invention comprises a cell line selected from eukaryotic plants, algae, cyanobacteria, green-sulfur bacteria, green non-sulfur bacteria, purple sulfur bacteria, purple non-sulfur bacteria, extremophiles, yeast, fungi, proteobacteria, engineered organisms thereof, and synthetic organisms.

In certain embodiments, such organisms are chemoautotrophic microorganisms that include, but are not limited to, one or more of the following: Acetoanaerobium sp., Acetobacterium sp., Acetogenium sp., Achromobacter sp., Acidianus sp., Acinetobacter sp., Actinomadura sp., Aeromonas sp., Alcaligenes sp., Alcaligenes sp., Arcobacter sp., Aureobacterium sp., Bacillus sp., Beggiatoa sp., Butyribacterium sp., Carboxydothermus sp., Clostridium sp., Comamonas sp., Dehalobacter sp., Dehalococcoide sp., Dehalospirillum sp., Desulfobacterium sp., Desulfomonile sp., Desulfotomaculum sp., Desulfovibrio sp., Desulfurosarcina sp., Ectothiorhodospira sp., Enterobacter sp., Eubacterium sp., Ferroplasma sp., Halothibacillus sp., Hydrogenobacter sp., Hydrogenomonas sp., Leptospirillum sp., Metallosphaera sp., Methanobacterium sp., Methanobrevibacter sp., Methanococcus sp., Methanosarcina sp., Micrococcus sp., Nitrobacter sp., Nitrosococcus sp., Nitrosolobus sp., Nitrosomonas sp., Nitrosospira sp., Nitrosovibrio sp., Nitrospina sp., Oleomonas sp., Paracoccus sp., Peptostreptococcus sp., Planctomycetes sp., Pseudomonas sp., Ralstonia sp., Rhodobacter sp., Rhodococcus sp., Rhodocyclus sp., Rhodomicrobium sp., Rhodopseudomonas sp., Rhodospirillum sp., Shewanella sp., Streptomyces sp., Sulfobacillus sp., Sulfolobus sp., Thiobacillus sp., Thiomicrospira sp, Thioploca sp., Thiosphaera sp., Thiothrix sp. Also chemoautotrophic microorganisms that are generally categorized as sulfur-oxidizers, hydrogen-oxidizers, iron-oxidizers, acetogens, and methanogens, as well as a consortiums of microorganisms that include chemoautotrophs.

Such organisms also include but are not limited to extremophiles that can withstand extremes in various environmental parameters such as temperature, radiation, pressure, gravity, vacuum, desiccation, salinity, pH, oxygen tension, and chemicals. They include hyperthermophiles, such as Pyrolobus fumarii; thermophiles, such as Synechococcus lividis: mesophiles, and psychrophiles, such as Psychrobacter. Radiation tolerant organisms include Deinococcus radiodurans. Pressure tolerant organisms include piezophiles or barophiles. Dessicant tolerant and anhydrobiotic organisms include xerophiles such as Artemia salina; microbes and fungi. Salt tolerant organisms include halophiles, such as Halobacteriacea and Dunaliella salina. pH tolerant organisms include alkaliphiles such as Natronobacterium, Bacillus firmus OF4, Spirulina spp., and acidophiles such as Cyanidium caldarium, Ferroplasma sp. Gas tolerant organisms, which tolerate pure CO₂ include Cyanidium caldarium and metal tolerant organisms include metalotolerants such as Ferroplasma acidarmanus, Ralstonia sp.

Such organisms also include algae and cyanobacteria, which include, but are not limited to the following genera: Acanthoceras, Acanthococcus, Acaryochloris, Achnanthes, Achnanthidium, Actinastrum, Actinochloris, Actinocyclus, Actinotaenium, Amphichrysis, Amphidinium, Amphikrikos, Amphipleura, Amphiprora, Amphithrix, Amphora, Anabaena, Anabaenopsis, Aneumastus, Ankistrodesmus, Ankyra, Anomoeoneis, Apatococcus, Aphanizomenon, Aphanocapsa, Aphanochaete, Aphanothece, Apiocystis, Apistonema, Arthrodesmus, Artherospira, Ascochloris, Asterionella, Asterococcus, Audouinella, Aulacoseira, Bacillaria, Balbiania, Bambusina, Bangia, Basichlamys, Batrachospermum, Binuclearia, Bitrichia, Blidingia, Botrdiopsis, Botrydium, Botryoroccus, Botryosphaerella, Brachiomonas, Brachysira, Brachytrichia, Brebissonia, Bulbochaete, Bumilleria, Bumilleriopsis, Caloneis, Calothrix, Campylodiscus, Capsosiphon, Carteria, Catena, Cavinula, Centritractus, Centronella, Ceratium, Chaetoceros, Chaetochloris, Chaetomorpha, Chaetonella, Chaetonema, Chaetopeltis, Chaetophora, Chaetosplaeridium, Chramaesiphon, Chara, Characiochloris, Characiopsis, Characium, Charales, Chilomonas, Chlainomonas, Chlamydoblepharis, Chlamydocapsa, Chlamydomonas, Chlamydomonopsis, Chlamydomyxa, Chlamydonephris, Chlorangiella, Chlorangiopsis, Chlorella, Chlorombtrys, Chlorobrachis, Chlorochytrium, Chlorococcum, Chlorgloea, Chlorogloeopsis, Chlorogonium, Chlorolobion, Chloromonas, Chlorophysema, Chlorophyta, Chlorosaccus, Chlorosarcina, Choricystis, Chromophyton, Chromulina, Chroococcidiopsis, Chroococcus, Chroodactylon, Chroomonas, Chroothece, Chrysamoeba, Chrysapsis, Chrysidiastrum, Chrysocapsa, Chrysocapsella, Chrysochaaete, Chrysochromulina, Chrysococcus, Chrysocrinus, Chrysolepidomonas, Chrysolykos, Chrysonebula, Chrysophyta, Chrysopyxis, Chrysosaccus, Chrysophaerella, Chrysostephanosphaera, Clodophora, Clastidium, Closteriopsis, Closterium, Coccomyxa, Cocconeis, Coelastrella, Coelastrum, Coelosphaerium, Coenochloris, Coenococcus, Coenocystis, Colacium, Coleochaete, Collodictyon, Compsogonopsis, Compsopogon, Conjugatophyta, Conochaete, Coronastrum, Cosmarium, Cosmioneis, Cosmocladium, Crateriportula, Craticula, Crinalium, Crucigenia, Crucigeniella, Cryptoaulax, Cryptomonas, Cryptophyta, Ctenophora, Cyanodictyon, Cyanonephron, Cyanophora, Cyanophyta, Cyanothece, Cyanothomonas, Cyclonexis, Cyclostephanos, Cyclotella, Cylindrocapsa, Cylindrocystis, Cylindrospermum, Cylindrotheca, Cymatopleura, Cymbella, Cymbellonitzschia, Cystodinium Dactylococcopsis, Debarya, Denticula, Dermatochrysis, Dermocarpa, Dermocarpella, Desmatractum, Desmidium, Desmococcus, Desmonema, Desmosiphion, Diacanthos, Diacronema, Diadesmis, Diatoma, Diatomella, Dicellula, Dichothrix, Dichotomococcus, Dicranochaete, Dictyochloris, Dictyococcus, Dictyosphaerium, Didymocystis, Didymogenes, Didymosphenia, Dilabifilum, Dimorphococcus, Dinobryon, Dinococcus, Diplochloris, Diploneis, Diplostauron, Distrionella, Docidium, Draparnaldia, Dunaliella, Dysmorphococcus, Ecballocystis, Elakatothrix, Ellerbeckia, Encyonema, Enteromorpha, Entocladia, Entomoneis, Entophysalis, Epichrysis, Epipyxis, Epithemia, Ermosphaera, Euastropsis, Euastrum, Eucapsis, Eucocconeis, Eudorina, Euglena, Euglenophyta, Eunotia, Eustigmatophyta, Eutreptia, Fallacia, Fischerella, Fragilaria, Fragilariforma, Franceia, Frustulia, Curcilla, Geminella, Genicularia, Glaucocystis, Glaucophyta, Glenodiniopsis, Glenodinium, Gloeocapsa, Gloeochaete, Gleoochrysis, Gloeococcus, Gloeocystis, Gloeodendron, Gloeomonas, Gloeoplax, Gloeothece, Gloeotila, Gloeotrichia, Gloiodictyon, Golenkinia, Golenkiniopsis, Gomontia, Gomphocymbella, Gomphonema, Gomphosphaeria, Gonatozygon, Gongrosia, Gongrosira, Goniochloris, Gonium, Gonyostomum, Granulochloris, Granulocystopsis, Groenbladia, Gymnodinium, Gymnozyga, Gyrosigma, Haematococcus, Hafniomonas, Hallassia, Hammatoidea, Hannaea, Hantzschia, Hapalosiphon, Haplotaenium, Haptophyta, Haslea, Hemidinium, Hemitonia, Heribaudiella, Heteromastix, Heterothrix, Hibberdia, Hildenbrandia, Hillea, Holopedium, Homoeothrix, Hormanthonema, Hormotila, Hyalobrachion, Hyalocardium, Hyalodiscus, Hyalogonium, Hyalotheca, Hydrianum, Hydrococcus, Hydrocoleum, Hydrocoryne, Hydrodictyon, Hydrosera, Hydrurus, Hyella, Hymenomonas, Isthmochloron, Johannesbaptistia, Juranyiella, Karayevia, Kathablepharis, Katodinium, Kephyrion, Keratococcus, Kirchneriella, Klebsormidium, Kolbesia, Koliella, Komarekia, Korshikoviella, Kraskella, Lagerheimia, Lagynion, Lamprothamnium, Lemanea, Lepocinclis, Leptosira, Lobococcus, Lobocystis, Lobomonas, Luticola, Lyngbya, Malleochloris, Mallomonas, Mantoniella, Marssoniella, Martyana, Mastigocoleus, Gastogloia, Melosira, Merismopedia, Mesostigma, Mesotaenium, Micractinium, Micrasterias, Microchaete, Microcoleus, Microcystis, Microglena, Micromnonas, Microspora, Microthamnion, Mischococcus, Monochrysis, Monodus, Monomastix, Monoraphidium, Monostroma, Mougeotia, Mougeotiopsis, Myochloris, Myromecia, Myxosarcina, Naegeliella, Nannochloris, Nautococcus, Navicula, Neglectella, Neidium, Nephroclamys, Nephrocylium, Nephrodiella, Nephroselmis, Netrium, Nitella, Nitellopsis, Nitzschia, Nodularia, Nostoc, Ochromonas, Oedogonium, Oligochaetophora, Onychonema, Oocardium, Oocystis, Opephora, Ophiocytium, Orthoseira, Oscillatoria, Oxyneis, Pachycladella, Palmella, Palmodictyon, Pnadorina, Pannus, Paralia, Pascherina, Paulschulzia, Pediastrum, Pedinella, Pedinomonas, Pedinopera, Pelagodictyon, Penium, Peranema, Peridiniopsis, Peridinium, Peronia, Petroneis, Phacotus, Phacus, Phaeaster, Phaeodermatium, Phaeophyta, Phaeosphaera, Phaeothamnion, Phormidium, Phycopeltis, Phyllariochloris, Phyllocardium, Phyllomitas, Pinnularia, Pitophora, Placoneis, Planctonema. Plankiosphaeria, Planothidium, Plectonema, Pleodorina, Pleurastrum, Pleurocapsa, Pleurocladia, Pleurodiscus, Pleurosigma, Pleurosira, Pleurotaenium, Pocillomonas, Podohedra, Polyblepharides, Polychaetophora, Polyedriella, Polyedriopsis, Polygoniochloris, Polyepidomonas, Polytaenia, Polytoma, Polytomella, Porphyridium, Posteriochromonas, Prasinochloris, Prasinocladus, Prasinophyta, Prasiola, Prochlorphyta, Prochlorothrix, Protoderma, Protosiphon, Provasoliella, Prymnesium, Psammodictyon, Psammothidium, Pseudanabaena, Pseudenoclonium, Psuedocarteria, Pseudochate, Pseudocharacium, Pseudococcomyxa, Pseudodictyosphaerium, Pseudokephyrion, Pseudoncobyrsa, Pseudoquadrigula, Pseudosphaerocystis, Pseudostaurastrum, Pseudostaurosira, Pseudotetrastrum, Pteromonas, Punctastruata, Pyramichlamys, Pyranumonas, Pyrrophyta, Quadrichloris, Quadricoccus, Quadrigula, Radiococcus, Radiofilum, Raphidiopsis, Raphidocelis, Raphidonema, Raphidophyta, Peimeria, Rhabdoderma, Rhabdomonas, Rhizoclonium, Rhodomonas, Rhodophyta, Rhoicosphenia, Rhopalodia, Rivularia, Rosenvingiella, Rossithidium, Roya, Scenedesmus, Scherffelia, Schizochlamydella, Schizochlamys, Schizomeris, Schizothrix, Schoederia, Scolioneis, Scotiella, Scotiellopsis, Scourfieldia, Scytonema, Selenastrum, Selenochloris, Sellaphora, Semiorbis, Siderocelis, Diderocystopsis, Dimonsenia, Siphononema, Sirocladium, Sirogonium, Skeletonema, Sorastrum, Spermatozopsis, Sphaerellocystis, Sphaerellopsis, Sphaerodinium, Sphaeroplea, Sphaerozosma, Spiniferomonas, Spirogyra, Spirotaenia, Spirulina, Spondylomorum, Spondylosium, Sporotetras, Spumella, Staurastrum, Stauerodesmus, Stauroneis, Staurosira, Staurosirella, Stenopterobia, Stephanocostis, Stephanodiscus, Stephanoporos, Stephanosphaera, Stichococcus, Stichogloea, Stigeoclonium, Stigonema, Stipitococcus, Stokesiella, Strombomonas, Stylochrysalis, Stylodinium, Styloyxis, Stylosphaeridium, Surirella, Sykidion, Symploca, Synechococcus, Synechocystis, Synedra, Synochromonas, Synura, Tabellaria, Tabularia, Teilingia, Temnogametum, Tetmemorus, Tetrachlorella, Tetracyclus, Tetradesmus, Tetraedriella, Tetraedron, Tetraselmis, Tetraspora, Tetrastrum, Thalassiosira, Thamniochaete, Thorakochloris, Thorea, Tolypella, Tolypothrix, Trachelomonas, Trachydiscus, Trebouxia, Trentepholia, Treubaria, Tribonema, Trichodesmium, Trichodiscus, Trochiscia, Tryblionella, Ulothrix, Uroglena, Uronema, Urosolenia, Urospora, Uva, Vacuolaria, Vaucheria, Volvox, Volvulina, Westella, Woloszynskia, Xanthidium, Xanthophyta, Xenococcus, Zygnema, Zygnemopsis, and Zygonium.

Such organisms also include green non-sulfur bacteria, which include but are not limited to the following genera: Chloroflexus, Chloronema, Oscillochloris, Heliothrix, Herpetosiphon, Roseiflexus, and Thermomicrobium.

Such organisms also include green sulfur bacteria, which include but are not limited to the following genera: Chlorobium, Clathrochloris, and Prosthecochloris.

Such organisms also include purple sulfur bacteria, which include but are not limited to the following genera: Allochromatium, Chromatium, Halochromatium, Isochromatium, Marichromatium, Rhodovulum, Thermochromatium, Thiocapsa, Thiorhodococcus, and Thiocyslis.

Such organisms also include purple non-sulfur bacteria, which include but are not limited to the following genera: Phaeospirillum, Rhodobaca, Rhodobacter, Rhodomicrobium, Rhodopila, Rhodopseudomonas, Rhodothalassium, Rhodospirillum, Rodovibrio, and Roseospira.

Such organisms also include aerobic chemolithotrophic bacteria, which include but are not limited to nitrifying bacteria such as Nitrobacrteraceae sp., Nitrobacter sp., Nitrospina sp., Nitrococcus sp. Nitrospira sp., Nitrosomonas sp., Nitrosococcus sp., Nitrosospira sp., Nitrosolobus sp., Nitrosovibrio sp.; colorless sulfur bacteria such as, Thiovulum sp. Thiobacillus sp., Thiomicrospira sp., Thiosphaera sp., Thermothrix sp.; obligately chemolithotrophic hydrogen bacteria such as Hydrogenobacter sp., iron and manganese-oxidizing and/or depositing bacteria such as Siderococcus sp., and magnetotactic bacteria such as Aquaspirillum sp.

Such organisms also include archaeobacteria, which include but are not limited to methanogenic archaeobacteria such as Methanobacterium sp., Methanobrevibacter sp. Methanothermus sp., Methanococcus sp., Methanomicrobium sp., Methanospirillum sp., Methanogenium sp., Methanosarcina sp., Methanolobus sp. Methanothrix sp., Methanococcoides sp., Methanoplanus sp.; extremely thermophilic sulfur-metabolizers such as Thermoproteus sp., Pyrodictium sp. Sulfolobus sp. Acidianus sp.

In some embodiments of the invention a oxyhydrogen microorganism, such as but not limited to Ralstonia eutropha, Alcaligenes eutrophus or Cupriavidus necator, is grown up to a high cell density in micro aerobic conditions using syngas components as a carbon source and energy, including, but not limited to H2, CO2 and/or CO, and/or using methanol and/or using glycerol, including crude glycerol, which is a by-product of biodiesel or oleochemical manufacturing. Once a high cell density is achieved, feeding oxygen into the bioreactor is stopped and fementation continues under aneaorobic conditions and the microorganisms secrete 1,3 butanediol or 2,3 butanediol and/or other organic compounds, including, but not limited to 2-Oxoglutarate. 2-Oxo-3-methylbutanoate, cis-Aconitate, 3-Hydroxybutanoate, Butanoate, Acetate, Formate, Succinate. 2-methyl propanoate. 2-Methylbutanoate, 3-Methylbutanoate, meso-2,3-Butandiol, Acetoin, DL, 2,3-Butandiol, 2-Methylpropan-1-ol, Ethanol, 1-Propanol, and/or Lactate.

Exemplary oxyhydrogen microorganisms that can be used in one or more process steps of certain embodiments of the present invention include but are not limited to one or more of the following: purple non-sulfur photosynthetic bacteria including but not limited to Rhodopseudomonas palustris, Rhodopseudomonas capsulata, Rhodopseudomonas viridis, Rhodopseudomonas sulfoviridis, Rhodopseudomonas blastica, Rhodopseudomonas spheroides, Rhodopseudomonas acidophila and other Rhodopseudomonas sp., Rhodospirillum rubrum, and other Rhodospirillum sp.; Rhodococcus opacus and other Rhodococcus sp.; Rhizobium japonicum and other Rhizobium sp.; Thiocapsa roseopersicina and other Thiocapsa sp.; Pseudomonas hydrogenovora, Pseudomonas hydrogenothermophila, and other Pseudomonas sp.; Hydrogenomonas pantotropha, Hydrogenomonas eutropha, Hydrogenomonas facilis, and other Hydrogenomonas sp.; Hydrogenobacter thermophilus and other Hydrogenobacter sp.; Hydrogenovibrio marinus and other Hydrogenovibrio sp.; Helicobacter pylori and other Helicobacter sp.; Xanthobacter sp.; Hydrogenophaga sp.; Bradyrhizobium japonicum and other Bradyrhizobium sp.; Ralstonia eutropha and other Ralstonia sp.; Alcaligenes eutrophus and other Alcaligenes sp.; Variovorax paradoxus, and other Variovorax sp.; Acidororax facilis, and other Acidovorax sp.; cyanobacteria including but not limited to Anabaena oscillarioides, Anabaena spiroides, Anabaena cylindrica, and other Anabaena sp. green algae including but not limited to Scenedesmus obliquus and other Scenedesmus sp. Chlamydomonas reinhardii and other Chlamydomonas sp., Ankistrodesmus sp., Rhaphidium polymorphium and other Rhaphidium sp.; as well as a consortiums of microorganisms that include oxyhydrogen microorganisms.

One feature of certain embodiments of the present invention is the inclusion of one or more process steps within a chemical process for the conversion of C1 carbon sources including but not limited to carbon monoxide, methane, methanol, formate, or formic acid, and/or mixtures containing C1 chemicals including but not limited to various syngas compositions generated from various gasified, pyrolyzed, or steam-reformed fixed carbon feedstocks, that utilize oxyhydrogen microorganisms and/or enzymes from oxyhydrogen microorganisms as a biocatalyst for the conversion of C1 chemicals into longer chain organic chemicals (i.e. C2 or longer and, in some embodiments. C5 or longer carbon chain molecules). In some such embodiments C1 containing syngas, or process gas, or C1 chemicals in a pure liquid form or dissolved in solution is pumped or otherwise added to a vessel or enclosure containing nutrient media and oxyhydrogen microorganisms. In some such cases oxyhydrogen microorganisms perform biochemical synthesis to elongate C1 chemicals into longer carbon chain organic chemicals using the chemical energy stored in the C1 chemical, and/or molecular hydrogen and/or valence or conduction electrons in solid state electrode materials and/or one or more of the following list of electron donors pumped or otherwise provided to the nutrient media including but not limited to: ammonia; ammonium; carbon monoxide; dithionite: elemental sulfur; hydrocarbons: metabisulfites; nitric oxide; nitrites; sulfates such as thiosulfates including but not limited to sodium thiosulfate (Na2S2O3) or calcium thiosulfate (CaS2O3); sulfides such as hydrogen sulfide; sulfites; thionate: thionite; transition metals or their sulfides, oxides, chalcogenides, halides, hydroxides, oxyhydroxides, sulfates, or carbonates, in soluble or solid phases. The electron donors can be oxidized by electron acceptors in a chemosynthetic reaction. Electron acceptors that may be used at this reaction step include oxygen and/or other electron acceptors including but not limited to one or more of the following: carbon dioxide, ferric iron or other transition metal ions, nitrates, nitrites, oxygen, or holes in solid state electrode materials.

The chemosynthetic reaction step or steps of the process whereby carbon dioxide and/or inorganic carbon is fixed into organic carbon in the form of organic compounds and biomass and/or the reaction steps converting C1 chemicals to longer chain organic chemicals whereby a C1 chemical such as but not limited to carbon monoxide, methane, methanol, formate, or formic acid, and/or mixtures containing C1 chemicals including but not limited to various syngas compositions generated from various gasified, pyrolyzed, or steam-reformed fixed carbon feedstocks, are biochemically converted into longer chain organic chemicals (i.e. C2 or longer and, in some embodiments, C5 or longer carbon chain molecules) can be performed in aerobic, microaerobic, anoxic, anaerobic conditions, or facultative conditions. A facultative environment is considered to be one having aerobic upper layers and anaerobic lower layers caused by stratification of the water column.

The present invention relates to the engineering of microorganisms, including but not limited to hydrogen oxidizing and/or carbon monoxide oxidizing knallgas microorganisms, with a natural capability to grow and synthesize biomass on gaseous carbon sources such as syngas and/or CO₂, such that the natural or engineered microorganisms synthesize targeted products, including chemicals and fuels, under gas cultivation.

In some embodiments, the composition comprises a microorganism that can naturally grow on H2/CO2 and/or syngas, and wherein the microorganism can naturally accumulate polyhydroxybutyrate (PHB) or polyhydroxyalkanoate (PHA) to 50% or more of the cell biomass by weight. In some embodiments the microorganisms have a native ability to direct a high flux of carbon through the acetyl-CoA metabolic intermediate, which can lead into fatty acid biosynthesis, along with a number of other synthetic pathways including PHA and PHB synthesis. A microorganism is considered to direct a high flux of carbon through acetyl-CoA if a product of a synthesis pathway going through the acetyl-CoA metabolic intermediate, including but not limited to polyhydroxybutyrate (PHB) or polyhydroxyalkanoate (PHA), can represent 50% or more of the cell biomass by weight. In some embodiments the microorganism exhibiting these traits is Cupriavidus necator (DSM 531 or DSM 541).

Aspects of the invention relate to a bacterial cell comprising at least a first exogenous nucleic acid sequence wherein the cell converts gaseous CO₂ and/or gaseous H₂ and/or syngas into one or more lipids or hydrocarbons.

In some embodiments, the first exogenous nucleic acid sequence encodes a protein selected from the group consisting of a fatty acid acyl-ACP reductase and a fatty acid aldehyde decarbonylase. In some embodiments, the first exogenous nucleic acid sequence encodes a CYP52A protein. In certain embodiments, the first exogenous nucleic acid sequence encodes a protein selected from the group consisting of a CYP709C1 and CYP81B1. In some embodiments, the first exogenous nucleic acid sequence encodes a thioesterase protein.

In some embodiments, the cell further comprises a second exogenous nucleic acid sequence. In some embodiments, the first exogenous nucleic acid sequence encodes a fatty acid acyl-ACP reductase and the second exogenous nucleic acid sequence encodes a fatty acid aldehyde decarbonylase. In some embodiments, the cell comprises a first and second exogenous nucleic acid wherein the second exogenous nucleic acid encodes a thioesterase protein or a fatty acyl-CoA ligase. In some embodiments, the cell further comprises a third exogenous nucleic acid sequence that encodes a thioesterase.

In some embodiments, the bacterial cell is of the suborder corynebacterineae. In some embodiments, the bacterial cell is of the family burkholderiaceae. In some embodiments, the cell is of the genera Rhodococcus or Gordonia. In certain embodiments, the cell is a Rhodococcus opacus. In some embodiments, the bacterial cell is an oxyhydrogen microorganisms including oxyhydrogen microorganisms selected from one or more of the following genera: Rhodopseudomonas sp.; Rhodospirillum sp.; Rhodococcus sp.; Nocardia sp.; Mycobacterium sp.; Gordonia sp.; Tsukamurella sp.; Rhodobacter sp.; Rhizobium sp.; Thiocapsa sp.; Pseudomonas sp.; Hydrogenomonas sp.; Hydrogenobacter sp.; Hydrogenovibrio sp.; Helicobacter sp.; Oleomonas sp.; Xanthobacter sp.; Hydrogenophaga sp.; Bradyrhizobium sp.; Ralstonia sp.; Alcaligenes sp.; Variovorax sp.; Acidovorax sp.; Anabaena sp.; Scenedesmus sp.; Chlamydomonas sp., Ankistrodesmus sp., and Rhaphidium sp. [all oxyhydrogen] subset of hydrogen oxidizers.

In some embodiments, the bacterial cell produces and/or secretes at least 10% of one or more lipids or hydrocarbons by weight. In some embodiments, the bacterial cell produces and/or secretes one or more lipids or hydrocarbons, wherein at least 50% of the one or more lipids or hydrocarbons have 6 to 30 carbon atoms. In some embodiments, less than 10% by weight of the lipids or hydrocarbons is methane. In some embodiments, less than 10% by weight of the lipids or hydrocarbons is organic acid.

In some embodiments, the one or more lipids or hydrocarbons comprise at least one organic molecule having a carbon chain length of at least 8 carbon atoms and at least one carbon-carbon double bond. In some embodiments, the one or more lipids or hydrocarbons comprise at least one diacid acid molecule having a carbon chain length of at least 6 carbon atoms. In some embodiments, the one or more lipids or hydrocarbons comprise at least one desaturated hydrocarbon molecule having a carbon chain length of at least 6 carbon atoms.

In some embodiments, the one or more lipids or hydrocarbons comprise at least one fatty acid molecule having a carbon chain length of at least 6 carbon atoms. In some embodiments, the one or more lipids or hydrocarbons comprise at least one unsaturated fatty acid molecule having a carbon chain length of at least 6 carbon atoms. In some embodiments, the one or more lipids or hydrocarbons comprise at least one hydroxyl acid molecule having a carbon chain length of at least 6 carbon atoms. In some embodiments, the one or more lipids or hydrocarbons comprise at least one dicarboxylic acid molecule having a carbon chain length of at least 6 carbon atoms.

In some embodiments, the one or more lipids or hydrocarbons comprise at least one alkane, alkene, alkyne, fatty alcohol, and/or fatty aldehyde at a level higher than the quantity of the alkane, alkene, alkyne, fatty alcohol, and or fatty aldehyde in the same microorganism not comprising the exogenous nucleic acid sequences. In some embodiments, the one or more lipids or hydrocarbons comprise at least one component of or one precursor to a component of jet fuel, diesel fuel, or biodiesel fuel.

Further aspects of the invention relate to a method of producing a lipid or a hydrocarbon or a mixture of lipids or hydrocarbons, including culturing a bacterial cell in a feedstock comprising syngas and/or gaseous CO₂ and/or a mixture of CO₂ gas and H₂ gas. In some embodiments, the H₂ is generated or recycled using renewable, alternative, or conventional sources of power that are low in greenhouse gas emissions, and wherein said sources of power are selected from at least one of photovoltaics, solar thermal, wind power, hydroelectric, nuclear, geothermal, enhanced geothermal, ocean thermal, ocean wave power, and tidal power. In some embodiments, the syngas is generated from lignocellulosic energy crops, crop residue, bagasse, saw dust, forestry residue, food waste, municipal solid waste, biogas, landfill gas, or stranded natural gas.

In some embodiments, the lipid or hydrocarbon or mixture of lipids or hydrocarbons produced is one or more alkane, alkene, alkyne, fatty alcohol, and/or fatty aldehyde. In some embodiments, at least one exogenous nucleic acid sequences of the bacterial cell is operably linked to a promoter that is inducible in response to a first stimulus, and wherein the method further comprises culturing a population of the bacterial cell of claim 1 for a first period of time in the presence of a first stimulus to produce one or more lipids or hydrocarbons.

Further aspects of the invention relate to culturing of a bacterial cell in a feedstock comprising syngas and/or gaseous CO₂ and/or a mixture of CO₂ gas and H₂ gas in a reaction vessel or a bioreactor wherein the one or more lipids or hydrocarbons are separated from the reaction vessel or bioreactor. In some embodiments, the method further comprises purifying the one or more lipids or hydrocarbons or a mixture of lipids or hydrocarbons after separation from the reaction vessel or bioreactor.

Further aspects of the invention relate to a microorganism comprising at least a first exogenous nucleic acid sequence wherein the microorganism converts gaseous CO₂ and/or gaseous H₂ and/or syngas into one or more hydroxylated fatty acids. In some embodiments, the first exogenous nucleic acid sequence encodes a hydroxylating ezyme. In some embodiments the cell further comprises a second exogenous nucleic acid sequence encoding a thioesterase enzyme. In some embodiments, the microorganism is the genera Rhodococcus or Gordonia. In certain embodiments, the microorganism is the species Rhodococcus sp. DSM 3346 or DSM 364. In some embodiments, the microorganism is Rhodococcus opacus. In certain embodiments, the microorganism is Rhodococcus opacus (DSM 43205) or Rhodococcus opacus (DSM 43206) or Rhodococcus opacus (DSM 44193). In some embodiments, the microorganism is family Burkholderiaceae. In some embodiments, the microorganism is Cupriavidus necator. In some embodiments, the microorganism is Cupriavidus metallidurans. In some embodiments, the microorganism is a knallgas microorganism, also known as an oxyhydrogen microorganism. In some embodiments, herein the microorganism is a chemoautotrophic microbe.

In some embodiments, the wild-type or mutant of the microorganism naturally has a capability for accumulating and/or synthesizing high quantities of triacylglycerol where a high quantity is considered to be 10% or more of the dry cell mass. In some embodiments, the microorganism is a hydrogen-oxidizing chemoautotroph. In some embodiments, the microorganism is capable of growing on syngas as the sole energy and carbon source. In some embodiments, the microorganism is capable of growing on untreated crude glycerol as the sole energy and carbon source.

Further aspects of the invention relate to a method for producing hydroxylated fatty acids including in a bioreactor or solution, culturing an engineered microorganism or a natural strain in a feedstock comprising syngas and/or gaseous CO₂ and/or a mixture of CO₂ gas and H₂ gas. In some embodiments, the method further comprises the step of up-regulating an endogenous or exogenous thioesterase gene of the microorganism. In some embodiments, the method further comprises the step of down-regulating production of an endogenous or exogenous thioesterase gene of the microorganism. In some embodiments, the method further comprises the step of down regulating an endogenous or exogenous acyl carrier protein gene of the microorganism.

Aspects of the invention relate to a microorganism comprising at least a first exogenous nucleic acid sequence wherein the microorganism converts gaseous CO₂ and/or gaseous H₂ and/or syngas into one or more shorter-chain fatty acids. In some embodiments, the first exogenous nucleic acid sequence encodes a fatty acyl-CoA binding protein. In some embodiments, the microorganism further comprises a second exogenous nucleic acid sequence encoding a thioesterase enzyme. In some embodiments, the microorganism is of the genera Rhodococcus or Gordonia. In certain embodiments, the microorganism is the species Rhodococcus sp. DSM 3346 or DSM 364. In some embodiments, the microorganism is a Rhodococcus opacus. In some embodiments, the microorganism is a Rhodococcus opacus (DSM 43205) or a Rhodococcus opacus (DSM 43206) or a Rhodococcus opacus (DSM 44193). In some embodiments, the microorganism is family burkholderiaceae. In some embodiments, the microorganism is Cupriavidus necator. In some embodiments, the microorganism is Cupriavidus metallidurans. In some embodiments, the microorganism is a knallgas microorganism, also known as an oxyhydrogen microorganism. In some embodiments, the microorganism is a chemoautotrophic microbe.

In some embodiments, the wild-type or mutant of the microorganism naturally has a capability for accumulating and/or synthesizing high quantities of triacylglycerol where a high quantity is considered to be 10% or more of the dry cell mass. In some embodiments, the microorganism is a hydrogen-oxidizing chemoautotroph. In some embodiments, the microorganism is capable of growing on syngas as the sole energy and carbon source. In some embodiments, the microorganism is capable of growing on untreated crude glycerol as the sole energy and carbon source.

Further aspects of the invention relate to a method for producing shorter-chain fatty acids including in a bioreactor or solution, culturing an engineered microorganism as in claim 55 or a natural strain with a feedstock comprising syngas and/or gaseous CO₂ and/or a mixture of CO₂ gas and H₂ gas. In some embodiments, the method further comprises the step of enhancing expression of enzymes through heat. In some embodiments, the method further comprises the step of up-regulating an endogenous or exogenous thioesterase gene of the microorganism. In some embodiments, the method further comprise the step of down-regulating an endogenous or exogenous thioesterase gene of the microorganism. In some embodiments, the method further comprises the step of down regulating an endogenous or exogenous acyl carrier protein gene of the microorganism.

Further aspects of the invention relate to a method of producing butanediol, or other biochemical precursors to butanediol by microbial fermentation under microaerophilic or anaerobic conditions, including: supplying an inorganic substrate as a primary source of metabolic energy, whereby the substrate consists of one or more electron donors and one Of more electron acceptors; and fermentation in a bioreactor containing a culture of microorganisms utilizing an inorganic substrate as a primary source of metabolic energy and carbon dioxide or other inorganic carbon as the primary source of carbon.

In some embodiments, the inorganic substrate comprises hydrogen (H2). In some embodiments, the butanediol product is 2,3-butanediol, 1,4 butanediol or 1,3 butanediol. In some embodiments, the level of hydrogen is supplied at a level such that butanediol is produced. In some embodiments, the level of CO₂ is supplied at a level such that butanediol is produced. In some embodiments, the culture is propagated in the bioreactor in which oxygen is introduced at a certain flow rate, and the oxygen level is subsequently changed to a lower flow rate such that butanediol is produced at enhanced levels.

In some embodiments, the electron donors include but are not limited to one or more of the following reducing agents: ammonia; ammonium; carbon monoxide; dithionite; elemental sulfur; hydrogen; metabisulfites; nitric oxide; nitrites; sulfates such as thiosulfates including but not limited to sodium thiosulfate (Na₂S₂O₃) or calcium thiosulfate (CaS₂O₃); sulfides such as hydrogen sulfide; sulfites; thionate; thionite and said electron acceptors include but are not limited to one or more of the following oxidizing agents: carbon dioxide, ferric iron or other transition metal ions, nitrates, nitrites, oxygen, or holes in solid state electrode materials.

In some embodiments, the primary fermentation microbe is of the genera Rhodococcus or Gordonia. In some embodiments, the primary fermentation microbe is the species Rhodococcus sp. DSM 3346 or DSM 364. In some embodiments, the primary fermentation microbe is a Rhodococcus opacus. In some embodiments, the primary fermentation microbe is a Rhodococcus opacus (DSM 43205) or a Rhodococcus opacus (DSM 43206) or a Rhodococcus opacus (DSM 44193). In some embodiments, the primary fermentation microbe is family burkholderiaceae. In some embodiments, the primary fermentation microbe is Cupriavidus necator. In some embodiments, the primary fermentation microbe is Cupriavidus metallidurans. In some embodiments, the primary fermentation microbe is a knallgas microorganism, also known as an oxyhydrogen microorganism. In some embodiments, the primary fermentation microbe is a chemoautotrophic microbe.

In some embodiments, the wild-type or mutant of the primary fermentation microbe naturally has a capability for accumulating and/or synthesizing high quantities of triacylglycerol where a high quantity is considered to be 10% or more of the dry cell mass. In some embodiments, the primary fermentation microbe is a hydrogen-oxidizing chemoautotroph. In some embodiments, the primary fermentation microbe is capable of growing on syngas as the sole energy and carbon source. In some embodiments, the primary fermentation microbe is capable of growing on untreated crude glycerol as the sole energy and carbon source.

In some embodiments, the method further comprises the step of up-regulating an endogenous or exogenous gene regulating the pathway for the production of butanediol. In some embodiments, the method further comprises the step of down-regulating an endogenous or exogenous gene regulating the pathway for the production of butanediol.

### BRIEF DESCRIPTION OF THE DRAWINGS

Non-limiting embodiments of the present invention will be described by way of example with reference to the accompanying figures, which are schematic and are not intended to be drawn to scale. For purposes of clarity, not every component is labeled in every figure, nor is every component of each embodiment of the invention shown where illustration is not necessary to allow those of ordinary skill in the art to understand the invention. In the figures:

FIG. 1 describes the taxonomic names afforded to the chemoautotrophic and oleaginous microorganisms used in selected embodiments of the invention.

FIG. 2 shows the 16S rRNA gene based-rooted phylogenetic tree of gordoniaceae, mycobacteriaceae, nocardiaceae and burkholderiaceae.

Bar, 0.01% estimated sequence divergence.

FIG. 3 shows the sequence similarity of Rhodococcus opacus (DSM 43205) 16S rRNA gene (NR_{—}026186.1) to members of the family gordoniaceae, mycobacteriaceae, nocardiaceae and burkholderiaceae. The Genbank accession numbers, DNA length and % identity of analyzed genes are indicated.

FIG. 4 describes the nucleotide sequence alignment of the 16S rRNA genes SEQ ID NOs: 20-49.

FIG. 5 demonstrates the growth of chemotrophic and oleaginous microorganisms on different carbon sources. Bacterial growth was measured using optical density (OD) detection at 650 nm after the indicated days (in parentheses). Media and growth conditions described in the Examples section below. ND, not done.

FIG. 6 describes the measured lipid content of microorganisms on heterotrophic and chemoautotrophic growth conditions as a percentage of total cellular dry matter (CDM). Cells were grown under conditions described in FIG. 5, harvested after 72 hr (unless otherwise indicated) and analyzed by gas chromatography. For CDM, total dry weight was determined gravimetrically.

FIG. 7 describes the fatty acid profile of R. opacus (DSM 44193) under heterotrophic growth conditions. Cells were harvested after 72 hr and analyzed by gas chromatography.

FIG. 8 describes the fatty acid profile R. opacus (DSM43205) under heterotrophic (A) and chemoautotrophic (B) growth conditions. Cells were harvested after 72 hours of growth and analyzed by gas chromatography.

FIG. 9 describes the fatty acid profile Rhodococcus sp. (DSM 3346) under heterotrophic (A) chemoautotrophic (B) growth conditions. Cells were harvested after 72 hr and analyzed by gas chromatography.

FIG. 10 describes shuttle vectors (A) and genetic elements (B) for transformation and gene expression of in chemoautotrophic and oleaginous microorganisms. MCS: multiple cloning site.

FIG. 11 describes the map of the plasmids pSeqCO1 (A; SEQ ID: 01), pSeqCO2 (B; SEQ ID: O₂), pVer1 (C; SEQ ID: 03) and pVer2 (D; SEQ ID: 04) described in FIG. 10. The genetic elements are indicated.

FIG. 12 describes the transformation of chemoautotrophic and oleaginous microorganisms with shuttle vectors described in FIG. 10.

FIG. 13 describes the growth of Cupriavidus necator (DSM531) transformed with the plasmid (Y) pSeqCO2 (SEQ ID:2) and untransformed (N) on different kanamycin concentrations. Single colony of transformants and control were grown LB medium (per 1 L: 10 g Bacto-tryptone, 5 g yeast extract, 10 g NaCl pH=7.0) at 30° C. in the indicated kanamycin concentrations. The growth was measured using O.D₆₅₀ after the indicated number of days.

FIG. 14 describes the formation of fatty alcohols in oleaginous bacteria. The role of the fatty acyl-CoA reductases (FAR) gene in the biosynthesis pathway is shown. The Arabidopsis genes FAR1 (SEQ ID: 05), FAR2 (SEQ ID: 06) and FAR3 (SEQ ID: 07) were cloned into pSeqCO2 plasmid using the indicated restriction sites to give pSeqCO2::FAR1, pSeqCO2::FAR2, pSeqCO2::FAR3.

FIG. 15 describes the pathway for formation of fatty alcohols in burkholderiaceae using of the fatty acyl-CoA reductases (FAR) gene.

FIG. 16 describes the cloning strategy of FAR gene into pSeqCO2 plasmids. The Arabidopsis genes FAR1 (SEQ ID: 05), FAR2 (SEQ ID: 06) and FAR3 (SEQ ID: 07) were cloned into pSeqCO2 plasmid using the indicated restriction sites to give pSeqCO2::FAR1, pSeqCO2::FAR2, pSeqCO2::FAR3.

FIG. 17 describes the effect of FAR genes expression on fatty acid synthesis in Cupriavidus necator. C. necator cells were transformed with pSeqCO2::FAR1 (Cn-F1), pSeqCO2::FAR2 (Cn-F2) and control pSEqCO2 (Cn-P). Cells were harvested (3,000×g for 20 min at 4° C.) and fatty acids were analyzed by gas chromatography.

FIG. 18 describes the pathway for formation of hydrocarbons in oleaginous bacteria using the enzymes fatty acid acyl-ACP reductase (FadDR) and fatty acid aldehyde decarbonylase by (FAD) genes. Genes from the cyanobacterium (Synechocystis sp. PCC 6803) used in the experiment were FadR (SEQ ID: 08) and FAD (SEQ ID: 09) driven by the Synechocystis sp. Rubisco large subunit promoter (SEQ ID: 09) were cloned into pSeqCO2 plasmid using the indicated restriction sites to give pSeqCO2::FUEL.

FIG. 19 describes the pathway for formation of hydrocarbons in burkholderiaceae using the enzymes fatty acid acyl-ACP reductase (FadDR) and fatty acid aldehyde decarbonylase by (FAD) genes

FIG. 20 describes the restriction map related to the cloning strategy of FadDR and Fad genes into pSeqCO2 plasmid transformed for the experiment. Genes from the cyanobacterium (Synechocystis sp. PCC 6803) used in the experiment were FadR (SEQ ID: 08) and FAD (SEQ ID: 09) driven by the Synechocystis sp. Rubisco large subunit promoter (SEQ ID: 10) were cloned into pSeqCO2 plasmid using the indicated restriction sites to give pSeqCO2::FUEL.

FIG. 21 describes the production of Alkanes in Cupriavidus necator transformed with pSeqCO2::FUEL (Cn_FUEL2.1) and empty vector (Cn-P). GC chromatogram of hydrocarbon (peaks indicated with label) extracted from transformants grown in 50 ml LB media under previously identified conditions.

FIG. 22 describes the hydrocarbon specific products and distribution (percentage in parentheses) from Cupriavidus necator transformed with pSeqCO2::FUEL (Cn_FUEL2.1 and Cn_FUEL2.2) and empty vector (Cn-P).

FIG. 23 describes the effect of pSeqCO2::FUEL (Cn_FUEL2.1 and 2.2) and empty vector (Cn-P) on the fatty acids distribution under the experimental conditions described previously.

FIG. 24 describes the modification of the fatty acid chain length by the enzymatic action of thioesterase (TE) in oleaginous bacteria.

FIG. 25 describes the modification of the fatty acid chain length by the enzymatic action of fatty acyl-ACP thioesterase (TE) in burkholderiaceae.

FIG. 26 describes the similarity of Rhodococcus opacus (B4) thioesterases protein sequence (YP_{—}002784058.1) to other organisms. The Genbank accession numbers, amino acid length and % identity of analyzed proteins are indicated.

FIG. 27 describes the fluorescence intensity of Rhodococcus Sp exposed to 0, 5, 10, and 20 seconds of (FIGS. 27B, 27C, 27D and 27E respectively) of UV light and stained with Nile Red. FACS analysis of untreated cells (negative control; no Nile Red staining and no UV exposure) (FIG. 27F) and mutated population with increased lipid content (G; P3) are shown.

FIG. 28 describes the chemoautotrophic growth of Cupriavidus necator transformed with pSeqCO2::FUEL (Cn-FUEL2.1), empty vector (Cn-P) and untransformed (Cn). Bacterial growth was measured at O.D₆₅₀ after 12 days. Media and growth conditions described in FIG. 7.

FIG. 29 describes the affect of FAR genes expression on biosynthesis of cyclotetradecane in Cupriavidus necator. C. necator cells were transformed with pSeqCO2::FAR1 (Cn-F1), pSeqCO2::FAR2 (Cn-F2) and control pSEqCO2 (Cn-P). Cells were harvested (3,000×g for 10 min at 4° C.) and alkanes were analyzed by gas chromatography

FIG. 30 shows a schematic block flow diagram of a process for utilizing a gaseous C1 feedstock such as syngas to produce hydrocarbons using the microorganisms of the present invention.

FIG. 31 shows a schematic block flow diagram of a process for utilizing a gaseous C1 feedstock such as syngas to produce lipids using the microorganisms of the present invention with additional post-processing steps converting the lipids to drop-in fuels such as jet fuel and/or diesel.

FIG. 32 shows octadecanoic acid derivatives produced by at least one Kiverdi chemoautotrophic production strain. Experimental runs for fatty acid percent yields (grams of product/100 grams total fatty acid) from organisms Rhodococcus opacus (DSM 44193), Rhodococcus opacus (DSM 43205), and Cupriavidus necator.

FIG. 33 shows putative 12-hydroxylases culled by word searching Genbank.

FIG. 34 shows genes related to Vicia sativa P450 omega hydroxylases.

FIG. 35 shows a list of P450-dependent fatty acid omega hydroxylases.

FIG. 36 shows a list fatty acid hydroxylases.

FIG. 37 shows the percent fatty acid production for plasmid control (TKO4-P), thioesterase expression (TKO4-TE), and fatty acyl-CoA binding protein (TKO4-ACoA-BP).

FIG. 38 shows the percent fatty acid production for fatty acyl-CoA binding protein (TKO4-ACoABP) for T=22 C vs. T=30 C.

FIG. 39 shows (A) Fatty acid percentages (C12, C14, C16, and C18 chain lengths) for Cupriavidus necator (DSM531) organism with control plasmid pSeqCO2 (CN-P), with expression of exogenous thioesterase (CN-TE), and expression of fatty acyl-CoA binding protein (CN-ACBP). (B) Fatty acid percentages (C12 and C14) with expression of exogenous thioesterase (CN-TE), and expression of fatty acyl-CoA binding protein (CN-ACBP) compared with control (CN-P).

FIG. 40 shows Fatty acid percentages (C12, C14, C16, and C18 chain lengths) for Cupriavidus necator expressing ACBP at T=22° C. vs. T=30° C.

FIG. 41 shows the map of the plasmid pSeqCO2::ACBP. The genetic elements are indicated.

FIG. 42 shows growth (optical density) of Alcaligenes eutrophus on H2, CO2 and O2 to a cell density of 35 g/l (dry cell weight). Alcaligenes eutrophus was grown microaerobically. Several aspects involve growing Alcaligenes eutrophus or other oxyhydrogen microbes, either engineered or not engineered, to a high cell density microaerobically on syngas components (H2, CO2 and/or CO) then switching to anaerobic bioprocessing for the production of 1,3 butandiol and other organic compounds, which are secreted.

FIG. 43 shows 2.3 Butatadiol pathways.

FIG. 44 shows the pathway of introducing BDO metabolic pathway to a organism.

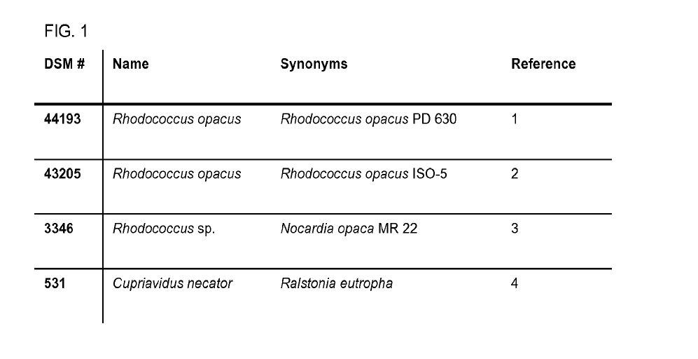

### DETAILED DESCRIPTION

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

As used in this specification and the appended claims, the singular forms “a,” “an,” and “the” include plural referents unless the content clearly dictates otherwise.

The term “about” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The terms “amino acid” refer to a molecule containing both an amine group and a carboxyl group that are bound to a carbon, which is designated the α-carbon. Suitable amino acids include, without limitation, both the D- and L-isomers of the naturally occurring amino acids, as well as non-naturally occurring amino acids prepared by organic synthesis or other metabolic routes. In some embodiments, a single “amino acid” might have multiple sidechain moieties, as available per an extended aliphatic or aromatic backbone scaffold. Unless the context specifically indicates otherwise, the term amino acid, as used herein, is intended to include amino acid analogs.

The term “biodiesel” refers to a biologically produced fatty acid alkyl ester suitable for use as a fuel in a diesel engine.

The term “biomass” refers to a material produced by growth and/or propagation of cells. Biomass may contain cells and/or intracellular contents as well as extracellular material, includes, but is not limited to, compounds secreted by a cell.

The term “bioreactor” or “fermentor” refers to a closed or partially closed vessel in which cells are grown and maintained. The cells may be, but are not necessarily held in liquid suspension. In some embodiments rather than being held in liquid suspension, cells may alternatively be growing and/or maintained in contact with, on, or within another non-liquid substrate including but not limited to a solid growth support material.

The term “catalyst” refers to a chemical actor, such as a molecule or macromolecular structure, which accelerates the speed at which a chemical reaction occurs where a reactant or reactants is converted into a product or products, while the catalyst is not turned into a product itself, or otherwise changed or consumed at the completion of the chemical reaction. After a catalyst participates in one chemical reaction, because it is unchanged, it may participate in further chemical reactions, acting on additional reactants to create additional products. To accelerate a chemical reaction a catalyst decreases the activation energy barrier across the reaction path allowing it to occur at a colder temperature, or faster at a given temperature. In this way a more rapid approach of the system to chemical equilibrium may be achieved. Catalysts subsume enzymes, which are protein catalysts.

The term “cellulosic material” refers to any material with a high amount of cellulose, which is a polysaccharide having the formula (C₆H₁₀O₅)ₙ, that generally consists of a linear chain of hundreds to thousands of β(1→4) linked D-glucose monomers. Sources of cellulosic material include but are not limited to cardboard, cotton, corn stover, paper, lumber chips, sawdust, sugar beet pulp, sugar cane bagasses, and switchgrass.

The term “CoA” or “coenzyme A” refers to an organic cofactor for condensing enzymes involved in fatty acid synthesis and oxidation, pyruvate oxidation, acetyl or other acyl group transfer, and in other acetylation.

The term “cofactor” subsumes all molecules needed by an enzyme to perform its catalytic activity. In some embodiments, the cofactor is any molecule apart from the substrate.

A “conservative amino acid substitution” is one in which the amino acid residue is replaced with an amino acid residue having a similar side chain. Families of amino acid residues having similar side chains have been defined in the art. These families include amino acids with basic side chains (e.g., K, R, H), acidic side chains (e.g., D, E), uncharged polar side chains (e.g., G, N, Q, S, T, Y, C, H), nonpolar side chains (e.g., G, A, V, L, I, P, F, M, W), beta-branched side chains (e.g., T, V, I) and aromatic side chains (e.g., Y, F, W, H). Thus, a predicted nonessential amino acid residue in an amino acid sequence encoded by an exogenous nucleic acid sequence, for example, is replaced with another amino acid residue from the same side chain family. Other examples of acceptable substitutions are substitutions based on isosteric considerations (e.g. norleucine for methionine) or other biochemical properties (e.g. 2-thienylalanine for phenylalanine).

As used herein, “enzyme fragment” is meant to refer to a fragment of an enzyme that includes the sequences sufficient to function substantially similar to the function of the wild-type enzyme upon which the fragment sequence is based. Fragments are generally 10 or more amino acids in length. Some preferred lengths of fatty acid reductase are at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210 at least 215, at least 220, at least 225, least 230 at least 235, at least 240, at least 245, at least 250, at least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, at least 355, at least 360, at least 365, at least 370, at least 375, at least 380, at least 385, at least 390, at least 395, at least 400, at least 405, at least 410, at least 415, at least 420, at least 425, or at least 430 amino acids in length. Some preferred lengths of fatty acid reductase fragments are 15 or fewer, 20 or fewer, 25 or fewer, 30 or fewer, 35 or fewer, 40 or fewer, 45 or fewer, 50 or fewer, 55 or fewer, 60 or fewer, 65 or fewer, 70 or fewer, 75 or fewer, 80 or fewer, 85 or fewer, 90 or fewer, 95 or fewer, 100 or fewer, 105 or fewer, 110 or fewer, 115 or fewer, 120 or fewer, 125 or fewer, 130 or fewer, 135 or fewer, 140 or fewer, 145 or fewer, 150 or fewer, 155 or fewer, 160 or fewer, 165 or fewer, 170 or fewer, 175 or fewer, 180 or fewer, 185 or fewer, 190 or fewer, 195 or fewer, 200 or fewer, 205 or fewer, 210 or fewer, 215 or fewer, 220 or fewer, 225 or fewer, 230 or fewer, 235 or fewer, 240 or fewer, 245 or fewer, 250 or fewer, 255 or fewer, 260 or fewer, 265 or fewer, 270 or fewer, 275 or fewer, 280 or fewer, 285 or fewer, 290 or fewer, 295 or fewer, 300 or fewer, 305 or fewer, 310 or fewer, 315 or fewer, 320 or fewer, 325 or fewer, 330 or fewer, 335 or fewer, 340 or fewer, 345 or fewer, 350 or fewer, 355 or fewer, 360 or fewer, 365 or fewer, 370 or fewer, 375 or fewer, 380 or fewer, 385 or fewer, 390 or fewer, 395 or fewer, 400 or fewer, 415 or fewer, 420 or fewer, 425 or fewer, 430 or fewer, or 435 or fewer. Some preferred lengths of fatty acid decarbonylase are at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210 at least 215, at least 220, at least 225, least 230 at least 235, at least 240, at least 245, at least 250, at least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, at least 355, at least 360, at least 365, at least 370, at least 375, at least 380, at least 385, at least 390, at least 395, at least 400, at least 405, at least 410, at least 415, or at least 420 amino acids long. In some embodiments, the lengths of the fatty acid decarbonylase fragments are 15 or fewer, amino acids, 20 or fewer, 25 or fewer, 30 or fewer, 35 or fewer, 40 or fewer, 45 or fewer, 50 or fewer, 55 or fewer, 60 or fewer, 65 or fewer, 70 or fewer, 75 or fewer, 80 or fewer, 85 or fewer, 90 or fewer, 95 or fewer, 100 or fewer, 105 or fewer, 110 or fewer, 115 or fewer, 120 or fewer, 125 or fewer, 130 or fewer, 135 or fewer, 140 or fewer, 145 or fewer, 150 or fewer, 155 or fewer, 160 or fewer, 165 or fewer, 170 or fewer, 175 or fewer, 180 or fewer, 185 or fewer, 190 or fewer, 195 or fewer, 200 or fewer, 205 or fewer, 210 or fewer, 215 or fewer, 220 or fewer, 225 or fewer, 230 or fewer, 235 or fewer, 240 or fewer, 245 or fewer, 250 or fewer, 255 or fewer, 260 or fewer, 265 or fewer, 270 or fewer, 275 or fewer, 280 or fewer, 285 or fewer, 290 or fewer, 295 or fewer, 300 or fewer, 305 or fewer, 310 or fewer, 315 or fewer, 320 or fewer, 325 or fewer, 330 or fewer, 335 or fewer, 340 or fewer, 345 or fewer, 350 or fewer, 355 or fewer, 360 or fewer, 365 or fewer, 370 or fewer, 375 or fewer, 380 or fewer, 385 or fewer, 390 or fewer, 395 or fewer, 400 or fewer, 415 or fewer, 422 or fewer. Some preferred lengths of thioesterase fragments are at least 10 amino acids, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210 at least 215, at least 220, at least 225, least 230 at least 235, at least 240, at least 245, at least 250 or at least 255. Some preferred lengths of thioesterase fragments are 15 or fewer, 20 or fewer, 25 or fewer, 30 or fewer, 35 or fewer, 40 or fewer, 45 or fewer, 50 or fewer, 55 or fewer, 60 or fewer, 65 or fewer, 70 or fewer, 75 or fewer, 80 or fewer, 85 or fewer, 90 or fewer, 95 or fewer, 100 or fewer, 105 or fewer, 110 or fewer, 115 or fewer, 120 or fewer, 125 or fewer, 130 or fewer, 135 or fewer, 140 or fewer, 145 or fewer, 150 or fewer, 155 or fewer, 160 or fewer, 165 or fewer, 170 or fewer, 175 or fewer, 180 or fewer, 185 or fewer, 190 or fewer, 195 or fewer, 200 or fewer, 205 or fewer, 210 or fewer, 215 or fewer, 220 or fewer, 225 or fewer, 230 or fewer, 235 or fewer, 240 or fewer, 245 or fewer, 250 or fewer, 255 or fewer or 260 or fewer amino acids. As used in the paragraph herein reference to preferred fragment sizes are intended to refer to all permutation of ranges between at least and less than such as ranges may be any number set forth as an “at least” size to any number set forth as an “less than t” size in order to provide a range of sizes such as 20-400, 20-30, 40-100, etc.

The terms “exogenous gene” or “exogenous nucleic acid” means a nucleic acid that has been recombinantly introduced into a cell, which encodes the synthesis of RNA and/or protein. In some embodiments, the exogenous gene is introduced by transformation. In some embodiments, the exogenous gene is introduced into the cell by electroporation. A transformed cell may be referred to as a recombinant cell, into which additional exogenous gene(s) may be introduced. The exogenous gene put into the host species may be taken from a different species (this is called heterologous), or it may naturally occur within the same species (this is homologous as defined below). Therefore, exogenous genes subsume homologous genes that are integrated within or introduced to regions of the genome, episome, or plasmid that differ from the locations where the gene naturally occurs. Multiple copies of the exogenous gene may be introduced into the cell. An exogenous gene may be present in more than one copy within the host cell or transformed cell. In some embodiments, the microorganism comprises between and including 1 and 1,000 copies of the nucleic acid that encodes an exogenous protein. In some embodiments, the microorganism comprises between and including 1 and 10,000 copies of the nucleic acid that encodes an exogenous protein. In some embodiments, the microorganism comprises between and including 1 and 500 copies of the nucleic acid that encodes an exogenous protein. In some embodiments, the exogenous gene is maintained by a cell as an insertion into the genome or as an episomal molecule. In some embodiments, the microorganism comprises no more than 1, 2, 3, 4, 5, 6, 7, 8, 9, 10, 20, 30, 40, 50, 60, 70, 80, 90, 100, or 1000 copies of the one or more nucleic acids that encode one or more exogenous proteins.

As used herein, the term “expressible form” refers to gene constructs that contain the necessary regulatory elements operably linked to a coding sequence that encodes an enzyme or fragment thereof capable of conferring enzymatic activity to a cell, such that when present in the cell, the coding sequence will be expressed. In some embodiments of the invention, the composition comprising the microorganisms or bacterial cells of the present invention comprise no more than ten expressible forms of exogenous nucleic acid sequences. In some embodiments of the invention, the composition comprising the microorganisms or bacterial cells of the present invention comprise no more than nine expressible forms of exogenous nucleic acid sequences. In some embodiments of the invention, the composition comprising the microorganisms or bacterial cells of the present invention comprise no more than eight expressible forms of exogenous nucleic acid sequences. In some embodiments of the invention, the composition comprising the microorganisms or bacterial cells of the present invention comprise no more than seven expressible forms of exogenous nucleic acid sequences. In some embodiments of the invention, the composition comprising the microorganisms or bacterial cells of the present invention comprise no more than six expressible forms of exogenous nucleic acid sequences. In some embodiments of the invention, the composition comprising the microorganisms or bacterial cells of the present invention comprise no more than five expressible forms of exogenous nucleic acid sequences. In some embodiments of the invention, the composition comprising the microorganisms or bacterial cells of the present invention comprise no more than four expressible forms of exogenous nucleic acid sequences. In some embodiments of the invention, the composition comprising the microorganisms or bacterial cells of the present invention comprise no more than three expressible forms of exogenous nucleic acid sequences. In some embodiments of the invention, the composition comprising the microorganisms or bacterial cells of the present invention comprise no more than two expressible forms of exogenous nucleic acid sequences. In some embodiments of the invention, the composition comprising the microorganisms or bacterial cells of the present invention comprise no more than one expressible form of an exogenous nucleic acid sequences. In other embodiments of the invention, the composition comprising the microorganisms or bacterial cells of the present invention comprises more than ten expressible forms of exogenous nucleic acid sequences.

SEQ ID NO:1 refers to Sequesco plasmid sequence 1.

SEQ ID NO:2 refers to Sequesco plasmid sequence 2.

SEQ ID NO: 3 refers to Sequesco plasmid Ver1 plasmid sequence.

SEQ ID NO:4 refers to Sequesco plasmid Ver2 plasmid sequence.

SEQ ID NO:5 refers to Arabidopsis gene FAR1.

SEQ ID NO: 6 refers to Arabidopsis gene FAR2.

SEQ ID NO: 7 refers to Arabidopsis gene FAR3.

SEQ ID NO:8 refers to cyanobacterium FadR.

SEQ ID NO:9 refers to cyanobacterium FAD.

SEQ ID NO:10 refers to cyanobacterium Rubisco large subunit promoter

SEQ ID NO:11, refers to the 16S rRNA sequence from the genus Rhodococcus opacus DSM43205

SEQ ID NO:12 refers to the 16S rRNA sequence from the genus Rhodococcus opacus B4.

SEQ ID NO:13 refers to the 16S rRNA sequence from the genus Ralstonia.

SEQ ID NO:14 refers to Rhodococcus opacus TE

The terms “fatty acyl-ACP thioesterase” (TE) mean an enzyme that catalyzes the cleavage of a fatty acid from an acyl carrier protein (ACP) during lipid synthesis.

The terms “fatty acyl-CoA reductase” (FAR) refers to an enzyme catalyzing the reaction that produces a fatty alcohol from an acyl-CoA molecule by reduction.

The terms “fatty acyl-ACP/acyl-CoA reductase” (FadR) refers to an enzyme catalyzing the reaction that produces a fatty aldehyde from an acyl-ACP or acyl-CoA molecule by reduction.

The terms “fatty aldehyde decarbonylase” (FAD) refers to an enzyme catalyzing the reaction that produces an alkane from a fatty aldehyde molecule by decarbonylization.

The terms “fatty aldehyde reductase” refers to an enzyme catalyzing the reaction that produces a fatty alcohol from a fatty aldehyde molecule by reduction.

As used herein, the term “functional fragment” is meant to refer to a fragment of any polypeptide or amino acid sequence that is encoded by an exogenous nucleic acid sequence of the present invention which retains its ability to function like the amino acid sequence to which the fragment is homologous. Functional fragments of enzymes are at least about 5 amino acids in length derived from enzyme and may comprise non-wild-type amino acid sequences. One having ordinary skill in the art can readily determine whether a protein or peptide is a functional fragment of a particular amino acid sequence by examining its sequence and testing its ability to function in a fashion similar to that function of the amino acid sequence upon which the fragment is based. Truncated versions of exogenous proteins may be prepared and tested using routine methods and readily available starting material. As used herein, the term “functional fragment” is also meant to refer to peptides, polypeptides, amino acid sequence linked by non-peptidal bonds, or proteins which comprise an amino acid sequence that is identical or substantially homologous to at least a portion of the exogenous amino acid sequence and which are capable of functioning in a similar function to the exogenous amino acid sequence to which the fragment is homologous. The term “substantially homologous” refers to an amino acid sequence that has conservative substitutions. One having ordinary skill in the art can produce functional fragments of the FAR, FadD, FAD, thioesterase, cytochrome P450 enzyme, desaturase, and hydroxylase amino acid sequences following the disclosure provided herein and well known techniques. The functional fragments thus identified may be used and formulated in place of full length FAR, FadD, FAD, thioesterase, cytochrome P450 enzyme, desaturase, and hydroxylase without undue experimentation.

The term “gasification” refers to a generally high temperature (>700° C.) process that converts carbonaceous materials into a mixture of gases including hydrogen, carbon monoxide, and carbon dioxide called syngas or producer gas. The process generally involves partial combustion and/or the application of externally generated heat along with the controlled addition of oxygen and/or steam.

As used herein, “homologous” refers to the sequences homology between two nucleic acid sequences or two amino acid sequences. Two nucleic acid sequences or two amino acid sequences that are sufficiently homologous to retain immunogenic function are “homologues.” Sequence homology for nucleotides and amino acids may be determined using FASTA, BLAST and Gapped BLAST (Altschul et al., Nuc. Acids Res., 1997, 25, 3389, which is incorporated herein by reference in its entirety) and PAUP* 4.0b10 software (D. L. Swofford, Sinauer Associates, Massachusetts). “Percentage of similarity” is calculated using PAUP* 4.0b10 software (D. L. Swofford, Sinauer Associates, Massachusetts). The average similarity of the enzymatic sequence or 16S rRNA sequence is calculated compared to all sequences in the phylogenic tree. Briefly, the BLAST algorithm, which stands for Basic Local Alignment Search Tool is suitable for determining sequence similarity (Altschul et al., J. Mol. Biol., 1990, 215, 403410, which is incorporated herein by reference in its entirety). Software for performing BLAST analyses is publicly available though the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, preferably less than about 0.1, more preferably less than about 0.01, and most preferably less than about 0.001.

The term “hydrocarbon” refers to a molecule composed exclusively of carbon and hydrogen atoms with the carbons bonded covalently in a branched, cyclic, linear, or partially cyclic chain and with hydrogen atoms covalently bonded to the carbons such that the chemical octet rule for the carbons is generally satisfied. In some hydrocarbons there may occur some number of double or triple bonds between adjacent carbon atoms in the chain. Thus, the label hydrocarbon subsumes branched, cyclic, linear, branched, or partially cyclic alkanes (also called paraffins), alkenes (also called olefins), and alkynes. The structure of hydrocarbon molecules range from the smallest, methane (CH₄), a primary component of natural gas, to high molecular weight complex molecules including asphaltenes present in bitumens crude oil, and petroleum. Other examples include dodecane (C12), hexadecane (C16), or octadecane (C18) etc. Hydrocarbons of the present invention may be in gaseous, liquid, or solid phases, either as singly or in multiply coexisting phases. In some embodiments, the hydrocarbons are selected from one or more of the following: linear, branched, cyclic, or partially cyclic alkanes, alkenes, alkynes, lipids, and paraffin. In some embodiments the hydrocarbon are selected from one or more of the following: octane, squalene Spiro[4.5]decane, Bicyclo[10.8.0]eicosane, cis,cis-1,6-Dimethylspiro[4.5]decane, 1,19-Eicosadiene, Cyclooctacosane, Bicyclo[10.8.0]eicosane, 1-Pentadecyne, 1-Pentadecyne, Heptacosyl acetate, 5-Cyclohexyl-1-pentene, 1-Hexadecyne and Cyclodecacyclotetradecene, -eicosahydro.

The term “hydrophobic fraction” gives the fraction of matter that has low solubility in water and greater solubility in a hydrophobic phase than in an aqueous phase. In some embodiments, the hydrophobic fraction is non-polar. In some embodiments, the genetically modified bacterial cells described herein increase the hydrophobic fraction in a cell as compared to the same cell that is not genetically modified.

The term “improve lipid yield” refers to an increase in the lipid production of an organism through any means. In some embodiments, the increase is caused by raising the cell dry weight density of a microbial culture and/or raising the fraction of cell mass that is composed of lipid and/or reducing the cell doubling time and/or the biomass doubling time, resulting in an overall increase in the lipid production rate per unit volume.

The terms “jet fuel” means a fuel useful for igniting in the engine of an aircraft comprising a mixture of kerosene (mixture of C9-C16 alkanes of a certain percentage) combined with typical additives. In some embodiments the jet fuel may comprise a mixture of ingredients specified by the Jet A-1, Jet A, Jet B, JP1, JP-2, JP-3, JP-4, JP-5, JP-6, JP-7, JP-8, or other similar compositions. In some embodiments, the jet fuels comprise at least one or more typical additive chosen from antioxidants (including phenolic antioxidants), static inhibitors, corrosion inhibitors, fuel system icing inhibitors, lubrication improvers, biocides, and thermal stability improvers (DOD 1992; IARC 1989; Pearson 1988). These additives are used only in specified amounts, as governed by military specifications (DOD 1992; IARC 1989). Straight-run kerosene, the basic component of the kerosene used for jet fuels, consists of hydrocarbons with carbon numbers mostly in the C9-C16 range. Like all jet fuels, straight-run kerosene consists of a complex mixture of aliphatic and aromatic hydrocarbons (LARC 1989). Aliphatic alkanes (paraffins) and cycloalkanes (naphthenes) are hydrogen saturated, clean burning, and chemically stable and together constitute the major part of kerosene (IARC 1989). In some embodiments, the jet fuel comprises from between about 10%-20% aromatics and less than 1% of olefins. In some embodiments, the boiling range of the jet fuels is well above the boiling point of benzene. In some embodiments, the jet fuel comprises less than or equal to 0.02% of benzene and less than or equal to 0.01% of PAHs.

The term “knallgas” refers to the mixture of molecular hydrogen and oxygen gas. A “knallgas microorganism” is a microbe that can use hydrogen as an electron donor and oxygen as an electron acceptor in the generation of intracellular energy carriers such as Adenosine-5′-triphosphate (ATP). The terms “oxyhydrogen” and “oxyhydrogen microorganism” can be used synonymously with “knallgas” and “knallgas microorganism” respectively.

The term “lignocellulosic material” is any material composed of cellulose, hemicellulose, and lignin where the carbohydrate polymers (cellulose and hemicelluloses) are tightly bound to lignin. Lignocellulosic materials subsume agricultural residues (including corn stover and sugarcane bagasse), most biomass energy crops, wood residues (including sawmill and paper mill discards), and a substantial fraction of municipal waste.

The terms “lipids” refers to category of molecules that can be dissolved in nonpolar solvents (such as chloroform and/or ether) and which also have low or no solubility in water. The hydrophobic character of lipids molecules typically results from the presence of long chain hydrocarbon sections within the molecule. Lipids subsume the following molecule types: hydrocarbons, fatty acids (saturated and unsaturated), fatty alcohols, fatty aldehydes, hydroxy acids, diacids, monoglycerides, diglycerides, triglycerides, phospholipids, sphingolipids, sterols such as cholesterol and steroid hormones, fat-soluble vitamins (such as vitamins A, D, E and K), polyketides, terpenoids, and waxes.

The term “lipid modification enzyme” corresponds to an enzyme that catalyzes a reaction changing a lipid's covalent bonds such as TE, FAR, FadR, FAD, fatty aldehyde reductase, lipase, cytochrome P450 enzyme, desaturase, or hydroxylase. Any enzyme that catalyzes a reaction step or steps in lipid synthesis, catabolism, or modification, including carrier proteins, is called a “lipid pathway enzyme”.

The term “lysate” refers to the liquid containing a mixture and/or a solution of cell contents that result from cell lysis. In some embodiments, the methods of the present invention comprise a purification of hydrocarbons or mixture of hydrocarbons in a cellular lysate. In some embodiments, the methods of the present invention comprise a purification of lipids and/or hydrocarbons and/or a mixture of hydrocarbons in a cellular lysate.

The term “lysis” refers to the rupture of the plasma membrane and if present the cell wall of a cell such that a significant amount of intracellular material escapes to the extracellular space. Lysis can be performed using electrochemical, mechanical, osmotic, thermal, or viral means. In some embodiments, the methods of the present invention comprise performing a lysis of cells or microorganisms described herein in order to separate a hydrocarbon or mixture of hydrocarbons from the contents of a bioreactor. In some embodiments, the methods of the present invention comprise performing a lysis of cells or microorganisms described herein in order to separate a lipid or hydrocarbon or mixture of lipids or hydrocarbons or a mixture of lipids and hydrocarbons from the contents of a bioreactor.

The terms “microorganism” and “microbe” mean microscopic single celled life forms.

The term “molecule” means any distinct or distinguishable structural unit of matter comprising one or more atoms, and includes for example hydrocarbons, lipids, polypeptides and polynucleotides.

The term “natural strain” means any wild-type or mutant organism that has not had exogenous genes encoded in it.

The term “oleaginous” refers to something that is rich in oil or produces oil in high quantities.

The term “organic compound” refers to any gaseous, liquid, or solid chemical compounds which contain carbon atoms with the following exceptions that are considered inorganic: carbides, carbonates, simple oxides of carbon, cyanides, and allotropes of pure carbon such as diamond and graphite.

The term “precursor to” or “precursor of” jet fuel, diesel fuel, or biodiesel fuel means a lipid intermediate of one or more of the components of jet, diesel fuel, or biodiesel fuel. For instance, jet fuel is a complex mixture of hydrocarbons that varies depending on crude source and manufacturing process. Consequently, it is impossible to define the exact composition of jet fuel. Specification of jet fuel has therefore evolved primarily as a performance specification rather than a compositional specification and the hydrocarbons typically range between 8 and 17 carbon atoms in hydrocarbon chain length. In some embodiments, a precursor to jet fuel may be composition comprising at least one hydrocarbon having a carbon chain length of 8, 9, 10, 11, 12, 13, 14, 15, 16, 17, 18, or more carbon atoms and having the commonly known specifications for Jet A-1, Jet A, Jet B, JP1, JP-2, JP-3, JP-4, JP-5, JP-6, JP-7, JP-8 fuel when in isolation or mixture with other hydrocarbons. In some embodiments, the precursor to jet fuel is a mixture of different carbon backbone lengths of 8, 9, 10, 11, 12, 13, 14, 15, 16, 17, 18, or more carbon atoms with the commonly known specifications for Jet A-1, Jet A, Jet B, JP1, JP-2, JP-3, JP-4, JP-5, JP-6, JP-7, JP-8 fuel, or other jet fuels. In some embodiments, the precursor to jet fuel may be one or more hydrocarbons that, when exposed to cracking and/or deoxygention and/or isomerization, may be used as a component of Jet A-1, Jet A, Jet B, JP1, JP-2, JP-3, JP-4, JP-5, JP-6, JP-7, JP-8 fuel or other jet fuels.

The term “producing” includes both the production of compounds intracellularly and extracellularly, which is to include the secretion of compounds from the cell.

“Promoter” is a control DNA sequence that regulates transcription. For purposes of the invention, a promoter may includes nucleic acid sequences near the start site of transcription that are required for proper function of the promoter, as for example, a TATA element for a promoter of polymerase II type. Promoters of the present invention can include distal enhancer or repressor elements that may lie in positions up to many thousands of base pairs away from the start site of transcription. The term “inducible promoter” refers to an operable linkage between a promoter and a nucleic acid where the promoter's mediation of nucleic acid transcription is sensitive to a specific stimulus. In some embodiments, the inducible promoter requires a cofactor which can be added to the environment of the composition comprising the nucleic acid sequence that contains the inducible promoter. An “operable linkage” refers to an operative connection between nucleic acid sequences, such as for example between a control sequence (e.g. a promoter) and another sequence that codes for a protein i.e. a coding sequence. If a promoter can regulate transcription of an exogenous gene then it is in operable linkage with the gene.

The term “syngas” (from synthetic gas or synthesis gas) refers to a gas mixture that contains various proportions of hydrogen, carbon monoxide, and carbon dioxide, and which typically also includes a variety of impurities such as methane, hydrogen sulfide, condensable gases, and tars. “Producer gas” is a related term that generally refers to gas mixes similar to syngas except for the presence of a large N₂ component that results from using air directly in the gasification process.

### Bacterial Species

The invention relates to chemotrophic bacterial strains that comprise one or more exogenous nucleic acid sequences. The present invention results from the discovery that chemotrophic bacteria and particular related microorganisms provide unforeseen advantages in the economic and large scale production of chemicals, oils, fuels, and other hydrocarbon or lipid substances from gaseous and waste carbon feedstocks, and also from the discovery of genetic techniques and systems for modifying these microorganisms for improved performance in these applications. The lipids and other biochemicals synthesized by the microorganisms of the present invention can be applied to uses including but not limited to transportation fuel, petrochemical substitutes, monomers, feedstock for the production of polymers, lubricants, as ingredients in animal feed, food, personal care, and cosmetic products. In some embodiments triglycerides produced in the present invention can be converted by transesterification to long-chain fatty acid esters useful as biodiesel fuel. In some embodiments of the present invention enzymatic and chemical processes can be utilized to produce alkanes, alkenes, alkynes, hydroxy acids, fatty aldehydes, fatty alcohols, fatty acids, diacids, and unsaturated fatty acids. Some embodiments enable the production of renewable jet fuel, diesel, or other hydrocarbons. In addition, the present invention gives methods for culturing and/or modifying chemotrophic bacteria for improved lipid yield and/or lower production costs. In some embodiments the genetically modified bacteria produce more of a certain type or types of lipid molecules as compared to the same bacteria that is not genetically modified.

The present invention relates to compositions comprising and methods of using genetically modified microorganisms to produce and/or secrete carbon-based products from conversion of gaseous carbon feedstocks including but not limited to syngas or producer gas. The present invention relates to methods and mechanisms to confer production and/or secretion of carbon-based products of interest including but not limited to ethylene, chemicals, monomers, polymers, n-alkanes, branched alkanes, cycloalkanes, alkenes, alkynes, hydroxy acids, fatty alcohols, fatty acids, diacids, unsaturated fatty acids, aldehydes, hydrocarbons, isoprenoids, proteins, polysaccharides, nutraceutical or pharmaceutical products or intermediates thereof in obligate or facultative chemotrophic organisms such that these organisms convert carbon dioxide and/or other forms of inorganic carbon and/or syngas and/or other C₁ compounds such as methanol and/or the liquid, gaseous, and solid products of pyrolytic reactions such as pyrolysis oil, into carbon-based products of interest, and in particular the use of such organisms for the commercial production of ethylene, chemicals, monomers, polymers, n-alkanes, branched alkanes, cycloalkanes, alkenes, alkynes, hydroxy acids, fatty alcohols, fatty acids, diacids, unsaturated fatty acids, fatty aldehydes, hydrocarbons, isoprenoids, proteins, polysaccharides, nutraceutical or pharmaceutical products or intermediates thereof.

Chemoautotrophs are capable of performing chemosynthetic reactions that fix CO2, and/or other forms of inorganic carbon, to organic compounds, using the potential energy stored in inorganic chemicals to drive the reaction, rather than radiant energy from light as in microorganisms performing photosynthesis [Shively et al, 1998; Smith et al, 1967; Hugler et al, 2005; Hugker et al., 2005; Scott and Cavanaugh, 2007]. Carbon fixing biochemical pathways that occur in chemoautotrophs include the reductive tricarboxylic acid cycle, the Calvin-Benson-Bassham cycle [Jessup Shively, Geertje van Kaulen, Wim Meijer, Annu. Rev. Microbiol., 1998, 191-230], and the Wood-Ljungdahl pathway [Ljungdahl, 1986; Gottschalk, 1989; Lee, 2008; Fischer, 2008].

The invention relates to compositions comprising and methods of using chemoautotrophic metabolism to produce ATP for the support of ATP consuming synthetic reactions and cellular maintenance, without the co-production of methane or short chain organic acids such as acetic or butyric acid, by means of energy conserving reactions for the production of ATP using inorganic electron donors, including but not limited to the oxyhydrogen reaction.

The production of hydrocarbons or other lipids with carbon chain lengths longer than C₄ is most commonly and efficiently accomplished biologically through fatty acid biosynthesis [Fischer, Klein-Marcuschamer, Stephanolpoulos, Metabolic Engineering (2008) 10, 295-304]. The initial molecule entering into the fatty acid biosynthesis pathway is acetyl-coenzyme A (acetyl-CoA), a central metabolite from which many high value biochemicals can be derived. In some embodiments, the invention utilizes microorganisms with a naturally occurring pathway for the conversion of CO, CO₂ and/or H₂ to acetyl-CoA. In some embodiments, the invention utilizes microorganisms that can fix CO and/or CO₂ through the reductive tricarboxylic acid cycle, the Calvin-Benson-Bassham cycle, and/or the Wood-Ljungdahl pathway. In some embodiments the invention utilizes microorganisms that fix C1 compounds through a methanotropic pathway. In some embodiments the microorganisms naturally produce enzymes that catalyze the fixation of gaseous inorganic carbon to produce acetyl-CoA, utilizing gaseous electron donors such as are present in syngas as reducing agents, with such enzymatic proteins including but not limited to acetyl-CoA synthase, acetyl-CoA synthase disulfide reductase, cobalamide corrinoid/iron-sulfur protein, carbon monoxide dehydrogenase, hydrogenase, and methyltransferase.

Unlike methanogenic, acetogenic and solventogenic pathways, present in methanogens and acetogens respectively, which can produce short chain organic compounds (C1-C4) with net ATP production or zero net consumption, fatty acid synthesis involves net ATP consumption. For example the following gives the net reaction for synthesis of Palmitic acid (C16) starting from Acetyl-CoA:

8Acetyl-CoA+7ATP+H₂O+14NADPH+14H⁺->Palmitic acid+8CoA+14NADP⁺+7ADP+7Pᵢ

A drawback with using an obligate methanogen or acetogen in a GTL process for the production of lipids, is the obligate use of CO2 as an electron acceptor for the production of ATP that is needed for fatty acid synthesis. If H₂ is the electron donor, the ATP produced per H₂ consumed in an acetogen or methanogen is relatively low: one ATP per 4H₂ for methane [Thauer, R. K., Kaster, A. K., Seedorf, H., Buckel, W. & Hedderich, R. Methanogenic archaea: ecologically relevant differences in energy conservation. Nat Rev Microbiol 6, 579-591, doi:nrmicro1931 [pii]] or acetic acid production, and one ATP per 10H₂ for butyric acid production [Papoutsakis, Biotechnology & Bioengineering (1984) 26, 174-187; Heise, Muller, Gottschalk, J. of Bacteriology (1989) 5473-5478; Lee, Park, Jang, Nielsen, Kim, Jung, Biotechnology & Bioengineering (2008) 101, 2, 209-228]. In some embodiments, the invention relates to a microorganism or compositions comprising a microorganism, wherein the microorganism produces ATP from an inorganic electron donor such as but not limited to H2 without synthesis of methane or short chain organic acids.

Hydrogen-oxidizing microorganisms that use more electronegative electron acceptors in energy conserving reactions for ATP production, such as but not limited to hydrogenotrophic oxyhydrogen or knallgas microbes that link the oxyhydrogen reaction, 2H₂+O₂->2H₂O, to ATP production, can produce more ATP per H₂ consumed than acetogens or methanogens. For example knallgas microorganisms can produce up to two ATP per H₂ consumed [Bongers, J. Bacteriology, (October 1970) 145-151], which is eight times more ATP produced per H₂ consumed than what can be produced in microorganisms undergoing methanogenesis or acetogenesis. For this reason using microorganisms that can utilize more electronegative electron acceptors in the production of ATP, such as but not limited to knallgas microbes, in fatty acid biosynthesis from syngas or H₂, can be more efficient for supporting fatty acid biosynthesis than using the acetogens or methanogens that are currently used in biological GTL technologies. In some embodiments, the invention relates to a microorganism or compositions comprising a microorganism, wherein the microorganism is a knallgas microbe and comprises at least one or more exogenous nucleic acid sequences that encodes one or more enzymes to enable fixation of a carbon-containing gas feedstock, including but not limited to syngas or producer gas, into useful carbon-based products of interest including but not limited to ethylene, chemicals, monomers, polymers, n-alkanes, branched alkanes, cycloalkanes, alkenes, alkynes, hydroxy acids, fatty alcohols, fatty acids, diacids, unsaturated fatty acids, fatty aldehydes, hydrocarbons, isoprenoids, polypeptides, polysaccharides, nutraceutical or pharmaceutical products. In some embodiments, the microorganism or composition comprising the microorganism comprises at least one or more exogenous nucleic acid sequences that encodes one or more enzymes that allows the microorganism to convert a carbon-containing gas feedstock, including but not limited to syngas or producer gas, into jet fuel, diesel fuel, biodiesel fuel, or a component or precursor thereof. The invention relates to a genetically modified microorganism and compositions comprising such a microorganism, wherein the microorganism comprises one or more exogenous genes and wherein the microorganism grows on carbon-containing gas or utilizes a gaseous feedstock selected from syngas, CO₂, H₂, CO, or mixtures of gas comprising one or more gases selected from syngas, CO₂, H₂, or CO.

The invention relates to a cell and compositions comprising a cell of the class Actinobacteria comprising at least one exogenous gene. The invention also relates to cells and compositions comprising cells of the family of Nocardiaceae comprising at least one exogenous gene. The invention relates to cells and compositions comprising cells of Corynebacterium, Gordonia, Rhodococcus, Mycobacterium and Tsukamurella comprising at least one exogenous gene. In some embodiments, the invention relate to cells of the family of Nocardiaceae comprising an exogenous gene, wherein the cell is not a cell of the genus Mycobacterium. In some embodiments, the invention provides a cell and compositions comprising a cell of the genus Rhodococcus comprising an exogenous gene, and in some embodiments the cell is a strain of the species Rhodococcus sp., Rhodococcus opacus, Rhodococcus aurantiacus; Rhodococcus baikonurensis; Rhodococcus boritolerans; Rhodococcus equi; Rhodococcus coprophilus; Rhodococcus corynebacterioides; Nocardia corynebacterioides (synonym: Nocardia corynebacterioides); Rhodococcus erythropolis; Rhodococcus fascians; Rhodococcus globerulus; Rhodococcus gordoniae; Rhodococcus jostii Rhodococcus koreensis; Rhodococcus kroppenstedtii; Rhodococcus maanshanensis; Rhodococcus marinonascens; Rhodococcus opacus; Rhodococcus percolatus; Rhodococcus phenolicus; Rhodococcus polyvorum; Rhodococcus pyridinivorans; Rhodococcus rhodochrous; Rhodococcus rhodnii; (synonym: Nocardia rhodnii); Rhodococcus ruber (synonym: Streptothrix rubra); Rhodococcus sp. RHA1; Rhodococcus triatomae; Rhodococcus tukisamuensis; Rhodococcus wratislaviensis (synonym: Tsukamurella wratislaviensis); Rhodococcus yunnanensis; Rhodococcus zopfii. In some embodiments the cell comprising one or more exogenous genes is strain Rhodococcus opacus DSM number 43205 or 43206. In some embodiments the cell comprising one or more exogenous genes is strain Rhodococcus sp. DSM number 3346. In some embodiments, the invention provides cells and compositions comprising a cell of the genus Rhodococcus comprising an exogenous gene, wherein the cell or composition comprising a cell of Rhodococcus is non-infectious to animals and/or plants. In some embodiments, the invention provides cells and compositions comprising a cell of the genus Rhodococcus comprising an exogenous gene, wherein the Rhodococcus cell or composition comprising a Rhodococcus cell is non-infectious to humans. In some embodiments, the invention provides cells and compositions comprising a cell of the genus Rhodococcus comprising an exogenous gene, wherein the Rhodococcus cell or composition comprising a Rhodococcus cell is non-infectious to plants. In some embodiments, the invention provides cells and compositions comprising cells of the genus Rhodococcus comprising an exogenous gene, wherein, if the cell is from Rhodococcus equi or Rhodococcus fascians species, the species is non-infectious to animals and/or plants. In some embodiments, the invention relates to a Rhodococcus cell or composition comprising a Rhodococcus cell, wherein the cell is not a species selected from Rhodococcus equi or Rhodococcus fascians.

In some embodiments, the invention relates to a Rhodococcus cell or composition comprising a Rhodococcus cell, wherein the cell is incapable of producing any acrylic acid or acrylamide. In some embodiments, the invention relates to a Rhodococcus cell or composition comprising a Rhodococcus cell, wherein the cell produces less than 10, 9, 8, 7, 6, 5, 4, 3, 2, or 1% of its weight of total dry cellular matter in acrylamide or acrylic/methylacrylic acid. In some embodiments, the invention relates to a Rhodococcus cell or composition comprising a Rhodococcus cell, wherein the cell is not from the species Rhodococcus rhodochrous. In some embodiments, the invention relates to Rhodococcus cell or composition comprising a Rhodococcus cell, wherein the cell is incapable of producing 10-hydroxy-12-octadecenoic acid. In some embodiments, the invention relates to a Rhodococcus cell or composition comprising a Rhodococcus cell, wherein the cell is unable to produce more than 10, 9, 8, 7, 6, 5, 4, 3, 2, or 1% of its weight of total dry cellular matter in 10-hydroxy-12-octadecenoic acid. In some embodiments, the invention relates to Rhodococcus cell or composition comprising a Rhodococcus cell, wherein the cell is incapable of producing optically-active 4-amino-3-hydroxybutyric acid. In some embodiments, the invention relates to a Rhodococcus cell or composition comprising a Rhodococcus cell, wherein the cell is unable to produce more than 10, 9, 8, 7, 6, 5, 4, 3, 2, or 1% of its weight of total dry cellular matter in optically-active 4-amino-3-hydroxybutyric acid.

In some embodiments, the cell or compositions comprising one of more cells is not E. coli. In some embodiments, the cell or compositions comprising one of more cells is from the genus Rhodococcus but is not for the species equi. In some embodiments, the cell of the present invention is not pathogenic to animals or plants. In some embodiments, the cell of the present invention is not pathogenic to humans. In some embodiments, the cell or compositions comprising one of more cells is from the genus Ralstonia. In some embodiments, the cell or compositions comprising one of more cells is from the species Ralstonia eutropha. In some embodiments the cell comprising one or more exogenous genes is strain Cupriavidus necator DSM number 531 or 541.

In some embodiments, the cell or compositions comprising the one or more cells have a 16S rRNA sequence with at least 50, 60, 70, 75, 80, 81, 82, 83, 84, 85, 86, 87, 88, 89, 90, 91, 92, 93, 94, 95, 96, 97, 98 or 99% nucleotide homology to one or more of SEQ ID NOs: 11 or 12. In some embodiments, the cell or compositions comprising the one or more cells have a 16S rRNA sequence with at least 70, 75, 80, 81, 82, 83, 84, 85, 86, 87, 88, 89, 90, 91, 92, 93, 94, 95, 96, 97, 98 or 99% nucleotide homology to one or more of SEQ ID NOs: 11. In some embodiments, the cell or compositions comprising the one or more cells have a 16S rRNA sequence with at least 70, 75, 80, 81, 82, 83, 84, 85, 86, 87, 88, 89, 90, 91, 92, 93, 94, 95, 96, 97, 98 or 99% nucleotide homology to one or more of SEQ ID NOs: 12. In some embodiments, the cell or compositions comprising the one or more cells have a 16S rRNA sequence with at least 70, 75, 80, 81, 82, 83, 84, 85, 86, 87, 88, 89, 90, 91, 92, 93, 94, 95, 96, 97, 98 or 99% nucleotide homology to one or more of SEQ ID NOs: 13.

In some embodiments the microorganism of the claimed invention is not dependent upon light to grow and/or metabolize and/or synthesize lipid molecules. In some embodiments, the microorganism of the claimed invention does not require any type of sugar to grow and/or metabolize and/or synthesize lipid molecules. In some embodiments, the microorganism of the claimed invention does not require any type of organic compound to grow and/or metabolize and/or synthesize lipid molecules. In some embodiments, the microorganism of the claimed invention does not require any type of fixed carbon to grow and/or metabolize and/or synthesize lipid molecules. In some embodiments, the microorganism can grow and/or metabolize lipids in a slightly anaerobic or extremely anaerobic environment. In some embodiments, the microorganism of the claimed invention is a facultative microorganism

Microbial culturing in the present invention is performed both for the sake of implementing genetic modifications, and for production of organic compounds, and specifically lipids and/or hydrocarbons (e.g., alkenes, alkynes, alkanes, unsaturated fatty acids, fatty acids, fatty alcohols, fatty aldehydes, triacylglycerols, hydroxy acids, diacids). Microbial culturing with the aim of genetic manipulation is generally performed at a small benchtop scale and often under conditions that select for genetically modified traits. Microbial culturing aimed at the commercial production of organic compounds and specifically lipids and/or hydrocarbons is typically performed in bioreactors at much greater scale (e.g., 500 L, 1,000 L 5,000 L, 10,000 L, 50,000 L, 100,000 L, 1,000,000 L bioreactor volumes and higher). In certain embodiments the chemoautotrophs of the present invention are grown in a liquid media inside a bioreactor using the methods of the invention. In some embodiments, the bioreactor containing the microorganisms is constructed of opaque materials that keep the culture in darkness. Bioreactors constructed out of opaque materials such as steel or reinforced concrete can be designed to have extremely big working volumes. In some embodiments of the present invention steel fermenters 50,000 liter and greater in volume are utilized. In some embodiments of the present invention egg-shape or cylindrical digesters 3,000,000 liters and greater in volume are utilized. In some embodiments, the bioreactor comprising the microorganism does not allow light to penetrate its interior.

The bioreactor or fermentor is used to culture cells through the various phases of their physiological cycle. A bioreactor is utilized for the cultivation of cells, which may be maintained at particular phases in their growth curve. The use of bioreactors is advantageous in many ways for cultivating chemoautotrophic growth. For certain embodiments, oleaginous cell mass, which is used to produce fuel, is grown to high densities in liquid suspension. Generally the control of growth conditions including control of dissolved carbon dioxide, oxygen, and other gases such as hydrogen, as well as other dissolved nutrients, trace elements, temperature and pH, is facilitated in a bioreactor.

Nutrient media as well as gases can be added to the bioreactor as either a batch addition, or periodically, or in response to a detected depletion or programmed set point, or continuously over the period the culture is grown and/or maintained. For certain embodiments, the bioreactor at inoculation is filled with a starting batch of nutrient media and/or gases at the beginning of growth, and no additional nutrient media and/or gases are added after inoculation. For certain embodiments, nutrient media and/or gases are added periodically after inoculation. For certain embodiments, nutrient media and/or gas is added after inoculation in response to a detected depletion of nutrient and/or gas. For certain embodiments, nutrient media and/or gas is added continuously after inoculation.

For certain embodiments the bioreactors have mechanisms to enable mixing of the nutrient media that include but are not limited to spinning stir bars, blades, impellers, or turbines, spinning, rocking, or turning vessels, gas lifts and sparging. The culture media may be mixed continuously or intermittently. The ports that are standard in bioreactors may be utilized to deliver, or withdraw, gases, liquids, solids, and/or slurries, into the bioreactor vessel enclosing the microbes of the present invention. Many bioreactors have multiple ports for different purposes (e.g. ports for media addition, gas addition, probes for pH and DO, sampling), and a given port may be used for various purposes during the course of a fermentation run. As an example, a port might be used to add nutrient media to the bioreactor at one point in time and at another time might be used for sampling. Preferably, the multiple use of a sampling port can be performed without introducing contamination or invasive species into the growth environment. A valve or other actuator enabling control of the sample flow or continuous sampling can be provided to a sampling port. For certain embodiments the bioreactors are equipped with at least one port suitable for culture inoculation that can additionally serve other uses including the addition of media or gas. Bioreactors ports enable control of the gas composition and flow rate into the culture environment. For example the ports can be used as gas inlets into the bioreactor through which gases are pumped. For some embodiments gases that may be pumped into a bioreactor include syngas, producer gas, hydrogen gas, CO2, air, air/CO₂ mixtures, ammonia, nitrogen, noble gases, such as argon, as well as other gases. In some embodiments that CO₂ may come from sources including but are not limited to: CO₂ from the gasification of organic matter; CO₂ from the calcination of limestone, CaCO₃, to produce quicklime, CaO; CO₂ from methane steam reforming, such as the CO₂ byproduct from ammonia or hydrogen production; combustion; CO₂ byproduct of sugar fermentation; CO₂ byproduct from sodium phosphate production; geologically or geothermally produced CO₂. Raising the gas flow rate into a bioreactor can enhance mixing of the culture and produce turbulence if the gas inlet is positioned under the surface of the liquid media such that gas bubbles or sparges up through the media. In some embodiments, a bioreactor comprises gas outlet ports for gas escape and pressure release. In some embodiments, gas inlets and outlets are preferably equipped with check valves to prevent gas backflow.

The present invention relates to bioreactors that comprise a cell, which comprises at least one exogenous nucleic acid sequences that encodes a lipid pathway enzyme. The present invention relates to a system of at least one bioreactor that comprise a cell, which comprises at least one exogenous nucleic acid sequences that encodes a lipid pathway enzyme. In some embodiments, the system comprises two or more, three or more, or four or more bioreactors, at least one of which comprise a cell, which comprises at least one exogenous nucleic acid sequences that encodes a lipid pathway enzyme. In some embodiments, the system of bioreactors comprises at least a first and second bioreactor, wherein the first bioreactor comprises a cell, which comprises at least one exogenous nucleic acid sequences that encodes a lipid pathway enzyme; and wherein the second bioreactor comprises a microorganism derived from a different species, wherein the microorganism from a different species comprises at least one exogenous nucleic acid sequence that encodes a lipid pathway enzyme. In some embodiments, the system of bioreactors comprises a first bioreactor that comprises the cell of the present invention and a second bioreactor comprising a microalgal, yeast, or bacterial cell.

In some embodiments, the cells of the present invention are capable of producing desaturated alkanes between 8 and 18 carbon atoms long at greater than 18 grams per liter volume of culture per three day period. In some embodiments, the cells of the present invention are capable of producing desaturated alkanes between 8 and 18 carbon atoms long at greater than or equal to 18 grams per liter volume of culture per three day period, wherein the desatruated alkanes are desatuated at a carbon position other than carbon-9.

### Genetic Modifications

The present invention relates to methods of modifying a bacterial cell to express one or more exogenous nucleic acid sequences that encodes one or more enzymes to enable fixation of a carbon-containing gas feedstock into useful carbon-based products of interest in an amount greater than an amount of carbon-based products produced by the same bacterial cell that does not express the exogenous nucleic acid sequences. Methods of selecting and manufacturing nucleic acid sequences for modification of bacterial cells are known and can be performed by transformation, electroporation, phage infection of bacteria, or other techniques for nucleic acid transfer generally known in the art. Standard recombinant DNA and molecular cloning techniques useful for the invention are well known in the art and are described by Sambrook, J., Fritsch, E. F. and Maniatis, T. Molecular Cloning: A Laboratory Manual; Cold Spring Harbor Laboratory Press: Cold Spring Harbor, (1989) (Maniatis) and by T. J. Silhavy, M. L. Bennan, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Assoc. and Wiley-Interscience (1987), all of which are incorporated by reference in their entireties.

The invention relates to genetic constructs comprising one or more exogenous genes that encode one or more amino acid sequences to enable fixation of a carbon-containing gas feedstock, including but not limited to syngas or producer gas, into useful carbon-based products of interest in an amount greater than an amount of carbon-based products produced by the same bacterial cell that does not express the exogenous nucleic acid sequence or sequences. Another aspect of the present invention relates to compositions that comprise at least one bacterial cell, which comprises at least one nucleic acid sequence that encodes at least one exogenous amino acid sequence that functions as a fatty acid acyl-ACP reductase, a fatty acid aldehyde decarbonylase and/or a thioesterase. In some embodiments, the bacterial cell is transformed with one or more, two or more, three or more, four or more, or five or more exogenous nucleic acid sequences that encode one or more amino acid sequences to enable fixation of a carbon-containing gas feedstock, including but not limited to syngas or producer gas, into useful carbon-based products of interest in an amount greater than an amount of carbon-based products produced by the same bacterial cell that does not express the exogenous nucleic acid sequence or sequences. According to the present invention, genetic material that encodes the enzyme is delivered to a bacterial cell in an expressible form. The genetic material, DNA or RNA, is taken up by the cells of the invention and expressed. The enzyme or enzymes that are thereby produced can biochemically modify lipid molecules to remove or add hydroxyl groups, remove or add carbonyl groups, remove or add carbon-carbon double bonds, remove or add carbon-carbon triple bonds, remove or add aldehyde groups, remove or add hydroxy groups, remove or add carboxylic acid groups, or remove or add ester groups to lipid molecules in lipid.

In some embodiments, the genetic constructs of the present invention comprise DNA, RNA, or combinations of both DNA and RNA. In some embodiments, the genetic construct of the present invention is a plasmid. It will be appreciated that, in some embodiments, the plasmid contains a variety of open reading frames (ORFs) encoding proteins of many diverse functions, including those enzymes that enable hydrocarbon or lipid modification, glutathione-S transferase (GST) activity, origins of replication, multiple cloning sites, promoters, and/or termination sequences. It is contemplated therefore that a host cell transformed with the plasmid will demonstrate the ability to modify a variety of lipids or hydrocarbons as well as maintain its copy number in the cytoplasm of the cell. The glutathione-S transferases (GSTs) represent a large group of detoxification enzymes. GSTs catalyze the conjugation of glutathione, homoglutathione and other glutathione-like analog via sulfhydryl group, to a large range of hydrophobic, electrophilic compounds. The conjugation can result in detoxification of these compounds. GST genes are found in both prokaryotic (e.g., E. coli) and eukaryotic organisms (e.g., yeast, plant and human). Although the homologies between the GSTs from prokaryotes and eukaryotes were low, many of the residues assigned to be important for the enzymatic function or structure in the eukaryotes were found to be conserved in prokaryotic GSTs (Nishida et al., J. Biol Chem 269:32536-32541 (1994)). It has been suggested that bacterial GST may represent a defense against the effects of antibiotics (Piccolomini et al., J Gen Microbiol 135:3119-3125 (1989)). Accordingly it is contemplated that a host strain transformed with the plasmid will have the ability detoxify harmful compounds via conjugation of those compounds to glutathione.

In some embodiments, the instant plasmid additionally encodes a variety of maintenance proteins, useful for maintaining, stabilizing and replicating the plasmid. It is contemplated that these genes may be used in conjunction with other bacterial plasmids deficient in these functions for the increased stabilization or robust maintenance of the plasmid. In some embodiments, the plasmid comprises maintenance proteins of particular interest including the REP origin of replication (encoded by ORF 38) the TRA proteins (TRAI, TRAJ and TRAK, encoded by ORF's 23, 24 and 25 respectively) and the VAG proteins (VAGD and VAGC, encoded by ORF's 33 and 34 respectively). The tra gene family is known to be involved in plasmid conjugation, a process that promotes DNA transfer from a donor to a recipient cell mediated by physical contact (Firth et al, Escherichia coli and Salmonella: Cellular and Molecular Biology, ASM press (1996)). Among tra gene products, TraI and TraK proteins are reported to be required for efficient plasmid site-specific recombination (Paterson et al. J. Bacteriol 181:2572-2583 (1999)). Furthermore, TraI is required for conjugal DNA transfer. Fukuda and Ohtsubo (Genes Cells 2:735-751 (1997)) reported that TraI has the activity of site- and strand-specific nicking of the supercoiled plasmid DNA. TraJ, traJ gene product, regulates transcription originating at the tra operon promoter P.sub.traY. (Firth et al., Escherichia coli and Salmonella: Cellular and Molecular Biology, ASM press (1996)). The stabilization proteins VAGC and VAGD encoded by vagC and vagD are involved in maintaining the plasmid as an autonomous replicating unit. Non-limiting examples of bacterial maintenance proteins of particular interest on the pSeq and pVer plasmids are represented by the following DNA and protein sequences:

SEQ ID: 01
TCGCGCGTTT CGGTGATGAC GGTGAAAACC TCTGACACAT GCAGCTCCCG GAGACGGTCA CAGCTTGTCT GTAAGCGGAT
GCCGGGAGCA GACAAGCCCG AGCGCGCAAA GCCACTACTG CCACTTTTGG AGACTGTGTA CGTCGAGGGC CTCTGCCAGT
GTCGAACAGA CATTCGCCTA CGGCCCTCGT CTGTTCGGGC TCAGGGCGCG TCAGCGGGTG TTGGCGGGTG TCGGGGCTGG
CTTAACTATG CGGCATCAGA GCAGATTGTA CTGAGAGTGC ACCATATGCG GTGTGAAATA AGTCCCGCGC AGTCGCCCAC
AACCGCCCAC AGCCCCGACC GAATTGATAC GCCGTAGTCT CGTCTAACAT GACTCTCACG TGGTATACGC CACACTTTAT
CCGCACAGAT GCGTAAGGAG AAAATACCGC ATCAGGCGCC ATTCGCCATT CAGGCTGCGC AACTGTTGGG AAGGGCGATC
GGTGCGGGCC TCTTCGCTAT GGCGTGTCTA CGCATTCCTC TTTTATGGCG TAGTCCGCGG TAAGCGGTAA GTCCGACGCG
TTGACAACCC TTCCCGCTAG CCACGCCCGG AGAAGCGATA TACGCCAGCT GGCGAAAGGG GGATGTGCTG CAAGGCGATT
AAGTTGGGTA ACGCCAGGGT TTTCCCAGTC ACGACGTTGT AAAACGACGG CCAGTGCCAA ATGCGGTCGA CCGCTTTCCC
CCTACACGAC GTTCCGCTAA TTCAACCCAT TGCGGTCCCA AAAGGGTCAG TGCTGCAACA TTTTGCTGCC GGTCACGGTT
GCTTGCATGC CTGCAGGTCG ACGGGCCCGG GATCCGATGC TCTTCCGCTA AGATCTGCCG CGGCCGCGTC CTCAGAAGAA
CTCGTCAAGA AGGCGATAGA CGAACGTACG GACGTCCAGC TGCCCGGGCC CTAGGCTACG AGAAGGCGAT TCTAGACGGC
GCCGGCGCAG GAGTCTTCTT GAGCAGTTCT TCCGCTATCT AGGCGATGCG CTGCGAATCG GGAGCGGCGA TACCGTAAAG
CACGAGGAAG CGGTCAGCCC ATTCGCCGCC AAGCTCTTCA GCAATATCAC GGGTAGCCAA TCCGCTACGC GACGCTTAGC
CCTCGCCGCT ATGGCATTTC GTGCTCCTTC GCCAGTCGGG TAAGCGGCGG TTCGAGAAGT CGTTATAGTG CCCATCGGTT
CGCTATGTCC TGATAGCGGT CCGCCACACC CAGCCGGCCA CAGTCGATGA ATCCAGAAAA GCGGCCATTT TCCACCATGA
TATTCGGCAA GCAGGCATCG GCGATACAGG ACTATCGCCA GGCGGTGTGG GTCGGCCGGT GTCAGCTACT TAGGTCTTTT
CGCCGGTAAA AGGTGGTACT ATAAGCCGTT CGTCCGTAGC CCATGGGTCA CGACGAGATC CTCGCCGTCG GGCATGCGCG
CCTTGAGCCT GGCGAACAGT TCGGCTGGCG CGAGCCCCTG ATGCTCTTCG TCCAGATCAT GGTACCCAGT GCTGCTCTAG
GAGCGGCAGC CCGTACGCGC GGAACTCGGA CCGCTTGTCA AGCCGACCGC GCTCGGGGAC TACGAGAAGC AGGTCTAGTA
CCTGATCGAC AAGACCGGCT TCCATCCGAG TACGTGCTCG CTCGATGCGA TGTTTCGCTT GGTGGTCGAA TGGGCAGGTA
GCCGGATCAA GCGTATGCAG GGACTAGCTG TTCTGGCCGA AGGTAGGCTC ATGCACGAGC GAGCTACGCT ACAAAGCGAA
CCACCAGCTT ACCCGTCCAT CGGCCTAGTT CGCATACGTC CCGCCGCATT GCATCAGCCA TGATGGATAC TTTCTCGGCA
GGAGCAAGGT GGGATGACAG GAGATCCTGC CCCGGCACTT CGCCCAATAG CAGCCAGTCC GGCGGCGTAA CGTAGTCGGT
ACTACCTATG AAAGAGCCGT CCTCGTTCCA CCCTACTGTC CTCTAGGACG GGGCCGTGAA GCGGGTTATC GTCGGTCAGG
CTTCCCGCTT CAGTGACAAC GTCGAGCACA GCTGCGCAAG GAACGCCCGT CGTGGCCAGC CACGATAGCC GCGCTGCCTC
GTCCTGCAGT TCATTCAGGG GAAGGGCGAA GTCACTGTTG CAGCTCGTGT CGACGCGTTC CTTGCGGGCA GCACCGGTCG
GTGCTATCGG CGCGACGGAG CAGGACGTCA AGTAAGTCCC CACCGGACAG GTCGGTCTTG ACAAAAAGAA CCGGGCGCCC
CTGCGCTGAC AGCCGGAACA CGGCGGCATC AGAGCAGCCG ATTGTCTGTT GTGCCCAGTC GTGGCCTGTC CAGCCAGAAC
TGTTTTTCTT GGCCCGCGGG GACGCGACTG TCGGCCTTGT GCCGCCGTAG TCTCGTCGGC TAACAGACAA CACGGGTCAG
ATAGCCGAAT AGCCTCTCCA CCCAAGCGGC CGGAGAACCT GCGTGCAATC CATCTTGTTC AATCATGATA TCCCTTAATT
AACCGTTAAC ACTAGTTCAG TATCGGCTTA TCGGAGAGGT GGGTTCGCCG GCCTCTTGGA CGCACGTTAG GTAGAACAAG
TTAGTACTAT AGGGAATTAA TTGGCAATTG TGATCAAGTC TCCATCTCGC CGTGTATGCG GGCCTGACGG ATCAACGTTC
CCACCGAGCC AGTCGAGATG TTCATCTGGT CGGCGATCTG CCGGTACTTC AAACCTTGTT AGGTAGAGCG GCACATACGC
CCGGACTGCC TAGTTGCAAG GGTGGCTCGG TCAGCTCTAC AAGTAGACCA GCCGCTAGAC GGCCATGAAG TTTGGAACAA
TGCGCAGTTC CACAGCCTTC TTGCGGCGTT CCTGCGCACG AGCGATGTAG TCGCCTCGGT CTTCGGCGAC GAGCCGTTTG
ATGGTGCTTT TCGAGACGCC ACGCGTCAAG GTGTCGGAAG AACGCCGCAA GGACGCGTGC TCGCTACATC AGCGGAGCCA
GAAGCCGCTG CTCGGCAAAC TACCACGAAA AGCTCTGCGG GAACTTGTCA GCCAACTCCT GCGCGGTCTG CGTGCGACGC
ATCACGCGTT CTGCAGCACC CATCAGTCCG TCCCCTCTGC TGCTGCGAAC AGTGCCGATC CTTGAACAGT CGGTTGAGGA
CGCGCCAGAC GCACGCTGCG TAGTGCGCAA GACGTCGTGG GTAGTCAGGC AGGGGAGACG ACGACGCTTG TCACGGCTAG
GATCGACCTT CTTGAGCTTC GGCCGCGGCG CGGTGGCGTT CTTCCGTACC GCTTCCGTTT TTGCGCTGCT GCTCACTTTG
CCGCGGCGTG CCTGGATTTT CTAGCTGGAA GAACTCGAAG CCGGCGCCGC GCCACCGCAA GAAGGCATGG CGAAGGCAAA
AACGCGACGA CGAGTGAAAC GGCGCCGCAC GGACCTAAAA CGAGAACTCG GCGGCGGTGA AGGTGCGGTG GGTCCAGTGG
GCGACTGATT TGCCGATCTG CTCGGCCTCG GCCCGACTCA TGGGGCCGAT CCCGTCGTTG GCTCTTGAGC CGCCGCCACT
TCCACGCCAC CCAGGTCACC CGCTGACTAA ACGGCTAGAC GAGCCGGAGC CGGGCTGAGT ACCCCGGCTA GGGCAGCAAC
GCGTCGAGGG TGAAGTTGGT CAGGGCGGTG AAGTCGGTGA CCATCTGCCG CCACACAGTG ATCGACGGGT AGTTCTGTTT
CCGGATCTCG CGGTAGGCCC CGCAGCTCCC ACTTCAACCA GTCCCGCCAC TTCAGCCACT GGTAGACGGC GGTGTGTCAC
TAGCTGCCCA TCAAGACAAA GGCCTAGAGC GCCATCCGGG ATTCCCGGGT GCGGTCGAAC AGTTCGACGT TCCGGCCCGT
TTCGGTCCTG ACCTGTGTCT TGCGGCCGTA GTCCGGTGGG GCGGGGAAAC GGTCACCGAG TAAGGGCCCA CGCCAGCTTG
TCAAGCTGCA AGGCCGGGCA AAGCCAGGAC TGGACACAGA ACGCCGGCAT CAGGCCACCC CGCCCCTTTG CCAGTGGCTC
CGCTTTTGCG AGGCCTTTGA GCGAGTACGG ATCCGAGGGA CCCCAGACCG TCGTCCAGTG CGGGTGGATC GGGTTCTGGG
TGAGCTGCTG CGCGTAGCCC GCGAAAACGC TCCGGAAACT CGCTCATGCC TAGGCTCCCT GGGGTCTGGC AGCAGGTCAC
GCCCACCTAG CCCAAGACCC ACTCGACGAC GCGCATCGGG TGATCGGCGC CGACCACCGA GGCGATCAGC CCCTGGTTCA
CCCGGTCGTA GAGCCGCAGC GGGCCCTGTC GGGCTGCCTG GAGGGTGTAG ACCGGGCTTT ACTAGCCGCG GCTGGTGGCT
CCGCTAGTCG GGGACCAAGT GGGCCAGCAT CTCGGCGTCG CCCGGGACAG CCCGACGGAC CTCCCACATC TGGCCCGAAA
CGAGCAGCCA CCACAGGTGC GCGTGCTCGG TCGCGGGATT GATCGTCATC ACGGTCGGAT CGGGCAGATC CGCGTTACGT
GCGGCCCACT GCGCCTGGTC GCTCGTCGGT GGTGTCCACG CGCACGAGCC AGCGCCCTAA CTAGCAGTAG TGCCAGCCTA
GCCCGTCTAG GCGCAATGCA CGCCGGGTGA CGCGGACCAG GTCGTCCACG TCGAGCACCA AGCCCAACCT GATCGACGGG
GTGCGGGCCG CAATGTAGCG GCGGGTGAGC GCCTCCGCGC GCGGCTGCGG CCACTGCCCG CAGCAGGTGC AGCTCGTGGT
TCGGGTTGGA CTAGCTGCCC CACGCCCGGC GTTACATCGC CGCCCACTCG CGGAGGCGCG CGCCGACGCC GGTGACGGGC
TCCCGGACGT AGTCATCCGT CGCGTGCGGG TATTTGAACC GCCAGCGGTC CAACCAGGCG TCAACAGCAG CGGTCATGAC
CGCCAAGCTA GGGCCGGATC AGGGCCTGCA TCAGTAGGCA GCGCACGCCC ATAAACTTGG CGGTCGCCAG GTTGGTCCGC
AGTTGTCGTC GCCAGTACTG GCGGTTCGAT CCCGGCCTAG TGTACCGATC GGGGGAGGCG CGCCGCAAAT TATTTAAGAG
TCTCGCTAGC AAACCATGTC AGGTGTTGCG GTGGGTTCCG GGTAAACCTC CACCCGAATT ACATGGCTAG CCCCCTCCGC
GCGGCGTTTA ATAAATTCTC AGAGCGATCG TTTGGTACAG TCCACAACGC CACCCAAGGC CCATTTGGAG GTGGGCTTAA
ATTTAAGAGT CTCGCTAGCT AAGCCCTATC TGATGCTGCG CGGGGGGTCC TTCGCACTGA ATCTCAAAGG TGGCCGGCTG
AATTTCGTCG CGCGAAAACC TAAATTCTCA GAGCGATCGA TTCGGGATAG ACTACGACGC GCCCCCCAGG AAGCGTGACT
TAGAGTTTCC ACCGGCCGAC TTAAAGCAGC GCGCTTTTGG TCCCTGGACA GTTCTGGAAT TCAGCAAGAG GTGTGTCTGA
ACTTCGGTGT TTTTTTGGGG GGTGACTCCA GCGGGGTGGG CACAACGCGA ACAGAGACCT AGGGACCTGT CAAGACCTTA
AGTCGTTCTC CACACAGACT TGAAGCCACA AAAAAACCCC CCACTGAGGT CGCCCCACCC GTGTTGCGCT TGTCTCTGGA
TGTGTGTACG ACGGCGGGAG GTAAGTCGGG TACGGCTCGG ACTGCGGTAG AGCAACCGTC GAATCGATTT CGAGCAGAGC
GAGCAGAGCA AGATATTCCA ACACACATGC TGCCGCCCTC CATTCAGCCC ATGCCGAGCC TGACGCCATC TCGTTGGCAG
CTTAGCTAAA GCTCGTCTCG CTCGTCTCGT TCTATAAGGT AAACTCCGGG GTTCCTCGGC GGCCTCCCCC GTCTGTTTGC
TCAACCGAGG GAGACCTGGC GGTCCCGCGT TTCCGGACGC GCGGGACCGC CTACCGCTCG TTTGAGGCCC CAAGGAGCCG
CCGGAGGGGG CAGACAAACG AGTTGGCTCC CTCTGGACCG CCAGGGCGCA AAGGCCTGCG CGCCCTGGCG GATGGCGAGC
AGAGCGGAAG AGCATCTAGA TGCATTCGCG AGGTACCGAG CTCGAATTCG TAATCATGGT CATAGCTGTT TCCTGTGTGA
AATTGTTATC CGCTCACAAT TCTCGCCTTC TCGTAGATCT ACGTAAGCGC TCCATGGCTC GAGCTTAAGC ATTAGTACCA
GTATCGACAA AGGACACACT TTAACAATAG GCGAGTGTTA TCCACACAAC ATACGAGCCG GAAGCATAAA GTGTAAAGCC
TGGGGTGCCT AATGAGTGAG CTAACTCACA TTAATTGCGT TGCGCTCACT GCCCGCTTTC AGGTGTGTTG TATGCTCGGC
CTTCGTATTT CACATTTCGG ACCCCACGGA TTACTCACTC GATTGAGTGT AATTAACGCA ACGCGAGTGA CGGGCGAAAG
CAGTCGGGAA ACCTGTCGTG CCAGCTGCAT TAATGAATCG GCCAACGCGC GGGGAGAGGC GGTTTGCGTA TTGGGCGCTC
TTCCGCTTCC TCGCTCACTG GTCAGCCCTT TGGACAGCAC GGTCGACGTA ATTACTTAGC CGGTTGCGCG CCCCTCTCCG
CCAAACGCAT AACCCGCGAG AAGGCGAAGG AGCGAGTGAC ACTCGCTGCG CTCGGTCGTT CGGCTGCGGC GAGCGGTATC
AGCTCACTCA AAGGCGGTAA TACGGTTATC CACAGAATCA GGGGATAACG CAGGAAAGAA TGAGCGACGC GAGCCAGCAA
GCCGACGCCG CTCGCCATAG TCGAGTGAGT TTCCGCCATT ATGCCAATAG GTGTCTTAGT CCCCTATTGC GTCCTTTCTT
CATGTGAGCA AAAGGCCAGC AAAAGGCCAG GAACCGTAAA AAGGCCGCGT TGCTGGCGTT TTTCCATAGG CTCCGCCCCC
CTGACGAGCA TCACAAAAAT GTACACTCGT TTTCCGGTCG TTTTCCGGTC CTTGGCATTT TTCCGGCGCA ACGACCGCAA
AAAGGTATCC GAGGCGGGGG GACTGCTCGT AGTGTTTTTA CGACGCTCAA GTCAGAGGTG GCGAAACCCG ACAGGACTAT
AAAGATACCA GGCGTTTCCC CCTGGAAGCT CCCTCGTGCG CTCTCCTGTT CCGACCCTGC GCTGCGAGTT CAGTCTCCAC
CGCTTTGGGC TGTCCTGATA TTTCTATGGT CCGCAAAGGG GGACCTTCGA GGGAGCACGC GAGAGGACAA GGCTGGGACG
CGCTTACCGG ATACCTGTCC GCCTTTCTCC CTTCGGGAAG CGTGGCGCTT TCTCATAGCT CACGCTGTAG GTATCTCAGT
TCGGTGTAGG TCGTTCGCTC GCGAATGGCC TATGGACAGG CGGAAAGAGG GAAGCCCTTC GCACCGCGAA AGAGTATCGA
GTGCGACATC CATAGAGTCA AGCCACATCC AGCAAGCGAG CAAGCTGGGC TGTGTGCACG AACCCCCCGT TCAGCCCGAC
CGCTGCGCCT TATCCGGTAA CTATCGTCTT GAGTCCAACC CGGTAAGACA CGACTTATCG GTTCGACCCG ACACACGTGC
TTGGGGGGCA AGTCGGGCTG GCGACGCGGA ATAGGCCATT GATAGCAGAA CTCAGGTTGG GCCATTCTGT GCTGAATAGC
CCACTGGCAG CAGCCACTGG TAACAGGATT AGCAGAGCGA GGTATGTAGG CGGTGCTACA GAGTTCTTGA AGTGGTGGCC
TAACTACGGC TACACTAGAA GGTGACCGTC GTCGGTGACC ATTGTCCTAA TCGTCTCGCT CCATACATCC GCCACGATGT
CTCAAGAACT TCACCACCGG ATTGATGCCG ATGTGATCTT GGACAGTATT TGGTATCTGC GCTCTGCTGA AGCCAGTTAC
CTTCGGAAAA AGAGTTGGTA GCTCTTGATC CGGCAAACAA ACCACCGCTG GTAGCGGTGG CCTGTCATAA ACCATAGACG
CGAGACGACT TCGGTCAATG GAAGCCTTTT TCTCAACCAT CGAGAACTAG GCCGTTTGTT TGGTGGCGAC CATCGCCACC
TTTTTTTGTT TGCAAGCAGC AGATTACGCG CAGAAAAAAA GGATCTCAAG AAGATCCTTT GATCTTTTCT ACGGGGTCTG
ACGCTCAGTG GAACGAAAAC AAAAAAACAA ACGTTCGTCG TCTAATGCGC GTCTTTTTTT CCTAGAGTTC TTCTAGGAAA
CTAGAAAAGA TGCCCCAGAC TGCGAGTCAC CTTGCTTTTG TCACGTTAAG GGATTTTGGT CATGAGATTA TCAAAAAGGA
TCTTCACCTA GATCCTTTTA AATTAAAAAT GAAGTTTTAA ATCAATCTAA AGTATATATG AGTGCAATTC CCTAAAACCA
GTACTCTAAT AGTTTTTCCT AGAAGTGGAT CTAGGAAAAT TTAATTTTTA CTTCAAAATT TAGTTAGATT TCATATATAC
AGTAAACTTG GTCTGACAGT TACCAATGCT TAATCAGTGA GGCACCTATC TCAGCGATCT GTCTATTTCG TTCATCCATA
GTTGCCTGAC TCCCCGTCGT TCATTTGAAC CAGACTGTCA ATGGTTACGA ATTAGTCACT CCGTGGATAG AGTCGCTAGA
CAGATAAAGC AAGTAGGTAT CAACGGACTG AGGGGCAGCA GTAGATAACT ACGATACGGG AGGGCTTACC ATCTGGCCCC
AGTGCTGCAA TGATACCGCG AGACCCACGC TCACCGGCTC CAGATTTATC AGCAATAAAC CATCTATTGA TGCTATGCCC
TCCCGAATGG TAGACCGGGG TCACGACGTT ACTATGGCGC TCTGGGTGCG AGTGGCCGAG GTCTAAATAG TCGTTATTTG
CAGCCAGCCG GAAGGGCCGA GCGCAGAAGT GGTCCTGCAA CTTTATCCGC CTCCATCCAG TCTATTAATT GTTGCCGGGA
AGCTAGAGTA AGTAGTTCGC GTCGGTCGGC CTTCCCGGCT CGCGTCTTCA CCAGGACGTT GAAATAGGCG GAGGTAGGTC
AGATAATTAA CAACGGCCCT TCGATCTCAT TCATCAAGCG CAGTTAATAG TTTGCGCAAC GTTGTTGCCA TTGCTACAGG
CATCGTGGTG TCACGCTCGT CGTTTGGTAT GGCTTCATTC AGCTCCGGTT CCCAACGATC GTCAATTATC AAACGCGTTG
CAACAACGGT AACGATGTCC GTAGCACCAC AGTGCGAGCA GCAAACCATA CCGAAGTAAG TCGAGGCCAA GGGTTGCTAG
AAGGCGAGTT ACATGATCCC CCATGTTGTG CAAAAAAGCG GTTAGCTCCT TCGGTCCTCC GATCGTTGTC AGAAGTAAGT
TGGCCGCAGT GTTATCACTC TTCCGCTCAA TGTACTAGGG GGTACAACAC GTTTTTTCGC CAATCGAGGA AGCCAGGAGG
CTAGCAACAG TCTTCATTCA ACCGGCGTCA CAATAGTGAG ATGGTTATGG CAGCACTGCA TAATTCTCTT ACTGTCATGC
CATCCGTAAG ATGCTTTTCT GTGACTGGTG AGTACTCAAC CAAGTCATTC TGAGAATAGT TACCAATACC GTCGTGACGT
ATTAAGAGAA TGACAGTACG GTAGGCATTC TACGAAAAGA CACTGACCAC TCATGAGTTG GTTCAGTAAG ACTCTTATCA
GTATGCGGCG ACCGAGTTGC TCTTGCCCGG CGTCAATACG GGATAATACC GCGCCACATA GCAGAACTTT AAAAGTGCTC
ATCATTGGAA AACGTTCTTC CATACGCCGC TGGCTCAACG AGAACGGGCC GCAGTTATGC CCTATTATGG CGCGGTGTAT
CGTCTTGAAA TTTTCACGAG TAGTAACCTT TTGCAAGAAG GGGGCGAAAA CTCTCAAGGA TCTTACCGCT GTTGAGATCC
AGTTCGATGT AACCCACTCG TGCACCCAAC TGATCTTCAG CATCTTTTAC TTTCACCAGC CCCCGCTTTT GAGAGTTCCT
AGAATGGCGA CAACTCTAGG TCAAGCTACA TTGGGTGAGC ACGTGGGTTG ACTAGAAGTC GTAGAAAATG AAAGTGGTCG
GTTTCTGGGT GAGCAAAAAC AGGAAGGCAA AATGCCGCAA AAAAGGGAAT AAGGGCGACA CGGAAATGTT GAATACTCAT
ACTCTTCCTT TTTCAATATT CAAAGACCCA CTCGTTTTTG TCCTTCCGTT TTACGGCGTT TTTTCCCTTA TTCCCGCTGT
GCCTTTACAA CTTATGAGTA TGAGAAGGAA AAAGTTATAA ATTGAAGCAT TTATCAGGGT TATTGTCTCA TGAGCGGATA
CATATTTGAA TGTATTTAGA AAAATAAACA AATAGGGGTT CCGCGCACAT TTCCCCGAAA TAACTTCGTA AATAGTCCCA
ATAACAGAGT ACTCGCCTAT GTATAAACTT ACATAAATCT TTTTATTTGT TTATCCCCAA GGCGCGTGTA AAGGGGCTTT
AGTGCCACCT GACGTCTAAG AAACCATTAT TATCATGACA TTAACCTATA AAAATAGGCG TATCACGAGG CCCTTTCGTC
TCACGGTGGA CTGCAGATTC TTTGGTAATA ATAGTACTGT AATTGGATAT TTTTATCCGC ATAGTGCTCC GGGAAAGCAG
SEQ ID: 02
GGGGAGCCGC GCCGAAGGCG TGGGGGAACC CCGCAGGGGT GCCCTTCTTT GGGCACCAAA GAACTAGATA TAGGGCGAAA
TGCGAAAGAC TTAAAAATCA CCCCTCGGCG CGGCTTCCGC ACCCCCTTGG GGCGTCCCCA CGGGAAGAAA CCCGTGGTTT
CTTGATCTAT ATCCCGCTTT ACGCTTTCTG AATTTTTAGT ACAACTTAAA AAAGGGGGGT ACGCAACAGC TCATTGCGGC
ACCCCCCGCA ATAGCTCATT GCGTAGGTTA AAGAAAATCT GTAATTGACT GCCACTTTTA TGTTGAATTT TTTCCCCCCA
TGCGTTGTCG AGTAACGCCG TGGGGGGCGT TATCGAGTAA CGCATCCAAT TTCTTTTAGA CATTAACTGA CGGTGAAAAT
CGCAACGCAT AATTGTTGTC GCGCTGCCGA AAAGTTGCAG CTGATTGCGC ATGGTGCCGC AACCGTGCGG CACCCTACCG
CATGGAGATA AGCATGGCCA GCGTTGCGTA TTAACAACAG CGCGACGGCT TTTCAACGTC GACTAACGCG TACCACGGCG
TTGGCACGCC GTGGGATGGC GTACCTCTAT TCGTACCGGT CGCAGTCCAG AGAAATCGGC ATTCAAGCCA AGAACAAGCC
CGGTCACTGG GTGCAAACGG AACGCAAAGC GCATGAGGCG TGGGCCGGGC TTATTGCGAG GCGTCAGGTC TCTTTAGCCG
TAAGTTCGGT TCTTGTTCGG GCCAGTGACC CACGTTTGCC TTGCGTTTCG CGTACTCCGC ACCCGGCCCG AATAACGCTC
GAAACCCACG GCGGCAATGC TGCTGCATCA CCTCGTGGCG CAGATGGGCC ACCAGAACGC CGTGGTGGTC AGCCAGAAGA
CACTTTCCAA GCTCATCGGA CTTTGGGTGC CGCCGTTACG ACGACGTAGT GGAGCACCGC GTCTACCCGG TGGTCTTGCG
GCACCACCAG TCGGTCTTCT GTGAAAGGTT CGAGTAGCCT CGTTCTTTGC GGACGGTCCA ATACGCAGTC AAGGACTTGG
TGGCCGAGCG CTGGATCTCC GTCGTGAAGC TCAACGGCCC CGGCACCGTG TCGGCCTACG GCAAGAAACG CCTGCCAGGT
TATGCGTCAG TTCCTGAACC ACCGGCTCGC GACCTAGAGG CAGCACTTCG AGTTGCCGGG GCCGTGGCAC AGCCGGATGC
TGGTCAATGA CCGCGTGGCG TGGGGCCAGC CCCGCGACCA GTTGCGCCTG TCGGTGTTCA GTGCCGCCGT GGTGGTTGAT
CACGACGACC AGGACGAATC ACCAGTTACT GGCGCACCGC ACCCCGGTCG GGGCGCTGGT CAACGCGGAC AGCCACAAGT
CACGGCGGCA CCACCAACTA GTGCTGCTGG TCCTGCTTAG GCTGTTGGGG CATGGCGACC TGCGCCGCAT CCCGACCCTG
TATCCGGGCG AGCAGCAACT ACCGACCGGC CCCGGCGAGG AGCCGCCCAG CCAGCCCGGC CGACAACCCC GTACCGCTGG
ACGCGGCGTA GGGCTGGGAC ATAGGCCCGC TCGTCGTTGA TGGCTGGCCG GGGCCGCTCC TCGGCGGGTC GGTCGGGCCG
ATTCCGGGCA TGGAACCAGA CCTGCCAGCC TTGACCGAAA CGGAGGAATG GGAACGGCGC GGGCAGCAGC GCCTGCCGAT
GCCCGATGAG CCGTGTTTTC TAAGGCCCGT ACCTTGGTCT GGACGGTCGG AACTGGCTTT GCCTCCTTAC CCTTGCCGCG
CCCGTCGTCG CGGACGGCTA CGGGCTACTC GGCACAAAAG TGGACGATGG CGAGCCGTTG GAGCCGCCGA CACGGGTCAC
GCTGCCGCGC CGGTAGCACT TGGGTTGCGC AGCAACCCGT AAGTGCGCTG TTCCAGACTA ACCTGCTACC GCTCGGCAAC
CTCGGCGGCT GTGCCCAGTG CGACGGCGCG GCCATCGTGA ACCCAACGCG TCGTTGGGCA TTCACGCGAC AAGGTCTGAT
TCGGCTGTAG CCGCCTCGCC GCCCTATACC TTGTCTGCCT CCCCGCGTTG CGTCGCGGTG CATGGAGCCG GGCCACCTCG
ACCTGAATGG AAGCCGGCGG AGCCGACATC GGCGGAGCGG CGGGATATGG AACAGACGGA GGGGCGCAAC GCAGCGCCAC
GTACCTCGGC CCGGTGGAGC TGGACTTACC TTCGGCCGCC CACCTCGCTA ACGGATTCAC CGTTTTTATC AGGCTCTGGG
AGGCAGAATA AATGATCATA TCGTCAATTA TTACCTCCAC GGGGAGAGCC TGAGCAAACT GTGGAGCGAT TGCCTAAGTG
GCAAAAATAG TCCGAGACCC TCCGTCTTAT TTACTAGTAT AGCAGTTAAT AATGGAGGTG CCCCTCTCGG ACTCGTTTGA
GGCCTCAGGC ATTTGAGAAG CACACGGTCA CACTGCTTCC GGTAGTCAAT AAACCGGTAA ACCAGCAATA GACATAAGCG
GCTATTTAAC GACCCTGCCC CCGGAGTCCG TAAACTCTTC GTGTGCCAGT GTGACGAAGG CCATCAGTTA TTTGGCCATT
TGGTCGTTAT CTGTATTCGC CGATAAATTG CTGGGACGGG TGAACCGACG ACCGGGTCGA ATTTGCTTTC GAATTTCTGC
CATTCATCCG CTTATTATCA CTTATTCAGG CGTAGCACCA GGCGTTTAAG GGCACCAATA ACTTGGCTGC TGGCCCAGCT
TAAACGAAAG CTTAAAGACG GTAAGTAGGC GAATAATAGT GAATAAGTCC GCATCGTGGT CCGCAAATTC CCGTGGTTAT
ACTGCCTTAA AAAAATTACG CCCCGCCCTG CCACTCATCG CAGTCGGCCT ATTGGTTAAA AAATGAGCTG ATTTAACAAA
AATTTAACGC GAATTTTAAC TGACGGAATT TTTTTAATGC GGGGCGGGAC GGTGAGTAGC GTCAGCCGGA TAACCAATTT
TTTACTCGAC TAAATTGTTT TTAAATTGCG CTTAAAATTG AAAATATTAA CGCTTACAAT TTCCATTCGC CATTCAGGCT
GCGCAACTGT TGGGAAGGGC GATCGGTGCG GGCCTCTTCG CTATTACGCC AGCTGGCGAA TTTTATAATT GCGAATGTTA
AAGGTAAGCG GTAAGTCCGA CGCGTTGACA ACCCTTCCCG CTAGCCACGC CCGGAGAAGC GATAATGCGG TCGACCGCTT
AGGGGGATGT GCTGCAAGGC GATTAAGTTG GGTAACGCCA GGGTTTTCCC AGTCACGACG TTGTAAAACG ACGGCCAGTG
AGCGCGCGTA ATACGACTCA TCCCCCTACA CGACGTTCCG CTAATTCAAC CCATTGCGGT CCCAAAAGGG TCAGTGCTGC
AACATTTTGC TGCCGGTCAC TCGCGCGCAT TATGCTGAGT CTATAGGGCG AATTGGAGCT CCACCGCGGT GGCGGCCGCT
CTAGAACTAG TGGATCCCCC GGGCTGCAGG AATTCGATAT CAAGCTTATC GATACCGTCG GATATCCCGC TTAACCTCGA
GGTGGCGCCA CCGCCGGCGA GATCTTGATC ACCTAGGGGG CCCGACGTCC TTAAGCTATA GTTCGAATAG CTATGGCAGC
ACCTCGAGGG GGGGCCCGGT ACCCAGCTTT TGTTCCCTTT AGTGAGGGTT AATTGCGCGC TTGGCGTAAT CATGGTCATA
GCTGTTTCCT GTGTGAAATT TGGAGCTCCC CCCCGGGCCA TGGGTCGAAA ACAAGGGAAA TCACTCCCAA TTAACGCGCG
AACCGCATTA GTACCAGTAT CGACAAAGGA CACACTTTAA GTTATCCGCT CACAATTCCA CACAACATAC GAGCCGGAAG
CATAAAGTGT AAAGCCTGGG GTGCCTAATG AGTGAGCTAA CTCACATTAA TTGCGTTGCG CAATAGGCGA GTGTTAAGGT
GTGTTGTATG CTCGGCCTTC GTATTTCACA TTTCGGACCC CACGGATTAC TCACTCGATT GAGTGTAATT AACGCAACGC
CTCACTGCCC GCTTTCCAGT CGGGAAACCT GTCGTGCCAG CTGCATTAAT GAATCGGCCA ACGCGCGGGG AGAGGCGGTT
TGCGTATTGG GCGCATGCAT GAGTGACGGG CGAAAGGTCA GCCCTTTGGA CAGCACGGTC GACGTAATTA CTTAGCCGGT
TGCGCGCCCC TCTCCGCCAA ACGCATAACC CGCGTACGTA AAAAACTGTT GTAATTCATT AAGCATTCTG CCGACATGGA
AGCCATCACA AACGGCATGA TGAACCTGAA TCGCCAGCGG CATCAGCACC TTGTCGCCTT TTTTTGACAA CATTAAGTAA
TTCGTAAGAC GGCTGTACCT TCGGTAGTGT TTGCCGTACT ACTTGGACTT AGCGGTCGCC GTAGTCGTGG AACAGCGGAA
GCGTATAATA TTTGCCCATG GGGGTGGGCG AAGAACTCCA GCATGAGATC CCCGCGCTGG AGGATCATCC AGCCGGCGTC
CCGGAAAACG ATTCCGAAGC CGCATATTAT AAACGGGTAC CCCCACCCGC TTCTTGAGGT CGTACTCTAG GGGCGCGACC
TCCTAGTAGG TCGGCCGCAG GGCCTTTTGC TAAGGCTTCG CCAACCTTTC ATAGAAGGCG GCGGTGGAAT CGAAATCTCG
TGATGGCAGG TTGGGCGTCG CTTGGTCGGT CATTTCGAAC CCCAGAGTCC CGCTCAGAAG GGTTGGAAAG TATCTTCCGC
CGCCACCTTA GCTTTAGAGC ACTACCGTCC AACCCGCAGC GAACCAGCCA GTAAAGCTTG GGGTCTCAGG GCGAGTCTTC
AACTCGTCAA GAAGGCGATA GAAGGCGATG CGCTGCGAAT CGGGAGCGGC GATACCGTAA AGCACGAGGA AGCGGTCAGC
CCATTCGCCG CCAAGCTCTT TTGAGCAGTT CTTCCGCTAT CTTCCGCTAC GCGACGCTTA GCCCTCGCCG CTATGGCATT
TCGTGCTCCT TCGCCAGTCG GGTAAGCGGC GGTTCGAGAA CAGCAATATC ACGGGTAGCC AACGCTATGT CCTGATAGCG
GTCCGCCACA CCCAGCCGGC CACAGTCGAT GAATCCAGAA AAGCGGCCAT TTTCCACCAT GTCGTTATAG TGCCCATCGG
TTGCGATACA GGACTATCGC CAGGCGGTGT GGGTCGGCCG GTGTCAGCTA CTTAGGTCTT TTCGCCGGTA AAAGGTGGTA
GATATTCGGC AAGCAGGCAT CGCCATGGGT CACGACGAGA TCCTCGCCGT CGGGCATGCG CGCCTTGAGC CTGGCGAACA
GTTCGGCTGG CGCGAGCCCC CTATAAGCCG TTCGTCCGTA GCGGTACCCA GTGCCGCCGT AGGAGCGGCA GCCCGTACGC
GCGGAACTCG GACCGCTTGT CAAGCCGACC GCGCTCGGGG TGATGCTCTT CGTCCAGATC ATCCTGATCG ACAAGACCGG
CTTCCATCCG AGTACGTGCT CGCTCGATGC GATGTTTCGC TTGGTGGTCG AATGGGCAGG ACTACGAGAA GCAGGTCTAG
TAGGACTAGC TGTTCTGGCC GAAGGTAGGC TCATGCACGA GCGAGCTACG CTACAAAGCG AACCACCAGC TTACCCGTCC
TAGCCGGATC AAGCGTATGC AGCCGCCGCA TTGCATCAGC CATGATGGAT ACTTTCTCGG CAGGAGCAAG GTGAGATGAC
AGGAGATCCT GCCCCGGCAC ATCGGCCTAG TTCGCATACG TCGGCGGCGT AACGTAGTCG GTACTACCTA TGAAAGAGCC
GTCCTCGTTC CACTCTACTG TCCTCTAGGA CGGGGCCGTG TTCGCCCAAT AGCAGCCAGT CCCTTCCCGC TTCAGTGACA
ACGTCGAGCA CAGCTGCGCA AGGAACGCCC GTCGTGGCCA GCCACGATAG CCGCGCTGCC AAGCGGGTTA TCGTCGGTCA
GGGAAGGGCG AAGTCACTGT TGCAGCTCGT GTCGACGCGT TCCTTGCGGG CAGCACCGGT CGGTGCTATC GGCGCGACGG
TCGTCCTGCA GTTCATTCAG GGCACCGGAC AGGTCGGTCT TGACAAAAAG AACCGGGCGC CCCTGCGCTG ACAGCCGGAA
CACGGCGGCA TCAGAGCAGC AGCAGGACGT CAAGTAAGTC CCGTGGCCTG TCCAGCCAGA ACTGTTTTTC TTGGCCCGCG
GGGACGCGAC TGTCGGCCTT GTGCCGCCGT AGTCTCGTCG CGATTGTCTG TTGTGCCCAG TCATAGCCGA ATAGCCTCTC
CACCCAAGCG GCCGGAGAAC CTGCGTGCAA TCCATCTTGT TCAATCATGC GAAACGATCC GCTAACAGAC AACACGGGTC
AGTATCGGCT TATCGGAGAG GTGGGTTCGC CGGCCTCTTG GACGCACGTT AGGTAGAACA AGTTAGTACG CTTTGCTAGG
TCATCCTGTC TCTTGATCAG ATCTTGATCC CCTGCGCCAT CAGATCCTTG GCGGCAAGAA AGCCATCCAG TTTACTTTGC
AGGGCTTCCC AACCTTACCA AGTAGGACAG AGAACTAGTC TAGAACTAGG GGACGCGGTA GTCTAGGAAC CGCCGTTCTT
TCGGTAGGTC AAATGAAACG TCCCGAAGGG TTGGAATGGT GAGGGCGCCC CAGCTGGCAA TTCCGGTTCG CTTGCTGTCC
ATAAAACCGC CCAGTCTAGC TATCGCCATG TAAGCCCACT GCAAGCTACC TGCTTTCTCT CTCCCGCGGG GTCGACCGTT
AAGGCCAAGC GAACGACAGG TATTTTGGCG GGTCAGATCG ATAGCGGTAC ATTCGGGTGA CGTTCGATGG ACGAAAGAGA
TTGCGCTTGC GTTTTCCCTT GTCCAGATAG CCCAGTAGCT GACATTCATC CCAGGTGGCA CTTTTCGGGG AAATGTGCGC
GCCCGCGTTC CTGCTGGCGC AACGCGAACG CAAAAGGGAA CAGGTCTATC GGGTCATCGA CTGTAAGTAG GGTCCACCGT
GAAAAGCCCC TTTACACGCG CGGGCGCAAG GACGACCGCG TGGGCCTGTT TCTGGCGCTG GACTTCCCGC TGTTCCGTCA
GCAGCTTTTC GCCCACGGCC TTGATGATCG CGGCGGCCTT GGCCTGCATA TCCCGATTCA ACCCGGACAA AGACCGCGAC
CTGAAGGGCG ACAAGGCAGT CGTCGAAAAG CGGGTGCCGG AACTACTAGC GCCGCCGGAA CCGGACGTAT AGGGCTAAGT
ACGGCCCCAG GGCGTCCAGA ACGGGCTTCA GGCGCTCCCG AAGGTCTCGG GCCGTCTCTT GGGCTTGATC GGCCTTCTTG
CGCATCTCAC GCGCTCCTGC TGCCGGGGTC CCGCAGGTCT TGCCCGAAGT CCGCGAGGGC TTCCAGAGCC CGGCAGAGAA
CCCGAACTAG CCGGAAGAAC GCGTAGAGTG CGCGAGGACG GGCGGCCTGT AGGGCAGGCT CATACCCCTG CCGAACCGCT
TTTGTCAGCC GGTCGGCCAC GGCTTCCGGC GTCTCAACGC GCTTTGAGAT TCCCAGCTTT CCGCCGGACA TCCCGTCCGA
GTATGGGGAC GGCTTGGCGA AAACAGTCGG CCAGCCGGTG CCGAAGGCCG CAGAGTTGCG CGAAACTCTA AGGGTCGAAA
TCGGCCAATC CCTGCGGTGC ATAGGCGCGT GGCTCGACCG CTTGCGGGCT GATGGTGACG TGGCCCACTG GTGGCCGCTC
CAGGGCCTCG TAGAACGCCT AGCCGGTTAG GGACGCCACG TATCCGCGCA CCGAGCTGGC GAACGCCCGA CTACCACTGC
ACCGGGTGAC CACCGGCGAG GTCCCGGAGC ATCTTGCGGA GAATGCGCGT GTGACGTGCC TTGCTGCCCT CGATGCCCCG
TTGCAGCCCT AGATCGGCCA CAGCGGCCGC AAACGTGGTC TGGTCGCGGG TCATCTGCGC CTTACGCGCA CACTGCACGG
AACGACGGGA GCTACGGGGC AACGTCGGGA TCTAGCCGGT GTCGCCGGCG TTTGCACCAG ACCAGCGCCC AGTAGACGCG
TTTGTTGCCG ATGAACTCCT TGGCCGACAG CCTGCCGTCC TGCGTCAGCG GCACCACGAA CGCGGTCATG TGCGGGCTGG
TTTCGTCACG GTGGATGCTG AAACAACGGC TACTTGAGGA ACCGGCTGTC GGACGGCAGG ACGCAGTCGC CGTGGTGCTT
GCGCCAGTAC ACGCCCGACC AAAGCAGTGC CACCTACGAC GCCGTCACGA TGCGATCCGC CCCGTACTTG TCCGCCAGCC
ACTTGTGCGC CTTCTCGAAG AACGCCGCCT GCTGTTCTTG GCTGGCCGAC TTCCACCATT CGGCAGTGCT ACGCTAGGCG
GGGCATGAAC AGGCGGTCGG TGAACACGCG GAAGAGCTTC TTGCGGCGGA CGACAAGAAC CGACCGGCTG AAGGTGGTAA
CCGGGCTGGC CGTCATGACG TACTCGACCG CCAACACAGC GTCCTTGCGC CGCTTCTCTG GCAGCAACTC GCGCAGTCGG
CCCATCGCTT CATCGGTGCT GGCCCGACCG GCAGTACTGC ATGAGCTGGC GGTTGTGTCG CAGGAACGCG GCGAAGAGAC
CGTCGTTGAG CGCGTCAGCC GGGTAGCGAA GTAGCCACGA GCTGGCCGCC CAGTGCTCGT TCTCTGGCGT CCTGCTGGCG
TCAGCGTTGG GCGTCTCGCG CTCGCGGTAG GCGTGCTTGA GACTGGCCGC CACGTTGCCC CGACCGGCGG GTCACGAGCA
AGAGACCGCA GGACGACCGC AGTCGCAACC CGCAGAGCGC GAGCGCCATC CGCACGAACT CTGACCGGCG GTGCAACGGG
ATTTTCGCCA GCTTCTTGCA TCGCATGATC GCGTATGCCG CCATGCCTGC CCCTCCCTTT TGGTGTCCAA CCGGCTCGAC
GGGGGCAGCG CAAGGCGGTG TAAAAGCGGT CGAAGAACGT AGCGTACTAG CGCATACGGC GGTACGGACG GGGAGGGAAA
ACCACAGGTT GGCCGAGCTG CCCCCGTCGC GTTCCGCCAC CCTCCGGCGG GCCACTCAAT GCTTGAGTAT ACTCACTAGA
CTTTGCTTCG CAAAGTCGTG ACCGCCTACG GCGGCTGCGG CGCCCTACGG GCTTGCTCTC GGAGGCCGCC CGGTGAGTTA
CGAACTCATA TGAGTGATCT GAAACGAAGC GTTTCAGCAC TGGCGGATGC CGCCGACGCC GCGGGATGCC CGAACGAGAG
CGGGCTTCGC CCTGCGCGGT CGCTGCGCTC CCTTGCCAGC CCGTGGATAT GTGGACGATG GCCGCGAGCG GCCACCGGCT
GGCTCGCTTC GCTCGGCCCG GCCCGAAGCG GGACGCGCCA GCGACGCGAG GGAACGGTCG GGCACCTATA CACCTGCTAC
CGGCGCTCGC CGGTGGCCGA CCGAGCGAAG CGAGCCGGGC TGGACAACCC TGCTGGACAA GCTGATGGAC AGGCTGCGCC
TGCCCACGAG CTTGACCACA GGGATTGCCC ACCGGCTACC CAGCCTTCGA CCACATACCC ACCTGTTGGG ACGACCTGTT
CGACTACCTG TCCGACGCGG ACGGGTGCTC GAACTGGTGT CCCTAACGGG TGGCCGATGG GTCGGAAGCT GGTGTATGGG
ACCGGCTCCA ACTGCGCGGC CTGCGGCCTT GCCCCATCAA TTTTTTTAAT TTTCTCTGGG GAAAAGCCTC CGGCCTGCGG
CCTGCGCGCT TCGCTTGCCG TGGCCGAGGT TGACGCGCCG GACGCCGGAA CGGGGTAGTT AAAAAAATTA AAAGAGACCC
CTTTTCGGAG GCCGGACGCC GGACGCGCGA AGCGAACGGC GTTGGACACC AAGTGGAAGG CGGGTCAAGG CTCGCGCAGC
GACCGCGCAG CGGCTTGGCC TTGACGCGCC TGGAACGACC CAAGCCTATG CGAGTGGGGG CAACCTGTGG TTCACCTTCC
GCCCAGTTCC GAGCGCGTCG CTGGCGCGTC GCCGAACCGG AACTGCGCGG ACCTTGCTGG GTTCGGATAC GCTCACCCCC
CAGTCGAAGG CGAAGCCCGC CCGCCTGCCC CCCGAGCCTC ACGGCGGCGA GTGCGGGGGT TCCAAGGGGG CAGCGCCACC
TTGGGCAAGG CCGAAGGCCG GTCAGCTTCC GCTTCGGGCG GGCGGACGGG GGGCTCGGAG TGCCGCCGCT CACGCCCCCA
AGGTTCCCCC GTCGCGGTGG AACCCGTTCC GGCTTCCGGC CGCAGTCGAT CAACAAGCCC CGGAGGGGCC ACTTTTTGCC
GGAGGCGTCA GCTAGTTGTT CGGGGCCTCC CCGGTGAAAA ACGGCCTC
SEQ ID: 03
GGGGAGCCGC GCCGAAGGCG TGGGGGAACC CCGCAGGGGT GCCCTTCTTT GGGCACCAAA GAACTAGATA TAGGGCGAAA
TGCGAAAGAC TTAAAAATCA CCCCTCGGCG CGGCTTCCGC ACCCCCTTGG GGCGTCCCCA CGGGAAGAAA CCCGTGGTTT
CTTGATCTAT ATCCCGCTTT ACGCTTTCTG AATTTTTAGT ACAACTTAAA AAAGGGGGGT ACGCAACAGC TCATTGCGGC
ACCCCCCGCA ATAGCTCATT GCGTAGGTTA AAGAAAATCT GTAATTGACT GCCACTTTTA TGTTGAATTT TTTCCCCCCA
TGCGTTGTCG AGTAACGCCG TGGGGGGCGT TATCGAGTAA CGCATCCAAT TTCTTTTAGA CATTAACTGA CGGTGAAAAT
CGCAACGCAT AATTGTTGTC GCGCTGCCGA AAAGTTGCAG CTGATTGCGC ATGGTGCCGC AACCGTGCGG CACCCTACCG
CATGGAGATA AGCATGGCCA GCGTTGCGTA TTAACAACAG CGCGACGGCT TTTCAACGTC GACTAACGCG TACCACGGCG
TTGGCACGCC GTGGGATGGC GTACCTCTAT TCGTACCGGT CGCAGTCCAG AGAAATCGGC ATTCAAGCCA AGAACAAGCC
CGGTCACTGG GTGCAAACGG AACGCAAAGC GCATGAGGCG TGGGCCGGGC TTATTGCGAG GCGTCAGGTC TCTTTAGCCG
TAAGTTCGGT TCTTGTTCGG GCCAGTGACC CACGTTTGCC TTGCGTTTCG CGTACTCCGC ACCCGGCCCG AATAACGCTC
GAAACCCACG GCGGCAATGC TGCTGCATCA CCTCGTGGCG CAGATGGGCC ACCAGAACGC CGTGGTGGTC AGCCAGAAGA
CACTTTCCAA GCTCATCGGA CTTTGGGTGC CGCCGTTACG ACGACGTAGT GGAGCACCGC GTCTACCCGG TGGTCTTGCG
GCACCACCAG TCGGTCTTCT GTGAAAGGTT CGAGTAGCCT CGTTCTTTGC GGACGGTCCA ATACGCAGTC AAGGACTTGG
TGGCCGAGCG CTGGATCTCC GTCGTGAAGC TCAACGGCCC CGGCACCGTG TCGGCCTACG GCAAGAAACG CCTGCCAGGT
TATGCGTCAG TTCCTGAACC ACCGGCTCGC GACCTAGAGG CAGCACTTCG AGTTGCCGGG GCCGTGGCAC AGCCGGATGC
TGGTCAATGA CCGCGTGGCG TGGGGCCAGC CCCGCGACCA GTTGCGCCTG TCGGTGTTCA GTGCCGCCGT GGTGGTTGAT
CACGACGACC AGGACGAATC ACCAGTTACT GGCGCACCGC ACCCCGGTCG GGGCGCTGGT CAACGCGGAC AGCCACAAGT
CACGGCGGCA CCACCAACTA GTGCTGCTGG TCCTGCTTAG GCTGTTGGGG CATGGCGACC TGCGCCGCAT CCCGACCCTG
TATCCGGGCG AGCAGCAACT ACCGACCGGC CCCGGCGAGG AGCCGCCCAG CCAGCCCGGC CGACAACCCC GTACCGCTGG
ACGCGGCGTA GGGCTGGGAC ATAGGCCCGC TCGTCGTTGA TGGCTGGCCG GGGCCGCTCC TCGGCGGGTC GGTCGGGCCG
ATTCCGGGCA TGGAACCAGA CCTGCCAGCC TTGACCGAAA CGGAGGAATG GGAACGGCGC GGGCAGCAGC GCCTGCCGAT
GCCCGATGAG CCGTGTTTTC TAAGGCCCGT ACCTTGGTCT GGACGGTCGG AACTGGCTTT GCCTCCTTAC CCTTGCCGCG
CCCGTCGTCG CGGACGGCTA CGGGCTACTC GGCACAAAAG TGGACGATGG CGAGCCGTTG GAGCCGCCGA CACGGGTCAC
GCTGCCGCGC CGGTAGCACT TGGGTTGCGC AGCAACCCGT AAGTGCGCTG TTCCAGACTA ACCTGCTACC GCTCGGCAAC
CTCGGCGGCT GTGCCCAGTG CGACGGCGCG GCCATCGTGA ACCCAACGCG TCGTTGGGCA TTCACGCGAC AAGGTCTGAT
TCGGCTGTAG CCGCCTCGCC GCCCTATACC TTGTCTGCCT CCCCGCGTTG CGTCGCGGTG CATGGAGCCG GGCCACCTCG
ACCTGAATGG AAGCCGGCGG AGCCGACATC GGCGGAGCGG CGGGATATGG AACAGACGGA GGGGCGCAAC GCAGCGCCAC
GTACCTCGGC CCGGTGGAGC TGGACTTACC TTCGGCCGCC CACCTCGCTA ACGGATTCAC CGTTTTTATC AGGCTCTGGG
AGGCAGAATA AATGATCATA TCGTCAATTA TTACCTCCAC GGGGAGAGCC TGAGCAAACT GTGGAGCGAT TGCCTAAGTG
GCAAAAATAG TCCGAGACCC TCCGTCTTAT TTACTAGTAT AGCAGTTAAT AATGGAGGTG CCCCTCTCGG ACTCGTTTGA
GGCCTCAGGC ATTTGAGAAG CACACGGTCA CACTGCTTCC GGTAGTCAAT AAACCGGTAA ACCAGCAATA GACATAAGCG
GCTATTTAAC GACCCTGCCC CCGGAGTCCG TAAACTCTTC GTGTGCCAGT GTGACGAAGG CCATCAGTTA TTTGGCCATT
TGGTCGTTAT CTGTATTCGC CGATAAATTG CTGGGACGGG TGAACCGACG ACCGGGTCGA ATTTGCTTTC GAATTTCTGC
CATTCATCCG CTTATTATCA CTTATTCAGG CGTAGCACCA GGCGTTTAAG GGCACCAATA ACTTGGCTGC TGGCCCAGCT
TAAACGAAAG CTTAAAGACG GTAAGTAGGC GAATAATAGT GAATAAGTCC GCATCGTGGT CCGCAAATTC CCGTGGTTAT
ACTGCCTTAA AAAAATTACG CCCCGCCCTG CCACTCATCG CAGTCGGCCT ATTGGTTAAA AAATGAGCTG ATTTAACAAA
AATTTAACGC GAATTTTAAC TGACGGAATT TTTTTAATGC GGGGCGGGAC GGTGAGTAGC GTCAGCCGGA TAACCAATTT
TTTACTCGAC TAAATTGTTT TTAAATTGCG CTTAAAATTG AAAATATTAA CGCTTACAAT TTCCATTCGC CATTCAGGCT
GCGCAACTGT TGGGAAGGGC GATCGGTGCG GGCCTCTTCG CTATTACGCC AGCTGGCGAA TTTTATAATT GCGAATGTTA
AAGGTAAGCG GTAAGTCCGA CGCGTTGACA ACCCTTCCCG CTAGCCACGC CCGGAGAAGC GATAATGCGG TCGACCGCTT
AGGGGGATGT GCTGCAAGGC GATTAAGTTG GGTAACGCCA GGGTTTTCCC AGTCACGACG TTGTAAAACG ACGGCCAGTG
AGCGCGCGTA ATACGACTCA TCCCCCTACA CGACGTTCCG CTAATTCAAC CCATTGCGGT CCCAAAAGGG TCAGTGCTGC
AACATTTTGC TGCCGGTCAC TCGCGCGCAT TATGCTGAGT CTATAGGGCG AATTGGAGCT CCACCGCGGT GGCGGCCGCT
CTAGAACTAG TGGATCCCCC GGGCTGCAGG AATTCGATAT CAAGCTTATC GATACCGTCG GATATCCCGC TTAACCTCGA
GGTGGCGCCA CCGCCGGCGA GATCTTGATC ACCTAGGGGG CCCGACGTCC TTAAGCTATA GTTCGAATAG CTATGGCAGC
ACGGGCCCGG GATCCGATGC TCTTCCGCTA AGATCTTTTA CTAGTTCAGT CCATCTCGCC GTGTATGCGG GCCTGACGGA
TCAACGTTCC CACCGAGCCA TGCCCGGGCC CTAGGCTACG AGAAGGCGAT TCTAGAAAAT GATCAAGTCA GGTAGAGCGG
CACATACGCC CGGACTGCCT AGTTGCAAGG GTGGCTCGGT GTCGAGATGT TCATCTGGTC GGCGATCTGC CGGTACTTCA
AACCTTGTTT GCGCAGTTCC ACAGCCTTCT TGCGGCGTTC CTGCGCACGA GCGATGTAGT CAGCTCTACA AGTAGACCAG
CCGCTAGACG GCCATGAAGT TTGGAACAAA CGCGTCAAGG TGTCGGAAGA ACGCCGCAAG GACGCGTGCT CGCTACATCA
CGCCTCGGTC TTCGGCGACG AGCCGTTTGA TGGTGCTTTT CGAGACGCCG AACTTGTCAG CCAACTCCTG CGCGGTCTGC
GTGCGACGCA TCACGCGTTC GCGGAGCCAG AAGCCGCTGC TCGGCAAACT ACCACGAAAA GCTCTGCGGC TTGAACAGTC
GGTTGAGGAC GCGCCAGACG CACGCTGCGT AGTGCGCAAG TGCAGCACCC ATCAGTCCGT CCCCTCTGCT GCTGCGAACA
GTGCCGATCG ATCGACCTTC TTGAGCTTCG GCCGCGGCGC GGTGGCGTTC TTCCGTACCG ACGTCGTGGG TAGTCAGGCA
GGGGAGACGA CGACGCTTGT CACGGCTAGC TAGCTGGAAG AACTCGAAGC CGGCGCCGCG CCACCGCAAG AAGGCATGGC
CTTCCGTTTT TGCGCTGCTG CTCACTTTGC CGCGGCGTGC CTGGATTTTC GAGAACTCGG CGGCGGTGAA GGTGCGGTGG
GTCCAGTGGG CGACTGATTT GAAGGCAAAA ACGCGACGAC GAGTGAAACG GCGCCGCACG GACCTAAAAG CTCTTGAGCC
GCCGCCACTT CCACGCCACC CAGGTCACCC GCTGACTAAA GCCGATCTGC TCGGCCTCGG CCCGACTCAT GGGGCCGATC
CCGTCGTTGG CGTCGAGGGT GAAGTTGGTC AGGGCGGTGA AGTCGGTGAC CATCTGCCGC CGGCTAGACG AGCCGGAGCC
GGGCTGAGTA CCCCGGCTAG GGCAGCAACC GCAGCTCCCA CTTCAACCAG TCCCGCCACT TCAGCCACTG GTAGACGGCG
CACACAGTGA TCGACGGGTA GTTCTGTTTC CGGATCTCGC GGTAGGCCCA TTCCCGGGTG CGGTCGAACA GTTCGACGTT
CCGGCCCGTT TCGGTCCTGA GTGTGTCACT AGCTGCCCAT CAAGACAAAG GCCTAGAGCG CCATCCGGGT AAGGGCCCAC
GCCAGCTTGT CAAGCTGCAA GGCCGGGCAA AGCCAGGACT CCTGTGTCTT GCGGCCGTAG TCCGGTGGGG CGGGGAAACG
GTCACCGAGC GCTTTTGCGA GGCCTTTGAG CGAGTACGGA TCCGAGGGAC CCCAGACCGT GGACACAGAA CGCCGGCATC
AGGCCACCCC GCCCCTTTGC CAGTGGCTCG CGAAAACGCT CCGGAAACTC GCTCATGCCT AGGCTCCCTG GGGTCTGGCA
CGTCCAGTGC GGGTGGATCG GGTTCTGGGT GAGCTGCTGC GCGTAGCCCT GATCGGCGCC GACCACCGAG GCGATCAGCC
CCTGGTTCAC CCGGTCGTAG GCAGGTCACG CCCACCTAGC CCAAGACCCA CTCGACGACG CGCATCGGGA CTAGCCGCGG
CTGGTGGCTC CGCTAGTCGG GGACCAAGTG GGCCAGCATC AGCCGCAGCG GGCCCTGTCG GGCTGCCTGG AGGGTGTAGA
CCGGGCTTTC GAGCAGCCAC CACAGGTGCG CGTGCTCGGT CGCGGGATTG ATCGTCATCA TCGGCGTCGC CCGGGACAGC
CCGACGGACC TCCCACATCT GGCCCGAAAG CTCGTCGGTG GTGTCCACGC GCACGAGCCA GCGCCCTAAC TAGCAGTAGT
CGGTCGGATC GGGCAGATCC GCGTTACGTG CGGCCCACTG CGCCTGGTCG TCGTCCACGT CGAGCACCAA GCCCAACCTG
ATCGACGGGG TGCGGGCCGC GCCAGCCTAG CCCGTCTAGG CGCAATGCAC GCCGGGTGAC GCGGACCAGC AGCAGGTGCA
GCTCGTGGTT CGGGTTGGAC TAGCTGCCCC ACGCCCGGCG AATGTAGCGG CGGGTGAGCG CCTCCGCGCG CGGCTGCGGC
CACTGCCCGT CCCGGACGTA GTCATCCGTC GCGTGCGGGT ATTTGAACCG CCAGCGGTCC TTACATCGCC GCCCACTCGC
GGAGGCGCGC GCCGACGCCG GTGACGGGCA GGGCCTGCAT CAGTAGGCAG CGCACGCCCA TAAACTTGGC GGTCGCCAGG
AACCAGGCGT CAACAGCAGC GGTCATGACC GCCAAGCTAG GGCCGGATCT GTACCGATCG GGGGAGGCGC GCCGCAAATT
ATTTAAGAGT CTCGCTAGCA TTGGTCCGCA GTTGTCGTCG CCAGTACTGG CGGTTCGATC CCGGCCTAGA CATGGCTAGC
CCCCTCCGCG CGGCGTTTAA TAAATTCTCA GAGCGATCGT AACCATGTCA GGTGTTGCGG TGGGTTCCGG GTAAACCTCC
ACCCGAATTA TTTAAGAGTC TCGCTAGCTA AGCCCTATCT GATGCTGCGC GGGGGGTCCT TTGGTACAGT CCACAACGCC
ACCCAAGGCC CATTTGGAGG TGGGCTTAAT AAATTCTCAG AGCGATCGAT TCGGGATAGA CTACGACGCG CCCCCCAGGA
TCGCACTGAA TCTCAAAGGT GGCCGGCTGA ATTTCGTCGC GCGAAAACCT CCCTGGACAG TTCTGGAATT CAGCAAGAGG
TGTGTCTGAA CTTCGGTGTT AGCGTGACTT AGAGTTTCCA CCGGCCGACT TAAAGCAGCG CGCTTTTGGA GGGACCTGTC
AAGACCTTAA GTCGTTCTCC ACACAGACTT GAAGCCACAA TTTTTGGGGG GTGACTCCAG CGGGGTGGGC ACAACGCGAA
CAGAGACCTT GTGTGTACGA CGGCGGGAGG TAAGTCGGGT ACGGCTCGGA CTGCGGTAGA AAAAACCCCC CACTGAGGTC
GCCCCACCCG TGTTGCGCTT GTCTCTGGAA CACACATGCT GCCGCCCTCC ATTCAGCCCA TGCCGAGCCT GACGCCATCT
GCAACCGTCG AATCGATTTC GAGCAGAGCG AGCAGAGCAA GATATTCCAA AACTCCGGGG TTCCTCGGCG GCCTCCCCCG
TCTGTTTGCT CAACCGAGGG CGTTGGCAGC TTAGCTAAAG CTCGTCTCGC TCGTCTCGTT CTATAAGGTT TTGAGGCCCC
AAGGAGCCGC CGGAGGGGGC AGACAAACGA GTTGGCTCCC AGACCTGGCG GTCCCGCGTT TCCGGACGCG CGGGACCGCC
TACCGCTCGA GAGCGGAAGA GCATCTAGAT GCATTCGCGA GGTACCCAGC TTTTGTTCCC TCTGGACCGC CAGGGCGCAA
AGGCCTGCGC GCCCTGGCGG ATGGCGAGCT CTCGCCTTCT CGTAGATCTA CGTAAGCGCT CCATGGGTCG AAAACAAGGG
TTTAGTGAGG GTTAATTGCG CGCTTGGCGT AATCATGGTC ATAGCTGTTT CCTGTGTGAA ATTGTTATCC GCTCACAATT
CCACACAACA TACGAGCCGG AAATCACTCC CAATTAACGC GCGAACCGCA TTAGTACCAG TATCGACAAA GGACACACTT
TAACAATAGG CGAGTGTTAA GGTGTGTTGT ATGCTCGGCC AAGCATAAAG TGTAAAGCCT GGGGTGCCTA ATGAGTGAGC
TAACTCACAT TAATTGCGTT GCGCTCACTG CCCGCTTTCC AGTCGGGAAA CCTGTCGTGC TTCGTATTTC ACATTTCGGA
CCCCACGGAT TACTCACTCG ATTGAGTGTA ATTAACGCAA CGCGAGTGAC GGGCGAAAGG TCAGCCCTTT GGACAGCACG
CAGCTGCATT AATGAATCGG CCAACGCGCG GGGAGAGGCG GTTTGCGTAT TGGGCGCATG CATAAAAACT GTTGTAATTC
ATTAAGCATT CTGCCGACAT GTCGACGTAA TTACTTAGCC GGTTGCGCGC CCCTCTCCGC CAAACGCATA ACCCGCGTAC
GGAAGCCATC ACAAACGGCA TGATGAACCT GAATCGCCAG CGGCATCAGC ACCTTGTCGC CTTGCGTATA ATATTTGCCC
ATGGGGGTGG GCGAAGAACT CCTTCGGTAG TGTTTGCCGT ACTACTTGGA CTTAGCGGTC GCCGTAGTCG TGGAACAGCG
GAACGCATAT TATAAACGGG TACCCCCACC CGCTTCTTGA CCAGCATGAG ATCCCCGCGC TGGAGGATCA TCCAGCCGGC
GTCCCGGAAA ACGATTCCGA AGCCCAACCT TTCATAGAAG GCGGCGGTGG AATCGAAATC GGTCGTACTC TAGGGGCGCG
ACCTCCTAGT AGGTCGGCCG CAGGGCCTTT TGCTAAGGCT TCGGGTTGGA AAGTATCTTC CGCCGCCACC TTAGCTTTAG
TCGTGATGGC AGGTTGGGCG TCGCTTGGTC GGTCATTTCG AACCCCAGAG TCCCGCTCAG AAGAACTCGT CAAGAAGGCG
ATAGAAGGCG ATGCGCTGCG AGCACTACCG TCCAACCCGC AGCGAACCAG CCAGTAAAGC TTGGGGTCTC AGGGCGAGTC
TTCTTGAGCA GTTCTTCCGC TATCTTCCGC TACGCGACGC AATCGGGAGC GGCGATACCG TAAAGCACGA GGAAGCGGTC
AGCCCATTCG CCGCCAAGCT CTTCAGCAAT ATCACGGGTA GCCAACGCTA TGTCCTGATA TTAGCCCTCG CCGCTATGGC
ATTTCGTGCT CCTTCGCCAG TCGGGTAAGC GGCGGTTCGA GAAGTCGTTA TAGTGCCCAT CGGTTGCGAT ACAGGACTAT
GCGGTCCGCC ACACCCAGCC GGCCACAGTC GATGAATCCA GAAAAGCGGC CATTTTCCAC CATGATATTC GGCAAGCAGG
CATCGCCATG GGTCACGACG CGCCAGGCGG TGTGGGTCGG CCGGTGTCAG CTACTTAGGT CTTTTCGCCG GTAAAAGGTG
GTACTATAAG CCGTTCGTCC GTAGCGGTAC CCAGTGCTGC AGATCCTCGC CGTCGGGCAT GCGCGCCTTG AGCCTGGCGA
ACAGTTCGGC TGGCGCGAGC CCCTGATGCT CTTCGTCCAG ATCATCCTGA TCGACAAGAC TCTAGGAGCG GCAGCCCGTA
CGCGCGGAAC TCGGACCGCT TGTCAAGCCG ACCGCGCTCG GGGACTACGA GAAGCAGGTC TAGTAGGACT AGCTGTTCTG
CGGCTTCCAT CCGAGTACGT GCTCGCTCGA TGCGATGTTT CGCTTGGTGG TCGAATGGGC AGGTAGCCGG ATCAAGCGTA
TGCAGCCGCC GCATTGCATC GCCGAAGGTA GGCTCATGCA CGAGCGAGCT ACGCTACAAA GCGAACCACC AGCTTACCCG
TCCATCGGCC TAGTTCGCAT ACGTCGGCGG CGTAACGTAG AGCCATGATG GATACTTTCT CGGCAGGAGC AAGGTGAGAT
GACAGGAGAT CCTGCCCCGG CACTTCGCCC AATAGCAGCC AGTCCCTTCC CGCTTCAGT TCGGTACTAC CTATGAAAGA
GCCGTCCTCG TTCCACTCTA CTGTCCTCTA GGACGGGGCC GTGAAGCGGG TTATCGTCGG TCAGGGAAGG GCGAAGTCAC
ACAACGTCGA GCACAGCTGC GCAAGGAACG CCCGTCGTGG CCAGCCACGA TAGCCGCGCT GCCTCGTCCT GCAGTTCATT
CAGGGCACCG GACAGGTCGG TGTTGCAGCT CGTGTCGACG CGTTCCTTGC GGGCAGCACC GGTCGGTGCT ATCGGCGCGA
CGGAGCAGGA CGTCAAGTAA GTCCCGTGGC CTGTCCAGCC TCTTGACAAA AAGAACCGGG CGCCCCTGCG CTGACAGCCG
GAACACGGCG GCATCAGAGC AGCCGATTGT CTGTTGTGCC CAGTCATAGC CGAATAGCCT AGAACTGTTT TTCTTGGCCC
GCGGGGACGC GACTGTCGGC CTTGTGCCGC CGTAGTCTCG TCGGCTAACA GACAACACGG GTCAGTATCG GCTTATCGGA
CTCCACCCAA GCGGCCGGAG AACCTGCGTG CAATCCATCT TGTTCAATCA TGCGAAACGA TCCTCATCCT GTCTCTTGAT
CAGATCTTGA TCCCCTGCGC GAGGTGGGTT CGCCGGCCTC TTGGACGCAC GTTAGGTAGA ACAAGTTAGT ACGCTTTGCT
AGGAGTAGGA CAGAGAACTA GTCTAGAACT AGGGGACGCG CATCAGATCC TTGGCGGCAA GAAAGCCATC CAGTTTACTT
TGCAGGGCTT CCCAACCTTA CCAGAGGGCG CCCCAGCTGG CAATTCCGGT TCGCTTGCTG GTAGTCTAGG AACCGCCGTT
CTTTCGGTAG GTCAAATGAA ACGTCCCGAA GGGTTGGAAT GGTCTCCCGC GGGGTCGACC GTTAAGGCCA AGCGAACGAC
TCCATAAAAC CGCCCAGTCT AGCTATCGCC ATGTAAGCCC ACTGCAAGCT ACCTGCTTTC TCTTTGCGCT TGCGTTTTCC
CTTGTCCAGA TAGCCCAGTA AGGTATTTTG GCGGGTCAGA TCGATAGCGG TACATTCGGG TGACGTTCGA TGGACGAAAG
AGAAACGCGA ACGCAAAAGG GAACAGGTCT ATCGGGTCAT GCTGACATTC ATCCCAGGTG GCACTTTTCG GGGAAATGTG
CGCGCCCGCG TTCCTGCTGG CGCTGGGCCT GTTTCTGGCG CTGGACTTCC CGCTGTTCCG CGACTGTAAG TAGGGTCCAC
CGTGAAAAGC CCCTTTACAC GCGCGGGCGC AAGGACGACC GCGACCCGGA CAAAGACCGC GACCTGAAGG GCGACAAGGC
TCAGCAGCTT TTCGCCCACG GCCTTGATGA TCGCGGCGGC CTTGGCCTGC ATATCCCGAT TCAACGGCCC CAGGGCGTCC
AGAACGGGCT TCAGGCGCTC AGTCGTCGAA AAGCGGGTGC CGGAACTACT AGCGCCGCCG GAACCGGACG TATAGGGCTA
AGTTGCCGGG GTCCCGCAGG TCTTGCCCGA AGTCCGCGA CCGAAGGTCT CGGGCCGTCT CTTGGGCTTG ATCGGCCTTC
TTGCGCATCT CACGCGCTCC TGCGGCGGCC TGTAGGGCAG GCTCATACCC CTGCCGAACC GGCTTCCAGA GCCCGGCAGA
GAACCCGAAC TAGCCGGAAG AACGCGTAGA GTGCGCGAGG ACGCCGCCGG ACATCCCGTC CGAGTATGGG GACGGCTTGG
GCTTTTGTCA GCCGGTCGGC CACGGCTTCC GGCGTCTCAA CGCGCTTTGA GATTCCCAGC TTTTCGGCCA ATCCCTGCGG
TGCATAGGCG CGTGGCTCGA CGAAAACAGT CGGCCAGCCG GTGCCGAAGG CCGCAGAGTT GCGCGAAACT CTAAGGGTCG
AAAAGCCGGT TAGGGACGCC ACGTATCCGC GCACCGAGCT CCGCTTGCGG GCTGATGGTG ACGTGGCCCA CTGGTGGCCG
CTCCAGGGCC TCGTAGAACG CCTGAATGCG CGTGTGACGT GCCTTGCTGC CCTCGATGCC GGCGAACGCC CGACTACCAC
TGCACCGGGT GACCACCGGC GAGGTCCCGG AGCATCTTGC GGACTTACGC GCACACTGCA CGGAACGACG GGAGCTACGG
CCGTTGCAGC CCTAGATCGG CCACAGCGGC CGCAAACGTG GTCTGGTCGC GGGTCATCTG CGCTTTGTTG CCGATGAACT
CCTTGGCCGA CAGCCTGCCG GGCAACGTCG GGATCTAGCC GGTGTCGCCG GCGTTTGCAC CAGACCAGCG CCCAGTAGAC
GCGAAACAAC GGCTACTTGA GGAACCGGCT GTCGGACGGC TCCTGCGTCA GCGGCACCAC GAACGCGGTC ATGTGCGGGC
TGGTTTCGTC ACGGTGGATG CTGGCCGTCA CGATGCGATC CGCCCCGTAC TTGTCCGCCA AGGACGCAGT CGCCGTGGTG
CTTGCGCCAG TACACGCCCG ACCAAAGCAG TGCCACCTAC GACCGGCAGT GCTACGCTAG GCGGGGCATG AACAGGCGGT
GCCACTTGTG CGCCTTCTCG AAGAACGCCG CCTGCTGTTC TTGGCTGGCC GACTTCCACC ATTCCGGGCT GGCCGTCATG
ACGTACTCGA CCGCCAACAC CGGTGAACAC GCGGAAGAGC TTCTTGCGGC GGACGACAAG AACCGACCGG CTGAAGGTGG
TAAGGCCCGA CCGGCAGTAC TGCATGAGCT GGCGGTTGTG AGCGTCCTTG CGCCGCTTCT CTGGCAGCAA CTCGCGCAGT
CGGCCCATCG CTTCATCGGT GCTGCTGGCC GCCCAGTGCT CGTTCTCTGG CGTCCTGCTG TCGCAGGAAC GCGGCGAAGA
GACCGTCGTT GAGCGCGTCA GCCGGGTAGC GAAGTAGCCA CGACGACCGG CGGGTCACGA GCAAGAGACC GCAGGACGAC
GCGTCAGCGT TGGGCGTCTC GCGCTCGCGG TAGGCGTGCT TGAGACTGGC CGCCACGTTG CCCATTTTCG CCAGCTTCTT
GCATCGCATG ATCGCGTATG CGCAGTCGCA ACCCGCAGAG CGCGAGCGCC ATCCGCACGA ACTCTGACCG GCGGTGCAAC
GGGTAAAAGC GGTCGAAGAA CGTAGCGTAC TAGCGCATAC CCGCCATGCC TGCCCCTCCC TTTTGGTGTC CAACCGGCTC
GACGGGGGCA GCGCAAGGCG GTGCCTCCGG CGGGCCACTC AATGCTTGAG TATACTCACT GGCGGTACGG ACGGGGAGGG
AAAACCACAG GTTGGCCGAG CTGCCCCCGT CGCGTTCCGC CACGGAGGCC GCCCGGTGAG TTACGAACTC ATATGAGTGA
AGACTTTGCT TCGCAAAGTC GTGACCGCCT ACGGCGGCTG CGGCGCCCTA CGGGCTTGCT CTCCGGGCTT CGCCCTGCGC
GGTCGCTGCG CTCCCTTGCC TCTGAAACGA AGCGTTTCAG CACTGGCGGA TGCCGCCGAC GCCGCGGGAT GCCCGAACGA
GAGGCCCGAA GCGGGACGCG CCAGCGACGC GAGGGAACGG
SEQ ID: 04
GGGGAGCCGC GCCGAAGGCG TGGGGGAACC CCGCAGGGGT GCCCTTCTTT GGGCACCAAA GAACTAGATA TAGGGCGAAA
TGCGAAAGAC TTAAAAATCA CCCCTCGGCG CGGCTTCCGC ACCCCCTTGG GGCGTCCCCA CGGGAAGAAA CCCGTGGTTT
CTTGATCTAT ATCCCGCTTT ACGCTTTCTG AATTTTTAGT ACAACTTAAA AAAGGGGGGT ACGCAACAGC TCATTGCGGC
ACCCCCCGCA ATAGCTCATT GCGTAGGTTA AAGAAAATCT GTAATTGACT GCCACTTTTA TGTTGAATTT TTTCCCCCCA
TGCGTTGTCG AGTAACGCCG TGGGGGGCGT TATCGAGTAA CGCATCCAAT TTCTTTTAGA CATTAACTGA CGGTGAAAAT
CGCAACGCAT AATTGTTGTC GCGCTGCCGA AAAGTTGCAG CTGATTGCGC ATGGTGCCGC AACCGTGCGG CACCCTACCG
CATGGAGATA AGCATGGCCA GCGTTGCGTA TTAACAACAG CGCGACGGCT TTTCAACGTC GACTAACGCG TACCACGGCG
TTGGCACGCC GTGGGATGGC GTACCTCTAT TCGTACCGGT CGCAGTCCAG AGAAATCGGC ATTCAAGCCA AGAACAAGCC
CGGTCACTGG GTGCAAACGG AACGCAAAGC GCATGAGGCG TGGGCCGGGC TTATTGCGAG GCGTCAGGTC TCTTTAGCCG
TAAGTTCGGT TCTTGTTCGG GCCAGTGACC CACGTTTGCC TTGCGTTTCG CGTACTCCGC ACCCGGCCCG AATAACGCTC
GAAACCCACG GCGGCAATGC TGCTGCATCA CCTCGTGGCG CAGATGGGCC ACCAGAACGC CGTGGTGGTC AGCCAGAAGA
CACTTTCCAA GCTCATCGGA CTTTGGGTGC CGCCGTTACG ACGACGTAGT GGAGCACCGC GTCTACCCGG TGGTCTTGCG
GCACCACCAG TCGGTCTTCT GTGAAAGGTT CGAGTAGCCT CGTTCTTTGC GGACGGTCCA ATACGCAGTC AAGGACTTGG
TGGCCGAGCG CTGGATCTCC GTCGTGAAGC TCAACGGCCC CGGCACCGTG TCGGCCTACG GCAAGAAACG CCTGCCAGGT
TATGCGTCAG TTCCTGAACC ACCGGCTCGC GACCTAGAGG CAGCACTTCG AGTTGCCGGG GCCGTGGCAC AGCCGGATGC
TGGTCAATGA CCGCGTGGCG TGGGGCCAGC CCCGCGACCA GTTGCGCCTG TCGGTGTTCA GTGCCGCCGT GGTGGTTGAT
CACGACGACC AGGACGAATC ACCAGTTACT GGCGCACCGC ACCCCGGTCG GGGCGCTGGT CAACGCGGAC AGCCACAAGT
CACGGCGGCA CCACCAACTA GTGCTGCTGG TCCTGCTTAG GCTGTTGGGG CATGGCGACC TGCGCCGCAT CCCGACCCTG
TATCCGGGCG AGCAGCAACT ACCGACCGGC CCCGGCGAGG AGCCGCCCAG CCAGCCCGGC CGACAACCCC GTACCGCTGG
ACGCGGCGTA GGGCTGGGAC ATAGGCCCGC TCGTCGTTGA TGGCTGGCCG GGGCCGCTCC TCGGCGGGTC GGTCGGGCCG
ATTCCGGGCA TGGAACCAGA CCTGCCAGCC TTGACCGAAA CGGAGGAATG GGAACGGCGC GGGCAGCAGC GCCTGCCGAT
GCCCGATGAG CCGTGTTTTC TAAGGCCCGT ACCTTGGTCT GGACGGTCGG AACTGGCTTT GCCTCCTTAC CCTTGCCGCG
CCCGTCGTCG CGGACGGCTA CGGGCTACTC GGCACAAAAG TGGACGATGG CGAGCCGTTG GAGCCGCCGA CACGGGTCAC
GCTGCCGCGC CGGTAGCACT TGGGTTGCGC AGCAACCCGT AAGTGCGCTG TTCCAGACTA ACCTGCTACC GCTCGGCAAC
CTCGGCGGCT GTGCCCAGTG CGACGGCGCG GCCATCGTGA ACCCAACGCG TCGTTGGGCA TTCACGCGAC AAGGTCTGAT
TCGGCTGTAG CCGCCTCGCC GCCCTATACC TTGTCTGCCT CCCCGCGTTG CGTCGCGGTG CATGGAGCCG GGCCACCTCG
ACCTGAATGG AAGCCGGCGG AGCCGACATC GGCGGAGCGG CGGGATATGG AACAGACGGA GGGGCGCAAC GCAGCGCCAC
GTACCTCGGC CCGGTGGAGC TGGACTTACC TTCGGCCGCC CACCTCGCTA ACGGATTCAC CGTTTTTATC AGGCTCTGGG
AGGCAGAATA AATGATCATA TCGTCAATTA TTACCTCCAC GGGGAGAGCC TGAGCAAACT GTGGAGCGAT TGCCTAAGTG
GCAAAAATAG TCCGAGACCC TCCGTCTTAT TTACTAGTAT AGCAGTTAAT AATGGAGGTG CCCCTCTCGG ACTCGTTTGA
GGCCTCAGGC ATTTGAGAAG CACACGGTCA CACTGCTTCC GGTAGTCAAT AAACCGGTAA ACCAGCAATA GACATAAGCG
GCTATTTAAC GACCCTGCCC CCGGAGTCCG TAAACTCTTC GTGTGCCAGT GTGACGAAGG CCATCAGTTA TTTGGCCATT
TGGTCGTTAT CTGTATTCGC CGATAAATTG CTGGGACGGG TGAACCGACG ACCGGGTCGA ATTTGCTTTC GAATTTCTGC
CATTCATCCG CTTATTATCA CTTATTCAGG CGTAGCACCA GGCGTTTAAG GGCACCAATA ACTTGGCTGC TGGCCCAGCT
TAAACGAAAG CTTAAAGACG GTAAGTAGGC GAATAATAGT GAATAAGTCC GCATCGTGGT CCGCAAATTC CCGTGGTTAT
ACTGCCTTAA AAAAATTACG CCCCGCCCTG CCACTCATCG CAGTCGGCCT ATTGGTTAAA AAATGAGCTG ATTTAACAAA
AATTTAACGC GAATTTTAAC TGACGGAATT TTTTTAATGC GGGGCGGGAC GGTGAGTAGC GTCAGCCGGA TAACCAATTT
TTTACTCGAC TAAATTGTTT TTAAATTGCG CTTAAAATTG AAAATATTAA CGCTTACAAT TTCCATTCGC CATTCAGGCT
GCGCAACTGT TGGGAAGGGC GATCGGTGCG GGCCTCTTCG CTATTACGCC AGCTGGCGAA TTTTATAATT GCGAATGTTA
AAGGTAAGCG GTAAGTCCGA CGCGTTGACA ACCCTTCCCG CTAGCCACGC CCGGAGAAGC GATAATGCGG TCGACCGCTT
AGGGGGATGT GCTGCAAGGC GATTAAGTTG GGTAACGCCA GGGTTTTCCC AGTCACGACG TTGTAAAACG ACGGCCAGTG
AGCGCGCGTA ATACGACTCA TCCCCCTACA CGACGTTCCG CTAATTCAAC CCATTGCGGT CCCAAAAGGG TCAGTGCTGC
AACATTTTGC TGCCGGTCAC TCGCGCGCAT TATGCTGAGT CTATAGGGCG AATTGGAGCT CCACCGCGGT GGCGGCCGCT
CTAGAACTAG TGGATCCCCC GGGCTGCAGG AATTCGATAT CAAGCTTTTA CGCCCCGCCC GATATCCCGC TTAACCTCGA
GGTGGCGCCA CCGCCGGCGA GATCTTGATC ACCTAGGGGG CCCGACGTCC TTAAGCTATA GTTCGAAAAT GCGGGGCGGG
TGCCACTCAT CGCAGTACTG TTGTAATTCA TTAAGCATTC TGCCGACATG GAAGCCATCA CAAACGGCAT GATGAACCTG
AATCGCCAGC GGCATCAGCA ACGGTGAGTA GCGTCATGAC AACATTAAGT AATTCGTAAG ACGGCTGTAC CTTCGGTAGT
GTTTGCCGTA CTACTTGGAC TTAGCGGTCG CCGTAGTCGT CCTTGTCGCC TTGCGTATAA TATTTGCCCA TGGTGAAAAC
GGGGGCGAAG AAGTTGTCCA TATTGGCCAC GTTTAAATCA AAACTGGTGA AACTCACCCA GGAACAGCGG AACGCATATT
ATAAACGGGT ACCACTTTTG CCCCCGCTTC TTCAACAGGT ATAACCGGTG CAAATTTAGT TTTGACCACT TTGAGTGGGT
GGGATTGGCT GAGACGAAAA ACATATTCTC AATAAACCCT TTAGGGAAAT AGGCCAGGTT TTCACCGTAA CACGCCACAT
CTTGCGAATA TATGTGTAGA CCCTAACCGA CTCTGCTTTT TGTATAAGAG TTATTTGGGA AATCCCTTTA TCCGGTCCAA
AAGTGGCATT GTGCGGTGTA GAACGCTTAT ATACACATCT AACTGCCGGA AATCGTCGTG GTATTCACTC CAGAGCGATG
AAAACGTTTC AGTTTGCTCA TGGAAAACGG TGTAACAAGG GTGAACACTA TCCCATATCA TTGACGGCCT TTAGCAGCAC
CATAAGTGAG GTCTCGCTAC TTTTGCAAAG TCAAACGAGT ACCTTTTGCC ACATTGTTCC CACTTGTGAT AGGGTATAGT
CCAGCTCACC GTCTTTCATT GCCATACGAA ATTCCGGATG AGCATTCATC AGGCGGGCAA GAATGTGAAT AAAGGCCGGA
TAAAACTTGT GCTTATTTTT GGTCGAGTGG CAGAAAGTAA CGGTATGCTT TAAGGCCTAC TCGTAAGTAG TCCGCCCGTT
CTTACACTTA TTTCCGGCCT ATTTTGAACA CGAATAAAAA CTTTACGGTC TTTAAAAAGG CCGTAATATC CAGCTGAACG
GTCTGGTTAT AGGTACATTG AGCAACTGAC TGAAATGCCT CAAAATGTTC TTTACGATGC GAAATGCCAG AAATTTTTCC
GGCATTATAG GTCGACTTGC CAGACCAATA TCCATGTAAC TCGTTGACTG ACTTTACGGA GTTTTACAAG AAATGCTACG
CATTGGGATA TATCAACGGT GGTATATCCA GTGATTTTTT TCTCCATATG GTTAACCTTA ATTAAGGGGT CGACGGGCCC
GGGATCCGAT GCTCTTCCGC GTAACCCTAT ATAGTTGCCA CCATATAGGT CACTAAAAAA AGAGGTATAC CAATTGGAAT
TAATTCCCCA GCTGCCCGGG CCCTAGGCTA CGAGAAGGCG TAAGATCTTT TACTAGTTCA GTCCATCTCG CCGTGTATGC
GGGCCTGACG GATCAACGTT CCCACCGAGC CAGTCGAGAT GTTCATCTGG TCGGCGATCT ATTCTAGAAA ATGATCAAGT
CAGGTAGAGC GGCACATACG CCCGGACTGC CTAGTTGCAA GGGTGGCTCG GTCAGCTCTA CAAGTAGACC AGCCGCTAGA
GCCGGTACTT CAAACCTTGT TTGCGCAGTT CCACAGCCTT CTTGCGGCGT TCCTGCGCAC GAGCGATGTA GTCGCCTCGG
TCTTCGGCGA CGAGCCGTTT CGGCCATGAA GTTTGGAACA AACGCGTCAA GGTGTCGGAA GAACGCCGCA AGGACGCGTG
CTCGCTACAT CAGCGGAGCC AGAAGCCGCT GCTCGGCAAA GATGGTGCTT TTCGAGACGC CGAACTTGTC AGCCAACTCC
TGCGCGGTCT GCGTGCGACG CATCACGCGT TCTGCAGCAC CCATCAGTCC GTCCCCTCTG CTACCACGAA AAGCTCTGCG
GCTTGAACAG TCGGTTGAGG ACGCGCCAGA CGCACGCTGC GTAGTGCGCA AGACGTCGTG GGTAGTCAGG CAGGGGAGAC
CTGCTGCGAA CAGTGCCGAT CGATCGACCT TCTTGAGCTT CGGCCGCGGC GCGGTGGCGT TCTTCCGTAC CGCTTCCGTT
TTTGCGCTGC TGCTCACTTT GACGACGCTT GTCACGGCTA GCTAGCTGGA AGAACTCGAA GCCGGCGCCG CGCCACCGCA
AGAAGGCATG GCGAAGGCAA AAACGCGACG ACGAGTGAAA GCCGCGGCGT GCCTGGATTT TCGAGAACTC GGCGGCGGTG
AAGGTGCGGT GGGTCCAGTG GGCGACTGAT TTGCCGATCT GCTCGGCCTC GGCCCGACTC CGGCGCCGCA CGGACCTAAA
AGCTCTTGAG CCGCCGCCAC TTCCACGCCA CCCAGGTCAC CCGCTGACTA AACGGCTAGA CGAGCCGGAG CCGGGCTGAG
ATGGGGCCGA TCCCGTCGTT GGCGTCGAGG GTGAAGTTGG TCAGGGCGGT GAAGTCGGTG ACCATCTGCC GCCACACAGT
GATCGACGGG TAGTTCTGTT TACCCCGGCT AGGGCAGCAA CCGCAGCTCC CACTTCAACC AGTCCCGCCA CTTCAGCCAC
TGGTAGACGG CGGTGTGTCA CTAGCTGCCC ATCAAGACAA TCCGGATCTC GCGGTAGGCC CATTCCCGGG TGCGGTCGAA
CAGTTCGACG TTCCGGCCCG TTTCGGTCCT GACCTGTGTC TTGCGGCCGT AGTCCGGTGG AGGCCTAGAG CGCCATCCGG
GTAAGGGCCC ACGCCAGCTT GTCAAGCTGC AAGGCCGGGC AAAGCCAGGA CTGGACACAG AACGCCGGCA TCAGGCCACC
GGCGGGGAAA CGGTCACCGA GCGCTTTTGC GAGGCCTTTG AGCGAGTACG GATCCGAGGG ACCCCAGACC GTCGTCCAGT
GCGGGTGGAT CGGGTTCTGG CCGCCCCTTT GCCAGTGGCT CGCGAAAACG CTCCGGAAAC TCGCTCATGC CTAGGCTCCC
TGGGGTCTGG CAGCAGGTCA CGCCCACCTA GCCCAAGACC GTGAGCTGCT GCGCGTAGCC CTGATCGGCG CCGACCACCG
AGGCGATCAG CCCCTGGTTC ACCCGGTCGT AGAGCCGCAG CGGGCCCTGT CGGGCTGCCT CACTCGACGA CGCGCATCGG
GACTAGCCGC GGCTGGTGGC TCCGCTAGTC GGGGACCAAG TGGGCCAGCA TCTCGGCGTC GCCCGGGACA GCCCGACGGA
GGAGGGTGTA GACCGGGCTT TCGAGCAGCC ACCACAGGTG CGCGTGCTCG GTCGCGGGAT TGATCGTCAT CACGGTCGGA
TCGGGCAGAT CCGCGTTACG CCTCCCACAT CTGGCCCGAA AGCTCGTCGG TGGTGTCCAC GCGCACGAGC CAGCGCCCTA
ACTAGCAGTA GTGCCAGCCT AGCCCGTCTA GGCGCAATGC TGCGGCCCAC TGCGCCTGGT CGTCGTCCAC GTCGAGCACC
AAGCCCAACC TGATCGACGG GGTGCGGGCC GCAATGTAGC GGCGGGTGAG CGCCTCCGCG ACGCCGGGTG ACGCGGACCA
GCAGCAGGTG CAGCTCGTGG TTCGGGTTGG ACTAGCTGCC CCACGCCCGG CGTTACATCG CCGCCCACTC GCGGAGGCGC
CGCGGCTGCG GCCACTGCCC GTCCCGGACG TAGTCATCCG TCGCGTGCGG GTATTTGAAC CGCCAGCGGT CCAACCAGGC
GTCAACAGCA GCGGTCATGA GCGCCGACGC CGGTGACGGG CAGGGCCTGC ATCAGTAGGC AGCGCACGCC CATAAACTTG
GCGGTCGCCA GGTTGGTCCG CAGTTGTCGT CGCCAGTACT CCGCCAAGCT AGGGCCGGAT CTGTACCGAT CGGGGGAGGC
GCGCCGCAAA TTATTTAAGA GTCTCGCTAG CAAACCATGT CAGGTGTTGC GGTGGGTTCC GGCGGTTCGA TCCCGGCCTA
GACATGGCTA GCCCCCTCCG CGCGGCGTTT AATAAATTCT CAGAGCGATC GTTTGGTACA GTCCACAACG CCACCCAAGG
GGGTAAACCT CCACCCGAAT TATTTAAGAG TCTCGCTAGC TAAGCCCTAT CTGATGCTGC GCGGGGGGTC CTTCGCACTG
AATCTCAAAG GTGGCCGGCT CCCATTTGGA GGTGGGCTTA ATAAATTCTC AGAGCGATCG ATTCGGGATA GACTACGACG
CGCCCCCCAG GAAGCGTGAC TTAGAGTTTC CACCGGCCGA GAATTTCGTC GCGCGAAAAC CTCCCTGGAC AGTTCTGGAA
TTCAGCAAGA GGTGTGTCTG AACTTCGGTG TTTTTTTGGG GGGTGACTCC AGCGGGGTGG CTTAAAGCAG CGCGCTTTTG
GAGGGACCTG TCAAGACCTT AAGTCGTTCT CCACACAGAC TTGAAGCCAC AAAAAAACCC CCCACTGAGG TCGCCCCACC
GCACAACGCG AACAGAGACC TTGTGTGTAC GACGGCGGGA GGTAAGTCGG GTACGGCTCG GACTGCGGTA GAGCAACCGT
CGAATCGATT TCGAGCAGAG CGTGTTGCGC TTGTCTCTGG AACACACATG CTGCCGCCCT CCATTCAGCC CATGCCGAGC
CTGACGCCAT CTCGTTGGCA GCTTAGCTAA AGCTCGTCTC CGAGCAGAGC AAGATATTCC AAAACTCCGG GGTTCCTCGG
CGGCCTCCCC CGTCTGTTTG CTCAACCGAG GGAGACCTGG CGGTCCCGCG TTTCCGGACG GCTCGTCTCG TTCTATAAGG
TTTTGAGGCC CCAAGGAGCC GCCGGAGGGG GCAGACAAAC GAGTTGGCTC CCTCTGGACC GCCAGGGCGC AAAGGCCTGC
CGCGGGACCG CCTACCGCTC GAGAGCGGAA GAGCATCTAG ATGCATTCGC GAGGTACCCA GCTTTTGTTC CCTTTAGTGA
GGGTTAATTG CGCGCTTGGC GCGCCCTGGC GGATGGCGAG CTCTCGCCTT CTCGTAGATC TACGTAAGCG CTCCATGGGT
CGAAAACAAG GGAAATCACT CCCAATTAAC GCGCGAACCG GTAATCATGG TCATAGCTGT TTCCTGTGTG AAATTGTTAT
CCGCTCACAA TTCCACACAA CATACGAGCC GGAAGCATAA AGTGTAAAGC CTGGGGTGCC CATTAGTACC AGTATCGACA
AAGGACACAC TTTAACAATA GGCGAGTGTT AAGGTGTGTT GTATGCTCGG CCTTCGTATT TCACATTTCG GACCCCACGG
TAATGAGTGA GCTAACTCAC ATTAATTGCG TTGCGCTCAC TGCCCGCTTT CCAGTCGGGA AACCTGTCGT GCCAGCTGCA
TTAATGAATC GGCCAACGCG ATTACTCACT CGATTGAGTG TAATTAACGC AACGCGAGTG ACGGGCGAAA GGTCAGCCCT
TTGGACAGCA CGGTCGACGT AATTACTTAG CCGGTTGCGC CGGGGAGAGG CGGTTTGCGT ATTGGGCGCA TGCATAAAAA
CTGTTGTAAT TCATTAAGCA TTCTGCCGAC ATGGAAGCCA TCACAAACGG CATGATGAAC GCCCCTCTCC GCCAAACGCA
TAACCCGCGT ACGTATTTTT GACAACATTA AGTAATTCGT AAGACGGCTG TACCTTCGGT AGTGTTTGCC GTACTACTTG
CTGAATCGCC AGCGGCATCA GCACCTTGTC GCCTTGCGTA TAATATTTGC CCATGGGGGT GGGCGAAGAA CTCCAGCATG
AGATCCCCGC GCTGGAGGAT GACTTAGCGG TCGCCGTAGT CGTGGAACAG CGGAACGCAT ATTATAAACG GGTACCCCCA
CCCGCTTCTT GAGGTCGTAC TCTAGGGGCG CGACCTCCTA CATCCAGCCG GCGTCCCGGA AAACGATTCC GAAGCCCAAC
CTTTCATAGA AGGCGGCGGT GGAATCGAAA TCTCGTGATG GCAGGTTGGG CGTCGCTTGG GTAGGTCGGC CGCAGGGCCT
TTTGCTAAGG CTTCGGGTTG GAAAGTATCT TCCGCCGCCA CCTTAGCTTT AGAGCACTAC CGTCCAACCC GCAGCGAACC
TCGGTCATTT CGAACCCCAG AGTCCCGCTC AGAAGAACTC GTCAAGAAGG CGATAGAAGG CGATGCGCTG CGAATCGGGA
GCGGCGATAC CGTAAAGCAC AGCCAGTAAA GCTTGGGGTC TCAGGGCGAG TCTTCTTGAG CAGTTCTTCC GCTATCTTCC
GCTACGCGAC GCTTAGCCCT CGCCGCTATG GCATTTCGTG GAGGAAGCGG TCAGCCCATT CGCCGCCAAG CTCTTCAGCA
ATATCACGGG TAGCCAACGC TATGTCCTGA TAGCGGTCCG CCACACCCAG CCGGCCACAG CTCCTTCGCC AGTCGGGTAA
GCGGCGGTTC GAGAAGTCGT TATAGTGCCC ATCGGTTGCG ATACAGGACT ATCGCCAGGC GGTGTGGGTC GGCCGGTGTC
TCGATGAATC CAGAAAAGCG GCCATTTTCC ACCATGATAT TCGGCAAGCA GGCATCGCCA TGGGTCACGA CGAGATCCTC
GCCGTCGGGC ATGCGCGCCT AGCTACTTAG GTCTTTTCGC CGGTAAAAGG TGGTACTATA AGCCGTTCGT CCGTAGCGGT
ACCCAGTGCT GCTCTAGGAG CGGCAGCCCG TACGCGCGGA TGAGCCTGGC GAACAGTTCG GCTGGCGCGA GCCCCTGATG
CTCTTCGTCC AGATCATCCT GATCGACAAG ACCGGCTTCC ATCCGAGTAC GTGCTCGCTC ACTCGGACCG CTTGTCAAGC
CGACCGCGCT CGGGGACTAC GAGAAGCAGG TCTAGTAGGA CTAGCTGTTC TGGCCGAAGG TAGGCTCATG CACGAGCGAG
GATGCGATGT TTCGCTTGGT GGTCGAATGG GCAGGTAGCC GGATCAAGCG TATGCAGCCG CCGCATTGCA TCAGCCATGA
TGGATACTTT CTCGGCAGGA CTACGCTACA AAGCGAACCA CCAGCTTACC CGTCCATCGG CCTAGTTCGC ATACGTCGGC
GGCGTAACGT AGTCGGTACT ACCTATGAAA GAGCCGTCCT GCAAGGTGAG ATGACAGGAG ATCCTGCCCC GGCACTTCGC
CCAATAGCAG CCAGTCCCTT CCCGCTTCAG TGACAACGTC GAGCACAGCT GCGCAAGGAA CGTTCCACTC TACTGTCCTC
TAGGACGGGG CCGTGAAGCG GGTTATCGTC GGTCAGGGAA GGGCGAAGTC ACTGTTGCAG CTCGTGTCGA CGCGTTCCTT
CGCCCGTCGT GGCCAGCCAC GATAGCCGCG CTGCCTCGTC CTGCAGTTCA TTCAGGGCAC CGGACAGGTC GGTCTTGACA
AAAAGAACCG GGCGCCCCTG GCGGGCAGCA CCGGTCGGTG CTATCGGCGC GACGGAGCAG GACGTCAAGT AAGTCCCGTG
GCCTGTCCAG CCAGAACTGT TTTTCTTGGC CCGCGGGGAC CGCTGACAGC CGGAACACGG CGGCATCAGA GCAGCCGATT
GTCTGTTGTG CCCAGTCATA GCCGAATAGC CTCTCCACCC AAGCGGCCGG AGAACCTGCG GCGACTGTCG GCCTTGTGCC
GCCGTAGTCT CGTCGGCTAA CAGACAACAC GGGTCAGTAT CGGCTTATCG GAGAGGTGGG TTCGCCGGCC TCTTGGACGC
TGCAATCCAT CTTGTTCAAT CATGCGAAAC GATCCTCATC CTGTCTCTTG ATCAGATCTT GATCCCCTGC GCCATCAGAT
CCTTGGCGGC AAGAAAGCCA ACGTTAGGTA GAACAAGTTA GTACGCTTTG CTAGGAGTAG GACAGAGAAC TAGTCTAGAA
CTAGGGGACG CGGTAGTCTA GGAACCGCCG TTCTTTCGGT TCCAGTTTAC TTTGCAGGGC TTCCCAACCT TACCAGAGGG
CGCCCCAGCT GGCAATTCCG GTTCGCTTGC TGTCCATAAA ACCGCCCAGT CTAGCTATCG AGGTCAAATG AAACGTCCCG
AAGGGTTGGA ATGGTCTCCC GCGGGGTCGA CCGTTAAGGC CAAGCGAACG ACAGGTATTT TGGCGGGTCA GATCGATAGC
CCATGTAAGC CCACTGCAAG CTACCTGCTT TCTCTTTGCG CTTGCGTTTT CCCTTGTCCA GATAGCCCAG TAGCTGACAT
TCATCCCAGG TGGCACTTTT GGTACATTCG GGTGACGTTC GATGGACGAA AGAGAAACGC GAACGCAAAA GGGAACAGGT
CTATCGGGTC ATCGACTGTA AGTAGGGTCC ACCGTGAAAA CGGGGAAATG TGCGCGCCCG CGTTCCTGCT GGCGCTGGGC
CTGTTTCTGG CGCTGGACTT CCCGCTGTTC CGTCAGCAGC TTTTCGCCCA CGGCCTTGAT GCCCCTTTAC ACGCGCGGGC
GCAAGGACGA CCGCGACCCG GACAAAGACC GCGACCTGAA GGGCGACAAG GCAGTCGTCG AAAAGCGGGT GCCGGAACTA
GATCGCGGCG GCCTTGGCCT GCATATCCCG ATTCAACGGC CCCAGGGCGT CCAGAACGGG CTTCAGGCGC TCCCGAAGGT
CTCGGGCCGT CTCTTGGGCT CTAGCGCCGC CGGAACCGGA CGTATAGGGC TAAGTTGCCG GGGTCCCGCA GGTCTTGCCC
GAAGTCCGCG AGGGCTTCCA GAGCCCGGCA GAGAACCCGA TGATCGGCCT TCTTGCGCAT CTCACGCGCT CCTGCGGCGG
CCTGTAGGGC AGGCTCATAC CCCTGCCGAA CCGCTTTTGT CAGCCGGTCG GCCACGGCTT ACTAGCCGGA AGAACGCGTA
GAGTGCGCGA GGACGCCGCC GGACATCCCG TCCGAGTATG GGGACGGCTT GGCGAAAACA GTCGGCCAGC CGGTGCCGAA
CCGGCGTCTC AACGCGCTTT GAGATTCCCA GCTTTTCGGC CAATCCCTGC GGTGCATAGG CGCGTGGCTC GACCGCTTGC
GGGCTGATGG TGACGTGGCC GGCCGCAGAG TTGCGCGAAA CTCTAAGGGT CGAAAAGCCG GTTAGGGACG CCACGTATCC
GCGCACCGAG CTGGCGAACG CCCGACTACC ACTGCACCGG CACTGGTGGC CGCTCCAGGG CCTCGTAGAA CGCCTGAATG
CGCGTGTGAC GTGCCTTGCT GCCCTCGATG CCCCGTTGCA GCCCTAGATC GGCCACAGCG GTGACCACCG GCGAGGTCCC
GGAGCATCTT GCGGACTTAC GCGCACACTG CACGGAACGA CGGGAGCTAC GGGGCAACGT CGGGATCTAG CCGGTGTCGC
GCCGCAAACG TGGTCTGGTC GCGGGTCATC TGCGCTTTGT TGCCGATGAA CTCCTTGGCC GACAGCCTGC CGTCCTGCGT
CAGCGGCACC ACGAACGCGG CGGCGTTTGC ACCAGACCAG CGCCCAGTAG ACGCGAAACA ACGGCTACTT GAGGAACCGG
CTGTCGGACG GCAGGACGCA GTCGCCGTGG TGCTTGCGCC TCATGTGCGG GCTGGTTTCG TCACGGTGGA TGCTGGCCGT
CACGATGCGA TCCGCCCCGT ACTTGTCCGC CAGCCACTTG TGCGCCTTCT CGAAGAACGC AGTACACGCC CGACCAAAGC
AGTGCCACCT ACGACCGGCA GTGCTACGCT AGGCGGGGCA TGAACAGGCG GTCGGTGAAC ACGCGGAAGA GCTTCTTGCG
CGCCTGCTGT TCTTGGCTGG CCGACTTCCA CCATTCCGGG CTGGCCGTCA TGACGTACTC GACCGCCAAC ACAGCGTCCT
TGCGCCGCTT CTCTGGCAGC GCGGACGACA AGAACCGACC GGCTGAAGGT GGTAAGGCCC GACCGGCAGT ACTGCATGAG
CTGGCGGTTG TGTCGCAGGA ACGCGGCGAA GAGACCGTCG AACTCGCGCA GTCGGCCCAT CGCTTCATCG GTGCTGCTGG
CCGCCCAGTG CTCGTTCTCT GGCGTCCTGC TGGCGTCAGC GTTGGGCGTC TCGCGCTCGC TTGAGCGCGT CAGCCGGGTA
GCGAAGTAGC CACGACGACC GGCGGGTCAC GAGCAAGAGA CCGCAGGACG ACCGCAGTCG CAACCCGCAG AGCGCGAGCG
GGTAGGCGTG CTTGAGACTG GCCGCCACGT TGCCCATTTT CGCCAGCTTC TTGCATCGCA TGATCGCGTA TGCCGCCATG
CCTGCCCCTC CCTTTTGGTG CCATCCGCAC GAACTCTGAC CGGCGGTGCA ACGGGTAAAA GCGGTCGAAG AACGTAGCGT
ACTAGCGCAT ACGGCGGTAC GGACGGGGAG GGAAAACCAC TCCAACCGGC TCGACGGGGG CAGCGCAAGG CGGTGCCTCC
GGCGGGCCAC TCAATGCTTG AGTATACTCA CTAGACTTTG CTTCGCAAAG TCGTGACCGC AGGTTGGCCG AGCTGCCCCC
GTCGCGTTCC GCCACGGAGG CCGCCCGGTG AGTTACGAAC TCATATGAGT GATCTGAAAC GAAGCGTTTC AGCACTGGCG
CTACGGCGGC TGCGGCGCCC TACGGGCTTG CTCTCCGGGC TTCGCCCTGC GCGGTCGCTG CGCTCCCTTG CCAGCCCGTG
GATATGTGGA CGATGGCCGC GATGCCGCCG ACGCCGCGGG ATGCCCGAAC GAGAGGCCCG AAGCGGGACG CGCCAGCGAC
GCGAGGGAAC GGTCGGGCAC CTATACACCT GCTACCGGCG GAGCGGCCAC CGGCTGGCTC GCTTCGCTCG GCCCGTGGAC
AACCCTGCTG GACAAGCTGA TGGACAGGCT GCGCCTGCCC ACGAGCTTGA CCACAGGGAT CTCGCCGGTG GCCGACCGAG
CGAAGCGAGC CGGGCACCTG TTGGGACGAC CTGTTCGACT ACCTGTCCGA CGCGGACGGG TGCTCGAACT GGTGTCCCTA
TGCCCACCGG CTACCCAGCC TTCGACCACA TACCCACCGG CTCCAACTGC GCGGCCTGCG GCCTTGCCCC ATCAATTTTT
TTAATTTTCT CTGGGGAAAA ACGGGTGGCC GATGGGTCGG AAGCTGGTGT ATGGGTGGCC GAGGTTGACG CGCCGGACGC
CGGAACGGGG TAGTTAAAAA AATTAAAAGA GACCCCTTTT GCCTCCGGCC TGCGGCCTGC GCGCTTCGCT TGCCGGTTGG
ACACCAAGTG GAAGGCGGGT CAAGGCTCGC GCAGCGACCG CGCAGCGGCT TGGCCTTGAC CGGAGGCCGG ACGCCGGACG
CGCGAAGCGA ACGGCCAACC TGTGGTTCAC CTTCCGCCCA GTTCCGAGCG CGTCGCTGGC GCGTCGCCGA ACCGGAACTG
GCGCCTGGAA CGACCCAAGC CTATGCGAGT GGGGGCAGTC GAAGGCGAAG CCCGCCCGCC TGCCCCCCGA GCCTCACGGC
GGCGAGTGCG GGGGTTCCAA CGCGGACCTT GCTGGGTTCG GATACGCTCA CCCCCGTCAG CTTCCGCTTC GGGCGGGCGG
ACGGGGGGCT CGGAGTGCCG CCGCTCACGC CCCCAAGGTT GGGGGCAGCG CCACCTTGGG CAAGGCCGAA GGCCGCGCAG
TCGATCAACA AGCCCCGGAG GGGCCACTTT TTGCCGGAG CCCCCGTCGC GGTGGAACCC GTTCCGGCTT CCGGCGCGTC
AGCTAGTTGT TCGGGGCCTC CCCGGTGAAA AACGGCCTC
SEQ ID: 05
MEALFLSSSS SSIVASNKLT RLHNHCVWST VIRDKKRFGP TWCRVGGGGD GGRNSNAERP IRVSSLLKDR GQVLIREQSS
PAMDAETLVL SPNGNGRTIE INGVKTLMPF SGASMVGMKE GLGIISFLQG KKFLITGSTG FLAKVLIEKV LRMAPDVSKI
YLLIKAKSKE AAIERLKNEV LDAELFNTLK ETHGASYMSF MLTKLIPVTG NICDSNIGLQ ADSAEEIAKE VDVIINSAAN
TTFNERYDVA LDINTRGPGN LMGFAKKCKK LKLFLQVSTA YVNGQRQGRI MEKPFSMGDC IATENFLEGN RKALDVDREM
KLALEAARKG TQNQDEAQKM KDLGLERARS YGWQDTYVFT KAMGEMMINS TRGDVPVVII RPSVIESTYK DPFPGWMEGN
RMMDPIVLCY GKGQLTGFLV DPKGVLDVVP ADMVVNATLA AIAKHGMAMS DPEPEINVYQ IASSAINPLV FEDLAELLYN
HYKTSPCMDS KGDPIMVRLM KLFNSVDDFS DHLWRDAQER SGLMSGMSSV DSKMMQKLKF ICKKSVEQAK HLATIYEPYT
FYGGRFDNSN TQRLMENMSE DEKREFGFDV GSINWTDYIT NVHIPGLRRH VLKGRA
SEQ ID: 06
MATTNVLATS HAFKLNGVSY FSSFPRKPNH YMPRRRLSHT TRRVQTSCFY GETSFEAVTS LVTPKTETSR NSDGIGIVRF
LEGKSYLVTG ATGFLAKVLI EKLLRESLEI GKIFLLMRSK DQESANKRLY DEIISSDLFK LLKQMHGSSY EAFMKRKLIP
VIGDIEEDNL GIKSEIANMI SEEIDVIISC GGRTTFDDRY DSALSVNALG PGRLLSFGKG CRKLKLFLHF STAYVTGKRE
GTVLETPLCI GENITSDLNI KSELKLASEA VRKFRGREEI KKLKELGFER AQHYGWENSY TFTKAIGEAV IHSKRGNLPV
VIIRPSIIES SYNEPFPGWI QGTRMADPII LAYAKGQISD FWADPQSLMD IIPVDMVANA AIAAMAKHGC GVPEFKVYNL
TSSSHVNPMR AGKLIDLSHQ HLCDFPLEET VIDLEHMKIH SSLEGFTSAL SNTIIKQERV IDNEGGGLST KGKRKLNYFV
SLAKTYEPYT FFQARFDNTN TTSLIQEMSM EEKKTFGFDI KGIDWEHYIV NVHLPGLKKE FLSKKKTE
SEQ ID: 07
MESNCVQFLG NKTILITGAP GFLAKVLVEK ILRLQPNVKK IYLLLRAPDE KSAMQRLRSE VMEIDLFKVL RNNLGEDNLN
ALMREKIVPV PGDISIDNLG LKDTDLIQRM WSEIDIIINI AATTNFDERY DIGLGINTFG ALNVLNFAKK CVKGQLLLHV
STAYISGEQP GLLLEKPFKM GETLSGDREL DINIEHDLMK QKLKELQDCS DEEISQTMKD FGMARAKLHG WPNTYVFTKA
MGEMLMGKYR ENLPLVIIRP TMITSTIAEP FPGWIEGLKT LDSVIVAYGK GRLKCFLADS NSVFDLIPAD MVVNAMVAAA
TAHSGDTGIQ AIYHVGSSCK NPVTFGQLHD FTARYFAKRP LIGRNGSPII VVKGTILSTM AQFSLYMTLR YKLPLQILRL
INIVYPWSHG DNYSDLSRKI KLAMRLVELY QPYLLFKGIF DDLNTERLRM KRKENIKELD GSFEFDPKSI DWDNYITNTH
IPGLITHVLK Q
SEQ ID: 08
MPELAVRTEF DYSSEIYKDA YSRINAIVIE GEQEAYSNYL QMAELLPEDK EELTRLAKME NRHKKGFQAC GNNLQVNPDM
PYAQEFFAGL HGNFQHAFSE GKVVTCLLIQ ALIIEAFAIA AYNIYIPVAD DFARKITEGV VKDEYTHLNY GEEWLKANFA
TAKEELEQAN KENLPLVWKM LNQVQGDAKV LGMEKEALVE DFMISYGEAL SNIGFSTREI MRMSSYGLAG V
SEQ ID: 09
MFGLIGHLTS LEHAQAVAED LGYPEYANQG LDFWCSAPPQ VVDNFQVKSV TGQVIEGKYV ESCFLPEMLT QRRIKAAIRK
ILNAMALAQK VGLDITALGG FSSIVFEEFN LKQNNQVRNV ELDFQRFTTG NTHTAYVICR QVESGAKQLG IDLSQATVAV
CGATGDIGSA VCRWLDSKHQ VKELLLIARN RQRLENLQEE LGRGKIMDLE TALPQADIIV WVASMPKGVE IAGEMLKKPC
LIVDGGYPKN LDTRVKADGV HILKGGIVEH SLDITWEIMK IVEMDIPSRQ MFACFAEAIL LEFEGWRTNF SWGRNQISVN
KMEAIGEASV KHGFCPLVAL
SEQ ID: 10
CAGTCAATGG AGAGCATTGC CATAAGTAAA GGCATCCCCT GCGTGATAAG ATTACCTTCA GAAAACAGAT AGTTGCTGGG
TTATCGCAGA TTTTTCTCGC GTCAGTTACC TCTCGTAACG GTATTCATTT CCGTAGGGGA CGCACTATTC TAATGGAAGT
CTTTTGTCTA TCAACGACCC AATAGCGTCT AAAAAGAGCG AACCAAATAA CTGTAAATAA TAACTGTCTC TGGGGCGACG
GTAGGCTTTA TATTGCCAAA TTTCGCCCGT GGGAGAAAGC TAGGCTATTC AATGTTTATG TTGGTTTATT GACATTTATT
ATTGACAGAG ACCCCGCTGC CATCCGAAAT ATAACGGTTT AAAGCGGGCA CCCTCTTTCG ATCCGATAAG TTACAAATAC
GAGGACTCCT
SEQ ID: 11
CCTGGCTCAG GACGAACGCT GGCGGCGTGC TTAACACATG CAAGTCGAGC GGTAAGGCCC TTCGGGGTAC ACGAGCGGCG
AACGGGTGAG TAACACGTGG GGACCGAGTC CTGCTTGCGA CCGCCGCACG AATTGTGTAC GTTCAGCTCG CCATTCCGGG
AAGCCCCATG TGCTCGCCGC TTGCCCACTC ATTGTGCACC GTGATCTGCC CTGCACTTCG GGATAAGCCT GGGAAACTGG
GTCTAATACC GGATATGACC TTCGGCTGCA TGGCTGAGGG TGGAAAGGTT TACTGGTGCA CACTAGACGG GACGTGAAGC
CCTATTCGGA CCCTTTGACC CAGATTATGG CCTATACTGG AAGCCGACGT ACCGACTCCC ACCTTTCCAA ATGACCACGT
GGATGGGCCC GCGGCCTATC AGCTTGTTGG TGGGGTAATG GCCTACCAAG GCGACGACGG GTAGCCGACC TGAGAGGGTG
ACCGGCCACA CTGGGACTGA CCTACCCGGG CGCCGGATAG TCGAACAACC ACCCCATTAC CGGATGGTTC CGCTGCTGCC
CATCGGCTGG ACTCTCCCAC TGGCCGGTGT GACCCTGACT GACACGGCCC AGACTCCTAC GGGAGGCAGC AGTGGGGAAT
ATTGCACAAT GGGCGAAAGC CTGATGCAGC GACGCCGCGT GAGGGATGAC GGCCTTCGGG CTGTGCCGGG TCTGAGGATG
CCCTCCGTCG TCACCCCTTA TAACGTGTTA CCCGCTTTCG GACTACGTCG CTGCGGCGCA CTCCCTACTG CCGGAAGCCC
TTGTAAACCT CTTTCAGCAG GGACGAAGCG AAAGTGACGG TACCTGCAGA AGAAGCACCG GCCAACTACG TGCCAGCAGC
CGCGGTAATA CGTAGGGTGC AACATTTGGA GAAAGTCGTC CCTGCTTCGC TTTCACTGCC ATGGACGTCT TCTTCGTGGC
CGGTTGATGC ACGGTCGTCG GCGCCATTAT GCATCCCACG AAGCGTTGTC CGGAATTACT GGGCGTAAAG AGCTCGTAGG
CGGTTTGTCG CGTCGTCTGT GAAAACTCAN AGCTCAACCT CGAGCTTGCA GGCGATACGG TTCGCAACAG GCCTTAATGA
CCCGCATTTC TCGAGCATCC GCCAAACAGC GCAGCAGACA CTTTTGAGTN TCGAGTTGGA GCTCGAACGT CCGCTATGCC
GCAGACTTGA GTACTGCAGG GGAGACTGGA ATTCCTGGTG TAGCGGTGAA ATGCGCAGAT ATCAGGAGGA ACACCGGTGG
CGAAGGCGGG TCTCTGGGCA CGTCTGAACT CATGACGTCC CCTCTGACCT TAAGGACCAC ATCGCCACTT TACGCGTCTA
TAGTCCTCCT TGTGGCCACC GCTTCCGCCC AGAGACCCGT GTAACTGACG CTGAGGAGCG AAAGCGTGGG TAGCAAACAG
GATTAGATAC CCTGGTAGTC CACGCCGTAA ACGGTGGGCG CTAGGTGTGG GTTTCCTTCC CATTGACTGC GACTCCTCGC
TTTCGCACCC ATCGTTTGTC CTAATCTATG GGACCATCAG GTGCGGCATT TGCCACCCGC GATCCACACC CAAAGGAAGG
ACGGGATCCG TGCCGTAGTT AACGCATTAA GCGCCCCGCC TGGGGAGTAC GGCCGCAAGG TTAAAACTCA AAGGAATTGA
CGGGGGCCCG CACAAGCGGC TGCCCTAGGC ACGGCATCAA TTGCGTAATT CGCGGGGCGG ACCCCTCATG CCGGCGTTCC
AATTTTGAGT TTCCTTAACT GCCCCCGGGC GTGTTCGCCG GGAGCATGTG GATTAATTCG ATGCAACGCG AAGAACCTTA
CCTGGGTTTG ACATATACCG GAAAGCCGTA GAGATACCGC CCCCCTTGTG GTCGGTATAC CCTCGTACAC CTAATTAAGC
TACGTTGCGC TTCTTGGAAT GGACCCAAAC TGTATATGGC CTTTCGGCAT CTCTATGGCG GGGGGAACAC CAGCCATATG
AGGTGGTGCA TGGCTGTCGT CAGCTCGTGT CGTGAGATGT TGGGTTAAGT CCCGCAACGA GCGCAACCCT TGTCTTATGT
TGCCAGCACG TAATGGTGGG TCCACCACGT ACCGACAGCA GTCGAGCACA GCACTCTACA ACCCAATTCA GGGCGTTGCT
CGCGTTGGGA ACAGAATACA ACGGTCGTGC ATTACCACCC GACTCGTAAG AGACTGCCGG GGTCAACTCG GAGGAAGGTG
GGGACGACGT CAAGTCATCA TGCCCCTTAT GTCCAGGGCT TCACACATGC TACAATGGCC CTGAGCATTC TCTGACGGCC
CCAGTTGAGC CTCCTTCCAC CCCTGCTGCA GTTCAGTAGT ACGGGGAATA CAGGTCCCGA AGTGTGTACG ATGTTACCGG
GGTACAGAGG GCTGCGATAC CGTGAGGTGG AGCGAATCCC TTAAAGCCGG TCTCAGTTCG GATCGGGGTC TGCAACTCGA
CCCCGTGAAG TCGGAGTCGC CCATGTCTCC CGACGCTATG GCACTCCACC TCGCTTAGGG AATTTCGGCC AGAGTCAAGC
CTAGCCCCAG ACGTTGAGCT GGGGCACTTC AGCCTCAGCG TAGTAATCGC AGATCAGCAA CGCTGCGGTG AATACGTTCC
CGGGCCTTGT ACACACCGCC CGTCACGTCA TGAAAGTCGG TAACACCCGA AGCCGGTGGC ATCATTAGCG TCTAGTCGTT
GCGACGCCAC TTATGCAAGG GCCCGGAACA TGTGTGGCGG GCAGTGCAGT ACTTTCAGCC ATTGTGGGCT TCGGCCACCG
CTAACCCCTT GTGGGAGGGA GCCGTCGAAG GTGGGATCGG CGATTGGGAC GAAGTCGTAA CAAGGTAGCC GTACCGGAAG
GGATTGGGGA ACACCCTCCC TCGGCAGCTT CCACCCTAGC CGCTAACCCT GCTTCAGCAT TGTTCCATCG GCATGGCCTT
CC
SEQ ID: 12
TCAACGGAGA GTTTGATCCT GGCTCAGGAC GAACGCTGGC GGCGTGCTTA ACACATGCAA GTCGAGCGGT AAGGCCCTTC
GGGGTACACG AGCGGCGAAC AGTTGCCTCT CAAACTAGGA CCGAGTCCTG CTTGCGACCG CCGCACGAAT TGTGTACGTT
CAGCTCGCCA TTCCGGGAAG CCCCATGTGC TCGCCGCTTG GGGTGAGTAA CACGTGGGTG ATCTGCCCTG CACTTCGGGA
TAAGCCTGGG AAACTGGGTC TAATACCGGA TATGACCTTC GGCTGCATGG CCGTTGGTGG CCCACTCATT GTGCACCCAC
TAGACGGGAC GTGAAGCCCT ATTCGGACCC TTTGACCCAG ATTATGGCCT ATACTGGAAG CCGACGTACC GGCAACCACC
AAAGGTTTAC TGGTGCAGGA TGGGCCCGCG GCCTATCAGC TTGTTGGTGG GGTAATGGCC TACCAAGGCG ACGACGGGTA
GCCGACCTGA GAGGGTGACC TTTCCAAATG ACCACGTCCT ACCCGGGCGC CGGATAGTCG AACAACCACC CCATTACCGG
ATGGTTCCGC TGCTGCCCAT CGGCTGGACT CTCCCACTGG GGCCACACTG GGACTGAGAC ACGGCCCAGA CTCCTACGGG
AGGCAGCAGT GGGGAATATT GCACAATGGG CGAAAGCCTG ATGCAGCGAC GCCGCGTGAG CCGGTGTGAC CCTGACTCTG
TGCCGGGTCT GAGGATGCCC TCCGTCGTCA CCCCTTATAA CGTGTTACCC GCTTTCGGAC TACGTCGCTG CGGCGCACTC
GGATGACGGC CTTCGGGTTG TAAACCTCTT TCAGCAGGGA CGAAGCGAAA GTGACGGTAC CTGCAGAAGA AGCACCGGCC
AACTACGTGC CAGCAGCCGC CCTACTGCCG GAAGCCCAAC ATTTGGAGAA AGTCGTCCCT GCTTCGCTTT CACTGCCATG
GACGTCTTCT TCGTGGCCGG TTGATGCACG GTCGTCGGCG GGTAATACGT AGGGTGCAAG CGTTGTCCGG AATTACTGGG
CGTAAAGAGC TCGTAGGCGG TTTGTCGCGT CGTCTGTGAA AACTCGAGGC TCAACCTCGA CCATTATGCA TCCCACGTTC
GCAACAGGCC TTAATGACCC GCATTTCTCG AGCATCCGCC AAACAGCGCA GCAGACACTT TTGAGCTCCG AGTTGGAGCT
GCTTGCAGGC GATACGGGCA GACTTGAGTA CTGCAGGGGA GACTGGAATT CCTGGTGTAG CGGTGAAATG CGCAGATATC
AGGAGGAACA CCGGTGGCGA CGAACGTCCG CTATGCCCGT CTGAACTCAT GACGTCCCCT CTGACCTTAA GGACCACATC
GCCACTTTAC GCGTCTATAG TCCTCCTTGT GGCCACCGCT AGGCGGGTCT CTGGGCAGTA ACTGACGCTG AGGAGCGAAA
GCGTGGGTAG CGAACAGGAT TAGATACCCT GGTAGTCCAC GCCGTAAACG GTGGGCGCTA TCCGCCCAGA GACCCGTCAT
TGACTGCGAC TCCTCGCTTT CGCACCCATC GCTTGTCCTA ATCTATGGGA CCATCAGGTG CGGCATTTGC CACCCGCGAT
GGTGTGGGTT TCCTTCCACG GGATCCGTGC CGTAGCTAAC GCATTAAGCG CCCCGCCTGG GGAGTACGGC CGCAAGGCTA
AAACTCAAAG GAATTGACGG CCACACCCAA AGGAAGGTGC CCTAGGCACG GCATCGATTG CGTAATTCGC GGGGCGGACC
CCTCATGCCG GCGTTCCGAT TTTGAGTTTC CTTAACTGCC GGGCCCGCAC AAGCGGCGGA GCATGTGGAT TAATTCGATG
CAACGCGAAG AACCTTACCT GGGTTTGACA TATACCGGAA AGCTGCAGAG ATGTGGCCCC CCCGGGCGTG TTCGCCGCCT
CGTACACCTA ATTAAGCTAC GTTGCGCTTC TTGGAATGGA CCCAAACTGT ATATGGCCTT TCGACGTCTC TACACCGGGG
CCTTGTGGTC GGTATACAGG TGGTGCATGG CTGTCGTCAG CTCGTGTCGT GAGATGTTGG GTTAAGTCCC GCAACGAGCG
CAACCCTTGT CTTATGTTGC GGAACACCAG CCATATGTCC ACCACGTACC GACAGCAGTC GAGCACAGCA CTCTACAACC
CAATTCAGGG CGTTGCTCGC GTTGGGAACA GAATACAACG CAGCACGTAA TGGTGGGGAC TCGTAAGAGA CTGCCGGGGT
CAACTCGGAG GAAGGTGGGG ACGACGTCAA GTCATCATGC CCCTTATGTC CAGGGCTTCA GTCGTGCATT ACCACCCCTG
AGCATTCTCT GACGGCCCCA GTTGAGCCTC CTTCCACCCC TGCTGCAGTT CAGTAGTACG GGGAATACAG GTCCCGAAGT
CACATGCTAC AATGGCCGGT ACAGAGGGCT GCGATACCGT GAGGTGGAGC GAATCCCTTA AAGCCGGTCT CAGTTCGGAT
CGGGGTCTGC AACTCGACCC GTGTACGATG TTACCGGCCA TGTCTCCCGA CGCTATGGCA CTCCACCTCG CTTAGGGAAT
TTCGGCCAGA GTCAAGCCTA GCCCCAGACG TTGAGCTGGG CGTGAAGTCG GAGTCGCTAG TAATCGCAGA TCAGCAACGC
TGCGGTGAAT ACGTTCCCGG GCCTTGTACA CACCGCCCGT CACGTCATGA AAGTCGGTAA GCACTTCAGC CTCAGCGATC
ATTAGCGTCT AGTCGTTGCG ACGCCACTTA TGCAAGGGCC CGGAACATGT GTGGCGGGCA GTGCAGTACT TTCAGCCATT
CACCCGAAGC CGGTGGCCTA ACCCCTCGTG GGAGGGAGCC GTCGAAGGTG GGATCGGCGA TTGGGACGAA GTCGTAACAA
GGTAGCCGTA CCGGAAGGTG GTGGGCTTCG GCCACCGGAT TGGGGAGCAC CCTCCCTCGG CAGCTTCCAC CCTAGCCGCT
AACCCTGCTT CAGCATTGTT CCATCGGCAT GGCCTTCCAC CGGCTGGATC ACCTCCTTTC TGCCGACCTA GTGGAGGAAA
GA
SEQ ID: 13
ACGTGGCGGC ATGCCTTACA CATGCAAGTC GAACGGCAGC GCGGACTTCG GTCTGGCGGC GAGTGGCGAA CGGGTGAGTA
ATACATCGGA ACGTACCCTG TGCACCGCCG TACGGAATGT GTACGTTCAG CTTGCCGTCG CGCCTGAAGC CAGACCGCCG
CTCACCGCTT GCCCACTCAT TATGTAGCCT TGCATGGGAC TTGTGGGGGA TAACTAGTCG AAAGATTAGC TAATACCGCA
TACGACCTGA GGGTGAAAGT GGGGGACCGC AAGGCCTCAC GCAGCAGGAG CGGCCGATGT AACACCCCCT ATTGATCAGC
TTTCTAATCG ATTATGGCGT ATGCTGGACT CCCACTTTCA CCCCCTGGCG TTCCGGAGTG CGTCGTCCTC GCCGGCTACA
CTGATTAGCT AGTTGGTGGG GTAAAGGCCC ACCAAGGCGA CGATCAGTAG CTGGTCTGAG AGGACGATCA GCCACACTGG
GACTGAGACA CGGCCCAGAC GACTAATCGA TCAACCACCC CATTTCCGGG TGGTTCCGCT GCTAGTCATC GACCAGACTC
TCCTGCTAGT CGGTGTGACC CTGACTCTGT GCCGGGTCTG TCCTACGGGA GGCAGCAGTG GGGAATTTTG GACAATGGGG
GCAACCCTGA TCCAGCAATG CCGCGTGTGT GAAGAAGGCC TTCGGGTTGT AAAGCACTTT AGGATGCCCT CCGTCGTCAC
CCCTTAAAAC CTGTTACCCC CGTTGGGACT AGGTCGTTAC GGCGCACACA CTTCTTCCGG AAGCCCAACA TTTCGTGAAA
TGTCCGGAAA GAAATCGCGC TGGTTAATAC CTGCGTGATG ACGGTACCGG AAGAATAAGC ACCGGCTAAC TACGTGCCAG
CAGCCGCGGT AATACGTAGG ACAGGCCTTT CTTTAGCGCG ACCAATTATG GACGCACTAC TGCCATGGCC TTCTTATTCG
TGGCCGATTG ATGCACGGTC GTCGGCGCCA TTATGCATCC GTGCGAGCGT TAATCGGAAT TACTGGGCGT AAAGCGTGCG
CAGGCGGTTT TGTAAGACAG GCGTGAAATC CCCGGGCTTA ACCTGGGAAT TGCGCTTGTG CACGCTCGCA ATTAGCCTTA
ATGACCCGCA TTTCGCACGC GTCCGCCAAA ACATTCTGTC CGCACTTTAG GGGCCCGAAT TGGACCCTTA ACGCGAACAC
ACTGCAAGGC TAGAGTGCGT CAGAGGGGGG TAGAATTCCA CGTGTAGCAG TGAAATGCGT AGAGATGTGG AGGAATACCG
ATGGCGAAGG CGAGCCCCCT TGACGTTCCG ATCTCACGCA GTCTCCCCCC ATCTTAAGGT GCACATCGTC ACTTTACGCA
TCTCTACACC TCCTTATGGC TACCGCTTCC GCTCGGGGGA GGACCTTGAC TGACGCTCAT GCACGAAAGC GTGGGGAGCA
AACAGGATTA GATACCCTGG TAGTCCACGC CCTAAACGAT GTCAACTAGT TGTTGGGATT CCTGGAACTG ACTGCGAGTA
CGTGCTTTCG CACCCCTCGT TTGTCCTAAT CTATGGGACC ATCAGGTGCG GGATTTGCTA CAGTTGATCA ACAACCCTAA
CATTTTCTCA GTAACGTAGC TAACGCGTGA AGTTGACCGC CTGGGGAGTA CGGCTGCAAG ATTAAAACTC AAAGGAATTG
ACGGGGACCC GCACAAGCGG GTAAAAGAGT CATTGCATCG ATTGCGCACT TCAACTGGCG GACCCCTCAT GCCGACGTTC
TAATTTTGAG TTTCCTTAAC TGCCCCTGGG CGTGTTCGCC TGGATGATGT GGATTAATTC GATGCAACGC GAAAAACCTT
ACCTACCCTT GACATGCCCT AACGAAGCAG AGATGCATTA GTGCCCGCAA AGGGAAAGTG ACCTACTACA CCTAATTAAG
CTACGTTGCG CTTTTTGGAA TGGATGGGAA CTGTACGGGA TTGCTTCGTC TCTACGTAAT CACGGGCGTT TCCCTTTCAC
GGACACAGGT GCTGCATGGC TGTCGTCAGC TCGTGTCGTG AGATGTTGGG TTAAGTCCCG CAACGAGCGC AACCCTTGTC
TCTAGTTGCC TACGCAAGAG CCTGTGTCCA CGACGTACCG ACAGCAGTCG AGCACAGCAC TCTACAACCC AATTCAGGGC
GTTGCTCGCG TTGGGAACAG AGATCAACGG ATGCGTTCTC CACTCTAGAG AGACTGCCGG TGACAAACCG GAGGAAGGTG
GGGATGACGT CAAGTCCTCA TGGCCCTTAT GGGTAGGGCT TCACACGTCA TACAATGGTG GTGAGATCTC TCTGACGGCC
ACTGTTTGGC CTCCTTCCAC CCCTACTGCA GTTCAGGAGT ACCGGGAATA CCCATCCCGA AGTGTGCAGT ATGTTACCAC
CGTACAGAGG GTTGCCAACC CGCGAGGGGG AGCTAATCCC AGAAAACGCA TCGTAGTCCG GATCGTAGTC TGCAACTCGA
CTACGTGAAG CTGGAATCGC GCATGTCTCC CAACGGTTGG GCGCTCCCCC TCGATTAGGG TCTTTTGCGT AGCATCAGGC
CTAGCATCAG ACGTTGAGCT GATGCACTTC GACCTTAGCG TAGTAATCGC GGATCAGCAT GCCGCGGTGA ATACGTTCCC
GGGTCTTGTA CACACCGCCC GTCACACCAT GGGAGTGGGT TTTGCCAGAA GTAGTTAGCC ATCATTAGCG CCTAGTCGTA
CGGCGCCACT TATGCAAGGG CCCAGAACAT GTGTGGCGGG CAGTGTGGTA CCCTCACCCA AAACGGTCTT CATCAATCGG
TAACCGCAAG GAGGGCGATT ACCACGGCAG GGTTCATGAC TGGGGTGAAG TCGTAACAAG GTATTGGCGT TCCTCCCGCT
AATGGTGCCG TCCCAAGTAC TGACCCCACT TCAGCATTGT TCCA
SEQ ID 14
MASIEDILEL EALEKDIFRG AVHPSVLKRT FGGQVAGQSL VSAVRTVDER FEVHSLHGYF LRPGNPTEPT VYLVDRIRDG
RSFCTRRVTG IQDGKAIFTM SASFHSQDEG IEHQDTMPSV PEPEELVDAQ TVEEMAATDL YREWKEWDVR IVPAGCTGKT
PGIAAKQRVW MRYRNKLPDD QVFHICTLAY LSDMTLLGAS KVPHPGVVTQ TASLDHAMWF LRPFRADEWL LYDQTSPSAG
FGRALTQGRM FDRKGTMVAA VVQEGLTRIQ RDQDQRDIET GNMA

In some embodiments, the cell comprises a plasmid that contains one or more exogenous nucleic acid sequences encoding enzymes or proteins that include but are not limited to one or more of the following: an acyl carrier protein, a TE, a FAR, a FadR, a FAD, a fatty aldehyde reductase, a cytochrome P450 enzyme, a NADH or NADPH cytochrome P450 reductase, a desaturase, a hydroxylase, and an antibiotic resistance enabling protein; wherein the plasmid is at least 20, 30, 40, 50, 60, 70, 80, 81, 82, 83, 84, 85, 86, 87, 88, 89, 90, 91, 92, 93, 94, 95, 96, 97, 98, or 99% homologous to SEQ ID NO:1, SEQ ID NO:2, SEQ ID NO:3, or SEQ ID NO:4. In some embodiments, the exogenous nucleic acid sequence is incorporated into the genome of the cell. In some embodiments, the cell or composition comprising a cell comprises at least one exogenous nucleic acid that encodes a FAR or a functional fragment of a FAR derived from one of the following organisms: Arabidopsis thaliana, Arabidopsis lyrata, Vitis vinifera, Populus trichocarpa, Artermisia annua, Ricinus communis, Simmondsia chineis, Oryza sativa japonica, Hevea brasiliensis, Hordeum vulgare, Triticum aestivum, Sorghum bicolor, Zea mays, and Selaginella moelllendorff.

In one embodiment, the exogenous gene encodes a FAR. In some cases, the FAR encoded by the exogenous gene catalyzes the reduction of a 20 to 30-carbon fatty acyl-CoA to a corresponding primary alcohol. In some cases, the FAR encoded by the exogenous gene catalyzes the reduction of an 8 to 18-carbon fatty acyl-CoA to a corresponding primary alcohol. In some cases, the FAR encoded by the exogenous gene catalyzes the reduction of a 10 to 14-carbon fatty acyl-CoA to a corresponding primary alcohol. In one embodiment, the FAR encoded by the exogenous gene catalyzes the reduction of a 12-carbon fatty acyl-CoA to dodecanol.

In one embodiment, the exogenous gene encodes a FadR. In some cases, the reductase encoded by the exogenous gene catalyzes the reduction of an 8 to 18-carbon fatty acyl-CoA to a corresponding aldehyde. In one embodiment, the reductase encoded by the exogenous gene catalyzes the reduction of a 12-carbon fatty acyl-CoA to dodecanal.

In some embodiments, the invention relates to a bacterial cell or a compositions comprising at least one bacterial cell that comprises at least a first and a second exogenous nucleic acid sequence, wherein the first nucleic acid sequence encodes a FadR or a functional fragment of a FadR and the second exogenous nucleic acid sequence encodes a fatty acyl-CoA ligase or a functional fragment thereof. In some embodiments, the functional fragments of the enzymes encoded by the one or more exogenous nucleic acid sequences are at least 40, 45, 50, 55, 60, 65, 70, 75, 80, 81, 82, 83, 84, 85, 86, 87, 88, 89, 90, 91, 92, 93, 94, 95, 96, 97, 98, or 99% homologous to the nucleic acid sequences that encode the full-length amino acid sequence upon which the functional fragment is based. Any enzyme disclosed in this application and part of the invention may be replaced with a functional fragment or variant. Any composition or cell disclosed in the application may be used in any disclosed method of this application.

In some embodiments, the genetic constructs contain sequences directing transcription and translation of the relevant exogenous (either heterologous or homologous) gene, a selectable marker, and/or sequences allowing autonomous replication or chromosomal integration. In some embodiments, suitable vectors comprise a region 5′ of the gene or DNA fragment which harbors transcriptional initiation controls and a region 3′ of the gene or DNA fragment which controls transcriptional termination. It is most preferred when both control regions are derived from genes homologous to the transformed host cell, although it is to be understood that such control regions need not be derived from the genes native to the specific species chosen as a production host. In some cells the exogenous gene is coding sequence and is in operable linkage with a promoter, and in some embodiments the promoter is derived from a gene endogenous to a species of the genus Rhodococcus or Ralstonia. Initiation control regions or promoters, which are useful to drive expression of the instant ORFs in the desired host cell are numerous and familiar to those skilled in the art. Virtually any promoter capable of driving these genes is suitable for the present invention including but not limited to CYC1, HIS3, GAL1, GAL10, ADH1, PGK, PHO5, GAPDH, ADC1, TRP1, URA3, LEU2, ENO; and lac, ara, tet, trp, IP_{L}, IP_{R}, T7, tac, and trc as well as the amy, apr, npr promoters and various phage promoters useful for expression in the lipid-producing bacteria of the present invention. In other embodiments the promoter is upregulated in response to reduction or elimination of a cofactor in the culture media of the cell, such as at least a 3-fold upregulation as determined by transcript abundance in a cell when the cell is exposed to extracellular environment changes from containing at least 10 mM or 5 mM cofactor to containing no cofactor.

Termination control regions may also be derived from various genes native to the preferred hosts. Optionally, the genetic constructs of the present invention do not comprise a termination control region.

In some embodiments, the bacterial cell or the composition comprising the bacterial cell comprises at least one genetic construct, which comprises one or more coding sequences. In some embodiments, the invention relates to the bacterial cell or the composition comprising at least one bacterial cell wherein the at least one cell comprises two or more genetic constructs, three or more genetic constructs, or four or more genetic constructs, each comprising one or more coding sequences. In some embodiments, the coding sequences of the claimed invention encode at least one protein that modifies or accelerates lipid production in the host cell. In some embodiments the coding sequence encodes at least one protein that alters the levels of individual lipids or hydrocarbons produced by the cell as compared to the same cell not modified by an exogenous nucleic acid sequence. In some embodiments, the coding sequence may encode at least one protein that alters the amount of one specific lipid or hydrocarbon molecule of the cell as compared to the same cell not modified by the nucleic acid. For example, in one embodiment, the one or more exogenous nucleic acid sequence encodes at least one lipid pathway enzyme that causes an increase in the ratio of C14:C16:C18 lipids or hydrocarbons produced or secreted by the cell as compared to the C14:C16:C18 lipids or hydrocarbons produced or secreted by the same cell not transformed with the nucleic acid sequence that encodes the lipid pathway enzyme. In one embodiment, the one or more exogenous nucleic acid sequence encodes at least one lipid pathway enzyme that causes a decrease in the ratio of C14:C16:C18 lipids or hydrocarbons produced or secreted by the cell as compared to the C14:C16:C18 lipids or hydrocarbons produced or secreted by the same cell not transformed with the nucleic acid sequence that encodes the lipid pathway enzyme. In some embodiments, the one or more cells comprising one or more exogenous nucleic acid sequences produces at least 5% more C8 hydrocarbon as compared to the same one or more cells not transformed or modified with the one or more exogenous nucleic acid sequences. In some embodiments, the one or more cells comprising one or more exogenous nucleic acid sequences produces at least 5% more C8 hydrocarbon as compared to the same one or more cells not transformed or modified with the one or more exogenous nucleic acid sequences.

In some embodiments, the bacterial cell produces and/or secretes one or more unsaturated lipids or hydrocarbons in a ratio greater than the ratio of unsaturated lipids or hydrocarbons produced and/or secreted by the same cell not cells comprising one or more exogenous nucleic acid sequences.

In some embodiments, the bacterial cell produces and/or secretes at least 6% more C8 hydrocarbon as compared to the same one or more cells not transformed or modified with the one or more exogenous nucleic acid sequences.

In some embodiments, the one or more cells comprising one or more exogenous nucleic acid sequences produces at least 7% more C8 hydrocarbon as compared to the same one or more cells not transformed or modified with the one or more exogenous nucleic acid sequences.

In some embodiments, the one or more cells comprising one or more exogenous nucleic acid sequences produces at least 8% more C8 hydrocarbon as compared to the same one or more cells not transformed or modified with the one or more exogenous nucleic acid sequences.

In some embodiments, the one or more cells comprising one or more exogenous nucleic acid sequences produces at least 9% more C8 hydrocarbon as compared to the same one or more cells not transformed or modified with the one or more exogenous nucleic acid sequences.

In some embodiments, the one or more cells comprising one or more exogenous nucleic acid sequences produces at least 10% more C8 hydrocarbon as compared to the same one or more cells not transformed or modified with the one or more exogenous nucleic acid sequences.

In some embodiments, the one or more cells comprising one or more exogenous nucleic acid sequences produces at least 15% more C8 hydrocarbon as compared to the same one or more cells not transformed or modified with the one or more exogenous nucleic acid sequences.

In some embodiments, the one or more cells comprising one or more exogenous nucleic acid sequences produces at least 20% more C8 hydrocarbon as compared to the same one or more cells not transformed or modified with the one or more exogenous nucleic acid sequences.

In some embodiments, the one or more cells comprising one or more exogenous nucleic acid sequences produces at least 25% more C8 hydrocarbon as compared to the same one or more cells not transformed or modified with the one or more exogenous nucleic acid sequences.

In some embodiments, the one or more cells comprising one or more exogenous nucleic acid sequences produces at least 30% more C8 hydrocarbon as compared to the same one or more cells not transformed or modified with the one or more exogenous nucleic acid sequences.

In some embodiments, the one or more cells comprising one or more exogenous nucleic acid sequences produces at least 35% more C8 hydrocarbon as compared to the same one or more cells not transformed or modified with the one or more exogenous nucleic acid sequences.

In some embodiments, the one or more cells comprising one or more exogenous nucleic acid sequences produces at least 40% more C8 hydrocarbon as compared to the same one or more cells not transformed or modified with the one or more exogenous nucleic acid sequences.

In some embodiments, the one or more cells comprising one or more exogenous nucleic acid sequences produces at least 45% more C8 hydrocarbon as compared to the same one or more cells not transformed or modified with the one or more exogenous nucleic acid sequences.

In some embodiments, the one or more cells comprising one or more exogenous nucleic acid sequences produces at least 50% more C8 hydrocarbon as compared to the same one or more cells not transformed or modified with the one or more exogenous nucleic acid sequences.

In some embodiments, the one or more cells comprising one or more exogenous nucleic acid sequences produces at least 5% more C9 hydrocarbon as compared to the same one or more cells not transformed or modified with the one or more exogenous nucleic acid sequences. In some embodiments, the one or more cells comprising one or more exogenous nucleic acid sequences produces at least 6% more C9 hydrocarbon as compared to the same one or more cells not transformed or modified with the one or more exogenous nucleic acid sequences. In some embodiments, the one or more cells comprising one or more exogenous nucleic acid sequences produces at least 7% more C9 hydrocarbon as compared to the same one or more cells not transformed or modified with the one or more exogenous nucleic acid sequences. In some embodiments, the one or more cells comprising one or more exogenous nucleic acid sequences produces at least 8% more C9 hydrocarbon as compared to the same one or more cells not transformed or modified with the one or more exogenous nucleic acid sequences. In some embodiments, the one or more cells comprising one or more exogenous nucleic acid sequences produces at least 9% more C9 hydrocarbon as compared to the same one or more cells not transformed or modified with the one or more exogenous nucleic acid sequences. In some embodiments, the one or more cells comprising one or more exogenous nucleic acid sequences produces at least 10% more C9 hydrocarbon as compared to the same one or more cells not transformed or modified with the one or more exogenous nucleic acid sequences. In some embodiments, the one or more cells comprising one or more exogenous nucleic acid sequences produces at least 15% more C9 hydrocarbon as compared to the same one or more cells not transformed or modified with the one or more exogenous nucleic acid sequences. In some embodiments, the one or more cells comprising one or more exogenous nucleic acid sequences produces at least 20% more C9 hydrocarbon as compared to the same one or more cells not transformed or modified with the one or more exogenous nucleic acid sequences. In some embodiments, the one or more cells comprising one or more exogenous nucleic acid sequences produces at least 25% more C9 hydrocarbon as compared to the same one or more cells not transformed or modified with the one or more exogenous nucleic acid sequences. In some embodiments, the one or more cells comprising one or more exogenous nucleic acid sequences produces at least 30% more C9 hydrocarbon as compared to the same one or more cells not transformed or modified with the one or more exogenous nucleic acid sequences. In some embodiments, the one or more cells comprising one or more exogenous nucleic acid sequences produces at least 35% more C9 hydrocarbon as compared to the same one or more cells not transformed or modified with the one or more exogenous nucleic acid sequences. In some embodiments, the one or more cells comprising one or more exogenous nucleic acid sequences produces at least 40% more C9 hydrocarbon as compared to the same one or more cells not transformed or modified with the one or more exogenous nucleic acid sequences. In some embodiments, the one or more cells comprising one or more exogenous nucleic acid sequences produces at least 45% more C9 hydrocarbon as compared to the same one or more cells not transformed or modified with the one or more exogenous nucleic acid sequences. In some embodiments, the one or more cells comprising one or more exogenous nucleic acid sequences produces at least 50% more C9 hydrocarbon as compared to the same one or more cells not transformed or modified with the one or more exogenous nucleic acid sequences.

In some embodiments, the one or more cells comprising one or more exogenous nucleic acid sequences produces at least 5% more C10 hydrocarbon as compared to the same one or more cells not transformed or modified with the one or more exogenous nucleic acid sequences.

In some embodiments, the one or more cells comprising one or more exogenous nucleic acid sequences produces at least 6% more C10 hydrocarbon as compared to the same one or more cells not transformed or modified with the one or more exogenous nucleic acid sequences.

In some embodiments, the one or more cells comprising one or more exogenous nucleic acid sequences produces at least 7% more C10 hydrocarbon as compared to the same one or more cells not transformed or modified with the one or more exogenous nucleic acid sequences.

In some embodiments, the one or more cells comprising one or more exogenous nucleic acid sequences produces at least 8% more C10 hydrocarbon as compared to the same one or more cells not transformed or modified with the one or more exogenous nucleic acid sequences.

In some embodiments, the one or more cells comprising one or more exogenous nucleic acid sequences produces at least 9% more C10 hydrocarbon as compared to the same one or more cells not transformed or modified with the one or more exogenous nucleic acid sequences.

In some embodiments, the one or more cells comprising one or more exogenous nucleic acid sequences produces at least 10% more C10 hydrocarbon as compared to the same one or more cells not transformed or modified with the one or more exogenous nucleic acid sequences.

In some embodiments, the one or more cells comprising one or more exogenous nucleic acid sequences produces at least 15% more C10 hydrocarbon as compared to the same one or more cells not transformed or modified with the one or more exogenous nucleic acid sequences.

In some embodiments, the one or more cells comprising one or more exogenous nucleic acid sequences produces at least 20% more C10 hydrocarbon as compared to the same one or more cells not transformed or modified with the one or more exogenous nucleic acid sequences.

In some embodiments, the one or more cells comprising one or more exogenous nucleic acid sequences produces at least 25% more C10 hydrocarbon as compared to the same one or more cells not transformed or modified with the one or more exogenous nucleic acid sequences.

In some embodiments, the one or more cells comprising one or more exogenous nucleic acid sequences produces at least 30% more C10 hydrocarbon as compared to the same one or more cells not transformed or modified with the one or more exogenous nucleic acid sequences.

In some embodiments, the one or more cells comprising one or more exogenous nucleic acid sequences produces at least 35% more C10 hydrocarbon as compared to the same one or more cells not transformed or modified with the one or more exogenous nucleic acid sequences.

In some embodiments, the one or more cells comprising one or more exogenous nucleic acid sequences produces at least 40% more C10 hydrocarbon as compared to the same one or more cells not transformed or modified with the one or more exogenous nucleic acid sequences.

In some embodiments, the one or more cells comprising one or more exogenous nucleic acid sequences produces at least 45% more C10 hydrocarbon as compared to the same one or more cells not transformed or modified with the one or more exogenous nucleic acid sequences.

In some embodiments, the one or more cells comprising one or more exogenous nucleic acid sequences produces at least 50% more C10 hydrocarbon as compared to the same one or more cells not transformed or modified with the one or more exogenous nucleic acid sequences.

In some embodiments, the one or more cells comprising one or more exogenous nucleic acid sequences produces at least 5% more C11 hydrocarbon as compared to the same one or more cells not transformed or modified with the one or more exogenous nucleic acid sequences.

In some embodiments, the one or more cells comprising one or more exogenous nucleic acid sequences produces at least 6% more C11 hydrocarbon as compared to the same one or more cells not transformed or modified with the one or more exogenous nucleic acid sequences. In some embodiments, the one or more cells comprising one or more exogenous nucleic acid sequences produces at least 7% more C11 hydrocarbon as compared to the same one or more cells not transformed or modified with the one or more exogenous nucleic acid sequences. In some embodiments, the one or more cells comprising one or more exogenous nucleic acid sequences produces at least 8% more C11 hydrocarbon as compared to the same one or more cells not transformed or modified with the one or more exogenous nucleic acid sequences. In some embodiments, the one or more cells comprising one or more exogenous nucleic acid sequences produces at least 9% more C11 hydrocarbon as compared to the same one or more cells not transformed or modified with the one or more exogenous nucleic acid sequences. In some embodiments, the one or more cells comprising one or more exogenous nucleic acid sequences produces at least 10% more C11 hydrocarbon as compared to the same one or more cells not transformed or modified with the one or more exogenous nucleic acid sequences.

In some embodiments, the one or more cells comprising one or more exogenous nucleic acid sequences produces at least 15% more C11 hydrocarbon as compared to the same one or more cells not transformed or modified with the one or more exogenous nucleic acid sequences.

In some embodiments, the one or more cells comprising one or more exogenous nucleic acid sequences produces at least 20% more C11 hydrocarbon as compared to the same one or more cells not transformed or modified with the one or more exogenous nucleic acid sequences.

In some embodiments, the one or more cells comprising one or more exogenous nucleic acid sequences produces at least 25% more C11 hydrocarbon as compared to the same one or more cells not transformed or modified with the one or more exogenous nucleic acid sequences.

In some embodiments, the one or more cells comprising one or more exogenous nucleic acid sequences produces at least 30% more C11 hydrocarbon as compared to the same one or more cells not transformed or modified with the one or more exogenous nucleic acid sequences.

In some embodiments, the one or more cells comprising one or more exogenous nucleic acid sequences produces at least 35% more C11 hydrocarbon as compared to the same one or more cells not transformed or modified with the one or more exogenous nucleic acid sequences.

In some embodiments, the one or more cells comprising one or more exogenous nucleic acid sequences produces at least 40% more C11 hydrocarbon as compared to the same one or more cells not transformed or modified with the one or more exogenous nucleic acid sequences.

In some embodiments, the one or more cells comprising one or more exogenous nucleic acid sequences produces at least 45% more C11 hydrocarbon as compared to the same one or more cells not transformed or modified with the one or more exogenous nucleic acid sequences.

In some embodiments, the one or more cells comprising one or more exogenous nucleic acid sequences produces at least 50% more C11 hydrocarbon as compared to the same one or more cells not transformed or modified with the one or more exogenous nucleic acid sequences.

In some embodiments, the one or more cells comprising one or more exogenous nucleic acid sequences produces at least 5% more C12 hydrocarbon as compared to the same one or more cells not transformed or modified with the one or more exogenous nucleic acid sequences.

In some embodiments, the one or more cells comprising one or more exogenous nucleic acid sequences produces at least 6% more C12 hydrocarbon as compared to the same one or more cells not transformed or modified with the one or more exogenous nucleic acid sequences.

In some embodiments, the one or more cells comprising one or more exogenous nucleic acid sequences produces at least 7% more C12 hydrocarbon as compared to the same one or more cells not transformed or modified with the one or more exogenous nucleic acid sequences.

In some embodiments, the one or more cells comprising one or more exogenous nucleic acid sequences produces at least 8% more C12 hydrocarbon as compared to the same one or more cells not transformed or modified with the one or more exogenous nucleic acid sequences.

In some embodiments, the one or more cells comprising one or more exogenous nucleic acid sequences produces at least 9% more C12 hydrocarbon as compared to the same one or more cells not transformed or modified with the one or more exogenous nucleic acid sequences.

In some embodiments, the one or more cells comprising one or more exogenous nucleic acid sequences produces at least 10% more C12 hydrocarbon as compared to the same one or more cells not transformed or modified with the one or more exogenous nucleic acid sequences.

In some embodiments, the one or more cells comprising one or more exogenous nucleic acid sequences produces at least 15% more C12 hydrocarbon as compared to the same one or more cells not transformed or modified with the one or more exogenous nucleic acid sequences.

In some embodiments, the one or more cells comprising one or more exogenous nucleic acid sequences produces at least 20% more C12 hydrocarbon as compared to the same one or more cells not transformed or modified with the one or more exogenous nucleic acid sequences.

In some embodiments, the one or more cells comprising one or more exogenous nucleic acid sequences produces at least 25% more C12 hydrocarbon as compared to the same one or more cells not transformed or modified with the one or more exogenous nucleic acid sequences.

In some embodiments, the one or more cells comprising one or more exogenous nucleic acid sequences produces at least 30% more C12 hydrocarbon as compared to the same one or more cells not transformed or modified with the one or more exogenous nucleic acid sequences.

In some embodiments, the one or more cells comprising one or more exogenous nucleic acid sequences produces at least 35% more C12 hydrocarbon as compared to the same one or more cells not transformed or modified with the one or more exogenous nucleic acid sequences.

In some embodiments, the one or more cells comprising one or more exogenous nucleic acid sequences produces at least 40% more C12 hydrocarbon as compared to the same one or more cells not transformed or modified with the one or more exogenous nucleic acid sequences.

In some embodiments, the one or more cells comprising one or more exogenous nucleic acid sequences produces at least 45% more C12 hydrocarbon as compared to the same one or more cells not transformed or modified with the one or more exogenous nucleic acid sequences.

In some embodiments, the one or more cells comprising one or more exogenous nucleic acid sequences produces at least 50% more C12 hydrocarbon as compared to the same one or more cells not transformed or modified with the one or more exogenous nucleic acid sequences.

In some embodiments, the one or more cells comprising one or more exogenous nucleic acid sequences produces at least 5% more C13 hydrocarbon as compared to the same one or more cells not transformed or modified with the one or more exogenous nucleic acid sequences.

In some embodiments, the one or more cells comprising one or more exogenous nucleic acid sequences produces at least 6% more C13 hydrocarbon as compared to the same one or more cells not transformed or modified with the one or more exogenous nucleic acid sequences.

In some embodiments, the one or more cells comprising one or more exogenous nucleic acid sequences produces at least 7% more C13 hydrocarbon as compared to the same one or more cells not transformed or modified with the one or more exogenous nucleic acid sequences.

In some embodiments, the one or more cells comprising one or more exogenous nucleic acid sequences produces at least 8% more C13 hydrocarbon as compared to the same one or more cells not transformed or modified with the one or more exogenous nucleic acid sequences.

In some embodiments, the one or more cells comprising one or more exogenous nucleic acid sequences produces at least 9% more C13 hydrocarbon as compared to the same one or more cells not transformed or modified with the one or more exogenous nucleic acid sequences.

In some embodiments, the one or more cells comprising one or more exogenous nucleic acid sequences produces at least 10% more C13 hydrocarbon as compared to the same one or more cells not transformed or modified with the one or more exogenous nucleic acid sequences.

In some embodiments, the one or more cells comprising one or more exogenous nucleic acid sequences produces at least 15% more C13 hydrocarbon as compared to the same one or more cells not transformed or modified with the one or more exogenous nucleic acid sequences.

In some embodiments, the one or more cells comprising one or more exogenous nucleic acid sequences produces at least 20% more C13 hydrocarbon as compared to the same one or more cells not transformed or modified with the one or more exogenous nucleic acid sequences.

In some embodiments, the one or more cells comprising one or more exogenous nucleic acid sequences produces at least 25% more C13 hydrocarbon as compared to the same one or more cells not transformed or modified with the one or more exogenous nucleic acid sequences.

In some embodiments, the one or more cells comprising one or more exogenous nucleic acid sequences produces at least 30% more C13 hydrocarbon as compared to the same one or more cells not transformed or modified with the one or more exogenous nucleic acid sequences.

In some embodiments, the one or more cells comprising one or more exogenous nucleic acid sequences produces at least 35% more C13 hydrocarbon as compared to the same one or more cells not transformed or modified with the one or more exogenous nucleic acid sequences.

In some embodiments, the one or more cells comprising one or more exogenous nucleic acid sequences produces at least 40% more C13 hydrocarbon as compared to the same one or more cells not transformed or modified with the one or more exogenous nucleic acid sequences.

In some embodiments, the one or more cells comprising one or more exogenous nucleic acid sequences produces at least 45% more C13 hydrocarbon as compared to the same one or more cells not transformed or modified with the one or more exogenous nucleic acid sequences.

In some embodiments, the one or more cells comprising one or more exogenous nucleic acid sequences produces at least 50% more C13 hydrocarbon as compared to the same one or more cells not transformed or modified with the one or more exogenous nucleic acid sequences.

In some embodiments, the one or more cells comprising one or more exogenous nucleic acid sequences produces at least 5% more C14 hydrocarbon as compared to the same one or more cells not transformed or modified with the one or more exogenous nucleic acid sequences.

In some embodiments, the one or more cells comprising one or more exogenous nucleic acid sequences produces at least 6% more C14 hydrocarbon as compared to the same one or more cells not transformed or modified with the one or more exogenous nucleic acid sequences.

In some embodiments, the one or more cells comprising one or more exogenous nucleic acid sequences produces at least 7% more C14 hydrocarbon as compared to the same one or more cells not transformed or modified with the one or more exogenous nucleic acid sequences.

In some embodiments, the one or more cells comprising one or more exogenous nucleic acid sequences produces at least 8% more C14 hydrocarbon as compared to the same one or more cells not transformed or modified with the one or more exogenous nucleic acid sequences.

In some embodiments, the one or more cells comprising one or more exogenous nucleic acid sequences produces at least 9% more C14 hydrocarbon as compared to the same one or more cells not transformed or modified with the one or more exogenous nucleic acid sequences.

In some embodiments, the one or more cells comprising one or more exogenous nucleic acid sequences produces at least 10% more C14 hydrocarbon as compared to the same one or more cells not transformed or modified with the one or more exogenous nucleic acid sequences.

In some embodiments, the one or more cells comprising one or more exogenous nucleic acid sequences produces at least 15% more C14 hydrocarbon as compared to the same one or more cells not transformed or modified with the one or more exogenous nucleic acid sequences.

In some embodiments, the one or more cells comprising one or more exogenous nucleic acid sequences produces at least 20% more C14 hydrocarbon as compared to the same one or more cells not transformed or modified with the one or more exogenous nucleic acid sequences.

In some embodiments, the one or more cells comprising one or more exogenous nucleic acid sequences produces at least 25% more C14 hydrocarbon as compared to the same one or more cells not transformed or modified with the one or more exogenous nucleic acid sequences.

In some embodiments, the one or more cells comprising one or more exogenous nucleic acid sequences produces at least 30% more C14 hydrocarbon as compared to the same one or more cells not transformed or modified with the one or more exogenous nucleic acid sequences.

In some embodiments, the one or more cells comprising one or more exogenous nucleic acid sequences produces at least 35% more C14 hydrocarbon as compared to the same one or more cells not transformed or modified with the one or more exogenous nucleic acid sequences.

In some embodiments, the one or more cells comprising one or more exogenous nucleic acid sequences produces at least 40% more C14 hydrocarbon as compared to the same one or more cells not transformed or modified with the one or more exogenous nucleic acid sequences.

In some embodiments, the one or more cells comprising one or more exogenous nucleic acid sequences produces at least 45% more C14 hydrocarbon as compared to the same one or more cells not transformed or modified with the one or more exogenous nucleic acid sequences.

In some embodiments, the one or more cells comprising one or more exogenous nucleic acid sequences produces at least 50% more C14 hydrocarbon as compared to the same one or more cells not transformed or modified with the one or more exogenous nucleic acid sequences.

In some embodiments, the one or more cells comprising one or more exogenous nucleic acid sequences produces at least 5% more C15 hydrocarbon as compared to the same one or more cells not transformed or modified with the one or more exogenous nucleic acid sequences.

In some embodiments, the one or more cells comprising one or more exogenous nucleic acid sequences produces at least 6% more C15 hydrocarbon as compared to the same one or more cells not transformed or modified with the one or more exogenous nucleic acid sequences.

In some embodiments, the one or more cells comprising one or more exogenous nucleic acid sequences produces at least 7% more C15 hydrocarbon as compared to the same one or more cells not transformed or modified with the one or more exogenous nucleic acid sequences.

In some embodiments, the one or more cells comprising one or more exogenous nucleic acid sequences produces at least 8% more C15 hydrocarbon as compared to the same one or more cells not transformed or modified with the one or more exogenous nucleic acid sequences.

In some embodiments, the one or more cells comprising one or more exogenous nucleic acid sequences produces at least 9% more C15 hydrocarbon as compared to the same one or more cells not transformed or modified with the one or more exogenous nucleic acid sequences.

In some embodiments, the one or more cells comprising one or more exogenous nucleic acid sequences produces at least 10% more C15 hydrocarbon as compared to the same one or more cells not transformed or modified with the one or more exogenous nucleic acid sequences.

In some embodiments, the one or more cells comprising one or more exogenous nucleic acid sequences produces at least 15% more C15 hydrocarbon as compared to the same one or more cells not transformed or modified with the one or more exogenous nucleic acid sequences.

In some embodiments, the one or more cells comprising one or more exogenous nucleic acid sequences produces at least 20% more C15 hydrocarbon as compared to the same one or more cells not transformed or modified with the one or more exogenous nucleic acid sequences.

In some embodiments, the one or more cells comprising one or more exogenous nucleic acid sequences produces at least 25% more C15 hydrocarbon as compared to the same one or more cells not transformed or modified with the one or more exogenous nucleic acid sequences.

In some embodiments, the one or more cells comprising one or more exogenous nucleic acid sequences produces at least 30% more C15 hydrocarbon as compared to the same one or more cells not transformed or modified with the one or more exogenous nucleic acid sequences.

In some embodiments, the one or more cells comprising one or more exogenous nucleic acid sequences produces at least 35% more C15 hydrocarbon as compared to the same one or more cells not transformed or modified with the one or more exogenous nucleic acid sequences.

In some embodiments, the one or more cells comprising one or more exogenous nucleic acid sequences produces at least 40% more C15 hydrocarbon as compared to the same one or more cells not transformed or modified with the one or more exogenous nucleic acid sequences.

In some embodiments, the one or more cells comprising one or more exogenous nucleic acid sequences produces at least 45% more C15 hydrocarbon as compared to the same one or more cells not transformed or modified with the one or more exogenous nucleic acid sequences.

In some embodiments, the one or more cells comprising one or more exogenous nucleic acid sequences produces at least 50% more C15 hydrocarbon as compared to the same one or more cells not transformed or modified with the one or more exogenous nucleic acid sequences.

In some embodiments, the one or more cells comprising one or more exogenous nucleic acid sequences produces at least 5% more C16 hydrocarbon as compared to the same one or more cells not transformed or modified with the one or more exogenous nucleic acid sequences.

In some embodiments, the one or more cells comprising one or more exogenous nucleic acid sequences produces at least 6% more C16 hydrocarbon as compared to the same one or more cells not transformed or modified with the one or more exogenous nucleic acid sequences.

In some embodiments, the one or more cells comprising one or more exogenous nucleic acid sequences produces at least 7% more C16 hydrocarbon as compared to the same one or more cells not transformed or modified with the one or more exogenous nucleic acid sequences.

In some embodiments, the one or more cells comprising one or more exogenous nucleic acid sequences produces at least 8% more C16 hydrocarbon as compared to the same one or more cells not transformed or modified with the one or more exogenous nucleic acid sequences.

In some embodiments, the one or more cells comprising one or more exogenous nucleic acid sequences produces at least 9% more C16 hydrocarbon as compared to the same one or more cells not transformed or modified with the one or more exogenous nucleic acid sequences.

In some embodiments, the one or more cells comprising one or more exogenous nucleic acid sequences produces at least 10% more C16 hydrocarbon as compared to the same one or more cells not transformed or modified with the one or more exogenous nucleic acid sequences.

In some embodiments, the one or more cells comprising one or more exogenous nucleic acid sequences produces at least 15% more C16 hydrocarbon as compared to the same one or more cells not transformed or modified with the one or more exogenous nucleic acid sequences.

In some embodiments, the one or more cells comprising one or more exogenous nucleic acid sequences produces at least 20% more C16 hydrocarbon as compared to the same one or more cells not transformed or modified with the one or more exogenous nucleic acid sequences.

In some embodiments, the one or more cells comprising one or more exogenous nucleic acid sequences produces at least 25% more C16 hydrocarbon as compared to the same one or more cells not transformed or modified with the one or more exogenous nucleic acid sequences.

In some embodiments, the one or more cells comprising one or more exogenous nucleic acid sequences produces at least 30% more C16 hydrocarbon as compared to the same one or more cells not transformed or modified with the one or more exogenous nucleic acid sequences.

In some embodiments, the one or more cells comprising one or more exogenous nucleic acid sequences produces at least 35% more C16 hydrocarbon as compared to the same one or more cells not transformed or modified with the one or more exogenous nucleic acid sequences.

In some embodiments, the one or more cells comprising one or more exogenous nucleic acid sequences produces at least 40% more C16 hydrocarbon as compared to the same one or more cells not transformed or modified with the one or more exogenous nucleic acid sequences.

In some embodiments, the one or more cells comprising one or more exogenous nucleic acid sequences produces at least 45% more C16 hydrocarbon as compared to the same one or more cells not transformed or modified with the one or more exogenous nucleic acid sequences.

In some embodiments, the one or more cells comprising one or more exogenous nucleic acid sequences produces at least 50% more C16 hydrocarbon as compared to the same one or more cells not transformed or modified with the one or more exogenous nucleic acid sequences.

In some embodiments, the one or more cells comprising one or more exogenous nucleic acid sequences produces at least 5% more C17 hydrocarbon as compared to the same one or more cells not transformed or modified with the one or more exogenous nucleic acid sequences.

In some embodiments, the one or more cells comprising one or more exogenous nucleic acid sequences produces at least 6% more C17 hydrocarbon as compared to the same one or more cells not transformed or modified with the one or more exogenous nucleic acid sequences.

In some embodiments, the one or more cells comprising one or more exogenous nucleic acid sequences produces at least 7% more C17 hydrocarbon as compared to the same one or more cells not transformed or modified with the one or more exogenous nucleic acid sequences.

In some embodiments, the one or more cells comprising one or more exogenous nucleic acid sequences produces at least 8% more C17 hydrocarbon as compared to the same one or more cells not transformed or modified with the one or more exogenous nucleic acid sequences.

In some embodiments, the one or more cells comprising one or more exogenous nucleic acid sequences produces at least 9% more C17 hydrocarbon as compared to the same one or more cells not transformed or modified with the one or more exogenous nucleic acid sequences.

In some embodiments, the one or more cells comprising one or more exogenous nucleic acid sequences produces at least 10% more C17 hydrocarbon as compared to the same one or more cells not transformed or modified with the one or more exogenous nucleic acid sequences.

In some embodiments, the one or more cells comprising one or more exogenous nucleic acid sequences produces at least 15% more C17 hydrocarbon as compared to the same one or more cells not transformed or modified with the one or more exogenous nucleic acid sequences.

In some embodiments, the one or more cells comprising one or more exogenous nucleic acid sequences produces at least 20% more C17 hydrocarbon as compared to the same one or more cells not transformed or modified with the one or more exogenous nucleic acid sequences.

In some embodiments, the one or more cells comprising one or more exogenous nucleic acid sequences produces at least 25% more C17 hydrocarbon as compared to the same one or more cells not transformed or modified with the one or more exogenous nucleic acid sequences.

In some embodiments, the one or more cells comprising one or more exogenous nucleic acid sequences produces at least 30% more C17 hydrocarbon as compared to the same one or more cells not transformed or modified with the one or more exogenous nucleic acid sequences.

In some embodiments, the one or more cells comprising one or more exogenous nucleic acid sequences produces at least 35% more C17 hydrocarbon as compared to the same one or more cells not transformed or modified with the one or more exogenous nucleic acid sequences.

In some embodiments, the one or more cells comprising one or more exogenous nucleic acid sequences produces at least 40% more C17 hydrocarbon as compared to the same one or more cells not transformed or modified with the one or more exogenous nucleic acid sequences.

In some embodiments, the one or more cells comprising one or more exogenous nucleic acid sequences produces at least 45% more C17 hydrocarbon as compared to the same one or more cells not transformed or modified with the one or more exogenous nucleic acid sequences.

In some embodiments, the one or more cells comprising one or more exogenous nucleic acid sequences produces at least 50% more C17 hydrocarbon as compared to the same one or more cells not transformed or modified with the one or more exogenous nucleic acid sequences.

In some embodiments, the one or more cells comprising one or more exogenous nucleic acid sequences produces at least 5% more C18 hydrocarbon as compared to the same one or more cells not transformed or modified with the one or more exogenous nucleic acid sequences.

In some embodiments, the one or more cells comprising one or more exogenous nucleic acid sequences produces at least 6% more C18 hydrocarbon as compared to the same one or more cells not transformed or modified with the one or more exogenous nucleic acid sequences.

In some embodiments, the one or more cells comprising one or more exogenous nucleic acid sequences produces at least 7% more C18 hydrocarbon as compared to the same one or more cells not transformed or modified with the one or more exogenous nucleic acid sequences.

In some embodiments, the one or more cells comprising one or more exogenous nucleic acid sequences produces at least 8% more C18 hydrocarbon as compared to the same one or more cells not transformed or modified with the one or more exogenous nucleic acid sequences.

In some embodiments, the one or more cells comprising one or more exogenous nucleic acid sequences produces at least 9% more C18 hydrocarbon as compared to the same one or more cells not transformed or modified with the one or more exogenous nucleic acid sequences.

In some embodiments, the one or more cells comprising one or more exogenous nucleic acid sequences produces at least 10% more C18 hydrocarbon as compared to the same one or more cells not transformed or modified with the one or more exogenous nucleic acid sequences.

In some embodiments, the one or more cells comprising one or more exogenous nucleic acid sequences produces at least 15% more C18 hydrocarbon as compared to the same one or more cells not transformed or modified with the one or more exogenous nucleic acid sequences.

In some embodiments, the one or more cells comprising one or more exogenous nucleic acid sequences produces at least 20% more C18 hydrocarbon as compared to the same one or more cells not transformed or modified with the one or more exogenous nucleic acid sequences.

In some embodiments, the one or more cells comprising one or more exogenous nucleic acid sequences produces at least 25% more C18 hydrocarbon as compared to the same one or more cells not transformed or modified with the one or more exogenous nucleic acid sequences.

In some embodiments, the one or more cells comprising one or more exogenous nucleic acid sequences produces at least 30% more C18 hydrocarbon as compared to the same one or more cells not transformed or modified with the one or more exogenous nucleic acid sequences.

In some embodiments, the one or more cells comprising one or more exogenous nucleic acid sequences produces at least 35% more C18 hydrocarbon as compared to the same one or more cells not transformed or modified with the one or more exogenous nucleic acid sequences.

In some embodiments, the one or more cells comprising one or more exogenous nucleic acid sequences produces at least 40% more C18 hydrocarbon as compared to the same one or more cells not transformed or modified with the one or more exogenous nucleic acid sequences.

In some embodiments, the one or more cells comprising one or more exogenous nucleic acid sequences produces at least 45% more C18 hydrocarbon as compared to the same one or more cells not transformed or modified with the one or more exogenous nucleic acid sequences.

In some embodiments, the one or more cells comprising one or more exogenous nucleic acid sequences produces at least 50% more C18 hydrocarbon as compared to the same one or more cells not transformed or modified with the one or more exogenous nucleic acid sequences.

In some embodiments, the one or more exogenous nucleic acid sequence encodes at least one lipid pathway enzyme that causes an increase in the ratio of C12:C14:C16 lipids or hydrocarbons produced or secreted by the cell as compared to the C12:C14:C16 lipids or hydrocarbons produced or secreted by the same cell not transformed with the nucleic acid sequence that encodes the lipid pathway enzyme. In one embodiment, the one or more exogenous nucleic acid sequence encodes at least one lipid pathway enzyme that causes a decrease in the ratio of C12:C14:C16 lipids or hydrocarbons produced or secreted by the cell as compared to the C12:C14:C16 lipids or hydrocarbons produced or secreted by the same cell not transformed with the nucleic acid sequence that encodes the lipid pathway enzyme. In some embodiments, the one or more exogenous nucleic acid sequence encodes at least one lipid pathway enzyme that causes an increase in the ratio of C13:C15:C17 lipids or hydrocarbons produced or secreted by the cell as compared to the C13:C15:C17 lipids or hydrocarbons produced or secreted by the same cell not transformed with the nucleic acid sequence that encodes the lipid pathway enzyme. In one embodiment, the one or more exogenous nucleic acid sequence encodes at least one lipid pathway enzyme that causes a decrease in the ratio of C13:C15:C17 lipids or hydrocarbons produced or secreted by the cell as compared to the C13:C15:C17 lipids or hydrocarbons produced or secreted by the same cell not transformed with the nucleic acid sequence that encodes the lipid pathway enzyme. In some embodiments, the one or more exogenous nucleic acid sequence encodes at least one lipid pathway enzyme that causes an increase in the ratio of odd-numbered lipids or hydrocarbons produced or secreted by the cell as compared to the odd-numbered lipids or hydrocarbons produced or secreted by the same cell not transformed with the nucleic acid sequence that encodes the lipid pathway enzyme. In some embodiments, the one or more exogenous nucleic acid sequence encodes at least one lipid pathway enzyme that causes a decrease in the ratio of odd-numbered lipids or hydrocarbons produced or secreted by the cell as compared to the odd-numbered lipids or hydrocarbons produced or secreted by the same cell not transformed with the nucleic acid sequence that encodes the lipid pathway enzyme. In one embodiment, the one or more exogenous nucleic acid sequence encodes at least one lipid pathway enzyme that causes a decrease in the ratio of even:odd carbon numbered lipids or hydrocarbons produced or secreted by the cell as compared to the ratio of even:odd carbon numbered lipids or hydrocarbons produced or secreted by the same cell not transformed with the nucleic acid sequence that encodes the one or more lipid pathway enzymes. In one embodiment, the one or more exogenous nucleic acid sequence encodes at least one lipid pathway enzyme that causes a increase in the ratio of even:odd carbon numbered lipids or hydrocarbons produced or secreted by the cell as compared to the ratio of even:odd carbon numbered lipids or hydrocarbons produced or secreted by the same cell not transformed with the nucleic acid sequence that encodes the one or more lipid pathway enzymes.

In some embodiments, the one or more exogenous nucleic acid sequence encodes at least one lipid pathway enzyme that causes at least a 5% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme. In some embodiments, the one or more exogenous nucleic acid sequence encodes at least one lipid pathway enzyme that causes at least a 5% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 6% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 7% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 8% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 9% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 10% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 11% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 12% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 13% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 14% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 15% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 20% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 25% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 30% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 35% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 40% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 45% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 50% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 55% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 60% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 65% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 70% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 75% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 80% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 85% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 5% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme. In some embodiments, the one or more exogenous nucleic acid sequence encodes at least one lipid pathway enzyme that causes at least a 5% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 6% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 7% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 8% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 9% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 10% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 11% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 12% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 13% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 14% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 15% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 20% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 25% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 30% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 35% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 40% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 45% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 50% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 55% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 60% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 65% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 70% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 75% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 80% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 85% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments the exogenous gene or genes codes for enzymes or proteins including but not limited to one or more of the following: an acyl carrier protein, a TE, a FAR, a FadR, a FAD, a fatty aldehyde reductase, a cytochrome P450 enzyme, a NADH or NADPH cytochrome P450 reductase, a desaturase, a hydroxylase, and an antibiotic resistance enabling protein or a fragment or variant thereof. In some embodiments, the coding sequence comprises an exogenous nucleic acid sequence that encodes a TE that catalyzes hydrolysis of one or more fatty acyl-ACP substrates with chain lengths ranging over C8, C9, C10, C11, C12, C13, C14, C15, C16, C17, or C18. In some embodiments, the cell comprises a plasmid that contains one or more exogenous nucleic acid sequences that encode an amino acid sequence for an enzyme or protein such as but not limited to one or more of the following: an acyl carrier protein, a TE, a FAR, a FadR, a FAD, a fatty aldehyde reductase, a cytochrome P450 enzyme, a NADH or NADPH cytochrome P450 reductase, a desaturase, a hydroxylase, and an antibiotic resistance enabling protein or a fragment or variant thereof. In some embodiments, the one or more exogenous nucleic acid sequences comprise SEQ ID NO:5 or a functional fragment or variant thereof that is at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% homologous to SEQ ID NO:5. In some embodiments, the one or more exogenous nucleic acid sequences comprise SEQ ID NO:6 or a functional fragment thereof that is at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% homologous to SEQ ID NO:6. In some embodiments, the one or more exogenous nucleic acid sequences comprise SEQ ID NO:7 or a functional fragment thereof that is at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% homologous to SEQ ID NO:7. In some embodiments, the one or more exogenous nucleic acid sequences comprise SEQ ID NO:8 or a functional fragment thereof that is at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% homologous to SEQ ID NO:8. In some embodiments, the one or more exogenous nucleic acid sequences comprise SEQ ID NO:9 or a functional fragment thereof that is at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% homologous to SEQ ID NO:9.

In further embodiments, at least one coding sequence of the at least one exogenous nucleic acid sequence encodes a lipid pathway enzyme or a functional fragment or variant thereof. In some embodiments, the at least one coding sequence of the at least one exogenous nucleic acid sequence encodes a lipid modification enzyme or a functional fragment or variant thereof. In some embodiments, the composition or cell comprises a nucleic acid that encodes at least one fatty acid decarbonylase, at least one fatty acid reductase, a thioesterase, or any combination of any one more full-length lipid pathway enzymes or functional fragments or variants thereof. In some embodiments the one or more exogenous nucleic acid sequences are integrated into the genome of the cell. In some embodiments, the one or more exogenous nucleic acid sequences are on an episomal plasmid within the transformed host cell.

### Methods of Isolation and Purification

Following the methods of the present invention microorganisms are grown and maintained for the production of lipids in a medium containing a gaseous carbon source, such as but not limited to syngas or producer gas, in the absence of light; such growth is known as chemotrophic growth. In some embodiments, the invention relates to methods of cultivating oleaginous cells for the large scale production of oil and/or fuel. In some embodiments, the invention relates to methods of cultivating oleaginous cells in bioreactors 50,000 liters or greater in volume, which are conventionally constructed out of low cost, sturdy, and opaque materials such as steel or reinforced concrete or earthworks. The size, depth, and construction of such bioreactors dictate that the cells will be grown in near or total darkness. In some embodiments, the oleaginous microorganisms are cultured for the synthesis of lipids in accordance with the methods of the present invention in a medium containing gaseous inorganic carbon, such as but not limited to syngas or producer gas, as the primary or sole carbon source, and without any exposure to light. This type of growth is known as chemoautotrophic growth.

To give an illustration, a bioreactor containing nutrient medium is inoculated with of oleaginous bacterial cells; generally there will follow a lag phase prior to the cells beginning to double. After the lag phase, the cell doubling time decreases and the culture goes into the logarithmic phase. The logarithmic phase is eventually followed by an increase of the doubling time that, while not intending to be limited by theory, is thought to result from either a depletion of nutrients including nitrogen sources, or a rise in the concentration of inhibitory chemicals, or quorum sensing by the microbes. The growth slows down and then ceases when the culture goes into the stationary phase. In order to harvest cell mass with high lipid content, the culture is generally harvested late in the logarithmic phase or in the stationary phase. In some embodiments, the cells are harvested in logarithmic phase. In some embodiments, the cells are harvested in stationary phase. The accumulation of lipid can generally be triggered by the depletion of the nitrogen source or another key nutrient excepting the carbon or the energy source (e.g. hydrogen). This signals the cells to store lipids produced from the excess carbon and energy sources. Optimization of lipid production and the targeting of specific lipid distributions can be achieved by control of bioreactor conditions and/or nutrient levels and/or through genetic modifications of the cells. In some embodiments the lipid production and distribution of lipid molecules produced is optimized through one or more of the following: control of bioreactor conditions, control of nutrient levels, genetic modifications of the cells.

The synthesis of lipids by the microbes disclosed in the present invention can happen during the logarithmic phase and afterwards during the stationary phase when cell doubling has stopped provided there is an ample supply of carbon and energy sources,

In some embodiments, microorganisms grown using conditions described herein and known in the art comprise at least 20% lipid content by weight, but under chemotrophic conditions, comprise at least 10% lipid content by weight. In some embodiments, under chemotrophic conditions, the microorganisms of the present invention comprise at least about 10, 15, 20, 25, 30, 35, or 40% by weight of lipids, at least about 50% by weight, or at least about 60% by weight of lipids. Improved lipid yield and/or lower production costs can be achieved by controlling process parameters. In certain embodiments, a bacterium is grown in a nutrient media and/or gas mix having a nitrogen, oxygen, phosphorous, or sulfur limitation, while a gaseous carbon and energy source such as syngas is provided in excess. Lipid yield is generally higher in microbial cultures grown with a nitrogen limitation versus microbial cultures grown without nitrogen limitation. In certain embodiments, lipid yield rises by at least: 10%, 50%, 100%, 200%, 500%, or 1000%. The microbial growth can occur with nutrient limitation for a part or for all of the fermentation run. Feeding an excess of energy and carbon source to a population of oleaginous microbes, but little or no nitrogen, can produce a rise in cellular lipid content. In some embodiments, microbial growth occurs on limited amounts of nitrogen or in the complete absence of nitrogen.

Genes are well known in the art that code for cofactors useful in the present invention, or that are involved in synthesizing such cofactors.

In another embodiment, genes that code for cofactors useful in the present invention, or that are involved in synthesizing such cofactors, are put in oleaginous bacteria, using the constructs and methods such as described above. Lipid yield is improved in another embodiment by growing an oleaginous bacteria with one or more lipid pathway enzyme cofactor(s) added to the culture environment. The lipid yield is generally improved in the presence of a certain concentration of the cofactor(s) compared to lipid yield without supplemental cofactor(s). In some embodiments, the cofactor(s) are delivered to the culture by having a microbe (e.g., bacteria) present in the culture that contains an exogenous gene coding for the cofactor(s) at a concentration sufficient to increase lipid yield as compared to the lipid yield of the microbe in the absence of the cofactor. Cofactor(s) may also be delivered to a culture by having a microbe (e.g., bacteria) present in the culture that contains an exogenous gene that coding for a protein involved in the cofactor synthesis. In some embodiments, any vitamin needed for the proper function of a lipid pathway enzyme including biotin and/or pantothenate is included in the culture environment.

The specific examples of bioreactors, culture conditions, heterotrophic and chemotrophic growth, maintenance, and lipid production methods described herein can be combined in any suitable manner to improve efficiencies of microbial growth and lipid and/or protein production.

In another aspect of the invention, the invention relates to a method of producing a molecule or mixture of molecules in a microorganism population comprising the cell or the composition described herein, wherein the method comprises: culturing a population of microorganisms comprising the cell or the composition described herein in a feedstock comprising syngas and/or gaseous CO₂ and/or a mixture of CO₂ gas and H₂ gas.

In another aspect of the invention, the invention relates to a method of producing a hydrocarbon or mixture of hydrocarbons in a microorganism population comprising the cell or the composition described herein, wherein the method comprises: culturing a population of microorganisms comprising the cell or the composition described herein in a feedstock comprising syngas and/or gaseous CO₂ and/or a mixture of CO₂ gas and H₂ gas.

In another aspect of the invention, the invention relates to a method of producing a lipid or mixture of lipids in a microorganism population comprising the cell or the composition described herein, wherein the method comprises: culturing a population of microorganisms comprising the cell or the composition described herein in a feedstock comprising syngas and/or gaseous CO₂ and/or a mixture of CO₂ gas and H₂ gas.

In another aspect of the invention, the invention relates to a method of producing an alkane or mixture of alkanes in a microorganism population comprising the cell or the composition described herein, wherein the method comprises: culturing a population of microorganisms comprising the cell or the composition described herein in a feedstock comprising syngas and/or gaseous CO₂ and/or a mixture of CO₂ gas and H₂ gas.

In another aspect of the invention, the invention relates to a method of producing an alkene or mixture of alkenes in a microorganism population comprising the cell or the composition described herein, wherein the method comprises: culturing a population of microorganisms comprising the cell or the composition described herein in a feedstock comprising syngas and/or gaseous CO₂ and/or a mixture of CO₂ gas and H₂ gas.

In another aspect of the invention, the invention relates to a method of producing an alkyne or mixture of alkynes in a microorganism population comprising the cell or the composition described herein, wherein the method comprises: culturing a population of microorganisms comprising the cell or the composition described herein in a feedstock comprising syngas and/or gaseous CO₂ and/or a mixture of CO₂ gas and H₂ gas.

In some embodiments, the methods of the claimed invention do not rely on desulfonation to produce and/or secrete one or more hydrocarbons. In some embodiments, an exogenous nucleic acid is introduced into the cells of the claimed invention to silence or disrupt transcription of endogenous genes of the cell that encode enzymes capable of desulfonation of commercial surfactants under conditions and for a time period sufficient for growth of the cell with a gaseous feedstock comprising a gas comprising carbon.

In another aspect of the invention, the invention relates to a method of producing a primary alcohol in a microorganism population comprising the cell or the composition described herein, wherein the method comprises: culturing a population of microorganisms comprising the cell or the composition described herein in a feedstock comprising syngas and/or gaseous CO₂ and/or a mixture of CO₂ gas and H₂ gas. In some embodiments, the bacterial cell comprises a first and second exogenous nucleic acid sequence, wherein the first nucleic acid sequence encodes a FAR or functional fragment thereof and the second exogenous nucleic acid encodes a fatty-acyl-CoA ligase or functional fragment thereof.

In some embodiments, the feedstock does not include linoleic acid.

In addition to providing the new genes for post-production fatty acid hydroxylation, in order to boost yields of the desired hydroxylated products, one can increase the production of the C18 fatty acid precursors. Several ways have been identified to accomplish this: (1) upregulate the thioesterase gene responsible for production of C18 fatty acids; (2) downregulate production of endogenous thioesterases for other fatty acid chain lengths; and/or (3) down regulation of endogenous acyl carrier proteins.

Aspects of this invention involve the expression of fatty acyl-CoA binding protein in chemoautotrophic microbes for modification of the fatty acid profile. The fatty acyl-CoA binding protein exhibit broad specificity and sequester fatty acyl-CoA esters from the synthesizing machinery resulting in the production of shorter chain fatty acids.

Mikkelsen et al. identified a fatty acyl-CoA-binding protein (ACBP) with an apparent Mr of 7000 (Mogensen et al., 1987). This protein could bind and thereby induce medium-chain fatty acyl-CoA synthesis by goat mammary-gland fatty acid synthetase in vitro. “(Mikkelsen 1987)

In some embodiments, the production strain is in the genera Rhodococcus or Gordonia or Nocardia. In some embodiments, the production strain is Rhodococcus opacus. In some embodiments, the composition comprises a microorganism, wherein the microorganism is Rhodococcus opacus (DSM 43205) or Rhodococcus opacus (DSM 43206) or Rhodococcus opacus (DSM 44193). In some embodiments the production strain is Cupriavidus necator. In some embodiments the production strain is a knallgas microorganism, also known as an oxyhydrogen microorganism. In some embodiments the wild-type of the production strain naturally has a capability for accumulating and/or synthesizing high quantities of triacylglycerol where a high quantity is considered to be 10% or more of the dry cell mass; 20% or more of the dry cell mass; 30% or more of the dry cell mass; 40% or more of the dry cell mass; 50% or more of the dry cell mass; 60% or more of the dry cell mass; 70% or more of the dry cell mass. In some embodiments the production strain is a hydrogen-oxidizing chemoautotroph. In some embodiments the production strain is capable of growing on syngas as the sole energy and carbon source. In some embodiments the production strain is capable of growing on untreated crude glycerol as the sole energy and carbon source. In some embodiments the production strain is capable of growing on methanol as the sole energy and carbon source. In some embodiments the production strain is capable of growing on acetate as the sole energy and carbon sources. In some embodiments process conditions are used to enhance the effect on fatty acid chains lengths of the expressed enzymes. In some embodiments the process condition used to enhance the effect of the expressed enzymes is temperature.

The following documents are incorporated herein by reference in their entirety for all purposes:
U.S. Provisional Patent Application No. 61/616,560, filed Oct. 1, 2012 and entitled “PROCESS FOR GENERATING HYDROXYLATED FATTY ACIDS”; U.S. Provisional Patent Application No. 61/635,238, filed Apr. 18, 2012 and entitled “PROCESS FOR GENERATING SHORTER FATTY ACIDS WITH AN EXOGENOUS FATTY ACYL-COA BINDING PROTEIN”; U.S. Provisional Patent Application No. 61/708,057, filed Oct. 1, 2012 and entitled “PROCESS FOR PRODUCING CARBON-BASED CHEMICALS, INCLUDING BUTANEDIOL, USING CHEMOTROPHIC MICROBES”; U.S. Provisional Patent Application No. 61/542,823, filed Sep. 19, 2011 and entitled “Engineered CO2-Fixing Chemotrophic Microorganisms Producing Carbon-Based Products And Methods Of Using The Same”; International Patent Application Serial No. PCT/US2011/34218, filed May 27, 2011, entitled “Use Of Oxyhydrogen Microorganisms For Non-Photosynthetic Carbon Capture And Conversion Of Inorganic And/Or C1 Carbon Sources Into Useful Organic Compounds”; U.S. Provisional Patent Application No. 61/328,184, filed Apr. 27, 2010 and entitled “USE OF OXYHYDROGEN MICROORGANISMS FOR NON-PHOTOSYNTHETIC CARBON CAPTURE AND CONVERSION OF INORGANIC CARBON SOURCES INTO USEFUL ORGANIC COMPOUNDS”; International Patent Application Serial No. PCT/US2010/001402, filed May 12, 2010, entitled “BIOLOGICAL AND CHEMICAL PROCESS UTILIZING CHEMOAUTOTROPHIC MICROORGNISMS FOR THE CHEMOSYTHETIC FIXATION OF CARBON DIOXIDE AND/OR OTHER INORGANIC CARBON SOURCES INTO ORGANIC COMPOUNDS, AND THE GENERATION OF ADDITIONAL USEFUL PRODUCTS”; and U.S. Patent Application Publication No. 2010/0120104, filed Nov. 6, 2009, entitled “BIOLOGICAL AND CHEMICAL PROCESS UTILIZING CHEMOAUTOTROPHIC MICROORGNISMS FOR THE CHEMOSYTHETIC FIXATION OF CARBON DIOXIDE AND/OR OTHER INORGANIC CARBON SOURCES INTO ORGANIC COMPOUNDS, AND THE GENERATION OF ADDITIONAL USEFUL PRODUCTS.
Doan T T P, Carlsson A S, Hamberg M, Bulow L, Stymne S, Olsson P, Functional expression of five Arabidopsis fatty acyl-CoA reductase genes in Escherichia coli, J Plant Phys 166 (2008):787-96.
Kavanagh K L, Jornvall H, Persson B, Oppermann U, The SDR superfamily: functional and structural diversity within a family of metabolic and regulatory enzymes, Cell Mol Life Sci 65 (2008) 3895-3906.
Labesse G, Vidal-Cros A, Chomilier J, Gaudry M, Mornon J-P, Structural comparisons lead to the definition of a new superfamily of NAD(P)(H)-accepting oxidoreductases: the single-domain reductases/epimerases/dehydrogenases (the ‘RED’ family), Biochem J (1994) 304:95-99.
Benveniste I, Tijet N, Adas F, Phillips G, Salau\ddot{}n JP, Durst F. 1998 Biochem. Biophys. Res. Commun. 243: 688-693.
Cellini F, Cifarelli R A, Carriero F, Ricinus communis-origin gene encoding novel protein interacting with oleate 12-hydroxylase, Patent JP 2002543842-A4 24 Dec. 2002.
Cellini F, Cifarelli R A, Carriero F, Ricinus communis-origin gene encoding novel protein interacting with oleate 12-hydroxylase, Patent WO 0070052-A4 23 Nov. 2000.

Dauk M, Lam P, Kunst L, Smith M A. A FAD2 homologue from Lewquerella lindheimeri has predominantly fatty acid hydroxylase activity, 2007 J Plant Sci 173(1):43-49.
McKeon T A, Chen G Q, He X, Ahn Y-J, Lin J-T, The enzymology of Castor Oil biosynthesis, Eds. Janick J, Whipkey A, “Issues in new crops and new uses, ASHS Press, Alexandria, Va. (2007) 101-104.
Meesapyodsuk D, Qiu X. An oleate hydroxylase from the fungus Claviceps purpurea: cloning, functional analysis, and expression in Arabidopsis. Plant Physiol. 2008 147(3):1325-1333.
Meesapyodsuk D, Qiu X. Fatty acid desaturases and uses thereof. U.S. Pat. No. 8,003,853, Aug. 23, 2011.
Meesapyodsuk D, Qiu X. Fatty acid hydroxylases and uses thereof. U.S. Pat. No. 7,923,598, Apr. 12, 2011.
van de Loo F J, Broun P, Turner S, Somerville C. An oleate 12-hydroxylase from Ricinus communis L. is a fatty acyl desaturase homolog. Proc Natl Acad Sci USA. 1995 Jul. 18; 92(15):6743-7.

The following examples are provided to describe the invention in greater detail. They are intended to illustrate, not to limit, the invention. Various publications, including patents, published applications, technical articles and scholarly articles are cited throughout the specification. Each of these cited publications is incorporated by reference herein, in its entirety.

### EXAMPLES

### Example 1

### Characterization of Organisms Sharing High 16SrRNA Sequence Similarity

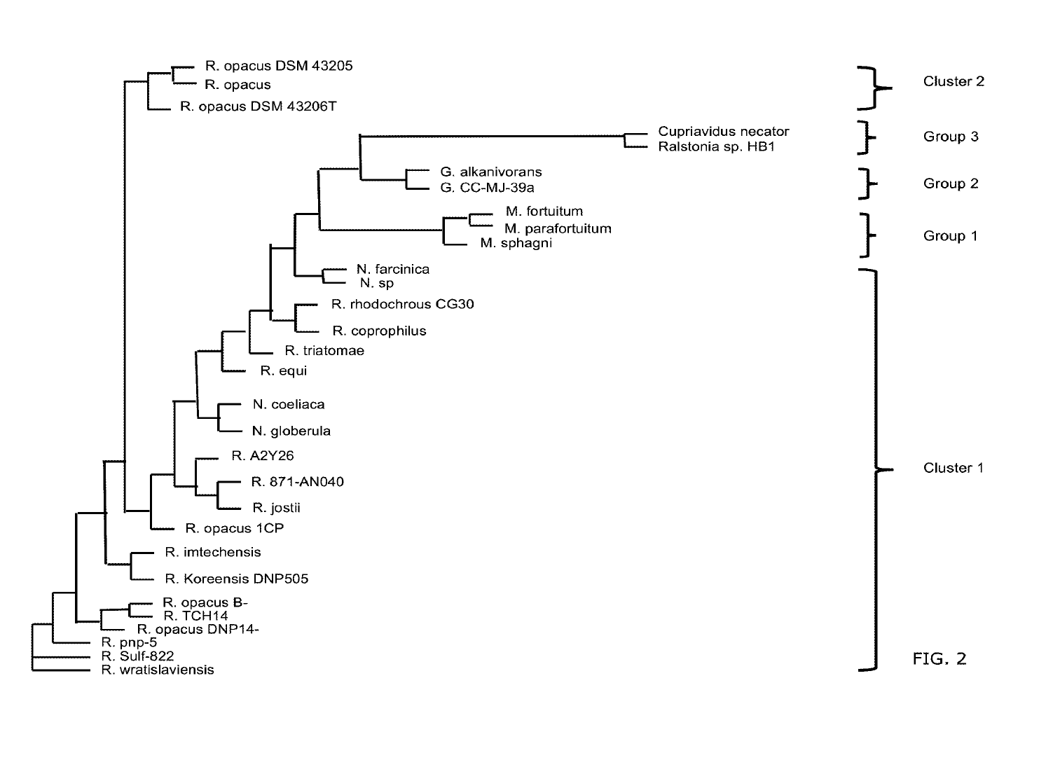

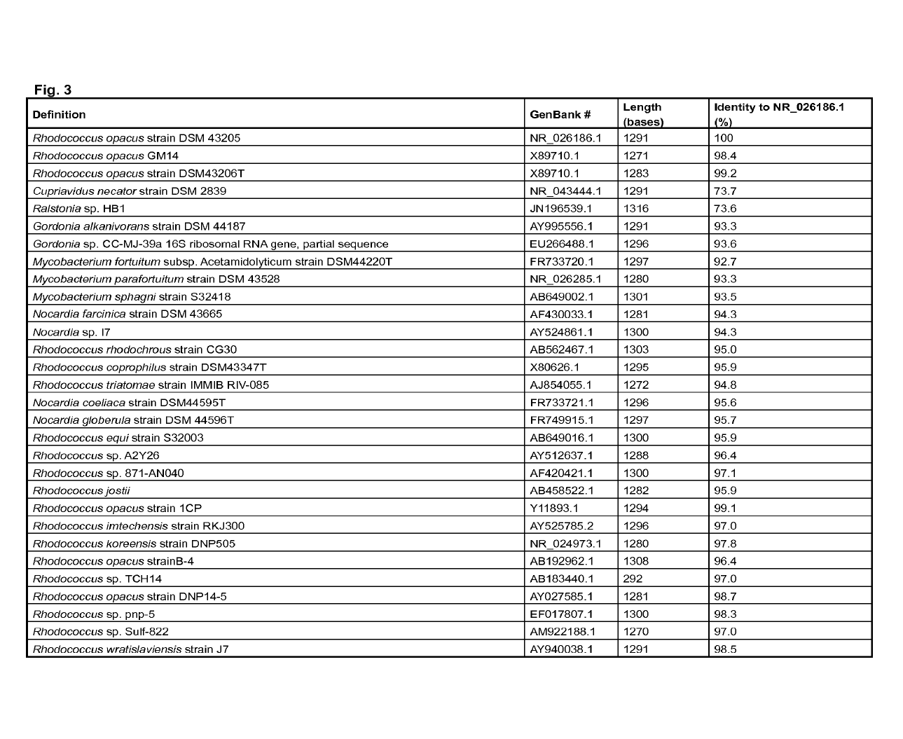

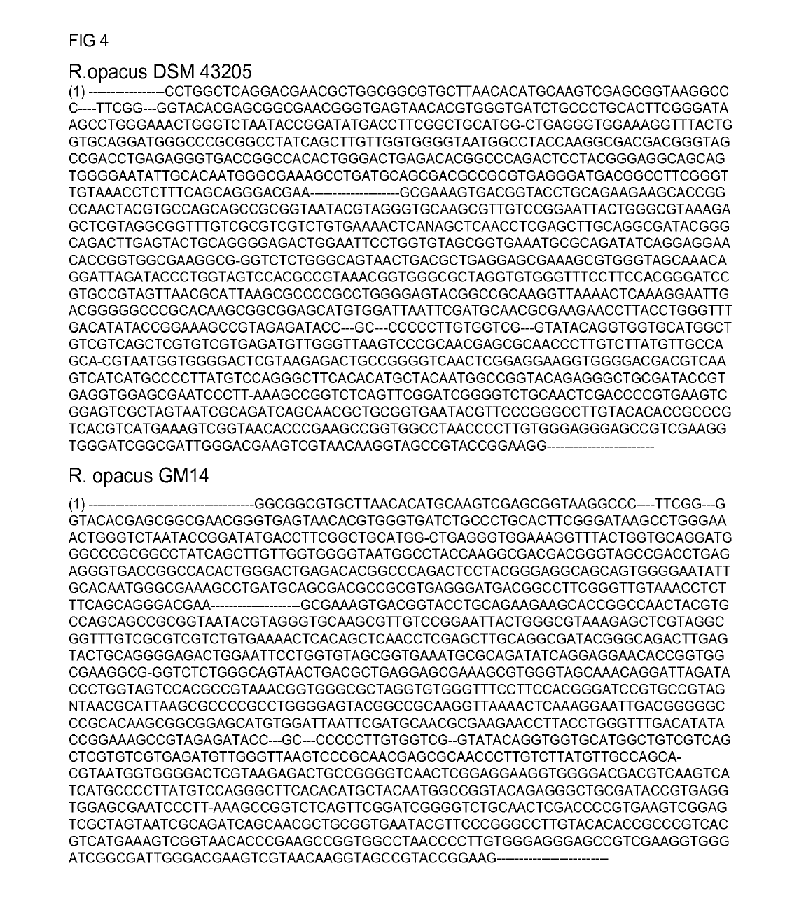

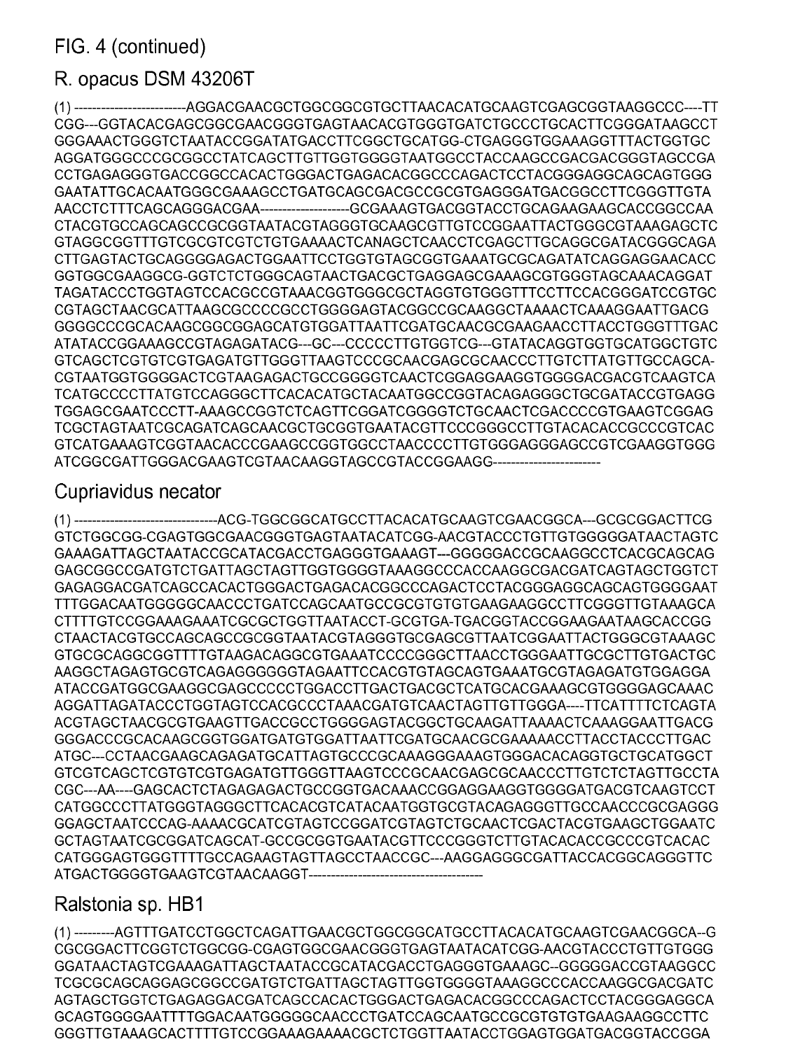

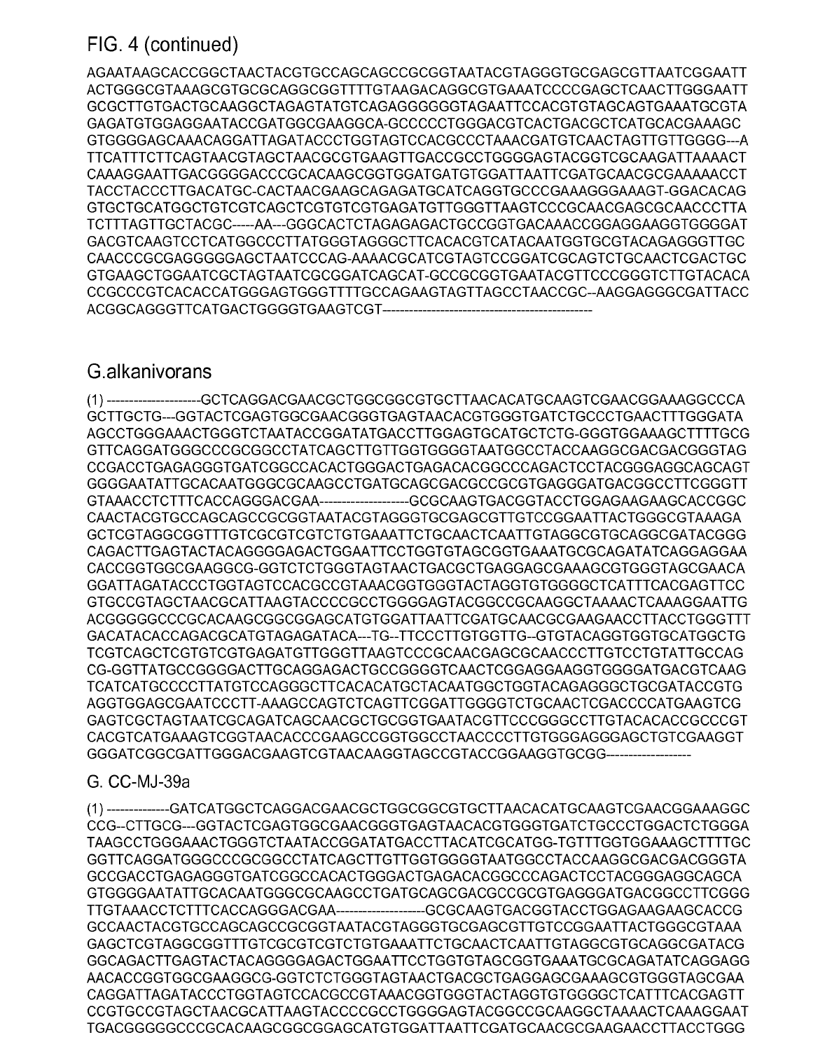

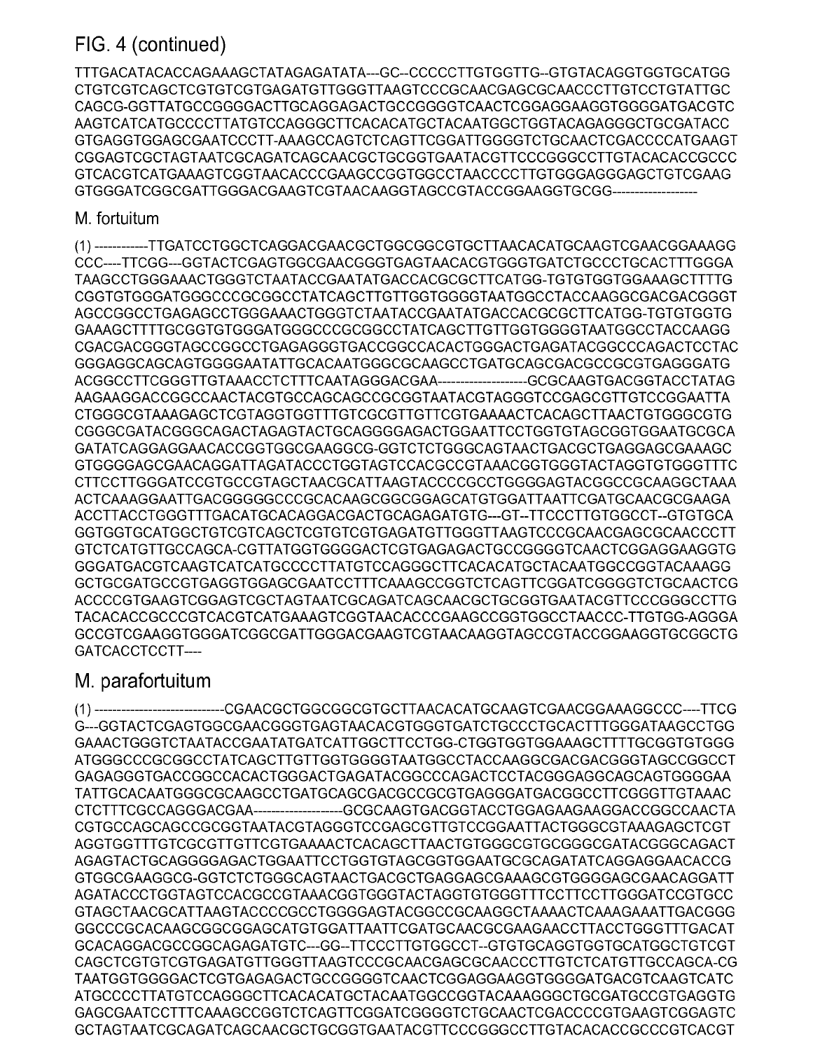

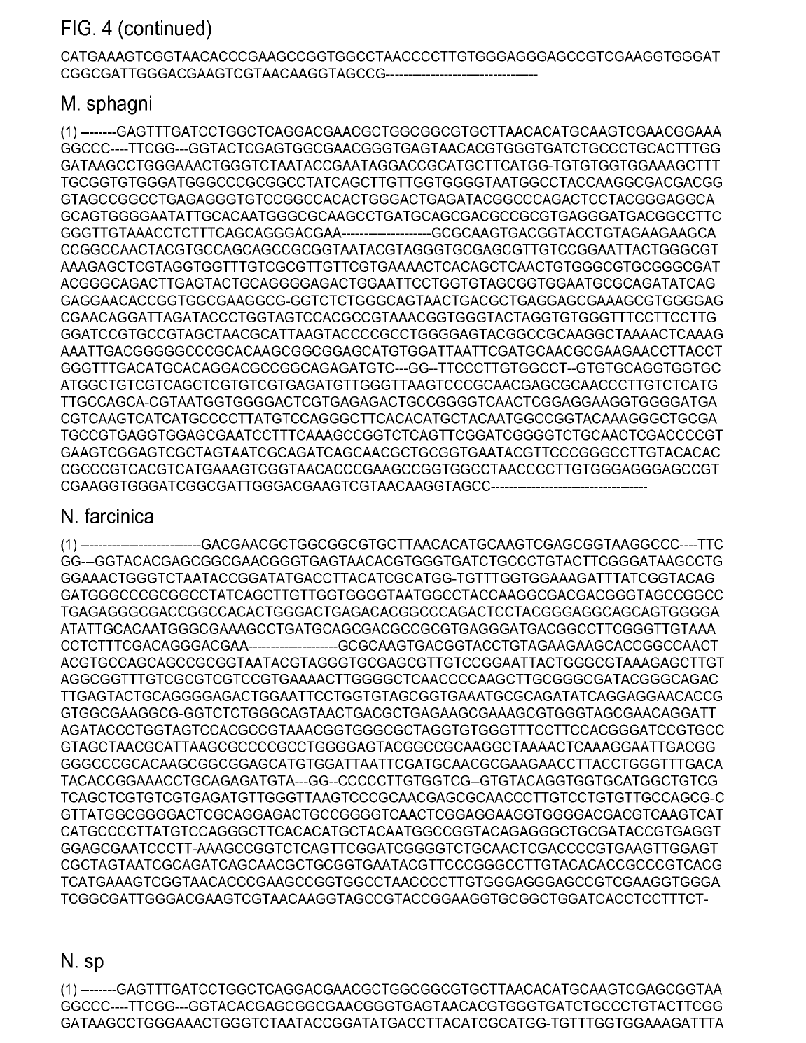

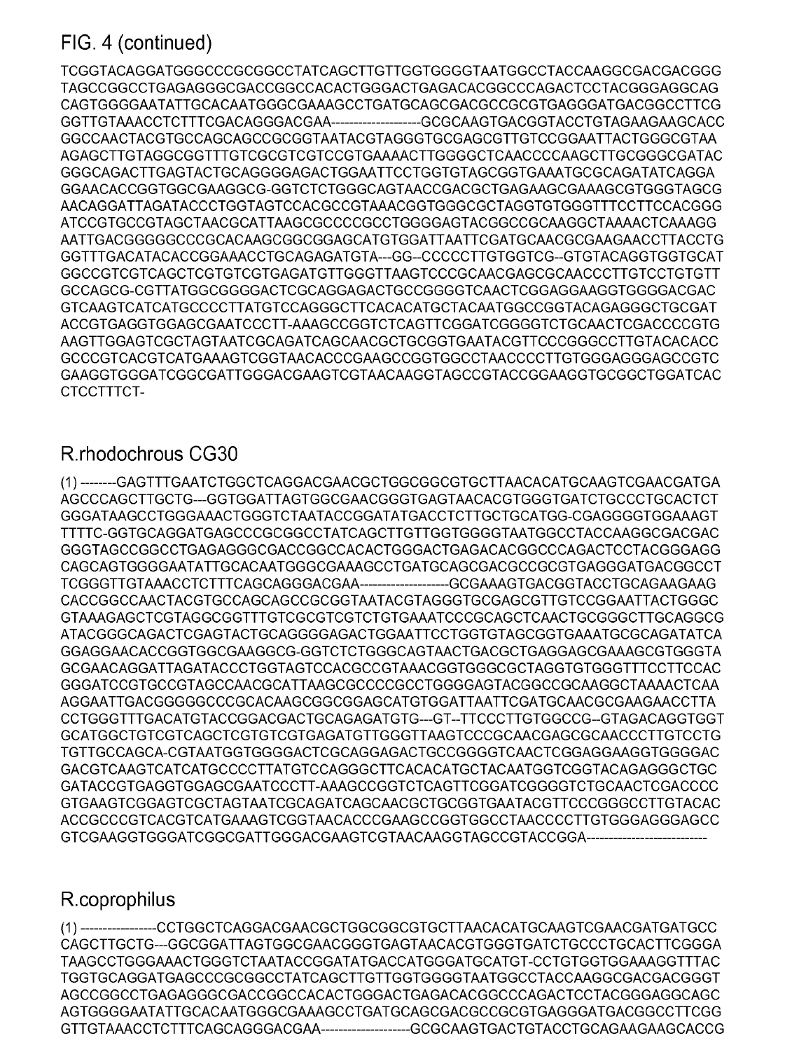

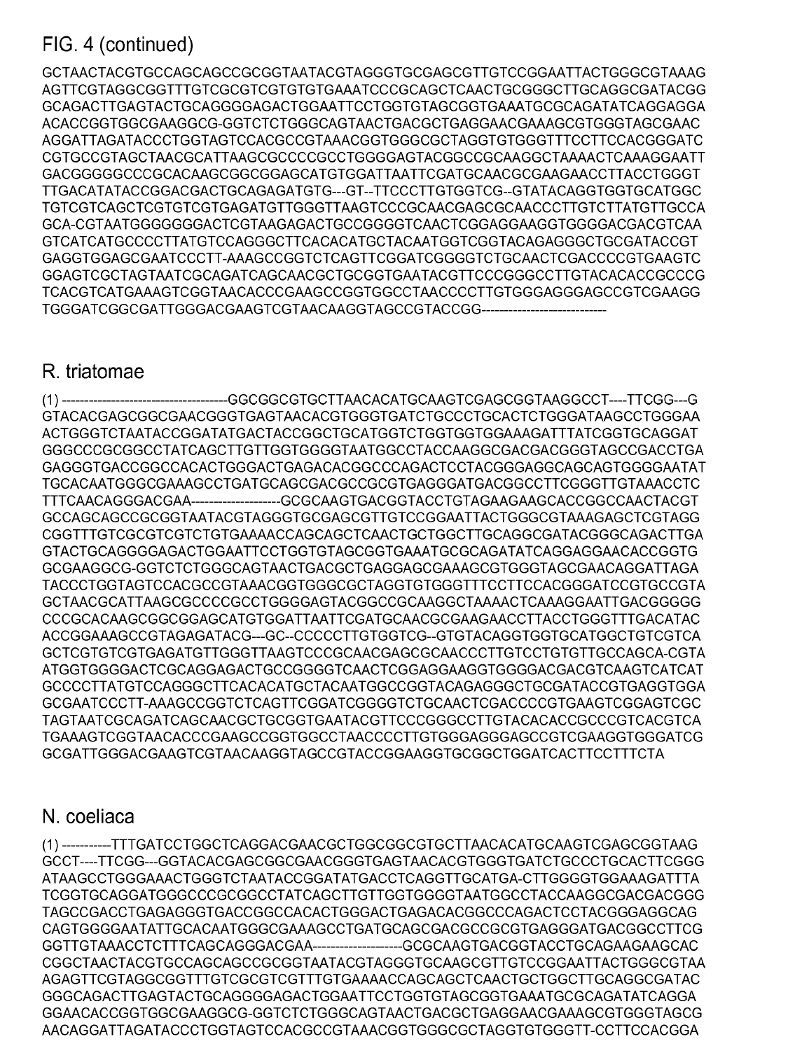

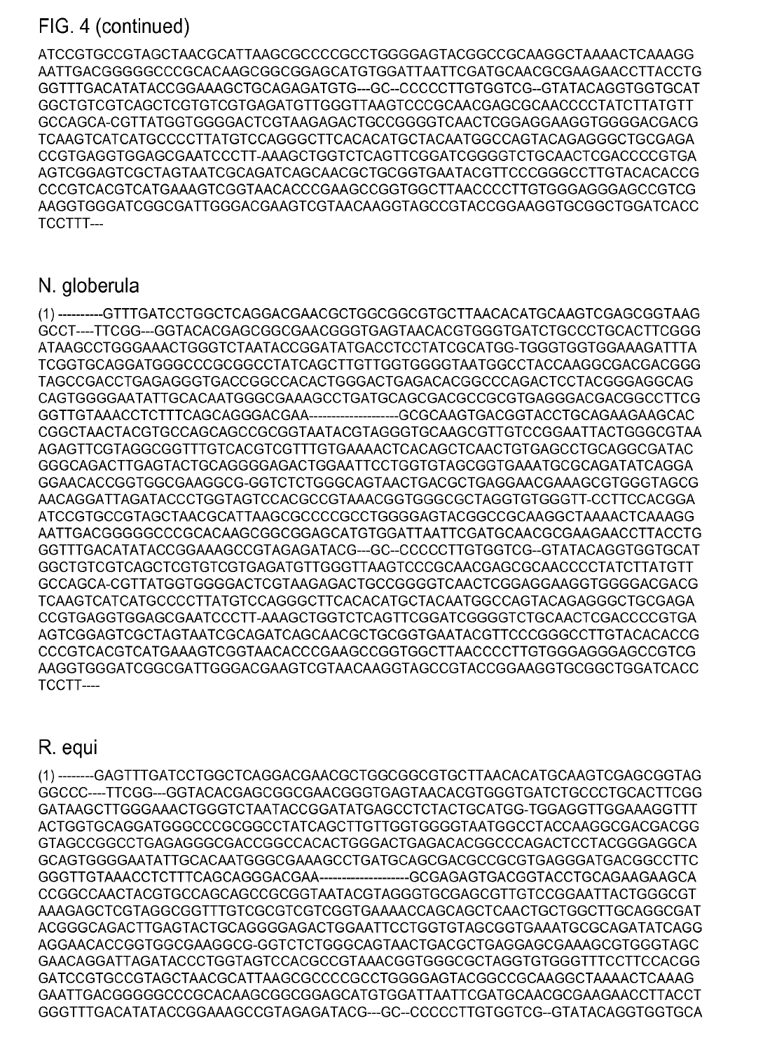

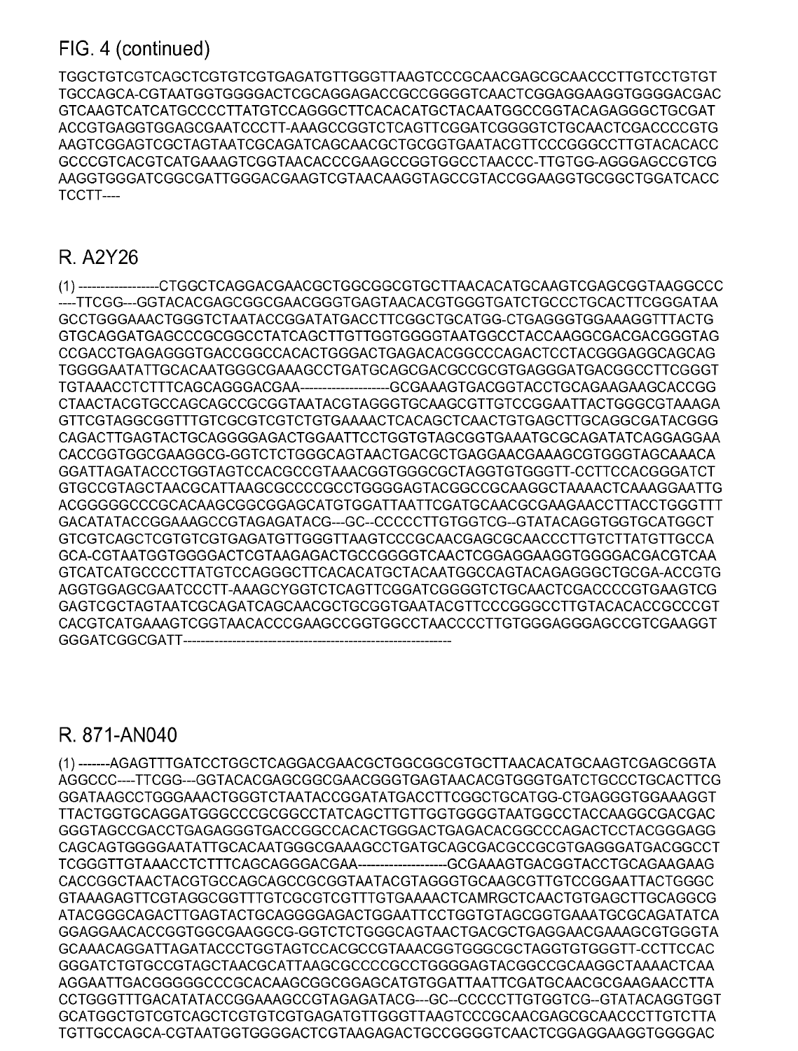

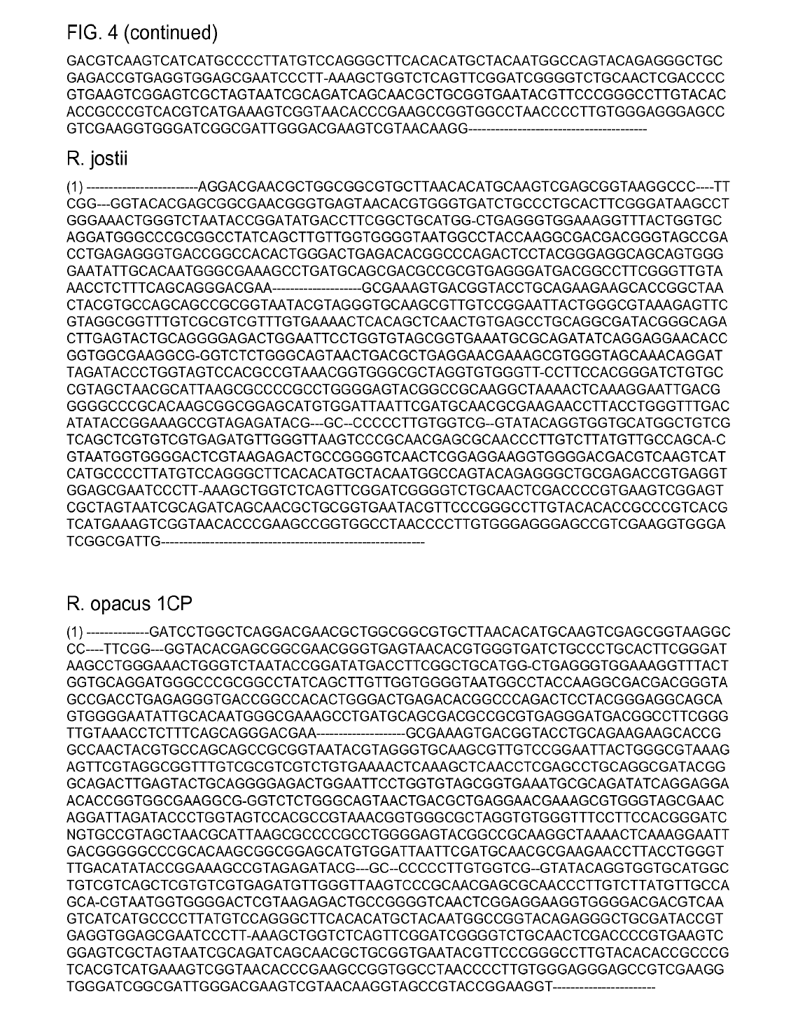

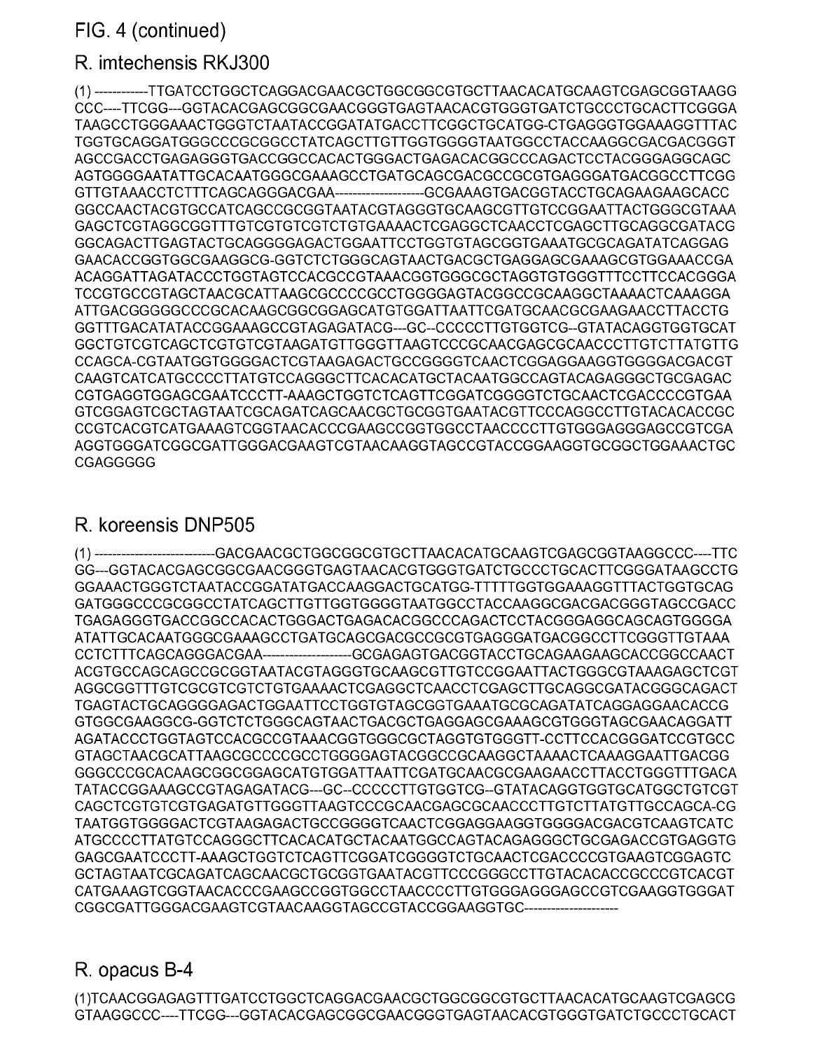

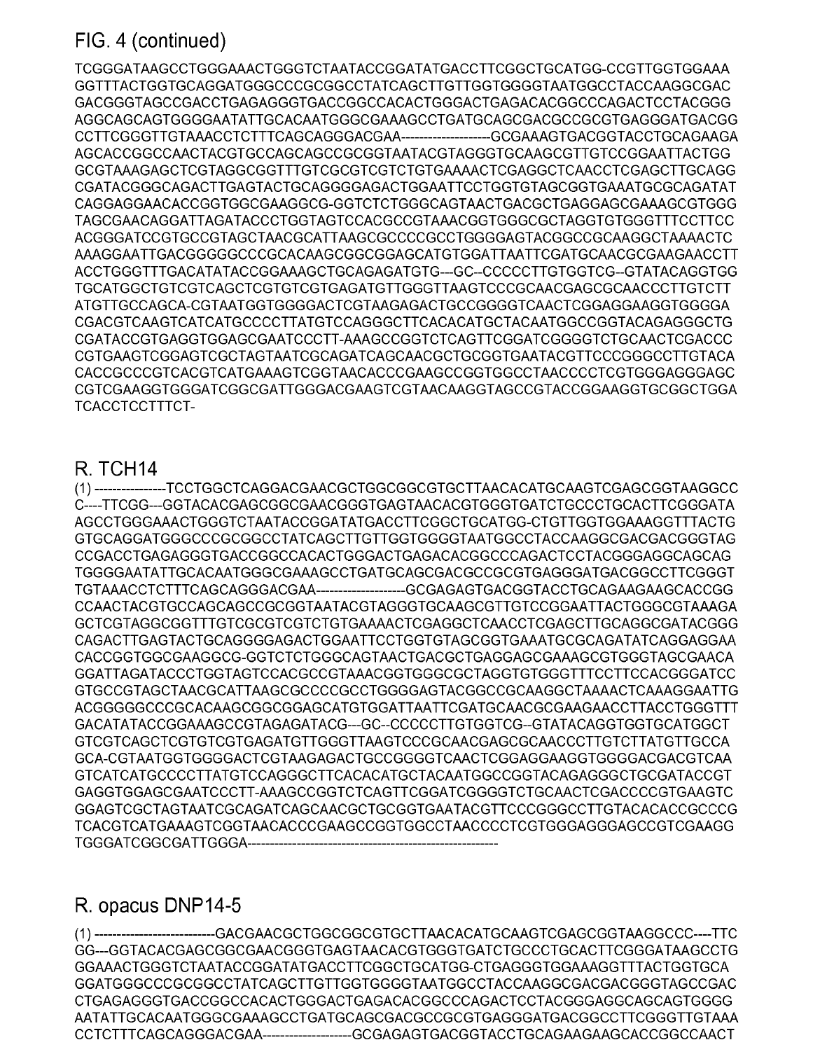

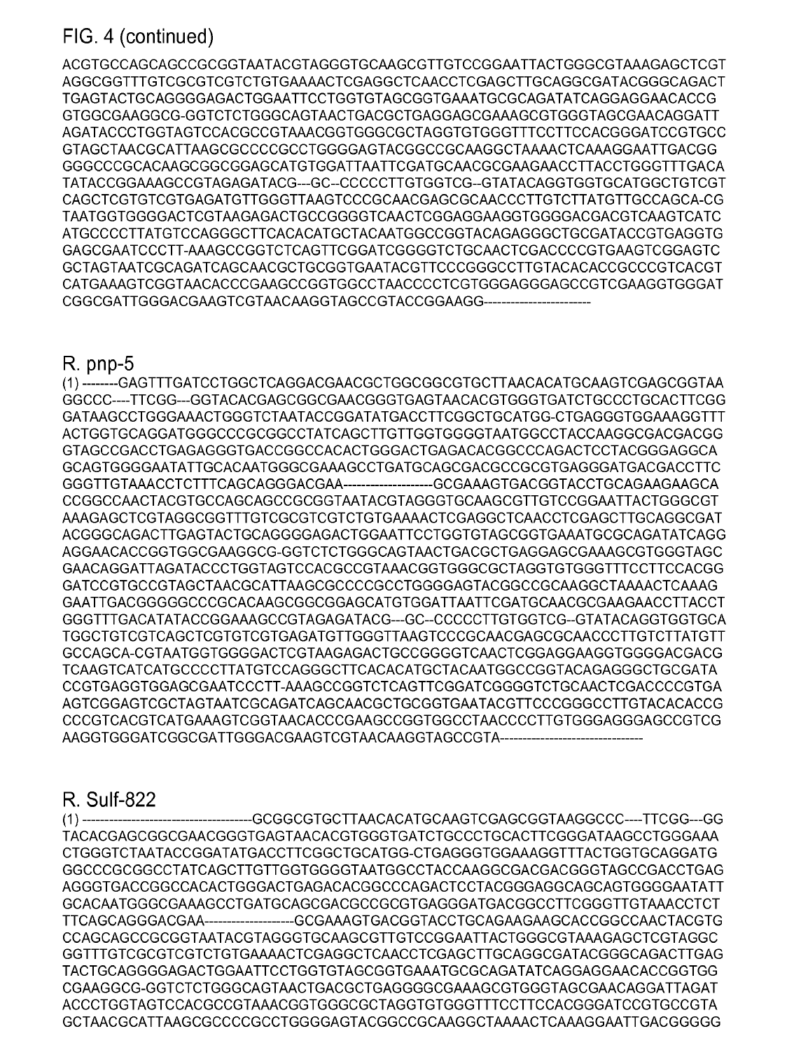

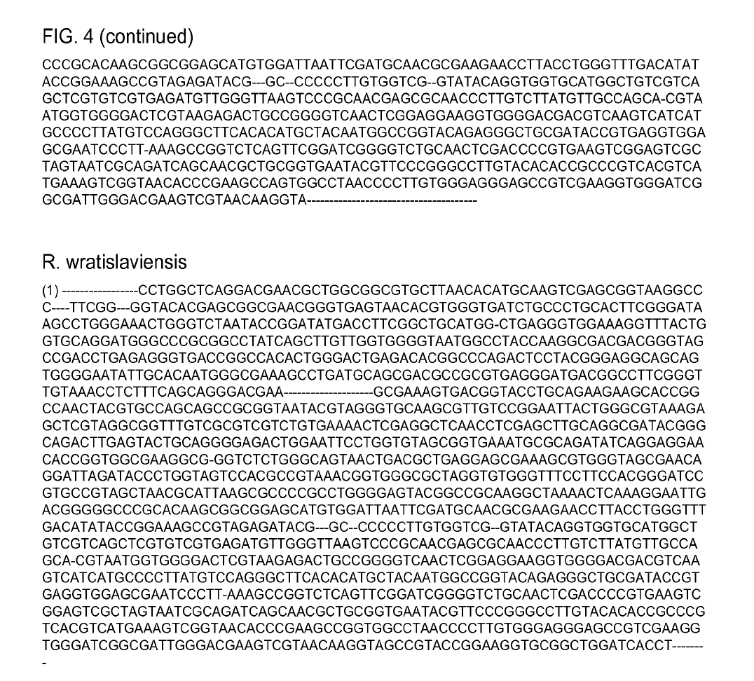

To identify organisms closely related to R. opacus strain (DSM43205), a basic local alignment search (BLAST^{R}) with the BLASTN programs search of nucleotide databases using the 16S rRNA (NR_{—}026186.1) was carried out. The phylogenetic relationships, based on the 16S rRNA gene sequence homology, between the tested strain and the reference strains of the suborder corynebacterineae (corynebacterium, gordoniaceae, mycobacteriaceae and nocardiaceae) and the family burkholderiaceae (genus cupriavidus and ralstonia) are shown in FIG. 2. The nocardiaceae are related and form two clusters of organisms: clusture1 that contains 20 organisms from the genus nocardia and rhodococcus and cluster 2 that contains 3 R. opacus strains (DSM43205, GM14 and DSM43206). The gordoniaceae, mycobacteriaceae and burkholderiaceae form 3 separated groups (1, 2 and 3). The gram positive chemoautotroph lipid accumulating strain R. opacus (DSM43205; NR_{—}026186.1) exhibits high sequence similarity to cluster 1 (94.3-99.1%) and to the gram positive groups 1 and 2 (92.7-93.5% and 93.3-93.6% respectively) (FIGS. 3 and 4). The sequence similarity to the gram negative chemoautotroph poly(3-hydroxybutyrate) (PHB) accumulating strains in group 3 is 73.7%.

### Plasmid Design and Construction

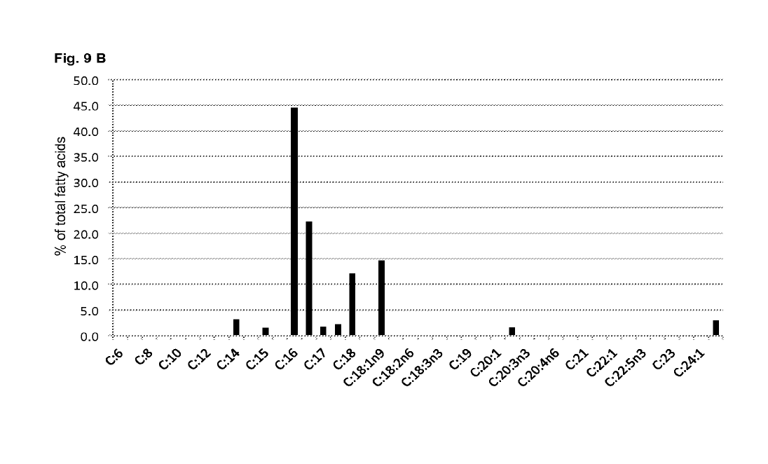

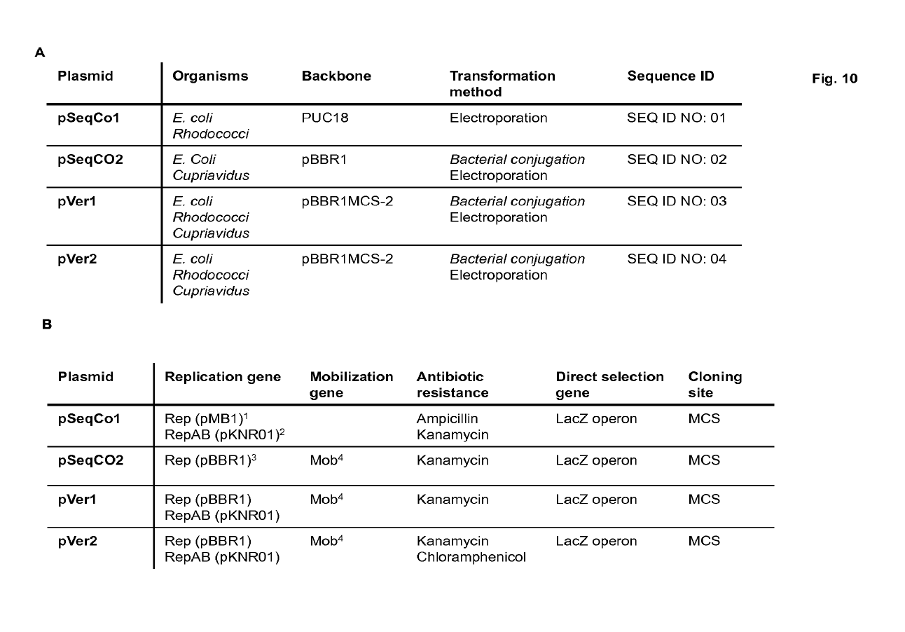

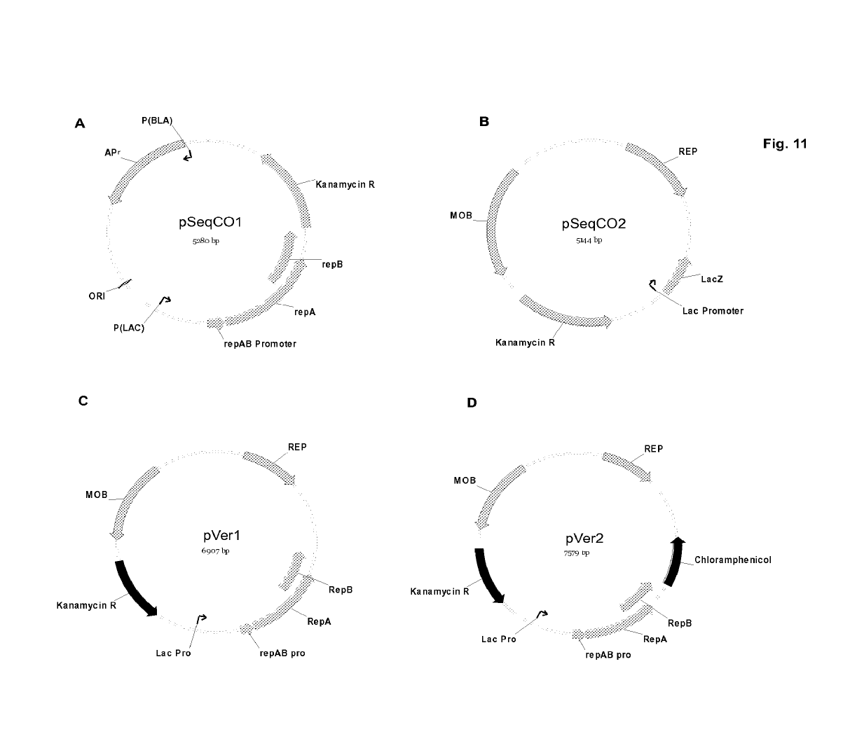

To generate an E. coli Rhodococci shuttle vector suitable for electroporation, the plasmid pSeqCO1 (SEQ ID: 01) was constructed with the genetic elements described in FIG. 10A. pSeqCO1 consists of the replication gene operon, ampicillin and kanamycin resistance genes, LacZ operon and the multiple cloning site as described in FIG. 10B and FIG. 11A. For replication in Rhodococci, the DNA fragment of the repAB operon (1744 bp downsteam from the XhoI restriction site in the native pKNR01 plasmid of the bacteria Rhodococcus opacus B4; Na et al. 2005, J Biosci Bioeng. 99: 408-414) was synthesized with the restriction sites KpnI and SalI and cloned into PUC18 digested with KpnI and SalI. The resultant vector was digested with Spa and BglII and ligated with the PCR product of the Kanamycin resistance gene from pBBR1MCS-2 (Kovach et al. 1995 Gene 166: 175-176) digested with the engineered restriction sites Spa and BglII to give pSeqCO1.

To generate an E. coli-cupriavidus shuttle vector suitable for electroporation and bacterial conjugation, the plasmid pSeqCO2 (SEQ ID: O₂) was used with the genetic elements described in FIG. 10A. pSeqCO2 (SEQ ID: O₂; FIGS. 10 and 11B) is the plasmid pBBR1MCS-2 described in Kovach et al. (1995 Gene 166: 175-176) that contains the IncQ like replication gene, Mob gene that mobilized when the RK2 transfer functions are provided in trans, kanamycin resistance gene, LacZ operon and the multiple cloning site as described in FIG. 10B and FIG. 11B.

Pver1 (SEQ ID: 03; FIGS. 10 and 11C) is an E. coli-cupriavidus-Rhodococci shuttle vector suitable for electroporation and bacterial conjugation. The plasmid was generated by cloning the repAB operon (described in pSeqCO1) into pSeqCO2 using the KpnI and SalI restriction sites.

Pver2 (SEQ ID: 04; FIGS. 10 and 11D) is an E. coli-cupriavidus-Rhodococci shuttle vector suitable for electroporation and bacterial conjugation. The plasmid was generated by cloning the synthesized chloramphenicol gene (Alton and Vapnek Nature 1979 282: 864-869) with the engineered restriction sites SalI and HindIII into Pver1.

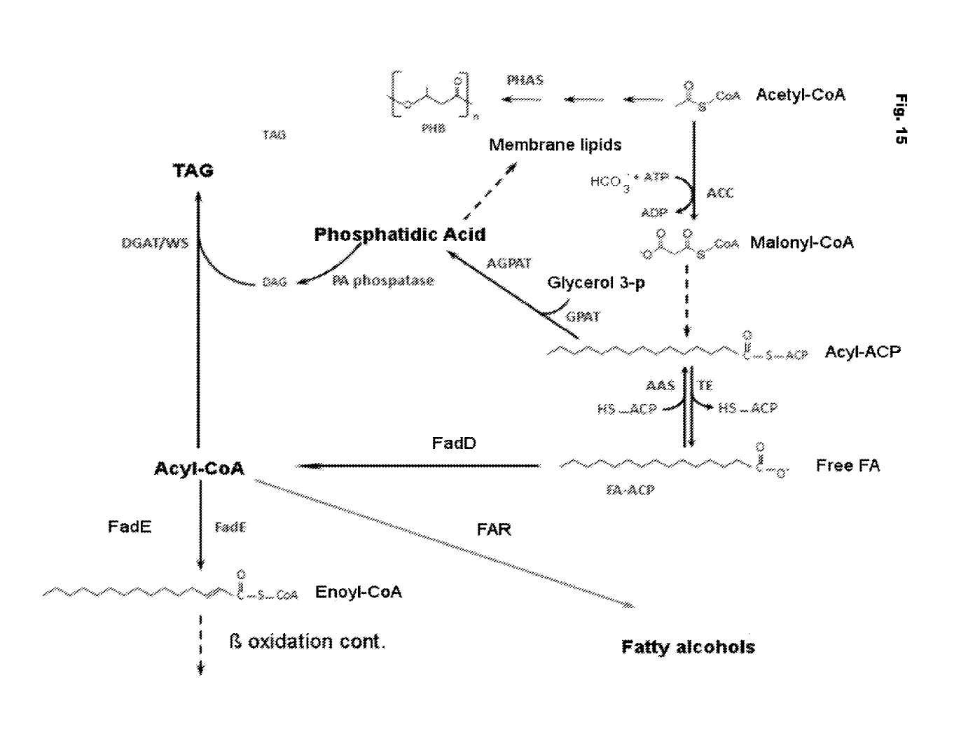

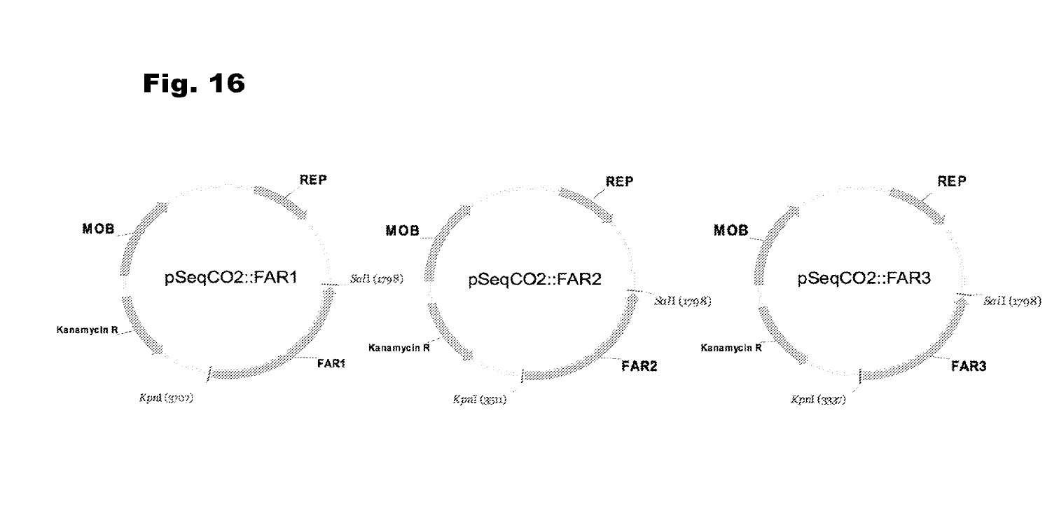

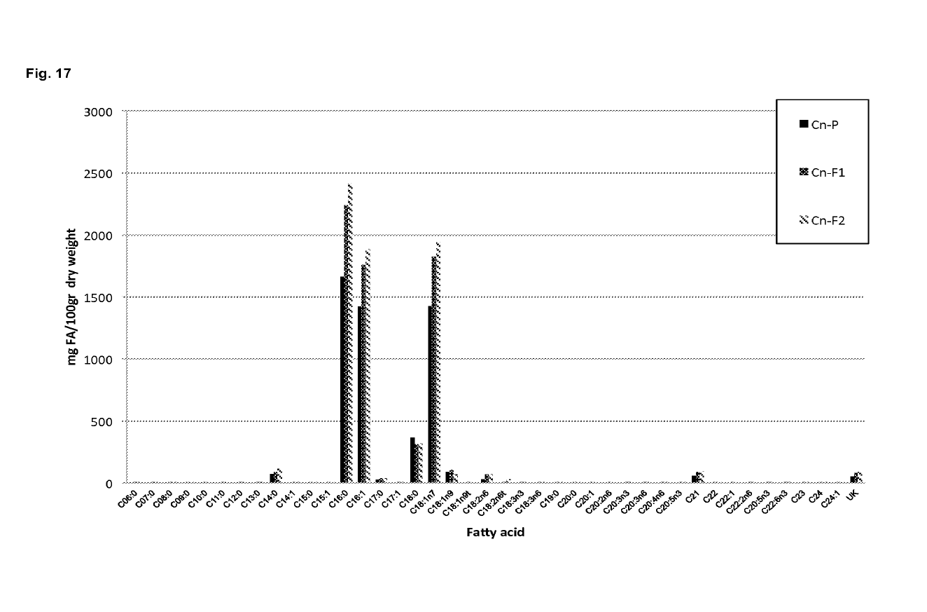

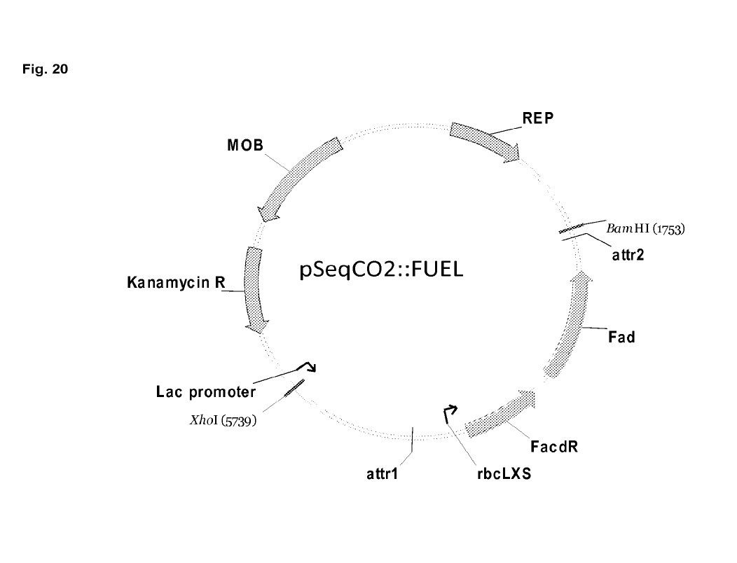

The arabidopsis genes FAR1 (SEQ ID: 05), FAR2 (SEQ ID: 06) and FAR3 (SEQ ID: 07): were synthesized and cloned into the plasmid pUC57. FAR1, FAR2 and FAR3 were rescued from PUC57 using the restriction enzymes KpnI and SalI and cloned into pSeqCO2 digested with KpnI and SalI to give pSeqCO2::FAR1, pSeqCO2::FAR2 and pSeqCO2::FAR3 respectively (FIG. 16). The genes FadDR (SEQ ID: 08) and Fad (SEQ ID: 09) and the rbcLXS promoter (SEQ ID: 10) were PCR amplified from the cyanobacterium Synechocystis sp. PCC 6803 genome and cloned into gateway plasmid to give pFUEL. A 4 kBp XhoI BamHI fragment that contains FadDR, Fad and rbcLXS was rescued from pFUEL and cloned into pSeqCO2 digested XhoI BamHI with to give pSeqCO2::FUEL (FIG. 20).

### Microorganism Mutagenesis and Screening for High Lipid Content

Rhodococcus sp. (DSM3346) was incubated for 2 days in LB medium (per 1 L: 10 g Bacto-tryptone, 5 g yeast extract, 10 g NaCl pH=7.0) at 30° C., 200 rpm, and approximately 7.2×10⁶ CFU (20 μl from O.D=1.2) were spread onto fresh LB plates. Two plates were immediately exposed to short-wave (254-nm) UV light for 0 (control), 5, 10 and 20 sec at a distance of 3.5 cm. Plates were then incubated at 30° C. for 48 h. Colonies from plates were collected in 1.5 ml eppendorf tubes by adding 1 ml LB into the plate and gentle scraping. The mutated colonies were spun down (10,000 rpm, 5 min at room temperature) and washed twice in PBS. Six μl of dilute Nile red DMSO stock solution (0.5 mg/ml) was added to final concentration of 0.75 μg/ml and incubated for 30 min at 4° C. Colonies were washed twice (10,000 rpm, 5 min at RT) with PBS (137 mM NaCl, 2.7 mM KCl, 4.3 mM Na₂HPO₄, 1.47 mM KH₂PO₄; pH of 7.4) and the final concentration was detected by O.D.₆₆₀. The Final colonies concentration for FACS analysis was set to approximately 1×10⁸ CFU/ml. For negative control (no NR), colonies from 0 sec treatment (control) were washed twice in PBS, incubated for 30 min at 4° C. and washed twice again. Analysis was carried out immediately after the staining by Fluorescence-activated cell sorting (FACS) (BD FACSAria™ II cell sorter). Fluorescence was detected with an excitation wavelength of 530 nm and an emission wavelength of 575 nm.

FIG. 27 shows the fluorescence intensity of Rhodococcus Sp exposed to 0, 5, 10, and 20 sec of UV light (B, C, D and E respectively). Exposure for 5 sec (FIG. 27C) increased the population that contains high lipid compared to the control (27B) while exposure for 10 and 20 second negatively affected the lipid content (FIG. 27D and FIG. 27E respectively). FACS analysis of untreated cells (negative control; no Nile Red staining and no UV exposure) indicated that Rhodococcus Sp autofluorescence does not overlap with Nile Red staining.

As shown in FIG. 27G, 100,000 mutants of Rhodococcus Sp with increased lipid content (100% to 115%) from 5 sec UV mutagenesis treatment (P3; purple) were selected by comparison to the untreated population (P2; orange). Negative control (no Nile Red staining and no UV exposure) is indicated in green.

### Microorganism Transformation

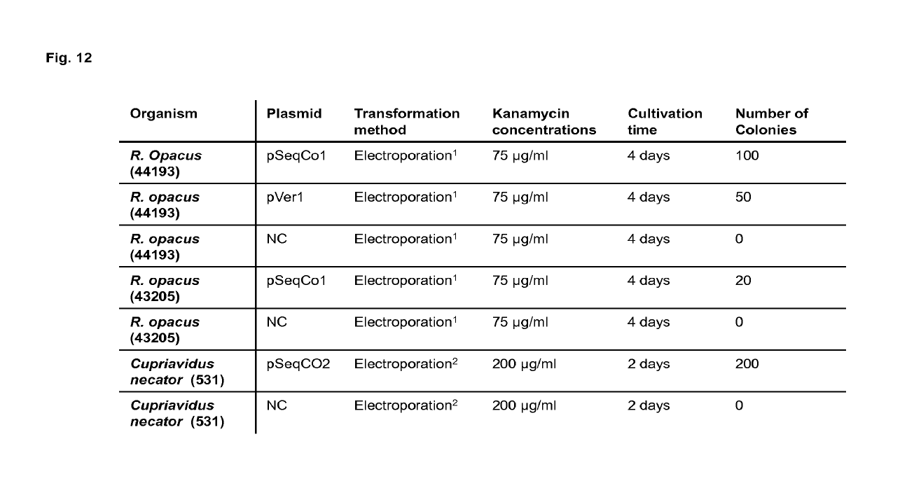

Transformation of Rhodococci was carried out using the plasmids pSeqCO1 and pVer1 (FIG. 12) as described below.

Rhodococci competent cells were prepared by incubating a single colony 2 ml NB medium (5 g/L peptone, 1 g/L meat extract, 2 g/L yeast extract, 5 g/L NaCl; pH=7.0±0.2) at 30° C. overnight. One ml was inoculated to 50 ml NB medium supplemented with 0.85% (w/v) glycine and 1% (w/v) sucrose in a 250 ml baffled Erlenmeyer Flask and incubated to a cell density of O.D₆₀₀=0.5. Cells were collected by centrifugation at 3,000×g for 10 min at 4° C. and washed 3 times with 50 ml (each) of sterile ice-cold double distilled water (ddH₂O). Cells were concentrated 20-fold by re-suspending the collected cells in 2.5 ml of ddH₂O and 4000 aliquots stored in 1.5 ml tube at −70° C. Electroporation was carried out by thawing the competent cells on ice and mixing with the plasmid DNA (final concentration 0.1-0.25 μg/ml). The competent cells and plasmid DNA mixture was incubated at 40° C. for 5 min, transferred into 0.2 cm width and electroporated using a single-pulse electroporation (10 kV/cm, 600Ω, 25 μF and 3-5 ms pulse time). The pulsed cells were regenerated at 30° C. for 4 h (DSM 44193) and 6 h (DSM 43205) in the presence of 600 μl NB. Transformants were selected after cultivation for 3-4 days at 30° C. on NB-agar plate containing kanamycin (75 μg/ml). As shown in FIG. 12, the plasmids pSeqCO1 and pVer1 confer resistance to kanamycin (75 μg/ml) in transformed R. opacus strains (44193 and 43205). Untransformed R. opacus strains (44193 and 43205) (NC) were sensitive to the concentration described above.

Transformation of genus cupriavidus was carried out using the plasmids pSeqCO2 (FIG. 12) as described below.

Cupriavidus necator (DSM531) competent cells were prepared by incubating a single colony in 5 ml NR medium (10 g/l polypeptone, 10 g/l yeast extract, 5 g/l beef extract and 5 g/l ammonium sulfate; pH 7.0) at 30° C. overnight. The pre-culture was inoculated into 100 ml of fresh NR medium and incubated to a cell density of O.D₆₀₀=0.8. Cells were collected by centrifugation at 3,000×g for 10 min at 4° C. and washed 3 times with 50 ml (each) of sterile ice-cold ddH₂O. The collected cells were re-suspended in 400 μl of 10% (v/v) sterile glycerol in sterile ice-cold ddH₂O and stored in 500 aliquots at −70° C.

For electroporation, the competent cells were thawed on ice, transferred into 0.2 cm width of ice cold cuvette and gently mixed with 1 μg of plasmid DNA. Cells were electroporated using a single-pulse electroporation (11.5 kV/cm, 25 μF and 5 ms pulse time). The pulsed cells were transferred into 1 ml of fresh NR medium and culture for 2 h at 30° C. Transformants were selected after cultivation for 48 h at 30° C. on NR-agar plate containing kanamycin (200 μg/ml). As shown in FIG. 12, the plasmid pSeqCO2 confers resistance to kanamycin (200 μg/ml) in transformed Cupriavidus necator (DSM531). Untransformed Cupriavidus necator (DSM531) cells (NC) were sensitive to the concentration described above.

### Inoculation and Growth Conditions

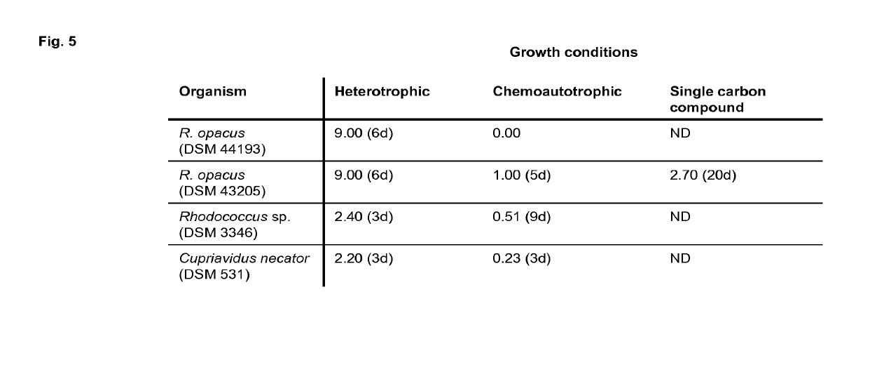

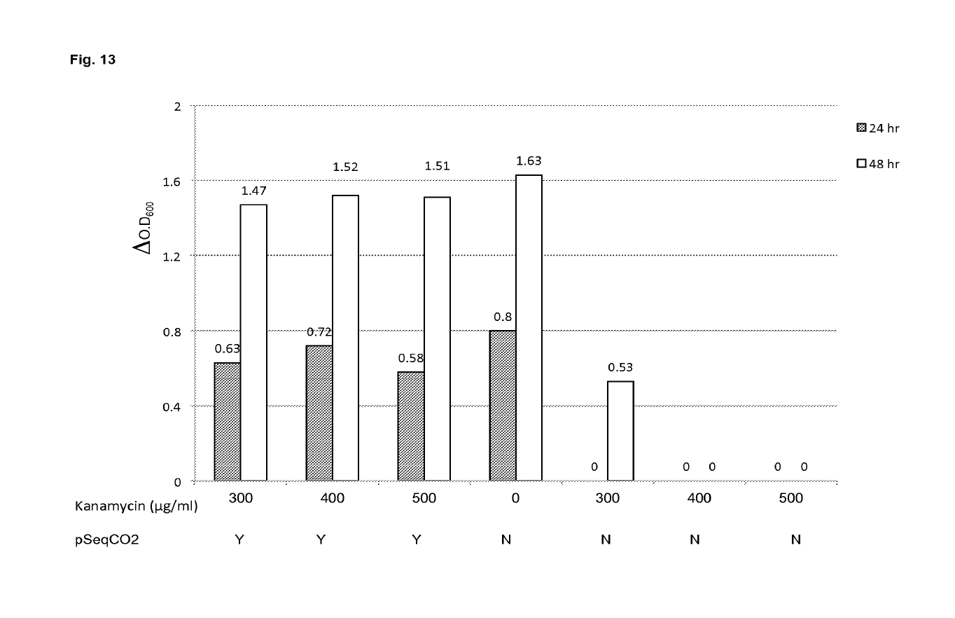

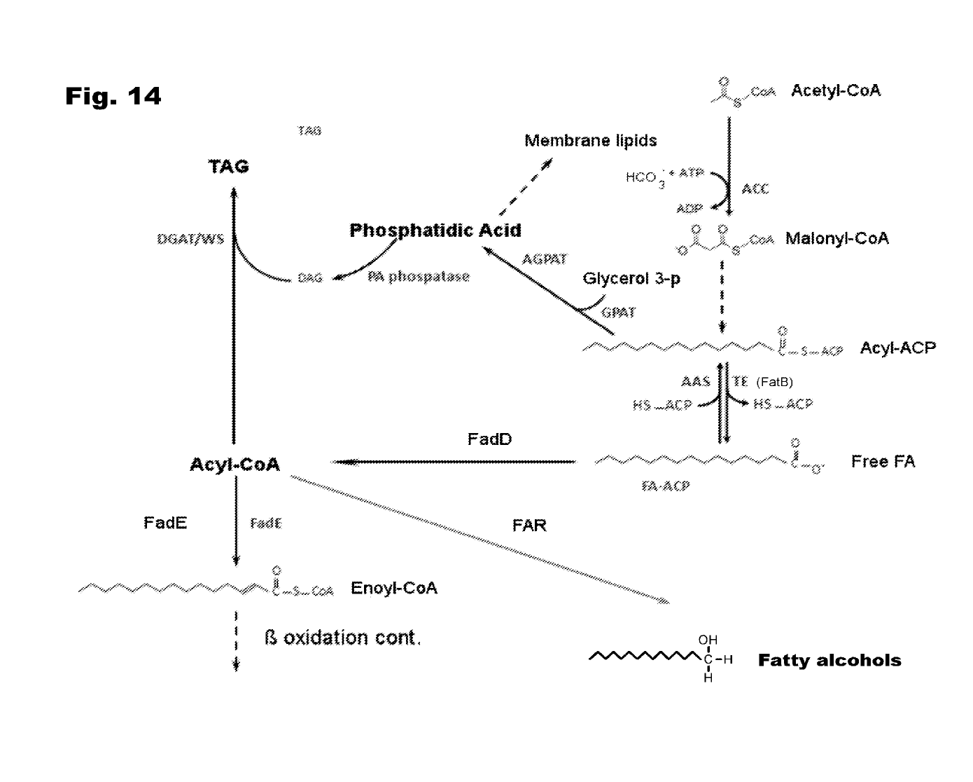

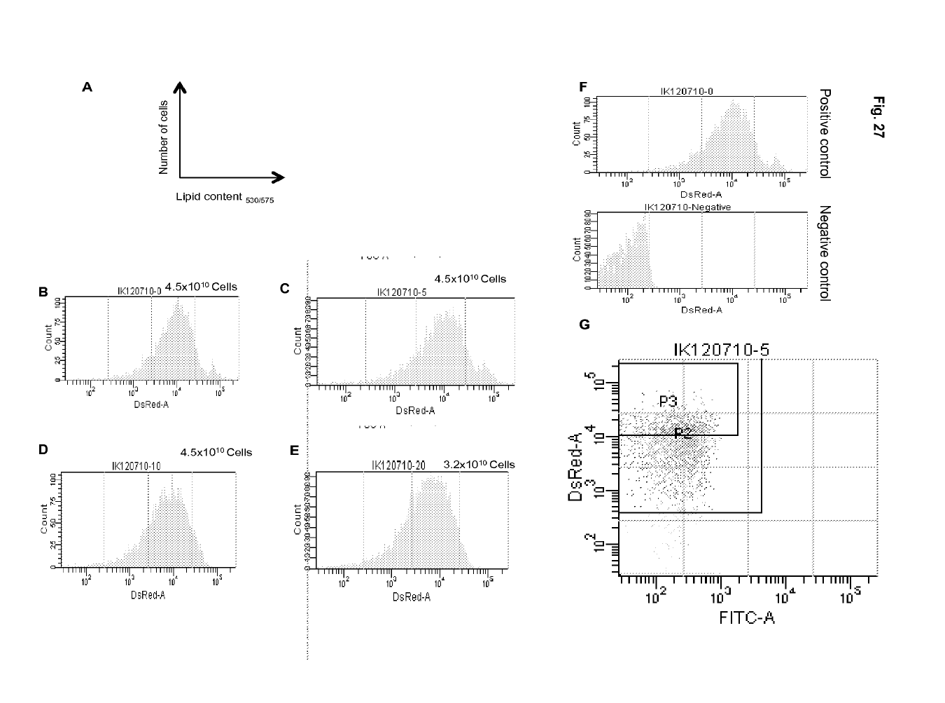

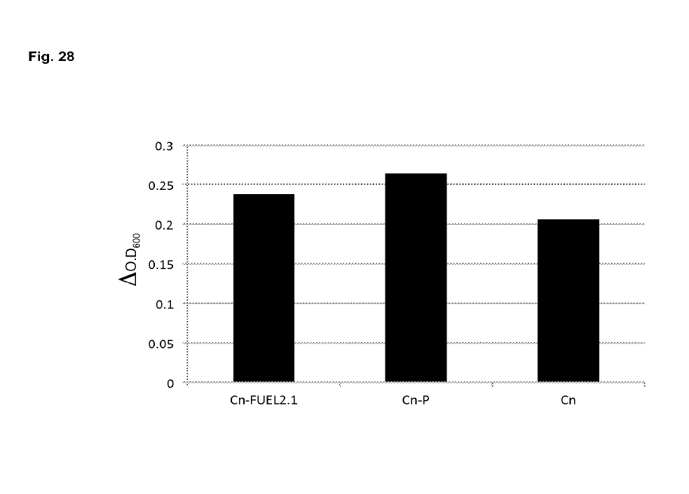

Organisms from the genus Rhodococcus and from the genus Cupriavidus were tested for their ability to grow on different carbon sources (FIG. 5). Colonies from strains grown on LB agar plates at 30° C. were transferred into flasks containing 10% (v/v) of the indicated media for 3-20 days at 30° C. and 250 rpm. R. opacus strain DSM 44193 exhibited growth only under heterotrophic growth conditions as measured by optical density (OD) at 650 nm on MSM medium (1 L Medium A: 9 g Na₂HPO₄12H₂O, 1.5 g H₂PO₄, 1.0 g NH₄Cl and 0.2 g MgSO₄.7H₂O per 1 L; 10 ml Medium B: 50 mg Ferric ammonium citrate and 100 mg CaCl₂ per 100 ml; 10 ml Medium C: 5 g NaHCO₃ per 100 ml; and 1 ml Trace Mineral Solution: 100 mg ZnSO₄.7H₂O, 30 mg MnCl₂. 4H₂O, 300 mg H₃BO₃, 200 mg COCL₂.6H₂O, 10 mg CuCl₂.2H₂O, 20 mg NiCl₂.6H₂O and 30 mg Na₂MoO₄.2H₂O per 1 L) supplemented with 40 g/L glucose. R. opacus strain DSM 43205 showed identical growth rates under heterotrophic conditions reaching O.D=9.0. Strain DSM 43205 was also able to grow on chemoautotrophic conditions (MSM medium supplemented with 66.7% H₂, 9.5% CO₂, 5% O₂ and 18.8% N₂) and heterotrophically on a single carbon compound as the solely carbon source (MSM medium supplemented with 25 g/1 methanol). Rhodococcus sp. (DSM 3346) exhibited growth under heterotrophic conditions and chemoautotrophic conditions (DSMZ Medium 81: 1 L of Mineral Medium for chemolithotrophic growth: 2.9 g Na₂HPO₄.2H₂O, 2.3 g KH₂PO₄, 1.0 g NH₄C1, 0.5 g MgSO₄.7H₂O, 0.5 g NaHCO₃, 0.01 g CaCl.2H₂O and 0.05 g Fe(NH₄) citrate per 1 L; and 5 ml Trace Mineral Solution, supplemented with 80% H₂, 10% CO₂ and 10% O₂). Cupriavidus necator (DSM 531) was able to grow under heterotrophic and chemoautotrophic conditions (media described for Strain DSM 43205) (FIG. 5 and FIG. 28). Cupriavidus necator (DSM 531) transformed with pSeqCO2 was able to grow on LB media supplemented with 300 400 and 500 μg/ml kanamycin exhibiting O.D₆₀₀ of 1.47, 1.52 and 1.51 respectively (FIG. 13). Untransformed cells exhibited growth on control (LB only) and some growth on 300 μg/ml kanamycin while no growth was detected on 400 and 500 μg/ml kanamycin.

### Example 2

### Lipid Profiles, Production of Fatty Acid

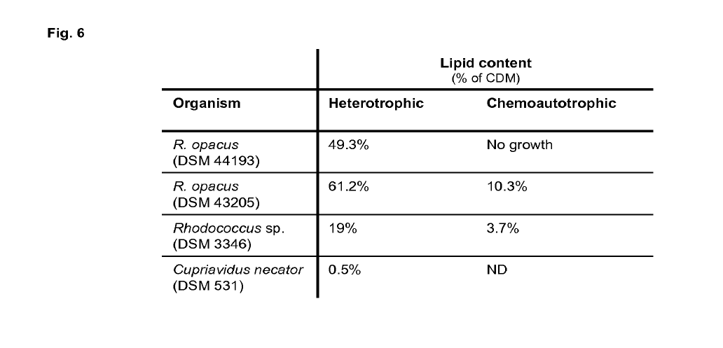

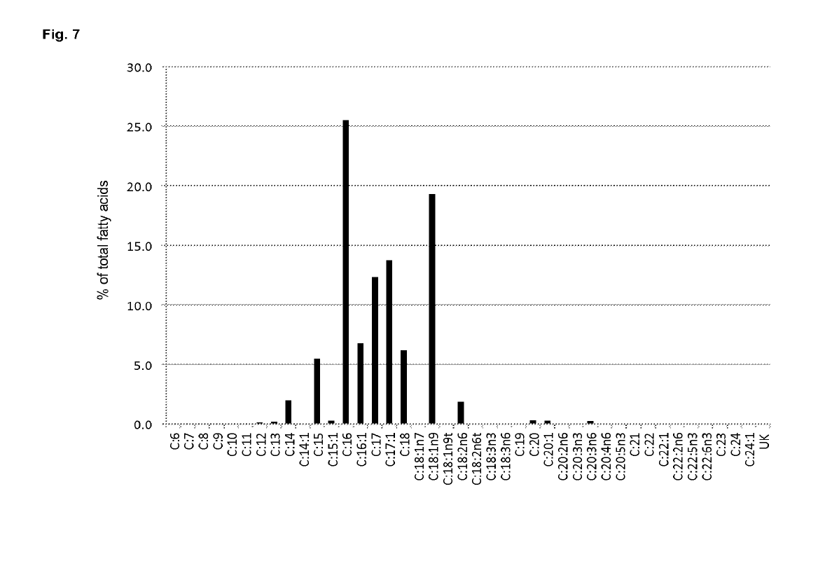

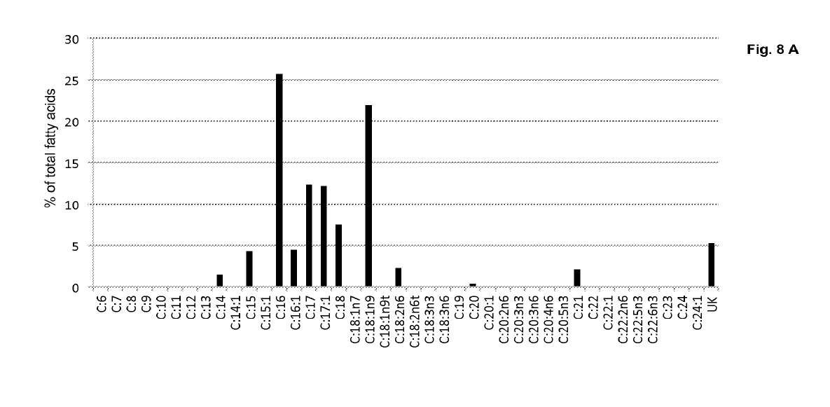

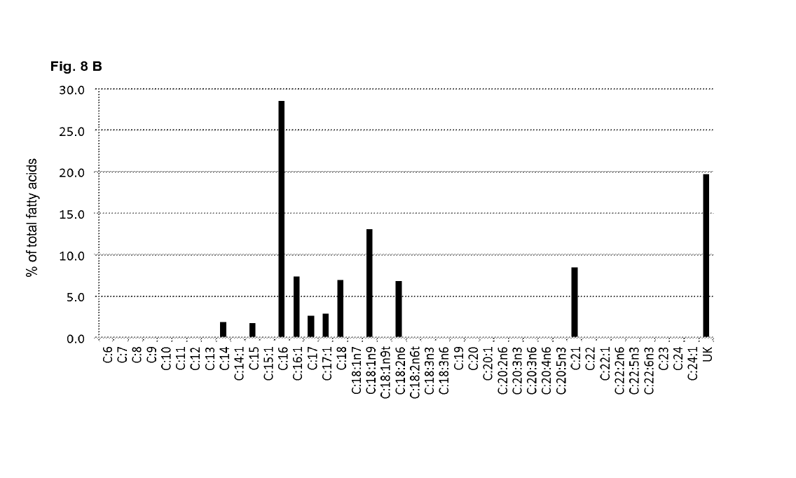

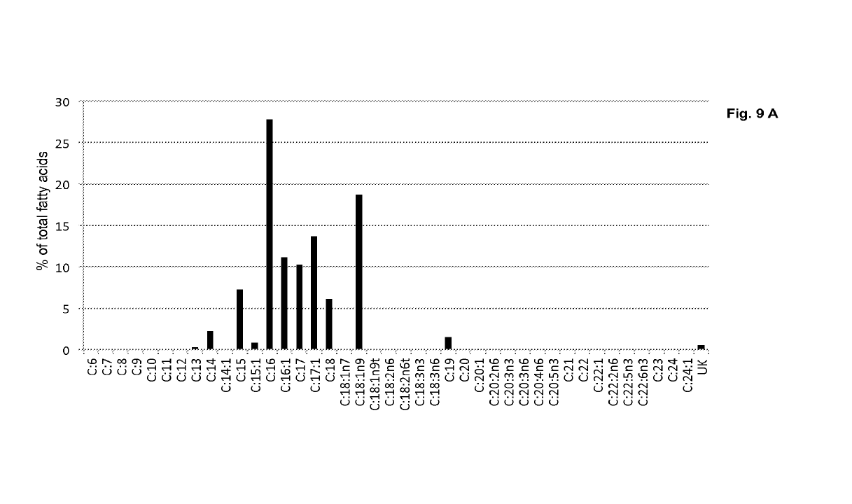

Under heterotrophic growth conditions strains DSM 44193, DSM 43205, DSM 3346 and DSM 531 produce lipid (FIG. 6). Lipid content determined by gas chromatography analysis of cells harvested after 72 hr (unless otherwise indicated) showed over 19% of cellular dry matter (CDM) determined gravimetrically for strains DSM 44193, DSM 43205 and DSM 3346. The lipid content of DSM 43205 was higher than 10% of under chemoautotrophic conditions. Under heterotrophic growth conditions DSM 44193 produces 32%, 26% and 21% of 16, 17 and 18-carbon fatty acid respectively (FIG. 7). DSM43205 produces similar amounts of 16, 17 and 18-carbon fatty acid (30%, 24% and 32% respectively) (FIG. 8A). Chemoautotrophic growth condition significantly reduces the 17-carbon fatty acid abundance (6%) and maintains similar levels of 16 and 18-carbon fatty acid (36% and 27% respectively) (FIG. 8B). DSM3346 exhibits similar fatty acid distribution of 16, 17 and 18-carbon fatty acid (39%, 24% and 25% respectively) (FIG. 9A) under heterotrophic growth. Chemoautotrophic growth condition significantly increases the 16-carbon fatty acid levels (66%) and reduces the 17 and 18-carbon fatty acid levels (4%, 14%) (FIG. 9B).

### Example 3

### Production of Alkanes

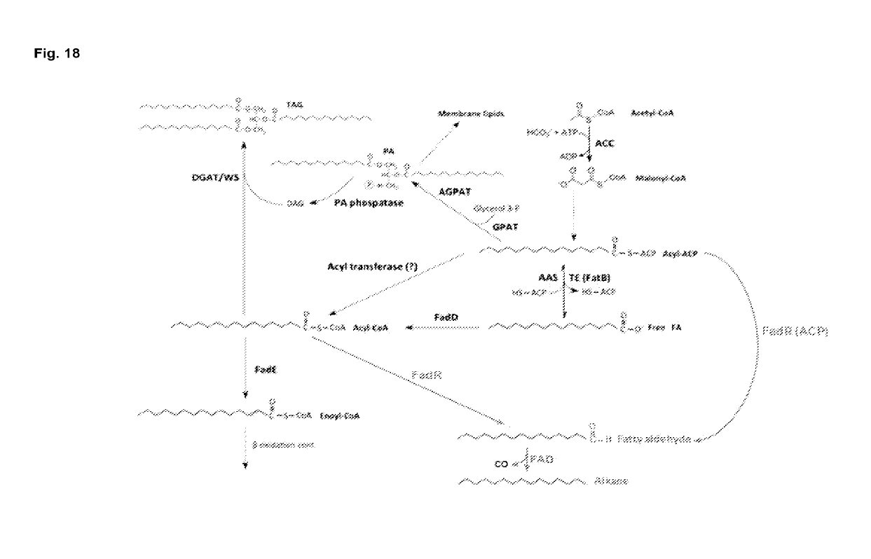

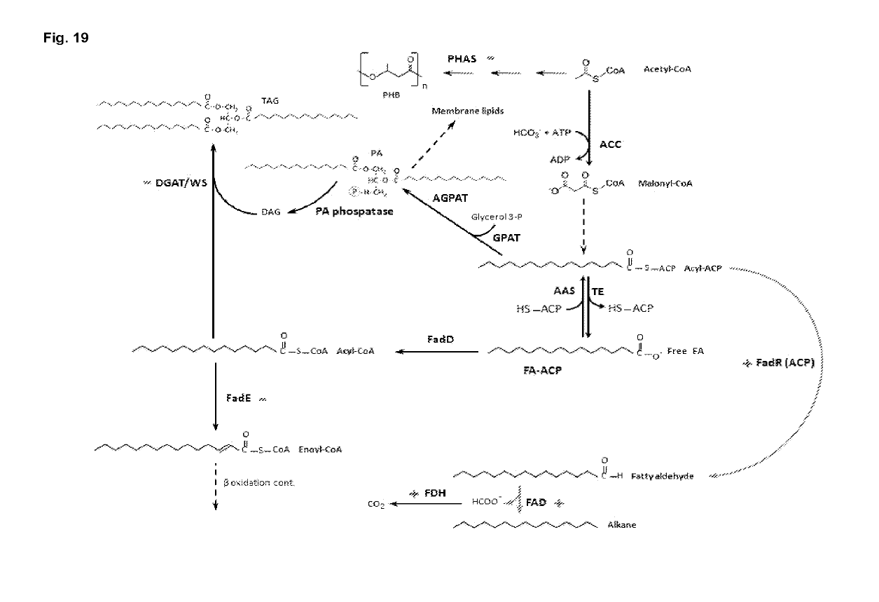

To redirect carbon flux from fatty acid toward alkanes biosynthesis, the genes Fatty acyl-CoA/Fatty acyl-ACP reductase (FadR) and Fatty aldehyde decarbonylase (FAD) from the decarbonylation pathway of cyanobacteria (indicated in red) were expressed in Cupriavidus necator (DSM 531) (FIG. 19).

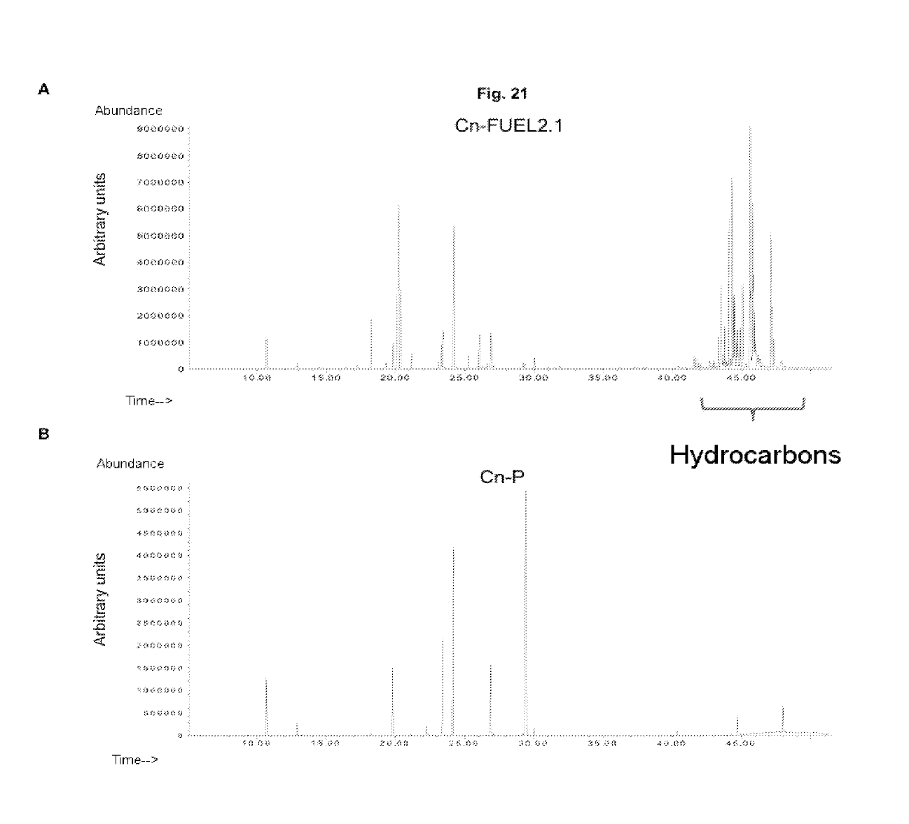

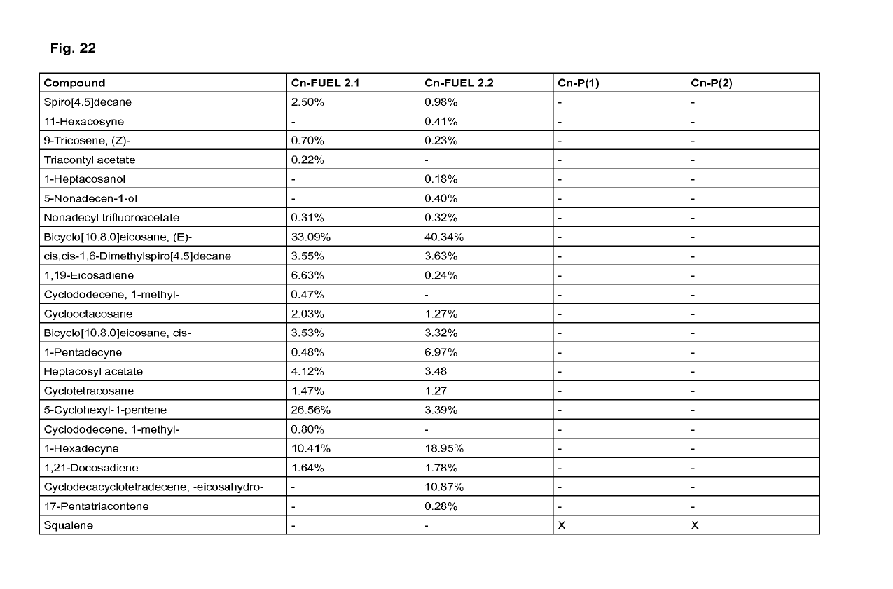

The plasmid pSeqCO2::FUEL (FIG. 20) described in the text was introduced into Cupriavidus necator (DSM 531) as described above and 2 independent transformants (Cn-FUEL2.1 and Cn-FUEL2.2) were selected. One hundred ml of Cn-FUEL2.1, Cn-FUEL2.2 and control cells (empty plasmid: Cn-P) were incubated on LB medium with 400 μg/ml kanamycin for 30 hr. Cells were harvested at 3,000×g for 10 min at 4° C. and pellet was analyzed by GC/MS. Cn-FUEL2.1 (FIG. 21A) and Cn-FUEL2.2 showed a specific peak at 45.00 min compared to control Cn-P (FIG. 21B) indicating the presence of hydrocarbons in the engineered strains. Cn-FUEL2.1, Cn-FUEL2.2 produced high levels (over 2%) of unique molecules such as: Spiro[4.5]decane, Bicyclo[10.8.0]eicosane, cis,cis-1,6-Dimethylspiro[4.5]decane, 1,19-Eicosadiene, Cyclooctacosane, Bicyclo[10.8.0]eicosane, 1-Pentadecyne, 1-Pentadecyne, Heptacosyl acetate, 5-Cyclohexyl-1-pentene, 1-Hexadecyne and Cyclodecacyclotetradecene, -eicosahydro (FIG. 22).

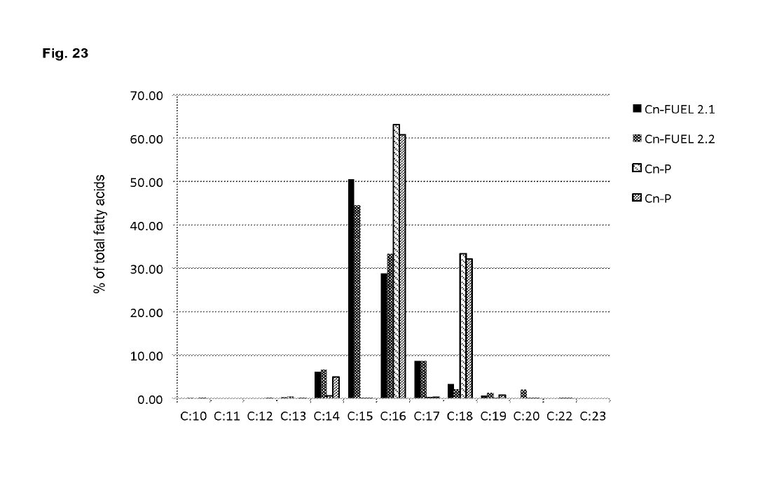

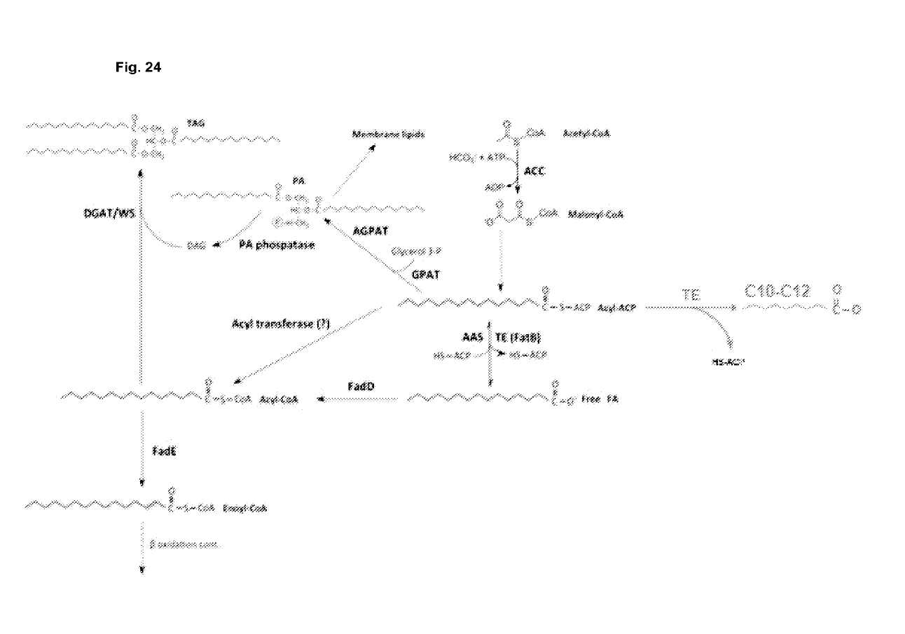

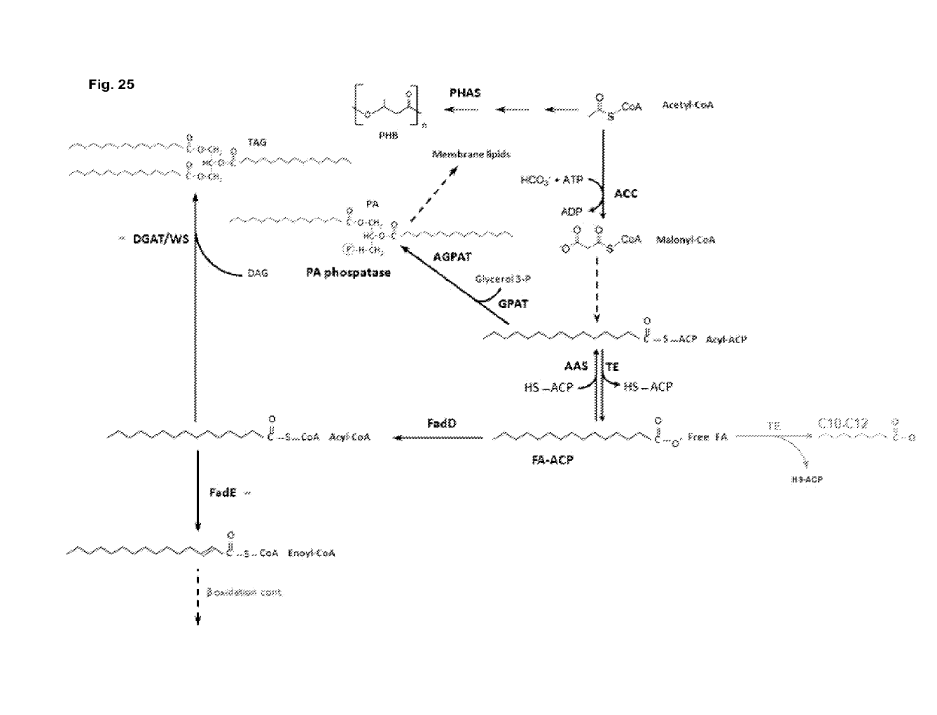

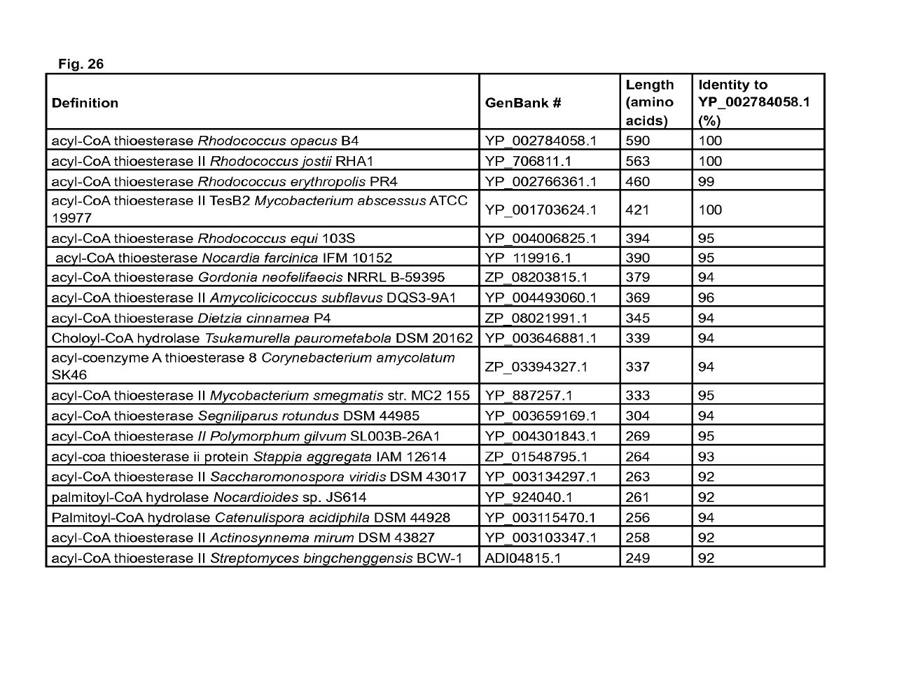

The effect of the production of alkanes on fatty acid distribution is shown in FIG. 23. The fatty acids profile of 2 independent control experiments (Cn-P) shows predominantly 16-carbon (63% and 61%) and 18-carbon (33% and 32%) fatty acids. In contrast, Cn-FUEL2.1 and Cn-FUEL2.2 exhibit significantly lower levels of 16-carbon (29%, 33% respectively) and 18-carbon (3% and 2% respectively) fatty acids. Cn-FUEL2.1 and Cn-FUEL2.2 show a significant increase in the 15-carbon fatty acid (50% and 45% respectively) compared to 0.08% and 0.09% in the control strains Cn-P.

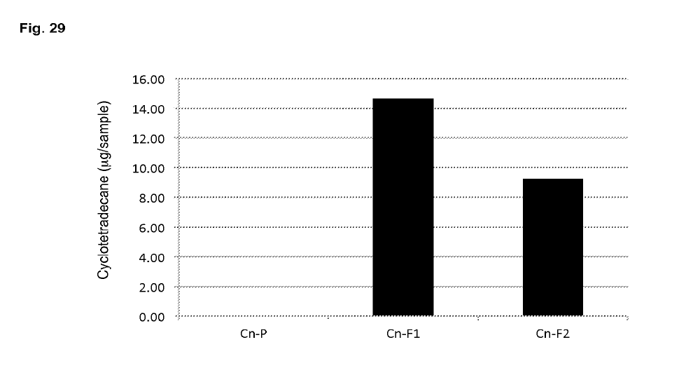

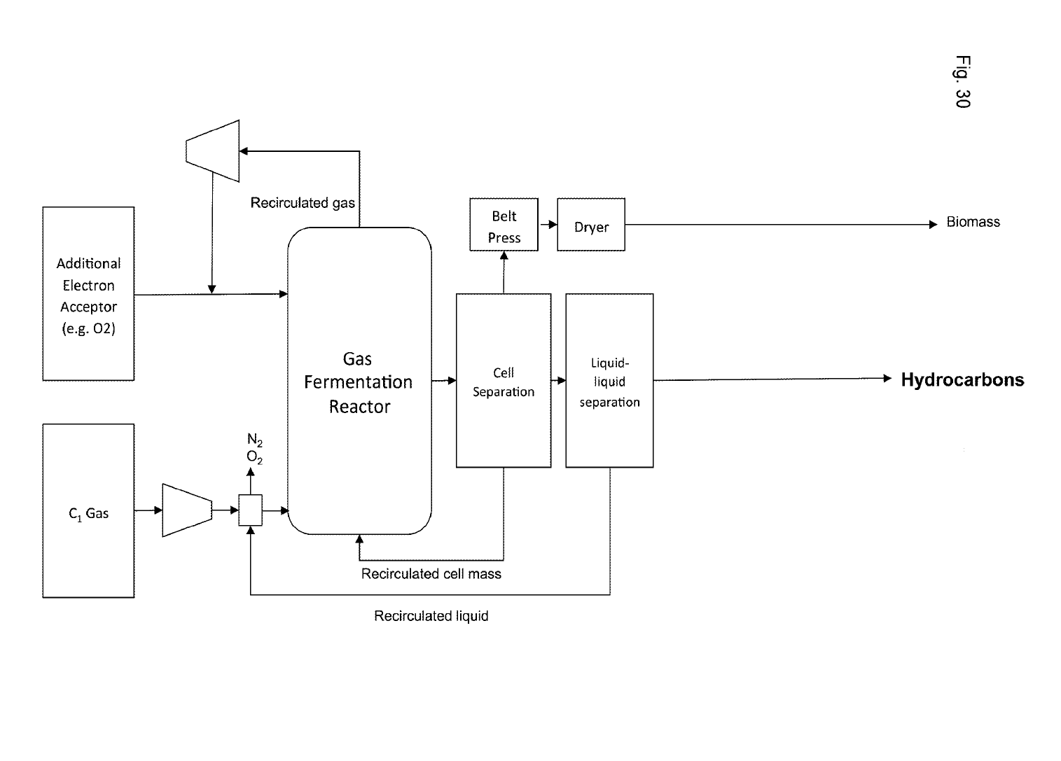

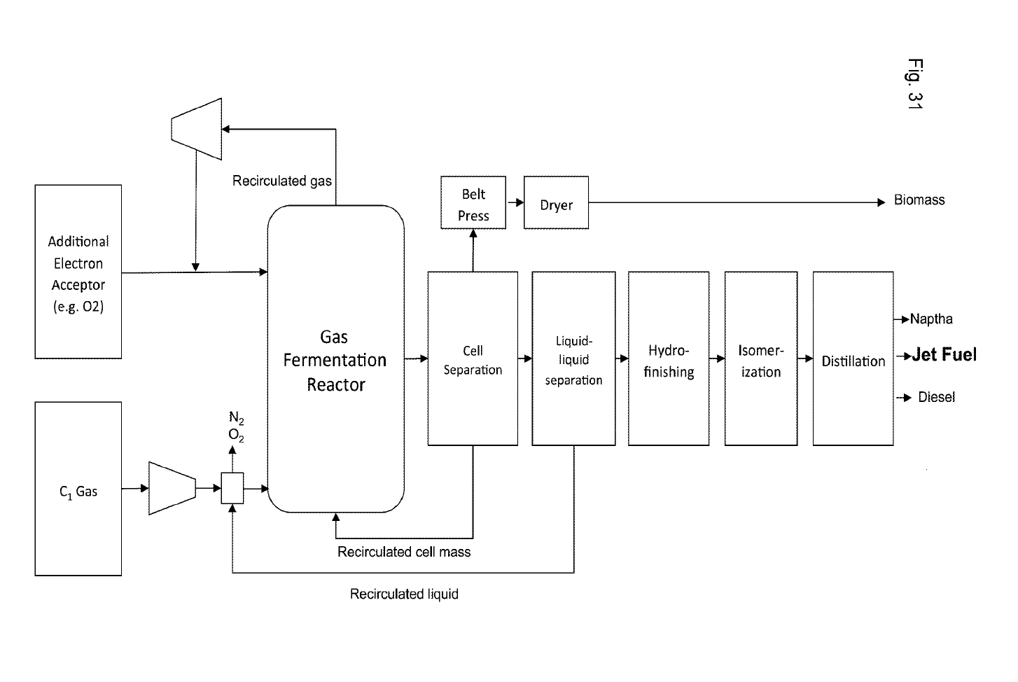

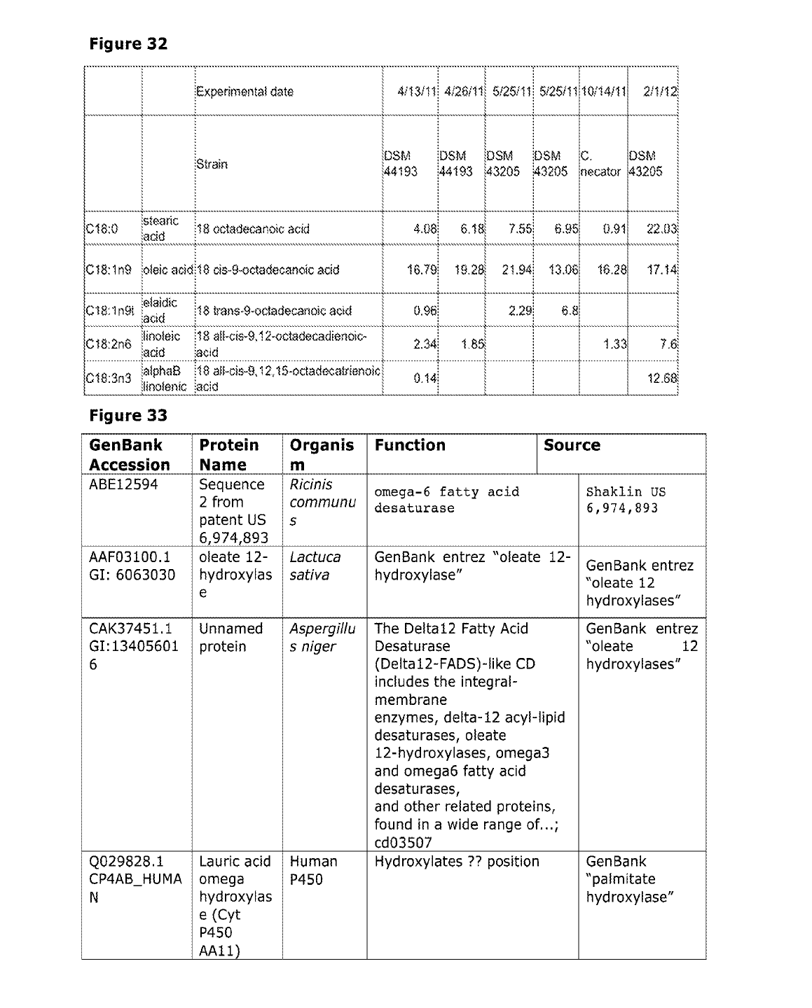

The formation of alkanes in Cupriavidus necator was demonstrated by the expression of fatty acyl-CoA reductases (FAR) genes. The Arabidopsis genes FAR1 (SEQ ID: 05), FAR2 (SEQ ID: 06) and FAR3 (SEQ ID: 07) were cloned into pSeqCO2 plasmid using the indicated restriction sites to give pSeqCO2::FAR1 and pSeqCO2::FAR2 respectively (FIG. 16). pSeqCO2::FAR1 and pSeqCO2::FAR2 and control (pSeqCO2, empty plasmid) were introduced into Cupriavidus necator (DSM 531) as described in the text. One hundred ml of transformants of pSeqCO2::FAR1 (Cn-F1), pSeqCO2::FAR2 (Cn-F2) and control cells (empty plasmid: Cn-P) were incubated on LB medium with 400 μg/ml kanamycin for 30 hr. Cells were harvested at 3,000×g for 10 min at 4° C. and pellet was analyzed by GC. Cn-F1 and Cn-F2 produced cyclotetradecane compared to control Cn-P (FIG. 29) indicating the presence of alkanes in the engineered strains. It is believed, without the present invention being limited to any particular theory, that cyclotetradecane is produced within Cupriavidus necator from a C14 fatty alcohol intermediate, that results from the introduction and expression of the FAR gene in Cupriavidus necator. The absence of cyclotetradecane in Cn-P is thought to be due to the lack of FAR gene and hence lack of C14 fatty alcohol intermediate in Cupriavidus necator, without the present invention being limited to any particular theory.

### Example 4

### Purification of Alkanes

To produce alkanes in bacteria, genes from the decarbonylation pathway of cyanobacteria, including but not limited to, the FadR (SEQ ID: 08) and FAD (SEQ ID: 09) genes are cloned into pVer2 (SEQ ID: 04) to give pVer2::FUEL. Bacteria, including but not limited to, R. opacus strain (DSM43205) are transformed with the plasmid pVer2::FUEL by electroporation and grown in 100 ml LB medium supplemented with 75 μg/ml kanamycin for 30 hr. The cells (2×50 ml) are harvested at 3,000×g for 10 min at 4° C. and the pellet and the supernatant are further analyzed. Analysis of alkanes from the cell pellet is carried out in 25 mm×150 mm glass tube in the presence of 50 μL of Eicosane standard (approx 200 μg/ml) and 50 μl lipid standard (˜200 ug/ml). The pellet is extracted with 5 mL chloroform, 10 ml methanol, 4 ml phosphate buffer (phosphate buffer reagent: 50 mM, pH 7.4, 8.7 g K₂HPO₄ in 1 L water, and about 2.5 ml 6N HCl to adjust pH=7.4, and 50 ml chloroform per 1 L buffer). The mixture is vortexed for 30 sec, sonicated for 2 min and incubated in dark for at least 3 hr. Phases are separated in the presence of 5 mL chloroform and 5 ml ddH₂O, vortexed and spun down 2000 rpm for 1 min. The bottom layer is transferred with a glass Pasteur pipette to clean 16 mm×125 mm glass tube with Teflon-lined screw top and dried under N2. The dried extract is re-suspended in hexane and analyzed by Gas Chromatography for the presence of hydrocarbons, including but not limited to 1-Hexadecyne.

### Example 5

### Purification of Fatty Alcohols

To produce fatty alcohols in bacteria, the fatty acyl-CoA reductases (FARs) that catalyze the formation of a fatty alcohol from an acyl-CoA, including but not limited to the FAR1 gene (SEQ ID: 05) are cloned into pVer2 (SEQ ID: 04) to give pVer2::FAR1. Bacteria including but not limited to R. opacus strain (DSM43205) are transformed with the plasmid pVer2::FAR1 by electroporation, grown in 100 ml LB medium supplemented with 75 μg/ml kanamycin for 30 hr. The cells (2×50 ml) are harvested at 3,000×g for 10 min at 4° C. and the pellet and the supernatant are further analyzed. Analysis of fatty alcohols from the cell pellet is carried out in 1.5 ml eppendorf tube in the presence of 500 pure HCl and 5000 ethyl acetate (EtAc). The mixture is vortexed for 10 sec and spun down at max speed for 1 min. The EtAc (top) layer is recovered and transferred to a glass GC vial. The sample is derivatized by adding 100 μl of MeOH:HCl (9:1) to the EtAc extract and mixing. About 50-100 μl of TMS-diazomethane (2M in hexanes) is mixed and incubated for 10-15 min. Aliquots of 50μ are analyzed by Gas Chromatography—Flame Ionization Detector (GC-FID) for the presence of alkanes, including but not limited to 1-tetradecanol.

### Example 6

### Purification of Fatty Acids

To modify the fatty acid distribution in bacteria, thioesterases that regulate the fatty acid chain length, including but not limited to the YP_{—}002784058.1 gene are cloned into pVer2 (SEQ ID: 04) to give pVer2::TE. Bacteria, including but not limited to, R. opacus strain (DSM43205) are transformed with the plasmid pVer2::TE by electroporation and grown in 100 ml LB medium supplemented with 75 μg/ml kanamycin for 30 hr. The cells (2×50 ml) are harvested at 3,000×g for 10 min at 4° C. and the pellet and the supernatant are further analyzed. Analysis of fatty acids from the cell pellet is carried out in 25 mm×150 mm glass tube in the presence of 50 μL of Eicosane standard (approx 200 μg/mL) and 50 μL lipid standard (˜200 ug/ml). The pellet is extracted with 5 ml chloroform, 10 ml methanol, 4 ml phosphate buffer (phosphate buffer reagent: 50 mM, pH 7.4, 8.7 g K₂HPO₄ in 1 L water, and about 2.5 mL 6N HCl to adjust pH=7.4, and 50 ml chloroform per 1 L buffer). The mixture is vortexed for 30 sec, sonicated for 2 min and incubated in dark for at least 3 hr. Phases are separated in the presence of 5 ml chloroform and 5 ml ddH₂O, vortexed and spun down 2000 rpm for 1 min. The bottom layer is transferred with a glass Pasteur pipette to clean 16 mm×125 mm glass tube with Teflon-lined screw top and dried under N2. The dried extract is re-suspended 1.5 ml of a 10:1:1 mixture of Methanol:CHCl3:concentrated HCl, vortexed and incubated in 60° C. for 14-16 hr (overnight). The extracts are cooled and 2 ml of ddH₂O and 2 ml of hexane are added, vortexed and centrifuged for 5 min at 2000 rpm for phase separation. The top hexane layer is transferred to clean 16 mm tube. Additional two hexane extraction (vortex, centrifugation and phase separation) is carried out in the extract tube. The hexane extracts are dried in a GC vial and analyzed by Gas Chromatography for the presence of fatty acids, including but not limited to dodecanoic acid.

Dicarboxylic Acids with Targeted Chain Length.

Bacteria from the suborder corynebacterineae or the family burkholderiaceae are genetically engineered to express thioesterases which yield different length fatty acids. For example, non-limiting embodiments include the YP_{—}002784058.1 gene discussed above or:

| UniProt Entry | Protein name | Organism | C length |
|---|---|---|---|
| FATB_GOSHI | Myristoyl-acyl carrier | Gossypium | 16:0 |
| | protein thioesterase | hirsutum | |
| FATB_UMBCA | Lauroyl-acyl carrier | Umbelliularia | 12:0 |
| | protein thioesterase | californica | |
| FATB_CINCA | Myristoyl-acyl carrier | Cinnamomum | 14:0 |
| | protein thioesterase | camphora | |
| FATA_CORSA | Oleoyl-acyl carrier | Coriandrum | 18:0 |
| | protein thioesterase | sativum | |
| FATB_CUPHO | Myristyl-acyl carrier | Cyphea | 16:0 |
| | protein thioesterase | hookeriana | |

Thioesterases generating shorter chain fatty acids (e.g., C10:0 or C12:0) are identified and incorporated into the bacteria from the suborder corynebacterineae and the family burkholderiaceae.

The resulting lipids are extracted and provided as the sole source of carbon to a culture of Candida tropicalis ATCC 20336, which contains the relevant enzymatic pathways to produce the alpha, omega-dicarboxylic acids. Dicarboxylic acid end products are identified and purified from the second culture.

Also, the cytochrome P450 pathway from Candida tropicalis is engineered into a host strain, including the CYP52A genes with NADPH cytochrome P450 reductase to generate dicarboxylic acid from the fatty acids. Craft et al. have identified genes for generation of alpha, omega-dicarboxylic acids in Candida tropicalis: CYP52A13, CYP52A14, CYP52A17, CYP52A18, and CYP52A12 along with the corresponding reductase (Craft 2003).

A single culture is performed, which generates appropriate length fatty acids, then modified to attach a second carboxylic acid.

### Dicarboxylic Acids.

The hyperthermophilic archaeon Pyrococcus furiosus is cultured in order to generate the dicarboxylic acids described in Carballeira et al. (Carballeira 1997). Genetic machinery for generating these dicarboxylic acids is determined, and the P furiosus genome is compared with bacteria from the suborder corynebacterineae and the family burkholderiaceae genomes. The relevant genetic modules are moved from P furiosus into bacteria from the suborder corynebacterineae and the family burkholderiaceae in order to post-process lipids into dicarboxylic acids. This can be combined with genes which produce shorter fatty acids through the appropriate thioesterases.

### Hydroxy-Acids

For generating omega-hydroxylated fatty acids, vicia sativa P450-dependent fatty acid omega hydroxylase is incorporated into bacteria from the suborder corynebacterineae and the family burkholderiaceae cell line. This enzyme hydroxylates myristic acid (C14), lauric acid (C12), pamitic acid (C16), but not oleic acid (C18).

For generating in-chain hydroxylated fatty acids, CYP81B1 (H tuberosus) or CYP709C1 (unknown) fatty acid hydroxylases are incorporated into bacteria from the suborder corynebacterineae and the family burkholderiaceae cell line. The CYP81B1 enzyme omega-1 and omega-5 mono-hydroxylates capric (C10:0), lauric (C12:0), and myristic (C14:0) (Pompon 1996). The CYP709C1 gene hydroxylates the omega-1 and omega-2 positions independent of chain length (Kandel 2005).

### Example 7

### Hydroxylation of Octadecanoic Acid to Produce 12-Hydroxy Octadecanoic Acid, Also Known as 12-Hydroxy Stearic Acid or 12-HSA

The Physaria lindheimeri oleate 12-hydroxylase ABQ01458.1 GI:146141441 can convert 9,12-octadecadienoic acid or the cis-9-cotadecenoic acid or trans-9 octadecanoic acid or octadecanoic acid (made by production strains) to 12-HSA, which is fully saturated and a hydroxyl group at the C12 position.

Octadecanoic acid is one modification away from 12-HSA. With a specialized enzyme, which adds a hydroxyl group to position 12, one can produce the 12-HSA product. Physaria lindheimeri, produces an oleate 12-hydroxylase ABQ01458.1 GI:146141441 (Dauk 2007) that is known to hydroxylate the 12-position.

A Basic Local Alignment Search Tool (BLAST) of protein sequence against the NCBI nr database (All non-redundant GenBank CDS

translations+PDB+SwissProt+PIR+PRF excluding environmental samples from WGS projects) yielded multiple hits against the 12-hydroxylase sequence itself (ABQ01458.1), and some bifunctional 12-hydroxylase/desaturases from Physaria of 91% identity. The closest related sequences beyond that are in the 80% range against Capsellsa rubells, lepidium campestre, and Arabidopsis lyurata.

The 12-hydroxylase gene from Physaria lindheimeri is synthesized, transfected and expressed in chemoautotrophic production strains described herein and the presence of 12-HSA is investigated.

### Example 8

### Hydroxylation of octadecanoic acid, cis-6-octadecanoic acid, or cis-6, cis-9-octadecanoic acid to produce ricinoleic acid or (9Z,12R)-12-Hydroxyoctadec-9-enoic acid or R12-Hydroxy-9-cis-octadecenoic acid

The Ricinus communis oleate 12-hydroxylase can convert 9,12-octadecadienoic acid or the cis-9-cotadecenoic acid or trans-9 octadecanoic acid or octadecanoic acid (made by production strains) to ricinoleic acid, which has a double bond at C9 and a hydroxyl group at the C12 position.

“In castor (Ricinus communis), where ricinoleic acid can account for up to 90% of the total fatty acids in seeds, biosynthesis of this fatty acid involves a membrane bound fatty acid hydroxylase-catalyzing hydroxylation at position 12 of oleic acid esterified to the sn-2 position of phosphatidylcholine, using cytochrome b5 and NADH as cofactors.” (Meesapyodsuk 2008).

Van de Loo et al. (van de Loo 1995) isolated oleate 12-hydroxylase genes from Ricinus communis. A search of Genbank for other genes annotated as such yield:

gi|722350|gb|U22378.1|RCU22378 Ricinus communis oleate 12-hydroxylase mRNA, complete cds
gi|187940238|gb|EU523112.1| Ricinus communis oleate 12-hydroxylase (FAH12) mRNA,
gi|255574427|ref|XM_{—}002528081.1| Ricinus communis oleate 12-hydroxylase, mRNA

Also found is an adjunct protein, which putatively binds the 12-hydroxylase enzymes (Cellini JP 2002543842-A 2002) (Cellini WO 0070052-A4 2000).

gi|33080346|dbj|BD270578.1| Ricinus communis-origin gene encoding novel protein interacting with oleate 12-hydroxylase]
gi|33080345|dbj|BD270577.1| Ricinus communis-origin gene encoding novel protein interacting with oleate 12-hydroxylase
gi|33080344|dbj|BD270576.1| Ricinus communis-origin gene encoding novel protein interacting with oleate 12-hydroxylase

### Example 9

### Hydroxylation of Oleic Acid with Oleate Hydroxylase from Fungus, Claviceps purpurea

The fatty acid hydroxylase gene GenBank: ACF37070.1 from Claviceps purpurea (Meesapyodsuk 2008) (Meesapyodsuk U.S. Pat. No. 8,003,853 2011) (Meesapyodsuk U.S. Pat. No. 7,923,598) contains both an oleate 12-hydroxylase and an omega-6 fatty acid desaturase. According to Meesapyodsuk and Qiu, biosynthesis of this fatty acid in C. purpurea involves a hydration process with linoleic acid as the substrate. Furthermore, their data indicate the biosynthesis of ricinoleic acid in C. purpurea is catalyzed by the fungal desaturase-like hydroxylase.

### Example 10

### Production of 12-HSA Using Other Plant Hydroxylases

More limited plants families (e.g., Ricinus communis) produce ricinoleic acid (D-12-hydroxyoctadec-cis-8-enoic acid) via oleoyl-12-hydroxylase (McKeon 2007) (an oleate hydroxylase) close in sequence homology to oleate desaturases. These hydroxylases do not appear in the ThYme database. They act on free C18 fatty acids, not TAGs.

### Other Fatty Acid 12-Hydroxylases

An array of relevant P450 genes is expressed in order to determine hydroxylation in production strains. (FIG. 33.)

### Example 11

### Hydroxy-Acids (Omega Hydroxylation with P450-Dependent Fatty Acid Hydroxylases

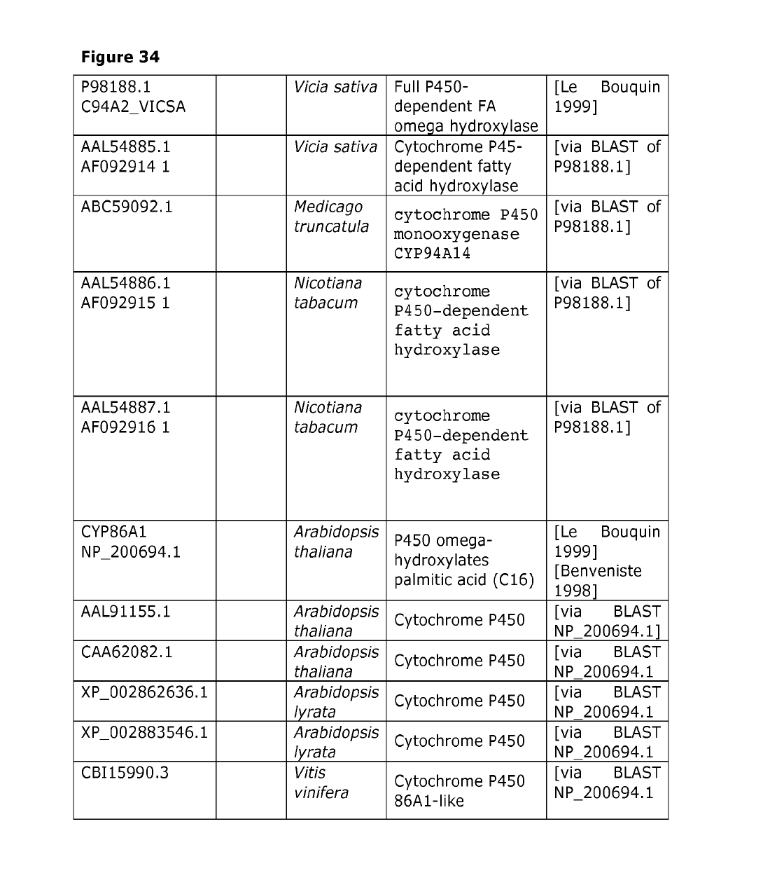

For generating omega-hydroxylated fatty acids, Vicia sativa P450-dependent fatty acid omega hydroxylase is incorporated into bacteria from the suborder corynebacterineae and the family burkholderiaceae cell line. This enzyme hydroxylates myristic acid (C14), lauric acid (C12), palmitic acid (C16), but not oleic acid (C18). Genes related to Vicia sativa P450 omega hydroxylases can also be incorporated; see FIG. 34 from BLAST runs below.

Vicia sativa contains a documented full P450-dependent fatty acid omega hydroxylase (Le Bouquin, 1999).

According to Le Bouquin et al., the hydroxylase in S. cerevisiae:

a. Hydroxylates myristic acid (C14)

b. Hydroxylates lauric acid (C12)

c. Hydroxylates palmitic acid (C16)

d. No hydroxylation of oleic acid (C18)

“ . . . only cytochrome P450 enzymes have been demonstrated to catalyze hydroxylation at the end of the aliphatic chain, i.e. at the omega-, (omega −1) and (omega −2) positions of saturated and unsaturated FAs of various chain lengths.
There is no cross talk of C94A1_VICSA with hydroxylation of non-FA substrates. Comparison of Vicia sativa P450 to other sequences:
a. BLASTP P98188.1→>100 hits with 4e-123; hits Ricinus communis: NCBI “Blast/sp|P98188.1| (513 letters).pdf”
b. Refining BLAST to only Ricinus→˜50 hits with <43-7×. All appear to be putative P450 genes.
   Hydroxy-Acids (Omega Hydroxylation with P450-Dependent Fatty Acid Hydroxylases).

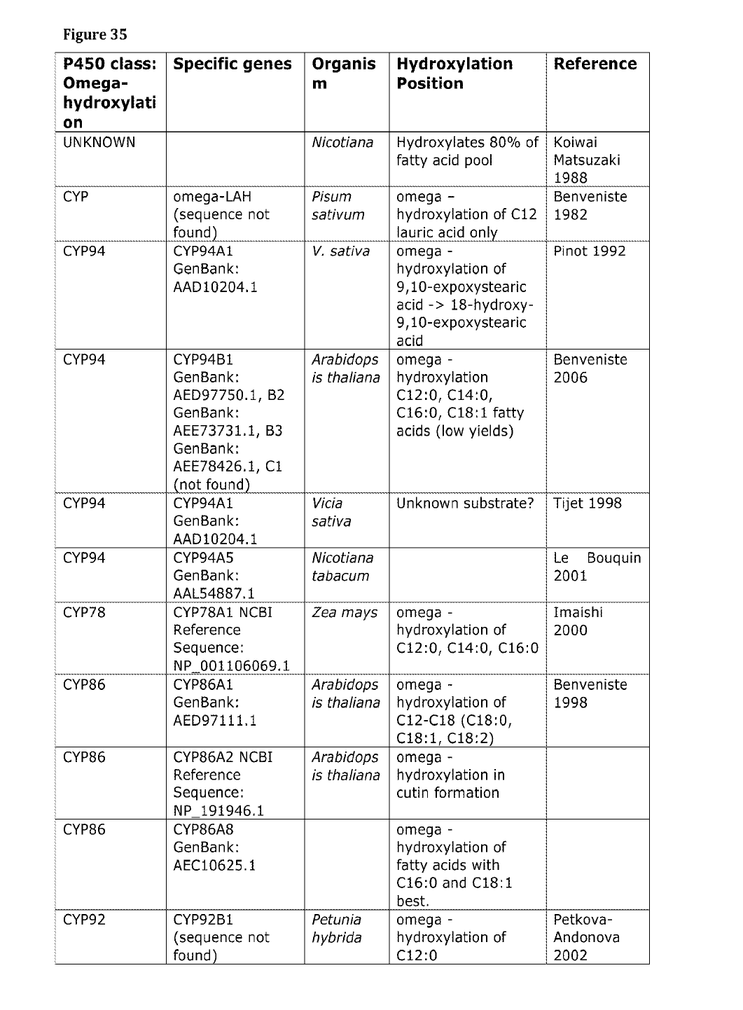

For generating omega-hydroxylated fatty acids, one of the P450-dependent fatty acid omega hydroxylase described herein (see FIG. 35) is incorporated into bacteria from the suborder corynebacterineae and the family burkholderiaceae cell line.

Kandel et al. review hydroxylation reactions/enzymes, providing cytochrome P450-dependent fatty acid hydroxylases in plants (Kandel_{—}2006).

Hydroxy-Acids (in-Chain Hydroxylation).

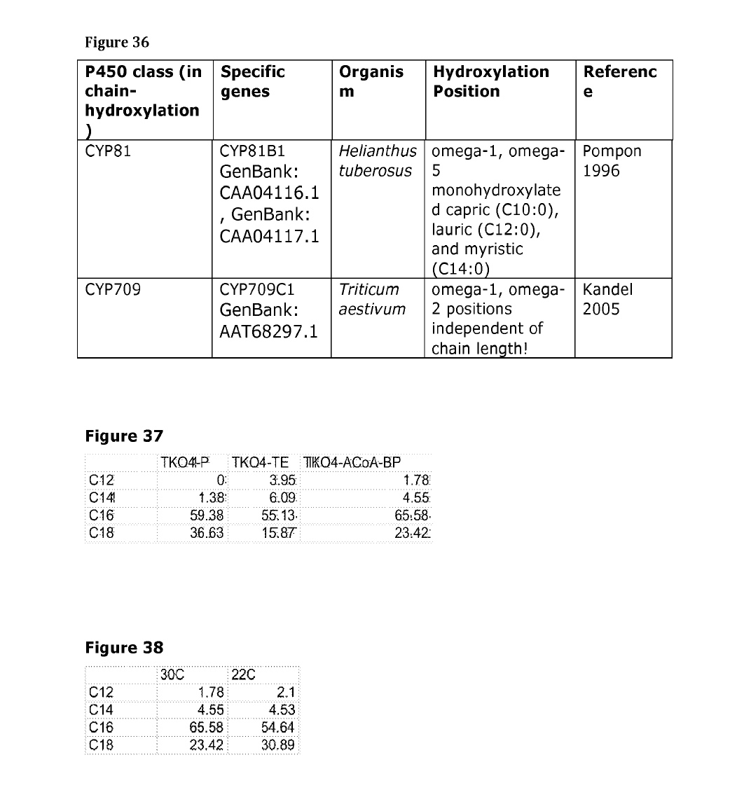

For generating in-chain hydroxylated fatty acids, CYP81B1 (H tuberosus) or CYP709C1 (unknown) fatty acid hydroxylases are incorporated into bacteria from the suborder corynebacterineae and the family burkholderiaceae cell line. The CYP81B1 enzyme omega-1 and omega-5 mono-hydroxylates capric (C10:0), lauric (C12:0), and myristic (C14:0) (Pompon 1996). The CYP709C1 gene hydroxylates the omega-1 and omega-2 positions independent of chain length (Kandel 2005). See FIG. 36.

### Example 12

### Expression of ACBP in Cupriavidus necator

Bos Taurus (cow) ACBP (SEQ ID: 01) was codon optimized for expression in Cupriavidus and Rhodococci and synthesized with the restriction sites KpnI and SalI (SEQ ID: 02). The resultant gene was cloned into pSeqCO2 (pBBR1MCS-2; Kovach et al. 1995) digested with KpnI and SalI to give pSeqCO2::ACBP (FIG. 41). Cupriavidus necator competent cells were prepared by incubating a single colony in 5 ml NR medium (10 g/l polypeptone, 10 g/l yeast extract, 5 g/l beef extract and 5 g/l ammonium sulfate; pH 7.0) at 30° C. overnight. The pre-culture was inoculated into 100 ml of fresh NR medium and incubated to a cell density of O.D₆₀₀=0.8. Cells were collected by centrifugation at 3,000×G for 10 min at 4° C. and washed 3 times with 50 ml (each) of sterile ice-cold ddH₂O. The collected cells were re-suspended in 400 μl of 10% (v/v) sterile glycerol in sterile ice-cold ddH₂O and stored in 50 μl aliquots at −80° C.

For electroporation, the competent cells were thawed on ice, transferred into 0.2 cm width of ice-cold cuvette and gently mixed with 1 μg of plasmid DNA. Cells were electroporated using a single-pulse electroporation (11.5 kV/cm, 25 μF and 5 ms pulse time). The pulsed cells were transferred into 1 ml of fresh NR medium and culture for 2 h at 30° C. Transformants were selected after cultivation for 48 h at 30° C. on NR-agar plate containing kanamycin (200 μg/ml).

For fatty acid analysis, transformants were grown in 100 ml LB media supplemented with 400 μg/ml kanamycin at 30° C., harvested after 48 hr and analyzed by gas chromatography.

Shifting of Fatty Acid Profile to Shorter Chain Lengths Through Expression of Fatty Acyl-CoA Binding Protein from Bovine Exogenous Gene (NP_{—}001106792).

It is hypothesized that expression of the Bos Taurus (cow) gene for the fatty acyl-CoA binding protein will result in a shorter chain fatty acid profile.

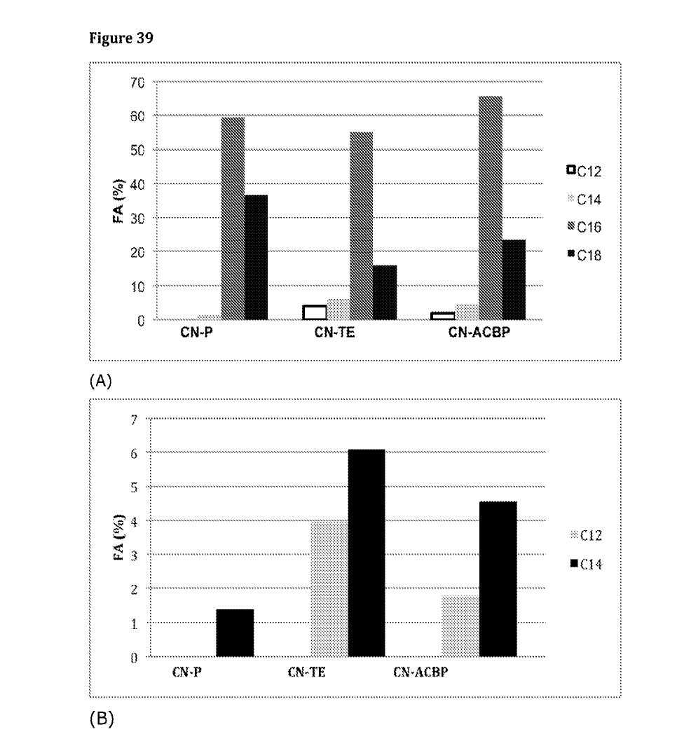

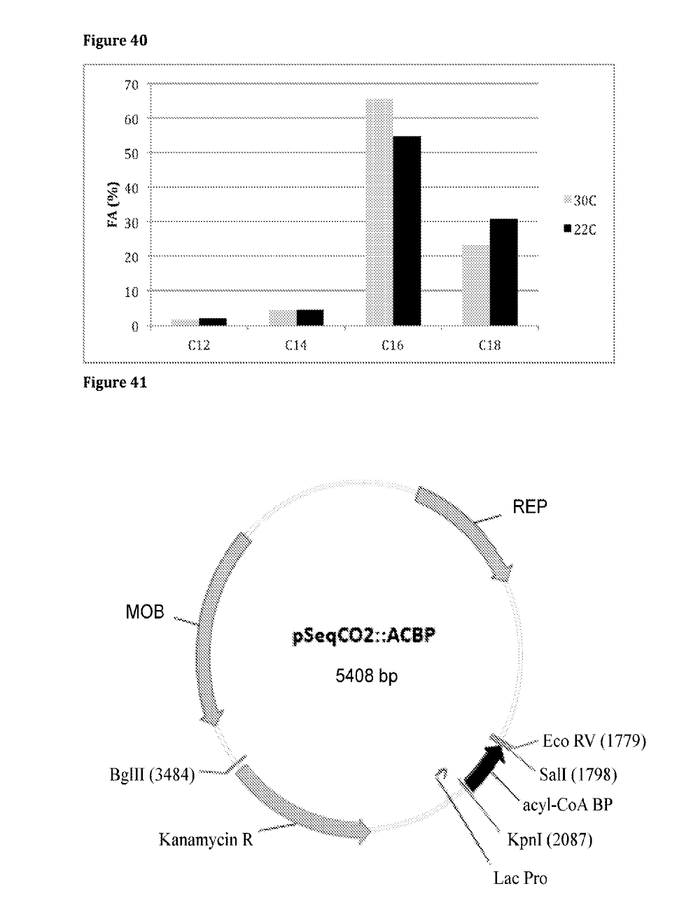

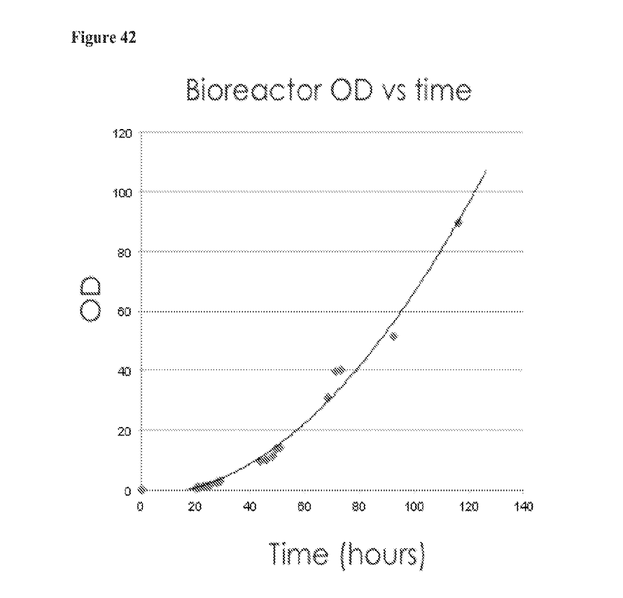

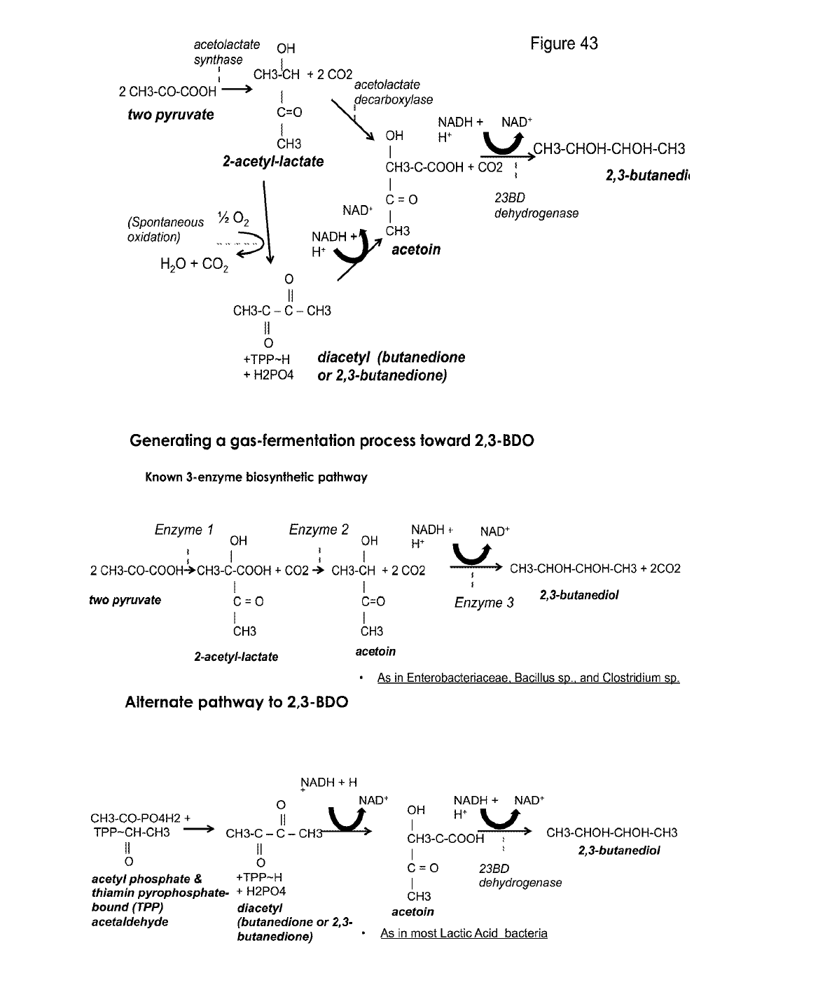

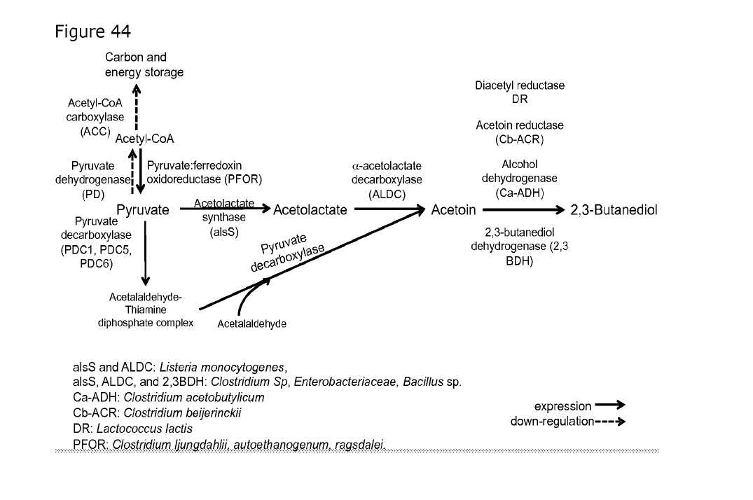

As shown in FIG. 39, the expression of a thioesterase (TKO4-TE) reduces production of C18 and C16, resulting in increased production of C12 (from 0% to 3.95%) and C14 (from 1.38% to 6.09%), compared to plasmid control (TKO4-P). The expression of the fatty acyl-CoA carrier protein results in reduced production of C18 and increase production of C12 (from 0% to 1.78%) and C14 (from 1.38% to 4.55%) compared to control.

Sample Sequences from GenBank.

Some organisms have multiple forms of these ACBP proteins. Bos Taurus appears to have a single short-chain form.

SEQID: 15
gi|164518978|ref|NP_001106792.1| acyl-CoA-binding protein [Bos
taurus]
MSQAEFDKAAEEVKHLKTKPADEEMLFIYSHYKQATVGDINTERPGMLDFKGKAKWDAWNEL
KGTSKEDA MKAYIDKVEELKKKYGI
SEQ 19 [BRnote]
gi|164518977|ref|NM_001113321.1| Bos taurus diazepam binding
inhibitor (GABA receptor modulator, acyl-CoA binding protein)
(DBI), mRNA
GAGCACCGGTGGAGAGGCCTAAGGTTGCGCTTCTAAAATCGCTGCCAGTTGAGTCTCTTGTG
CTGCTGCTACCTTCTCTTCGCCGCCTCCGCGGGCTTCCTGGAATCTTTGCAACACCGCCGGC
ATGTCTCAGGCTGAGT
TTGACAAAGCTGCTGAGGAAGTTAAGCATCTTAAGACCAAGCCAGCAGATGAGGAGATGCTG
TTCATCTA
CAGCCACTACAAACAAGCAACTGTGGGTGACATAAATACAGAACGTCCTGGAATGTTGGACT
TCAAAGGC
AAGGCCAAGTGGGATGCCTGGAATGAGCTGAAAGGGACTTCTAAAGAAGATGCCATGAAAGC
TTACATTG
ACAAAGTAGAAGAACTAAAGAAAAAATATGGAATATAAGAGACTGAGTTTGGCTGCCAGCCA
TTCATTTC
ACCTAAACTGATTTAATGCCTTGTTTTTCTAATACTGGGGATGAAGTTCATAAATAACTAGC
TAAGCCAGAAGCTCAAGACAGCCCAGGATATGACTAACAGATTAGGAGCTGAAACGGTTACT
AATCCTTGCTGAGTAA
TTTTTATCAGTAGATGAATTAAAAGTATCTTTGTTACTTTACTTCGAT
SEQID: 15: gi|164518978|ref|NP_001106792.1|acyl-CoA-binding
protein[Bos taurus]
SEQ ID: 15
MSQAEFDKAAEEVKHLKTKPADEEMLFIYSHYKQATVGDINTERPGMLDFKGKAK
WDAWNELKGTSKEDAMKAYIDKVEELKKKYGI
SEQ ID: 16
GGTACCGGGCCCCCCCTCGAGATGTCCCAGGCCGAGTTCGACAAGGCCGCCGAG
GAAGTTAAGCACCTCAAGACCAAGCCGGCAGACGAGGAGATGCTGTTCATCTAC
TCCCACTACAAGCAGGCAACCGTGGGTGACATCAACACAGAACGGCCCGGCATG
CTCGACTTCAAGGGCAAGGCCAAGTGGGATGCCTGGAATGAGCTGAAAGGGACC
TCCAAAGAAGATGCCATGAAGGCGTACATTGACAAGGTAGAAGAACTCAAGAA
AAAATACGGCATCTAGGTCGAC
The long-form ACBP:
gi|30794364|ref|NP_851381.1| acyl-CoA-binding domain-containing
protein 5 [Bos taurus]
MFQFHAGSWESWCCCCCLIPGDRPWDRGRRWRLEMRHTRSVHETRFEAAVKVIQS
LPKNGSFQPTNEMML
KFYSFYKQATEGPCKLSKPGFWDPVGRYKWDAWSSLGDMTKEEAMIAYVEEMKKI
LETMPMTEKVEELLH
VIGPFYEIVEDKKSGRSSDLTSVRLEKISKCLEDLGNVLASTPNAKTVNGKAESSDSG
AESEEEAAQEDP
KRPEPRDSDKKMMKKSADHKNLEIIVTNGYDKDSFVQGVQNSIHTSPSLNGRCTEEV
KSVDENLEQTGKT
VVFVHQDVNSDHVEDISGIQHLTSDSDSEVYCDSMEQFGQEESLDGFISNNGPFSYYL
GGNPSQPLESSG
FPEAVQGLPGNGSPEDMQGAVVEGKGEVKRGGEDGGSNSGAPHREKRAGESEEFSN
IRRGRGHRMQHLSE
GSKGRQVGSGGDGERWGSDRGSRGSLNEQIALVLMRLQEDMQNVLQRLHKLEMLA
ASQAKSSALQTSNQP
TSPRPSWWPFEMSPGALTFAIIWPFIAQWLVHLYYQRRRRKLN
gi|31341043|ref|NM_181038.2| Bos taurus acyl-CoA binding domain
containing 5 (ACBD5), mRNA
GAGGAGCTGACCAGCTGCGCTTTGGAGTCCTCCTCCCTTCGGGAATGTTGATCCG
CGGCTGCGCTCCATG
TTTCAGTTTCATGCAGGCTCCTGGGAAAGCTGGTGCTGCTGCTGCTGCCTGATTC
CAGGCGACAGACCTT
GGGACCGCGGCCGGCGCTGGCGGCTGGAGATGCGGCACACGAGATCCGTTCACG
AAACCCGGTTTGAGGC
GGCTGTGAAGGTGATACAGAGCTTGCCGAAAAATGGTTCATTCCAGCCAACAAA
TGAAATGATGCTCAAG
TTCTATAGCTTCTATAAGCAGGCAACTGAAGGACCTTGTAAACTGTCAAAGCCTG
GCTTCTGGGATCCTG
TTGGAAGATACAAATGGGATGCGTGGAGTTCTTTGGGTGATATGACCAAAGAGG
AAGCCATGATTGCTTA
TGTTGAAGAAATGAAAAAGATTCTTGAAACTATGCCGATGACTGAAAAAGTTGA
AGAATTGCTACATGTC
ATTGGTCCATTTTATGAAATTGTAGAAGACAAAAAAAGTGGCAGAAGTTCTGATT
TAACCTCAGTCCGAC
TGGAGAAAATCTCTAAATGCTTAGAAGATCTTGGTAATGTTCTAGCTTCTACTCC
AAATGCCAAAACTGT
TAATGGTAAAGCTGAAAGCAGTGATAGTGGAGCTGAATCTGAGGAAGAAGCAGC
CCAAGAAGACCCGAAA
AGACCAGAACCACGTGATAGCGATAAGAAAATGATGAAGAAATCTGCAGACCAT
AAGAATTTGGAAATCA
TTGTCACTAATGGCTATGATAAAGACAGCTTTGTGCAGGGCGTACAGAATAGCAT
TCATACCAGTCCTTC
CCTGAATGGCCGATGCACTGAGGAAGTAAAATCTGTAGATGAAAACTTGGAGCA
AACTGGAAAAACTGTT
GTCTTCGTTCACCAAGATGTAAACAGTGATCATGTTGAAGATATTTCAGGAATTC
AGCATTTGACAAGTG
ATTCAGACAGTGAAGTTTACTGTGATTCCATGGAGCAATTTGGGCAAGAAGAGTC
TTTAGACGGCTTTAT
ATCAAACAATGGACCATTTTCCTATTACTTGGGTGGTAATCCCAGTCAACCGTTG
GAAAGTTCTGGTTTT
CCTGAAGCTGTTCAAGGACTTCCTGGGAACGGCAGCCCTGAGGACATGCAGGGC
GCAGTGGTTGAAGGCA
AAGGTGAAGTAAAGCGTGGGGGAGAGGACGGCGGGAGTAACAGTGGAGCCCCG
CACCGCGAGAAACGGGC
TGGAGAAAGTGAGGAGTTCTCTAACATTAGGAGAGGGAGAGGGCACAGGATGC
AGCATTTGAGTGAAGGA
AGCAAGGGTCGGCAAGTGGGAAGTGGAGGTGATGGGGAACGCTGGGGTTCGGA
CAGAGGCTCAAGGGGCA
GCCTGAACGAGCAGATCGCGCTTGTGCTCATGCGCCTGCAGGAGGACATGCAGA
ACGTCCTCCAGAGACT
CCACAAACTGGAGATGCTGGCGGCATCACAGGCAAAATCATCAGCATTACAGAC
CAGTAATCAGCCCACT
TCACCGAGACCATCTTGGTGGCCCTTCGAGATGTCTCCTGGTGCATTAACCTTCG
CTATCATATGGCCTT
TTATTGCTCAGTGGTTGGTGCATTTATATTACCAAAGAAGGAGAAGAAAATTGAA
CTAAAGAAAATGACA
TTTTGTTGAAGAAATCTACTGGCCCTGGATAACCTCGGGATGATACCAATTGTGG
AGCTTACACGAGGGA
SEQ ID: 17
The long-form ACBP: gi|30794364|ref|NP_851381.1| acyl-CoA-binding
domain-containing protein 5[Bos taurus]
SEQ ID: 17
MFQFHAGSWESWCCCCCLIPGDRPWDRGRRWRLEMRHTRSVHETRFEAAVKVIQS
LPKNGSFQPTNEMML
KFYSFYKQATEGPCKLSKPGFWDPVGRYKWDAWSSLGDMTKEEAMIAYVEEMKKI
LETMPMTEKVEELLH
VIGPFYEIVEDKKSGRSSDLTSVRLEKISKCLEDLGNVLASTPNAKTVNGKAESSDSG
AESEEEAAQEDP
KRPEPRDSDKKMMKKSADHKNLEIIVTNGYDKDSFVQGVQNSIHTSPSLNGRCTEEV
KSVDENLEQTGKT
VVFVHQDVNSDHVEDISGIQHLTSDSDSEVYCDSMEQFGQEESLDGFISNNGPFSYYL
GGNPSQPLESSG
FPEAVQGLPGNGSPEDMQGAVVEGKGEVKRGGEDGGSNSGAPHREKRAGESEEFSN
IRRGRGHRMQHLSE
GSKGRQVGSGGDGERWGSDRGSRGSLNEQIALVLMRLQEDMQNVLQRLHKLEMLA
ASQAKSSALQTSNQP
TSPRPSWWPFEMSPGALTFAIIWPFIAQWLVHLYYQRRRRKLN
SEQ ID: 18
gi|31341043|ref|NM_181038.2|Bos taurus acyl-CoA binding domain
containing 5(ACBD5), mRNA
SEQ ID: 18
GAGGAGCTGACCAGCTGCGCTTTGGAGTCCTCCTCCCTTCGGGAATGTTGATCCG
CGGCTGCGCTCCATG
TTTCAGTTTCATGCAGGCTCCTGGGAAAGCTGGTGCTGCTGCTGCTGCCTGATTC
CAGGCGACAGACCTT
GGGACCGCGGCCGGCGCTGGCGGCTGGAGATGCGGCACACGAGATCCGTTCACG
AAACCCGGTTTGAGGC
GGCTGTGAAGGTGATACAGAGCTTGCCGAAAAATGGTTCATTCCAGCCAACAAA
TGAAATGATGCTCAAG
TTCTATAGCTTCTATAAGCAGGCAACTGAAGGACCTTGTAAACTGTCAAAGCCTG
GCTTCTGGGATCCTG
TTGGAAGATACAAATGGGATGCGTGGAGTTCTTTGGGTGATATGACCAAAGAGG
AAGCCATGATTGCTTA
TGTTGAAGAAATGAAAAAGATTCTTGAAACTATGCCGATGACTGAAAAAGTTGA
AGAATTGCTACATGTC
ATTGGTCCATTTTATGAAATTGTAGAAGACAAAAAAAGTGGCAGAAGTTCTGATT
TAACCTCAGTCCGAC
TGGAGAAAATCTCTAAATGCTTAGAAGATCTTGGTAATGTTCTAGCTTCTACTCC
AAATGCCAAAACTGT
TAATGGTAAAGCTGAAAGCAGTGATAGTGGAGCTGAATCTGAGGAAGAAGCAGC
CCAAGAAGACCCGAAA
AGACCAGAACCACGTGATAGCGATAAGAAAATGATGAAGAAATCTGCAGACCAT
AAGAATTTGGAAATCA
TTGTCACTAATGGCTATGATAAAGACAGCTTTGTGCAGGGCGTACAGAATAGCAT
TCATACCAGTCCTTC
CCTGAATGGCCGATGCACTGAGGAAGTAAAATCTGTAGATGAAAACTTGGAGCA
AACTGGAAAAACTGTT
GTCTTCGTTCACCAAGATGTAAACAGTGATCATGTTGAAGATATTTCAGGAATTC
AGCATTTGACAAGTG
ATTCAGACAGTGAAGTTTACTGTGATTCCATGGAGCAATTTGGGCAAGAAGAGTC
TTTAGACGGCTTTAT
ATCAAACAATGGACCATTTTCCTATTACTTGGGTGGTAATCCCAGTCAACCGTTG
GAAAGTTCTGGTTTT
CCTGAAGCTGTTCAAGGACTTCCTGGGAACGGCAGCCCTGAGGACATGCAGGGC
GCAGTGGTTGAAGGCA
AAGGTGAAGTAAAGCGTGGGGGAGAGGACGGCGGGAGTAACAGTGGAGCCCCG
CACCGCGAGAAACGGGC
TGGAGAAAGTGAGGAGTTCTCTAACATTAGGAGAGGGAGAGGGCACAGGATGC
AGCATTTGAGTGAAGGA
AGCAAGGGTCGGCAAGTGGGAAGTGGAGGTGATGGGGAACGCTGGGGTTCGGA
CAGAGGCTCAAGGGGCA
GCCTGAACGAGCAGATCGCGCTTGTGCTCATGCGCCTGCAGGAGGACATGCAGA
ACGTCCTCCAGAGACT
CCACAAACTGGAGATGCTGGCGGCATCACAGGCAAAATCATCAGCATTACAGAC
CAGTAATCAGCCCACT
TCACCGAGACCATCTTGGTGGCCCTTCGAGATGTCTCCTGGTGCATTAACCTTCG
CTATCATATGGCCTT
TTATTGCTCAGTGGTTGGTGCATTTATATTACCAAAGAAGGAGAAGAAAATTGAA
CTAAAGAAAATGACA
TTTTGTTGAAGAAATCTACTGGCCCTGGATAACCTCGGGATGATACCAATTGTGG
AGCTTACACGAGGGA

Specific preferred embodiments of the present invention have been described here in sufficient detail to enable those skilled in the art to practice the full scope of invention. However it is to be understood that many possible variations of the present invention, which have not been specifically described, still fall within the scope of the present invention and the appended claims. Hence these descriptions given herein are added only by way of example and are not intended to limit, in any way, the scope of this invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively.

What is claimed is:

1. A microorganism comprising at least a first exogenous nucleic acid sequence wherein the cell converts gaseous CO₂ and/or gaseous H₂ and/or syngas into one or more lipids or hydrocarbons.

2. The microorganism of claim 1, wherein the first exogenous nucleic acid sequence encodes a fatty acyl-CoA binding protein.

3. The microorganism of claim 1, wherein the first exogenous nucleic acid sequence encodes a CYP52A protein.

4. The microorganism of claim 1, wherein the first exogenous nucleic acid sequence encodes a protein selected from a CYP709C1, or a CYP81B1.

5. The microorganism of claim 2, further comprising a second exogenous nucleic acid sequence encoding a thioesterase protein enzyme.

6.-11. (canceled)

12. The according to claim 1, wherein the microorganism is of the genera Rhodococcus or Gordonia.

13. The bacterial cell according to claim 1, wherein the microorganism is a Rhodococcus opacus.

14.-38. (canceled)

39. The microorganism of claim 1, wherein the microorganism is the species Rhodococcus sp. DSM 3346 or DSM 364.

40. (canceled)

41. The microorganism of claim 1, wherein the microorganism is Rhodococcus opacus (DSM 43205) or Rhodococcus opacus (DSM 43206) or Rhodococcus opacus (DSM 44193).

42. The microorganism of claim 1, wherein the microorganism is family Burkholderaceae.

43. The microorganism of claim 1, wherein the microorganism is Cupriavidus necator.

44. The microorganism of claim 1, wherein the microorganism is Cupriavidus metallidurans.

45. The microorganism of claim 1, wherein the microorganism is a knallgas microorganism, also known as an oxyhydrogen microorganism.

46. (canceled)

47. The microorganism of claim 1, wherein the wild-type or mutant of the microorganism naturally has a capability for accumulating and/or synthesizing high quantities of triacylglycerol where a high quantity is considered to be 10% or more of the dry cell mass.

48. The microorganism of claim 1, wherein the microorganism is a hydrogen-oxidizing chemoautotroph.

49. The microorganism of claim 1, wherein the microorganism is capable of growing on syngas as the sole energy and carbon source.

50.-70. (canceled)

71. A method for producing shorter-chain fatty acids wherein the method comprises: in a bioreactor or solution, culturing a microorganism comprising at least a first exogenous nucleic acid sequence or a natural microorganism strain with a feedstock comprising syngas and/or gaseous CO₂ and/or a mixture of CO₂ gas and H₂ gas, wherein said microorganism converts said feedstock into one or more shorter-chain fatty acids.

72. (canceled)

73. The method of claim 71 further comprising the step of up-regulating an endogenous or exogenous thioesterase gene of the microorganism.

74. The method of claim 71 further comprising the step of down-regulating an endogenous or exogenous thioesterase gene of the microorganism.

75. The method of claim 71 further comprising the step of down regulating an endogenous or exogenous acyl carrier protein gene of the microorganism.

76.-99. (canceled)
